SEQUENCE LISTING (56 sequences)

SEQ ID NO: 1 - RNA, 11707 nt, Severe acute respiratory syndrome coronavirus 2
  source 1..11707: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 2 - RNA, 11707 nt, Severe acute respiratory syndrome coronavirus 2
  source 1..11707: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 3 - RNA, 11707 nt, Severe acute respiratory syndrome coronavirus 2
  source 1..11707: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcaggccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 4 - RNA, 11698 nt, Severe acute respiratory syndrome coronavirus 2
  source 1..11698: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacttcacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgccaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcggggccttccccaaggcttttctgctctcgaacccctcgtagacttgccaattgggataaatatcactcgctttcaaactttgcacatcagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaacattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttaagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgtggagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 5 - RNA, 11698 nt, Severe acute respiratory syndrome coronavirus 2
  source 1..11698: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaataagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagacgacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagatcagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattcccatcggcgagcacgatctgtagcatcccagtctattattgcctacactatgtcattgggcgccgagaatagcgtcgcatattcaaataattctattgcaatacccatcaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttggcccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactcacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 6 - RNA, 11701 nt, Severe acute respiratory syndrome coronavirus 2
  source 1..11701: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 7 - RNA, 11704 nt, Severe acute respiratory syndrome coronavirus 2
  source 1..11704: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 8 - protein, 455 aa, synthetic construct
QVQLVQSGAEVKKPGSSVKVSCKASGGTFSNYAISWVRQAPGQGLEWMGRIIPILGIANYAQKFQGRVTITADKSTSTAYMELSSLRSEDTAVYYCARGYYEARHYYYYYAMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 449 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 450 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 457 aa, synthetic construct
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 12 - RNA, 10571 nt, synthetic construct
  source 1..10571: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagttcggtcttagctgggtgtttcttgtcgccctgttcagaggggtacaatgccaagtgcaacttgtagagagtggcggcggcgtagttcgaccaggtgggagtctgaggctgtcatgtgcagcatccgggttcacctttaaaaactacgggattcactgggtgaggcaggctcctggtaagggaccagagtgggtcgccttcgtgcgctatgatgggaataacaaatattacgctgactccgtcaaaggtcgcttcacaatatccagggacaatgcaaaaaatacactgagcttgcaaatgaattctttgcgggtggaagatactgctgtatatttctgtgctcgggatccagaaacttttggagggtttgattattgggggcaagggacactcgttactgtcagtagcgcctccacaaagggtccaagtgtcttcccactggctcccagcagcaagagtacttcaggtgggactgcagctctcgggtgcctggtcaaggactactttcccgagcccgtaacagtatcttggaactccggtgctctgacaagtggagtgcatactttcccagctgtgttgcagtcaagcgggttgtactccctcagtagtgtagttactgtcccttcatcttcactggggactcaaacctacatttgtaacgtgaatcacaaaccaagcaatactaaagtagataagaaggtggagccaaaaagttgtgataaaactcatacttgtcccccctgtcctgcaccagagctgttgggcggtcccagtgtatttttgtttccccctaaacccaaagacacactgatgatttctcgaactcccgaagtgacctgcgtcgtcgttgatgtaagtcacgaagaccccgaggtaaaattcaattggtacgtagacggcgtagaggtgcataacgctaaaaccaaaccaagggaggaacaatacaacagcacttacagagtcgtatctgtactgactgttctccaccaagactggcttaatgggaaggagtataaatgcaaggtgtcaaacaaggctctgcctgcccccatagagaaaaccataagtaaagctaaaggacaacctagagagccccaggtttatactcttcccccctcccgagaagagatgaccaagaaccaagtttccttgacctgtctggttaagggtttttatccaagcgatatagccgtagaatgggagagcaacggacaacctgagaataactacaaaacaactcccccagtgctggactcagatggctcatttttcctgtattcaaagctcaccgtggacaaatctcggtggcagcaagggaatgtattctcctgttccgtcatgcacgaggcgctgcataatcactatacccagaaatctctgtccctttctcctggaaagtgataatctagacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggacatgcgagtccctgcccaactgctgggtcttctcctcctttggctgtcaggagcccgctgcgaaattgtaatgacacaatctccagcaacactttccgtcagtccaggcgagagagggaccttgtcatgcagggcttccgaaagcgtttccagcaaccttgcttggtatcaacaaaaacctggaaaggcccccagactgctgatctatggcgcttccacacgcgcaactggtattcctgacagattctccgggtctgggtctggcactgagttcacactcacaatttccagtctgcagtccgaggattttgctgtatattattgccaacagtattactatagtcctaggacctttggtcaagggactaaggtcgaggtaaagcggaccgtggctgcaccatccgtttttatttttccaccaagtgatgagcagcttaaaagtggtactgcctccgtggtgtgcttgttgaacaacttctacccacgcgaggccaaggtgcaatggaaggtagataatgccttgcagagtggaaattctcaagagtcagtcaccgaacaggatagtaaagactctacatattctcttagctctaccctcactttgtctaaagcagattatgaaaagcataaagtgtatgcatgcgaagtgacccaccaggggctgaggtctcctgtcaccaaaagttttaacaggggagagtgttgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 13 - RNA, 1407 nt, synthetic construct
  source 1..1407: mol_type = other RNA
atggagttcggtcttagctgggtgtttcttgtcgccctgttcagaggggtacaatgccaagtgcaacttgtagagagtggcggcggcgtagttcgaccaggtgggagtctgaggctgtcatgtgcagcatccgggttcacctttaaaaactacgggattcactgggtgaggcaggctcctggtaagggaccagagtgggtcgccttcgtgcgctatgatgggaataacaaatattacgctgactccgtcaaaggtcgcttcacaatatccagggacaatgcaaaaaatacactgagcttgcaaatgaattctttgcgggtggaagatactgctgtatatttctgtgctcgggatccagaaacttttggagggtttgattattgggggcaagggacactcgttactgtcagtagcgcctccacaaagggtccaagtgtcttcccactggctcccagcagcaagagtacttcaggtgggactgcagctctcgggtgcctggtcaaggactactttcccgagcccgtaacagtatcttggaactccggtgctctgacaagtggagtgcatactttcccagctgtgttgcagtcaagcgggttgtactccctcagtagtgtagttactgtcccttcatcttcactggggactcaaacctacatttgtaacgtgaatcacaaaccaagcaatactaaagtagataagaaggtggagccaaaaagttgtgataaaactcatacttgtcccccctgtcctgcaccagagctgttgggcggtcccagtgtatttttgtttccccctaaacccaaagacacactgatgatttctcgaactcccgaagtgacctgcgtcgtcgttgatgtaagtcacgaagaccccgaggtaaaattcaattggtacgtagacggcgtagaggtgcataacgctaaaaccaaaccaagggaggaacaatacaacagcacttacagagtcgtatctgtactgactgttctccaccaagactggcttaatgggaaggagtataaatgcaaggtgtcaaacaaggctctgcctgcccccatagagaaaaccataagtaaagctaaaggacaacctagagagccccaggtttatactcttcccccctcccgagaagagatgaccaagaaccaagtttccttgacctgtctggttaagggtttttatccaagcgatatagccgtagaatgggagagcaacggacaacctgagaataactacaaaacaactcccccagtgctggactcagatggctcatttttcctgtattcaaagctcaccgtggacaaatctcggtggcagcaagggaatgtattctcctgttccgtcatgcacgaggcgctgcataatcactatacccagaaatctctgtccctttctcctggaaagtga

SEQ ID NO: 14 - RNA, 714 nt, synthetic construct
  source 1..714: mol_type = other RNA
atggacatgcgagtccctgcccaactgctgggtcttctcctcctttggctgtcaggagcccgctgcgaaattgtaatgacacaatctccagcaacactttccgtcagtccaggcgagagagggaccttgtcatgcagggcttccgaaagcgtttccagcaaccttgcttggtatcaacaaaaacctggaaaggcccccagactgctgatctatggcgcttccacacgcgcaactggtattcctgacagattctccgggtctgggtctggcactgagttcacactcacaatttccagtctgcagtccgaggattttgctgtatattattgccaacagtattactatagtcctaggacctttggtcaagggactaaggtcgaggtaaagcggaccgtggctgcaccatccgtttttatttttccaccaagtgatgagcagcttaaaagtggtactgcctccgtggtgtgcttgttgaacaacttctacccacgcgaggccaaggtgcaatggaaggtagataatgccttgcagagtggaaattctcaagagtcagtcaccgaacaggatagtaaagactctacatattctcttagctctaccctcactttgtctaaagcagattatgaaaagcataaagtgtatgcatgcgaagtgacccaccaggggctgaggtctcctgtcaccaaaagttttaacaggggagagtgttgataa

SEQ ID NO: 15 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 443 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 449 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 449 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 451 aa, synthetic construct
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 449 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 222 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 31 - protein, 456 aa, synthetic construct
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 220 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERK

SEQ ID NO: 33 - protein, 446 aa, synthetic construct
VQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 226 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 35 - protein, 446 aa, synthetic construct
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - RNA, 7561 nt, Venezuelan equine encephalitis virus
  source 1..7561: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 39 - protein, 51 aa, Venezuelan equine encephalitis virus
RELPVLDSAAFNVECFKKYACNNEYWETFKENPIRLTEENVVNYITKLKGP

SEQ ID NO: 40 - protein, 41 aa, Venezuelan equine encephalitis virus
TQMRELPVLDSAAFNVECFKKYACNNEYWETFKENPIRLTE

SEQ ID NO: 41 - RNA, 8371 nt, synthetic construct
  source 1..8371: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatggggagaagattcctggtcaccgtgcggatccagagagctggcagacctctgcaagaaagagtgtttctggtcaagttcgtgcggagcagaaggcccagaacagcctcttgtgctctggccttcgtgaacatgctgctgagactggaaaggatcctgagaagaggccctcacagaaaccctggacctggcgacgatgacggccagagaagcagatcttctagcagcgcccagctgagatgcagattcgagctgaggggccctcactacttgcttccacctggcgctagaagaagcgccggtagacttcctggacatgctggcggagctgctagagtcagaggctctgctggctgtgctagatgtctgggctctcctgctgcaagactgggccctagagccggcacatctagacaccgggctatcttcgccttcagatgggtgctgttcgtgtttagatgggtcgtgtttgtgtaccgctgggagagaaggcctgacagaagggcttgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 42 - RNA, 8254 nt, synthetic construct
  source 1..8254: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgctcggcggatcttctgatgctggcctggctacagctgctgctagaggacaggtggaaacagtgcggcagctgcttgaagctggcgccgatcctaacgctctgaacagatttggcagacggcccatccaagtgatgatgatgggctctgctcaggtggccgaactgctgcttcttcacggcgctgagcctaactgtgccgatcctgccacactgaccagacctgtgcatgatgccgctagagagggcttcctggacacactggtggtgctgcatagagccggcgctagactggatgtgtgtgatgcttggggcagactgcctgtggatctggctgaggaacagggccacagagatatcgccagatacctgcacgccgccacaggtgattgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 43 - RNA, 8368 nt, synthetic construct
  source 1..8368: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatggctgagccttggggaaacgagctggcttctgctgctgctagaggcgacctggaacagctgacaagcctgctgcagaacaacgtgaacgtcaacgcccagaacggcttcggcagaacagccctgcaagtgatgaagctgggcaaccctgagatcgccagaaggctgcttctgagaggcgctaaccccaacctgaaggacggcacaggcttcgccgtgatccatgatgctgccagagccggcttcctggatacagtgcaggctctgctggaatttcaggccgacgtgaacatcgaggacaacgagggaaacctgcctctgcacctggctgccaaagagggacatctgcccgtcgtggaattcctgatgaagcacaccgcctgcaacgtgggccacagaaaccacaagggcgacacagccttcgacctggccagattctacggcagaaacgaagtgatcagcctgatggaagccaacggcgtcggcggagctacatctcttcagtgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 44 - RNA, 8362 nt, synthetic construct
  source 1..8362: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgctgctggaagaagtgtgcgtcggcgacagactttctggcgctgctgctagaggcgacgtgcaagaagttcggagactgctgcacagagaactggtgcaccctgacgctctgaacagattcggcaagacagccctgcaagtgatgatgttcggcagccctgctgtggccctggaactgcttaaacagggcgcctctcctaacgtgcaggacgcctctggaacaagccctgtgcatgatgccgccagaacaggcttcctggacaccctgaaagtgctggtggaacacggcgccgatgtgaacgctctggattctaccggcagcctgcctatccacctggccatcagagaaggccacagctccgtggtgtctttcctggctcctgagagcgatctgcaccacagagatgcctctggcctgacaccactggaactggctagacagagaggcgctcagaacctgatggacatcctgcagggacacatgatgatccccatgtgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 45 - RNA, 8341 nt, synthetic construct
  source 1..8341: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgtctaaccctggggatgttcggcccgtgcctcacagatccaaagtgtgcagatgcctgtttggccccgtggactctgagcagctgagaagagattgcgacgctctgatggccggctgtctgcaagaggctagagagagatggaacttcgacttcgtgaccgagacacccctggaaggcaacttcgtgtgggaaagagtcagaagcctgggcctgcctaaagtgtacctgtctcctggcagcagaagcagggacgatctcggcggagataagaggccttctacaagctctgctctgctgcagggacctgctcctgaggatcatgtggccctgagcctgagctgtaccctggtgtctgaaagacccgaggactctcctggcggccctggaacatctcagggcagaaagagaaggcagaccagcctgaccgacttctaccacagcaagaggcggctggtgttctgcaagcgaaagccttgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 46 - RNA, 8455 nt, synthetic construct
  source 1..8455: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgagcaatgtgcgggtgtccaacggcagccccagcctggaaagaatggatgccagacaggctgagcaccccaagcctagcgcttgcagaaacctgttcggccccgtgaaccacgaggaactgaccagagatctggaaaagcactgccgcgacatggaagaggccagccagagaaagtggaacttcgacttccaaaaccacaagcctctggaaggcagatacgagtggcaagaggtggaaagaggcagcctgcctgagttctactacagacctcctaggcctcctaagagcgcctgcaaggtgctggctcaagagtctcaggatgtgtccggcagcagacaggccgtgcctctgattggatctcaggccaacagcgaggacagacacctggtggaccagatgcctgacagcagcgataaccctgctggactggctgagcagtgccccggaatgagaaaaagacctgccgccgaggacagcagcagccagaacaagagagccaacagaaccgaggaaaacgtgtccgacggctctcctaacgccggcacagttgagcagacccctaagaaaccaggcctgagaaggcagacctgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 47 - RNA, 8908 nt, synthetic construct
  source 1..8908: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgggcatgagcgacgtgtacctgagaagcagaaccgccatggaaagactggccagcagcgacacattccccgtgatcgctagaagcagcgcctgcagatctctgttcggccctgtggatcacgaggaactgggcagagaactgagaatgagactggccgagctgaacgccgaggaccagaacagatgggacttcaacttccagcaggacgtgccccttagaggccctggtagactgcagtggatggaagtggacagcgagagcgtgccagccttctacagagaaaccgtgcaagtgggcagatgcagactgcagctgggacctagacctcctcctgtggctgtggccgtgattcctagatctggacctcctgctggcgaggctcctgatggacttgaggaagctcctgagcagcctccttctgctcctgcttctgctgtggtggctgagcctacacctccagctacaccagctccagccagcgacctgacaagcgaccctattcctgaagtgaccctggtggccactagcgacccaacacctgatcctattccagacgctaaccccgacgtggccacaagagatggcgaagaacaggtgcccgagcaggtttccgaacagggcgaagaatctggcgctgagcctggggatgagctgggaacagaacctgtgtctgagcaaggcgaggaacaaggcgccgagcctgtggaagagaaggacgaggaacccgaagaagaacaaggggctgagcccgttgaagaacagggcgctgaacctgtcgaggaacagaatggcgagccagttgaggaacaagacgagaatcaagagcagagaggccaagagctgaaggaccagcctctgtctggaatccctggcagacctgctcctggaacagctgccgctaacgccaacgacttcttcgctaagagaaagaggacagcccaagagaacaaggccagcaacgatgtgcctcctggctgcccttctcctaatgttgctcctggcgtgggcgccgtggaacagacacctagaaagagactgagatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 48 - protein, 8 aa, synthetic construct
GFTFKNYG

SEQ ID NO: 49 - protein, 8 aa, synthetic construct
VRYDGNNK

SEQ ID NO: 50 - protein, 12 aa, synthetic construct
ARDPETFGGFDY

SEQ ID NO: 51 - protein, 6 aa, synthetic construct
ESVSSN

SEQ ID NO: 52 - protein, 9 aa, synthetic construct
QQYYYSPRT

SEQ ID NO: 53 - protein, 214 aa, synthetic construct
DIQMTQSPSSLSASVGDRVTITCRASQSISSYLSWYQQKPGKAPKLLIYAASSLQSGVPSRFSGSGSGTDFTLTITSLQPEDFATYYCQQSYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 214 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 216 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 56 - protein, 214 aa, synthetic construct
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0252688 A1
(43) Pub. Date: Aug. 1, 2024

(54) COMPOSITIONS AND METHODS FOR ENHANCED PROTEIN PRODUCTION

(71) Applicant: HDT Bio Corp., Seattle, WA (US)

(72) Inventors: Amit Praful Khandhar, Issaquah, WA (US); Bryan Berube, Issaquah, WA (US); Kyle Krieger, Seattle, WA (US); Jesse Hong-Sae Erasmus, Port Orchard, WA (US); Gaurav Gulati, Shoreline, WA (US); Adrian Simpson, Shoreline, WA (US)

(21) Appl. No.: 18/612,003

(22) Filed: Mar. 21, 2024

Related U.S. Application Data
(63) Continuation of application No. PCT/US22/76820, filed on Sep. 21, 2022.
(60) Provisional application No. 63/247,113, filed on Sep. 22, 2021.

(51) Int. Cl. A61K 48/00; A61K 9/51; A61K 38/45; A61K 39/00; A61K 39/12

(52) U.S. Cl. CPC A61K 48/0066; A61K 9/5115; A61K 9/5123; A61K 9/5176; A61K 38/45; A61K 39/0011; A61K 39/12; A61K 48/0033

(57) ABSTRACT

The disclosure provides nanoparticle and compound compositions and methods of making and using the same to deliver a bioactive agent such as a nucleic acid encoding a protein, antibody, antigen, expression enhancer, or functional fragment thereof to a subject. Various nanoparticle carriers are described. Various compounds that increase protein expression are described. Various nucleic acids coding expression enhancers that increase protein expression are described. In some instances, the nanoparticle component may include a hydrophobic core, optionally having an inorganic particle, and a membrane having a cationic lipid.

### CROSS REFERENCE

This application is a continuation of International Application No. PCT/US2022/076820, filed Sep. 21, 2022, which claims the benefit of priority to U.S. Provisional Patent Application No. 63/247,113, filed Sep. 22, 2021, the contents of each of which are incorporated herein by reference in their entirety.

### STATEMENT AS TO FEDERALLY SPONSORED RESEARCH

This invention was made with government support under Contract number W81XWH2010588 awarded by the US Army Medical Research and Development Command. The government has certain rights in the invention.

### SEQUENCE LISTING

The instant application contains a Sequence Listing which has been submitted electronically in xml format and is hereby incorporated by reference in its entirety. Said xml copy, created on Mar. 14, 2024, is named 201953-705301-SL.xml and is 223,156 bytes in size.

### BACKGROUND

A challenge with nucleic acid encoded protein therapeutics is the protein (including antibody) expression levels in vivo. Therefore, there is a great unmet need for enhanced nucleic acid (including RNA)-encoded protein therapeutics that yield a therapeutically effective level of protein expression.

### BRIEF SUMMARY

Provided herein are compositions, wherein the compositions comprise: a nanoparticle; a nucleic acid coding for a protein or a functional fragment thereof; and a compound, wherein the compound enhances expression of the protein or the functional fragment thereof in mammalian cells. In some embodiments, the nanoparticle comprises a hydrophobic core. In some embodiments, the hydrophobic core comprises a liquid organic material and a solid inorganic material. In some embodiments, the hydrophobic core comprises the liquid organic material. In some embodiments, the hydrophobic core comprises the solid inorganic material. In some embodiments, the nanoparticle comprises a hydrophilic surface. In some embodiments, the nanoparticle is up to 200 nm in diameter. In some embodiments, the nanoparticle is 50 to 70 nm in diameter. In some embodiments, the nanoparticle is 40 to 80 nm in diameter. In some embodiments, the nanoparticle is dispersed in an aqueous solution. In some embodiments, the nanoparticle comprises a membrane. In some embodiments, the compound is dispersed in the hydrophobic core. In some embodiments, the compound is conjugated to the nanoparticle. In some embodiments, the nanoparticle comprises a cationic lipid. In some embodiments, the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP), dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. In some embodiments, the hydrophobic core comprises an oil. In some embodiments, the oil is in liquid phase. In some embodiments, the oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, solanesol, soy lecithin, soybean oil, sunflower oil, a triglyceride, or vitamin E. In some embodiments, the triglyceride is capric triglyceride, caprylic triglyceride, a caprylic and capric triglyceride, a triglyceride ester, or myristic acid triglycerine. In some embodiments, the hydrophobic core comprises a phosphate-terminated lipid. In some embodiments, the phosphate-terminated lipid is trioctylphosphine oxide (TOPO). In some embodiments, the nanoparticle comprises an inorganic particle. In some embodiments, the inorganic particle comprises a metal. In some embodiments, the metal comprises a metal salt, a metal oxide, a metal hydroxide, or a metal phosphate. In some embodiments, the metal oxide comprises aluminum oxide, aluminum oxyhydroxide, iron oxide, titanium dioxide, or silicon dioxide. In some embodiments, the nanoparticle comprises a surfactant. In some embodiments, the hydrophobic core comprises a surfactant. In some embodiments, the surfactant is a hydrophobic surfactant. In some embodiments, the hydrophobic surfactant is sorbitan monolaurate, sorbitan monopalmitate, sorbitan monostearate, sorbitan monooleate, or sorbitan trioleate. In some embodiments, the surfactant is a hydrophilic surfactant. In some embodiments, the hydrophilic surfactant is a polysorbate. In some embodiments, the surfactant is a phosphorous-terminated surfactant, a carboxylate-terminated surfactant, a sulfate-terminated surfactant, or an amine-terminated surfactant. In some embodiments, the surfactant is distearyl phosphatidic acid (DSPA), oleic acid, oleylamine or sodium dodecyl sulfate (SDS). In some embodiments, the nanoparticle comprises a cationic lipid, an oil, and an inorganic particle. In some embodiments, the nanoparticle comprises a cationic lipid, an oil, an inorganic particle, and a surfactant. In some embodiments, the hydrophobic core comprises one or more inorganic particles. In some embodiments, the hydrophobic core comprises a phosphate-terminated lipid and a surfactant. In some embodiments, each inorganic particle is coated with a capping ligand or the surfactant. In some embodiments, the compound comprises a plurality of compound. In some embodiments, the compound is a kinase inhibitor. In some embodiments, the kinase inhibitor is a casein kinase inhibitor, a cyclin-dependent kinase (CDK) inhibitor, an extracellular signal-regulated kinase (ERK) inhibitor, a growth factor inhibitor, a glycogen synthase kinase inhibitor, an immune checkpoint inhibitor, a Janus kinase (JAK) inhibitor, a IκB kinase (IKK) inhibitor, a glycogen synthase kinase-3β (GSK-3β) inhibitor, a lipid kinase inhibitor, a mitogen-activated protein kinase (MAPK) family inhibitor, a phosphatidylinositol 4-kinase (PI4K) inhibitor, a polo-like kinase (PLK) inhibitor, a protein kinase D (PKD) inhibitor, a tyrosine kinase inhibitor, a T-lymphokine-activated killer cell-originated protein kinase (TOPK) inhibitor, a salt inducible kinase (SIK) inhibitor, or a Wnt signaling inhibitor. In some embodiments, the kinase inhibitor is a CDK inhibitor. In some embodiments, the CDK inhibitor is (−)-5-fluoro-4-(4-fluoro-2-metboxyphenyl)-N-[4-[(methylsulfoniridoyl)methyl]pyridin-2-yl]pyridin-2-anine, (+)-5-fluoro-4-(4-fluoro-2-methoxyphenyl)-N′-[4-[(methylsulfonimidoyl)methyl]pyridin-2-yl]pyridin-2-anine, (+)-5-fluoro-4-(4-fluoro-2-methoxyphenyl)-N-[4-[(methylsulfonimidoyl)methyl]pyridin-2-yl]pyridin-2-amine, 2-[2-chloro-4-(trifluoromethyl)phenyl]-5,7-dihydroxy-8-[(2S,3R)-2-(hydroxymethyl)-1-methylpyrrolidin-3-yl]chromen-4-one; hydrochloride, 4-[(2,6-dichlorobenzoyl)amino]-N-piperidin-4-yl-1H-pyrazole-5-carboxamide; hydrochloride, 1-[4-(2-aminopyrimidin-4-yl)oxyphenyl]-3-[4-[(4-methylpiperazin-1-yl)methyl]-3-(trifluoromethyl)phenyl]urea, 4-(1-isopropyl-2-methyl-1H-imidazol-5-yl)-N-(4-(methylsulfonyl)phenyl)pyrimidin-2-amine, (1S,3R)-3-acetamido-N-[5-chloro-4-(5,5-dimethyl-4,6-dihy dropyrrolo[1,2-b]pyrazol-3-yl)pyridin-2-yl]cyclohexane-1-carboxamide, (3R)-N-[5-chloro-4-(5-fluoro-2-methoxyphenyl)pyridin-2-yl]piperidine-3-carboxamide, 2-[(2S)-1-[6-[(4,5-difluoro-1-benzimidazol-2-yl)methylamino]-9-propan-2-ylpurin-2-yl]piperidin-2-yl]ethanol, 1-N-[4-[[7-cyclopentyl-6-(dimethylcarbamoyl)pyrrolo[2,3-d]pyrimidin-2-yl]amino]phenyl]-1-V-(4-fluorophenyl)cyclopropane-1,1-dicarboxamide, 3-[[5-fluoro-4-[4-methyl-2-(methylamino)-1,3-thiazol-5-yl]pyrimidin-2-yl]amino]benzenesulfonamide, 2-[(2S)-1-[3-ethyl-7-[(1-oxidopyridin-1-ium-3-yl)methylamino]pyrazolo[1,5-a]pyrimidin-5-yl]piperidin-2-yl]ethanol, 2-(2-chlorophenyl)-5,7-dihydroxy-8-[(3S,4R)-3-hydroxy-1-methylpiperidin-4-yl]chromen-4-one, 5-amino-N-(2,6-difluorophenyl)-3-(4-sulfamoylanilino)-1,2,4-triazole-1-carbothioamide, (1S, 3S)-3-N-(5-pentan-3-ylpyrazolo[1,5-a]pyrimidin-7-yl)cyclopentane-1,3-diamine; dihydrochloride, 2-piperidin-3-yloxy-8-propan-2-yl-N-[(2-pyrazol-1-ylphenyl)methyl]pyrazol o[1,5-a][1,3,5]triazin-4-amine, LSN3106729, 4-N-[4-(2-methyl-3-propan-2-ylindazol-5-yl)pyrindin-2-yl]-1-N-(oxan-4-yl)cyclohexane-1,4-diamine, [4-amino-2-[[(1S′,2S,4R)-2-bicyclo[2,2,1]heptanyl]amino]-1,3-thiazol-5-yl]-(2-nitrophenyl)methanone, 4-[(2,6-dichlorobenzoyl)amino]-N-(1-methylsulfonylpiperidin-4-yl)-1H-pyrazole-5-carboxamide, 6-(difluoromethyl)-8-[(1R,2R)-2-hydroxy-2-methylcyclopentyl]-2-[(1-methylsulfonylpiperidin-4-yl)amino]pyrido[2,3-d]pyrimidin-7-one, 2-pyridin-4-yl-1,5,6,7-tetrahydropyrrolo[3,2-c]pyridin-4-one, N-[6,6-dimethyl-5-(1-methylpiperidine-4-carbonyl)-1,4-dihydropyrrolo[3,4-c]pyrazol-3-yl]-3-methylbutanamide, N-(5-cyclobutyl-1H-pyrazol-3-yl)-2-[4-[5-[2-(2,6-dioxopiperidin-3-yl)-1,3-dioxoisoindol-4-yl]oxypentoxy]phenyl]acetamide, 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrochloride, (2R)-2-[[6-(benzylamino)-9-propan-2-ylpurin-2-yl]amino]butan-1-ol, 2-[(2S)-1-azabicyclo[2,2,2]octan-2-yl]-6-(5-methyl-1H-pyrazol-4-yl)-3H-thieno[3,2-d]pyrimidin-4-one, N-[5-[(5-tert-butyl-1,3-oxazol-2-yl)methylsulfamyl]-1,3-thiazol-2-yl]piperidine-4-carboxamide, (3Z)-3-(1H-imidazol-5-ylmethylidene)-5-methoxy-1H-indol-2-one, N-[3-[[5-chloro-4-(1H-indol-3-yl)pyrimidin-2-yl]amino]phenyl]-3-[[(E)-4-(dimethylamino)but-2-enoyl]amino]benzamide, 2-[2-chloro-4-(trifluoromethyl)phenyl]-5,7-dihydroxy-8-[(2R,3S)-2-(hydroxymethyl)-1-methylpyrrolidin-3-yl]chromen-4-one, free base thereof, salt thereof, or combinations thereof. In some embodiments, the kinase inhibitor comprises (±)-5-fluoro-4-(4-fluoro-2-methoxyphenyl)-N-[4-[(methylsulfonimidoyl)methyl pyridin-2-yl]pyridin-2-anine, (1S,3R)-3-acetamido-N-[5-chloro-4-(5,5-dimethyl-4,6-dihydropyrrolo 1,2-b]pyrazol-3-yl)pyridin-2-yl]cyclohexane-1-carboxamide, (3R)-N-[5-chloro-4-(5-fluoro-2-methoxyphenyl)pyridin-2-yl]piperidine-3-carboxamide, 2-[(2S)-1-[6-[(4,5-difluoro-1H-benzimidazol-2-yl)methylamino]-9-propan-2-ylpurin-2-yl]piperidin-2-yl]ethanol, 3-[[5-fluoro-4-[4-methyl-2-(methylamino)-1,3-thiazol-5-yl]pyrimidin-2-yl]amino]benzenesulfonamide, 2-[(2S)-1-[3-ethyl-7-[(1-oxidopyridin-1-ium-3-yl)methylamino]pyrazolo[1,5-a]pyrimidin-5-yl]piperidin-2-yl]ethanol, 2-(2-chlorophenyl)-5,7-dihydroxy-8-[(3S,4R)-3-hydroxy-1-methylpiperidin-4-yl]chromen-4-one, 4-N-[4-(2-methyl-3-propan-2-ylindazol-5-yl)pyrimidin-2-yl]-1-N-(oxan-4-yl)cyclohexane-1,4-diamine, [4-amino-2-[[(1S,2S,4R)-2-bicyclo[2.2.1]heptanyl]amino]-1,3-thiazol-5-yl]-(2-nitrophenyl)methanone, 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrochloride, free base thereof, salt thereof, or combinations thereof. In some embodiments, the kinase inhibitor comprises 2-[2-chloro-4-(trifluoromethyl)phenyl]-5,7-dihydroxy-8-[(2S,3R)-2-(hydroxymethyl)-1-methylpyrrolidin-3-yl]chromen-4-one; hydrochloride, LSN3106729, 6-(difluoromethyl)-8-[(1R,2R)-2-hydroxy-2-methylcyclopentyl]-2-[(1-methylsulfonylpiperidin-4-yl)amino]pyrido[2,3-d]pyrimidin-7-one, 4-(1-isopropyl-2-methyl-1H-imidazol-5-yl)-N-(4-(methylsulfonyl)phenyl)pyrimidin-2-amine, (2R)-2-[[6-(benzylamino)-9-propan-2-ylpurin-2-yl]amino]butan-1-ol, 2-[(2S)-1-azabicyclo[2.2.2]octan-2-yl]-6-(5-methyl-TH-pyrazol-4-yl)-3H-thieno[3,2-d]pyrimidin-4-one, 4-[(2,6-dichlorobenzoyl)amino]-N-piperidin-4-yl-1H-pyrazole-5-carboxamide; hydrochloride, (3R)-N-[5-chloro-4-(5-fluoro-2-methoxyphenyl)pyridin-2-yl]piperidine-3-carboxamide, free base thereof, salt thereof, or combinations thereof. In some embodiments, the kinase inhibitor comprises 2-[(2S)-1-[3-ethyl-7-[(1-oxidopyridin-1-ium-3-yl)methylamino]pyrazolo[1,5-a]pyrimidin-5-yl]piperidin-2-yl]ethanol, free base thereof, salt thereof, or combinations thereof. In some embodiments, the kinase inhibitor comprises 3-[[5-fluoro-4-[4-methyl-2-(methylamino)-1,3-thiazol-5-yl]pyrimidin-2-yl]amino]benzenesulfonamide, free base thereof, salt thereof, or combinations thereof. In some embodiments, the kinase inhibitor comprises (1S,3R)-3-acetamido-N-[5-chloro-4-(5,5-dimethyl-4,6-dihydropyrrolo[1,2-b]pyrazol-3-yl)pyridin-2-yl]cyclohexane-1-carboxamide, free base thereof, salt thereof, or combinations thereof. In some embodiments, the kinase inhibitor is a MAP kinase inhibitor. In some embodiments, the MAP kinase inhibitor is 5-[4-(2-methoxyethoxy)phenyl]-7-phenyl-3H-pyrrolo[2,3-d]pyrimidin-4-one, 5-(4-cyclopropylimidazol-1-yl)-2-fluoro-4-methyl-N-[6-(4-propan-2-yl-1,2,4-triazol-3-yl)pyridin-2-ylbenzamide, 4-(12-(31-benzimidazol-5-ylamino)quinazolin-8-yl]oxycyclohexan-1-ol, 1-(5-tert-butyl-2-methylpyrazol-3-yl)-3-(4-pyridin-4-yloxyphenyl)urea, free base thereof, salt thereof, or combinations thereof. In some embodiments, the kinase inhibitor is growth factor inhibitor. In some embodiments, the growth factor inhibitor is 2-[4-[(E)-2-[5-[(1R)-1-(3,5-dichloropyridin-4-yl)ethoxy]-1H-indazol-3-yl]ethenyl]pyrazol-1-yl]ethanol, i-N-[4-[2-(cyclopropanecarbonylamino)pyridin-4-yl]oxy-2,5-difluorophenyl]-1-N-(4-fluorophenyl)cyclopropane-1,1-dicarboxamide, 6-chloro-N-(5-methyl-1H-pyrazol-3-yl)-2-(4-nitrophenoxy)pyrimidin-4-amine, 1-[4-[(4-ethylpiperazin-1-yl)methyl]-3-(trifluoromethyl)phenyl]-3-[4-[6-(methylamino)pyrimidin-4-yl]oxyphenyl]urea, N-[4-(2-amino-3-chloropyridin-4-yl)oxy-3-fluorophenyl]-5-(4-fluorophenyl)-4-oxo-1H-pyridine-3-carboxamide, 5-amino-N-(2,6-difluorophenyl)-3-(4-sulfamoylanilino)-1,2,4-triazole-1-carbothioamide, [3-[[4-(2-amino-3-chloropyridin-4-yl)oxy-3-fluorophenyl]carbamoyl]-5-(4-fluorophenyl)-4-oxopyridin-1-yl]methyl dihydrogen phosphate; 2-amino-2-(hydroxymethyl)propane-1,3-diol, (3Z)-5-[(1-ethylpiperidin-4-yl)amino]-3-[(3-fluorophenyl)-(5-methyl-1H-imidazol-2-yl)methylidene]-1H-indol-2-one, 2-N-[4-(3-aminopropylamino)phenyl]-4-N-(5-cyclopropyl-1H-pyrazol-3-yl)pyrimidine-2,4-diamine, 4-N-(5-cyclopropyl-1H-pyrazol-3-yl)-6-(4-methylpiperazin-1-yl)-2-N-[(3-propan-2-yl-1,2-oxazol-5-yl)methyl]pyrimidine-2,4-diamine, 1-[4-[methyl-[2-(3-sulfamoylanilino)pyrimidin-4-yl]amino]phenyl]-3-[4-(trifluoromethoxy)phenyl]urea, free base thereof, salt thereof, or combinations thereof. In some embodiments, the kinase inhibitor is a Janus kinase (JAK) inhibitor. In some embodiments, the JAK inhibitor is 5-fluoro-2-[[(1S)-1-(4-fluorophenyl)ethyl]amino]-6-[(5-methyl-1H-pyrazol-3-yl)amino]pyridine-3-carbonitrile, 6-N-[(1S)-1-(4-fluorophenyl)ethyl]-4-(1-methylpyrazol-4-yl)-2-N-pyrazin-2-ylpyridine-2,6-diamine, 6-N-[(1 S)-1-(4-fluorophenyl)ethyl]-4-(1-methylpyrazol-4-yl)-2-N-pyrazin-2-ylpyridine-2,6-dianine; hydrochloride, N-(cyanomethyl)-4-[2-(4-morpholin-4-ylanilino)pyrimidin-4-yl]benzamide, N-(cyanomethyl)-4-[2-(4-morpholin-4-ylanilino)pyrimidin-4-yl]benzarmide; sulfuric acid, 1-[3-[4-[[4-(2-methoxyethyl)piperazin-1-yl]menthyl]phenyl]-4-oxo-1H-indeno[1,2-c]pyrazol-5-yl]-3-morpholin-4-ylurea; dihydrochloride, free base thereof, salt thereof, or combinations thereof. In some embodiments, the kinase inhibitor is an extracellular signal-regulated kinase (ERK) inhibitor. In some embodiments, the ERK inhibitor is 1-[(1S)-1-(4-chloro-3-fluorophenyl)-2-hydroxyethyl]-4-[2-[(2-methylpyrazol-3-yl)amino]pyrimidin-4-yl]pyridin-2-one, 4-[2-(2-chloro-4-fluoroanilino)-5-methylpyrimidin-4-yl]-N-[(1S)-1-(3-chlorophenyl)-2-hydroxyethyl]-1H-pyrrole-2-carboxamide, free base thereof, salt thereof, or combinations thereof. In some embodiments, the kinase inhibitor is a polo-like kinase (PLK) inhibitor. In some embodiments, the PLK inhibitor is N-[[4-[(6-chloropyridin-3-yl)methoxy]-3-methoxyphenyl]methyl]-2-(3,4-dimethoxyphenyl)ethanamine, AN-(4-methoxyphenyl)sulfonyl-N-[2-[(PE)-2-(1-oxidopyridin-1-iun-4-yl)ethenyl]phenyl]acetamide, free base thereof, salt thereof, or combinations thereof. In some embodiments, the kinase inhibitor is a phosphatidylinositol 4-kinase (P14K) inhibitor. In some embodiments, the P14K inhibitor is 2-fluoro-4-[2-methyl-8-[(3-methylsulfonylphenyl)methylamino]imidazo[1,2-a]pyrazin-3-yl]phenol, free base thereof, salt thereof, or combinations thereof. In some embodiments, the kinase inhibitor is a tyrosine kinase inhibitor. In some embodiments, the tyrosine kinase inhibitor is 3-[[5-fluoro-2-(3-hydroxyanilino)pyrindin-4-yl]amino]phenol, free base thereof, salt thereof, or combinations thereof. In some embodiments, the kinase inhibitor is a T-lymphokine-activated killer cell-originated protein kinase (TOPK) inhibitor. In some embodiments, the TOPK inhibitor is 9-[4-[(2R)-1-aminopropan-2-yl]phenyl]-8-hydroxy-6-methyl-5H-thieno[2,3-c]quinolin-4-one, free base thereof, salt thereof, or combinations thereof. In some embodiments, the kinase inhibitor is a Wnt signaling pathway inhibitor. In some embodiments, the Wnt signaling inhibitor is 6-[2-[[4-(2,4-dichlorophenyl)-5-(5-methyl-1H-imidazol-2-yl)pyrimidin-2-yl]amino]ethylamino]pyridine-3-carbonitrile, 4-[2-(3H-benzimidazol-5-ylamino)quinazolin-8-yl]oxycyclohexan-1-ol, free base thereof, salt thereof, or combinations thereof. In some embodiments, the kinase inhibitor is a IκB kinase (IKK) inhibitor. In some embodiments, the IKK inhibitor is 2-amino-6-[2-(cyclopropylmethoxy)-6-hydroxyphenyl]-4-piperidin-4-ylpyridine-3-carbonitrile, 1-[4-[(1R)-1-[2-[[6-[6-(dimethylamino)pyrimidin-4-yl]-1H-benzimidazol-2-yl]amino]pyridin-4-yl]ethyl]piperazin-1-yl]-3,3,3-trifluoropropan-1-one, Y-(1,8-dimethylimidazo[1,2-a]quinoxalin-4-yl)ethane-1,2-diamine, free base thereof, salt thereof, or combinations thereof. In some embodiments, the kinase inhibitor is a protein kinase D (PKD) inhibitor. In some embodiments, the PKD inhibitor is 2-[4-[[(2R)-2-aminobutyl]amino]pyrimidin-2-yl]-4-(1-methylpyrazol-4-yl)phenol; dihydrochloride, 9-hydroxy-3,4-dihydro-2H-[1]benzothiolo[2,3-f][1,4]thiazepin-5-one, free base thereof, salt thereof, or combinations thereof. In some embodiments, the kinase inhibitor is a salt inducible kinase (SIK) inhibitor. In some embodiments, the SIK inhibitor is 3-(2,4-dimethoxyphenyl)-4-thiophen-3-yl-1H-pyrrolo[2,3-b]pyridine, free base thereof, salt thereof, or combinations thereof. In some embodiments, the kinase inhibitor is a casein kinase inhibitor. In some embodiments, the casein kinase inhibitor is 3-[3-[2-(3,4,5-trimethoxyanilino)pyrrolo[2,3-d]pyrimidin-7-yl]phenyl]propanenitrile, (3E)-3-[(2,4,6-trimethoxyphenyl)methylidene]-1H-indol-2-one, N-[(4,5-difluoro-11H-benzimidazol-2-yl)methyl]-9-(3-fluorophenyl)-2-morpholin-4-ylpurin-6-amine, free base thereof, salt thereof, or combinations thereof. In some embodiments, the kinase inhibitor is a glycogen synthase kinase-3β (GSK-3β) inhibitor. In some embodiments, the glycogen synthase kinase-3β (GSK-3β) inhibitor is 1-[(4-methoxyphenyl)methyl]-3-(5-nitro-1,3-thiazol-2-yl)urea, 6-[2-[[4-(2,4-difluorophenyl)-5-(5-methyl-1H-imidazol-2-yl)pyrimidin-2-yl]amino]ethylamino]pyridine-3-carbonitrile, CP21R7, GSK-3 inhibitor 1, Indirubin-3′-monoxime, 5-amino-N-(2,6-difluorophenyl)-3-(4-sulfamoylanilino)-1,2,4-triazole-1-carbothioamide, 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrochloride, free base thereof, salt thereof, or combinations thereof. In some embodiments, the compound is within the membrane. In some embodiments, the compound is conjugated to the membrane. In some embodiments, the membrane comprises a cationic lipid, an ionizable lipid, a polyethylene glycol (PEG) functionalized lipid, a cholesterol-functionalized lipid, a polylactic acid (PLA)-functionalized lipid, a polylactic-co-glycolic acid (PLGA)-functionalized lipid, or a liposome. In some embodiments, the nucleic acid is an RNA or a DNA. In some embodiments, the nucleic acid codes for an RNA polymerase. In some embodiments, the RNA polymerase is a Venezuelan equine encephalitis virus (VEEV) RNA polymerase. In some embodiments, the VEEV RNA polymerase comprises the amino acid sequence of SEQ ID NO: 39 OR SEQ ID NO: 40. In some embodiments, the nucleic acid coding the RNA polymerase comprises the nucleic acid sequence of SEQ ID NO: 38. In some embodiments, the nucleic acid is within the nanoparticle. In some embodiments, the nucleic acid is outside the nanoparticle. In some embodiments, the nucleic acid is in complex with the membrane. In some embodiments, the protein is an antigen or an antigen-binding protein. In some embodiments, the antigen is in a viral antigen. In some embodiments, the antigen is in a tumor antigen. In some embodiments, the nucleic acid coding for an antibody or a functional fragment thereof is in complex with the nanoparticle. In some embodiments, the protein is an antibody or a functional fragment thereof. In some embodiments, the antibody is a monoclonal antibody. In some embodiments, the antibody is a murine antibody, a humanized antibody, or a fully human antibody. In some embodiments, the antibody is an immunoglobulin (Ig) molecule. In some embodiments, the immunoglobulin molecule is an IgG, IgE, IgM, IgD, IgA, or an IgY isotype immunoglobulin molecule. In some embodiments, the immunoglobulin molecule is an IgG1, an IgG2, an IgG3, an IgG4, an IgGA1, or an IgGA2 subclass immunoglobulin molecule. In some embodiments, the antibody is a recombinant antibody, a chimeric antibody, or a multivalent antibody. In some embodiments, the multivalent antibody is a bispecific antibody, a trispecific antibody, or a multispecific antibody. In some embodiments, the antibody or functional fragment is an antigen-binding fragment (Fab), and Fab2 a F(ab′), a F(ab′)2, an dAb, an Fc, a Fv, a disulfide linked Fv, a scFv, a tandem scFv, a free LC, a half antibody, a single domain antibody (dAb), a diabody, or a nanobody. In some embodiments, the composition comprising the nanoparticle comprises a membrane and a hydrophobic core; wherein the compound is one or more compounds listed in Table 7; and wherein the compound is within the hydrophobic core. In some embodiments, the antibody or functional fragment thereof specifically binds to a tumor antigen or a microbial antigen. In some embodiments, the antibody or functional fragment thereof is a SARS-CoV-2 virus antibody. In some embodiments, the SARS-CoV-2 virus antibody is bamlanivimab, casirivimab, imdevimab, or sotrovimab. In some embodiments, the antibody or functional fragment thereof specifically binds to a viral antigen. In some embodiments, the viral antigen is a Zika virus antigen. In some embodiments, the Zika virus antigen is the envelope (E) protein. In some embodiments, the antibody or functional fragment thereof is a Zika virus antibody. In some embodiments, the Zika virus antibody is ZIKV-117, Z3L1, Z20, Z23, ZV67, Z006, or 2A10G6. In some embodiments, the Zika virus antibody or functional fragment thereof is a ZIKV-117 antibody. In some embodiments, the ZIKV-117 antibody or functional fragment comprises a heavy chain CDR1 amino acid sequence of GFTFKNYG (SEQ ID NO: 48), a heavy chain CDR2 amino acid sequence of VRYDGNNK (SEQ ID NO: 49), and a heavy chain CDR3 amino acid sequence of ARDPETFGGFDY (SEQ ID NO: 50), and a light chain CDR1 amino acid sequence of ESVSSN (SEQ ID NO: 51), light chain CDR2 amino acid sequence of GAS, and light chain CDR3 amino acid sequence of QQYYYSPRT (SEQ ID NO: 52). In some embodiments, the antibody or functional fragment thereof is a cancer therapeutic antibody. In some embodiments, the cancer therapeutic antibody is atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab. In some embodiments, the nanoparticle is a cationic lipid carrier, an ionizable lipid carrier, a gold carrier, a magnetic carrier, a polyethylene glycol (PEG)-functionalized carrier, a cholesterol-functionalized carrier, a polylactic acid (PLA)-functionalized carrier, a polylactic-co-glycolic acid (PLGA)-functionalized lipid carrier, or a liposome. In some embodiments, the composition is a modulator of a level or activity of NFκB relative to levels or activity interferon-α in the cell. In some embodiments, the compound is the modulator. In some embodiments, the protein is the modulator. In some embodiments, the composition is lyophilized. Provided herein are suspensions comprising compositions described herein. Provided herein are pharmaceutical compositions comprising compositions described herein and a pharmaceutical excipient.

Provided herein are compositions, wherein the compositions comprise: a nanoparticle; a first nucleic acid coding for a protein or a functional fragment thereof; a second nucleic acid coding for an expression enhancer or a functional fragment thereof, wherein the expression enhancer or the functional fragment thereof increases expression of the protein or the functional fragment thereof in mammalian cells. In some embodiments, the nanoparticle comprises a hydrophobic core. In some embodiments, the hydrophobic core comprises a liquid organic material, a solid inorganic material, or a combination thereof. In some embodiments, the hydrophobic core comprises the liquid organic material. In some embodiments, the hydrophobic core comprises the solid inorganic material. In some embodiments, the nanoparticle comprises a hydrophilic surface. In some embodiments, the nanoparticle is up to 200 nm in diameter. In some embodiments, the nanoparticle is 50 to 70 nm in diameter. In some embodiments, the nanoparticle is 40 to 80 nm in diameter. In some embodiments, the nanoparticle is dispersed in an aqueous solution. In some embodiments, the nanoparticle comprises a membrane. In some embodiments, the nanoparticle comprises a cationic lipid. In some embodiments, the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP), dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′, 9″″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. In some embodiments, the hydrophobic core comprises an oil. In some embodiments, the oil is in liquid phase. In some embodiments, the oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, solanesol, soy lecithin, soybean oil, sunflower oil, a triglyceride, or vitamin E. In some embodiments, the triglyceride is capric triglyceride, caprylic triglyceride, a caprylic and capric triglyceride, a triglyceride ester, or myristic acid triglycerine. In some embodiments, the hydrophobic core comprises a phosphate-terminated lipid. In some embodiments, the phosphate-terminated lipid is trioctylphosphine oxide (TOPO). In some embodiments, the nanoparticle comprises an inorganic particle. In some embodiments, the inorganic particle comprises a metal. In some embodiments, the metal comprises a metal salt, a metal oxide, a metal hydroxide, or a metal phosphate. In some embodiments, the metal oxide comprises aluminum oxide, aluminum oxyhydroxide, iron oxide, titanium dioxide, or silicon dioxide. In some embodiments, the nanoparticle comprises a surfactant. In some embodiments, the hydrophobic core comprises a surfactant. In some embodiments, the surfactant is a hydrophobic surfactant. In some embodiments, the hydrophobic surfactant is sorbitan monolaurate, sorbitan monopalmitate, sorbitan monostearate, sorbitan monooleate, or sorbitan trioleate. In some embodiments, the surfactant is a hydrophilic surfactant. In some embodiments, the hydrophilic surfactant is a polysorbate. In some embodiments, the surfactant is a phosphorous-terminated surfactant, a carboxylate-terminated surfactant, a sulfate-terminated surfactant, or an amine-terminated surfactant. In some embodiments, the surfactant is distearyl phosphatidic acid (DSPA), oleic acid, oleylamine or sodium dodecyl sulfate (SDS). In some embodiments, the nanoparticle comprises a cationic lipid, an oil, and an inorganic particle. In some embodiments, the nanoparticle comprises a cationic lipid, an oil, an inorganic particle, and a surfactant. In some embodiments, the hydrophobic core comprises one or more inorganic particles. In some embodiments, the hydrophobic core further comprises: a phosphate-terminated lipid; and a surfactant. In some embodiments, each inorganic particle is coated with a capping ligand or the surfactant. In some embodiments, the membrane comprises a lipid bilayer. In some embodiments, the membrane comprises a cationic lipid, an ionizable lipid, a polyethylene glycol (PEG) functionalized lipid, a cholesterol-functionalized lipid, a polylactic acid (PLA)-functionalized lipid, a polylactic-co-glycolic acid (PLGA)-functionalized lipid, or a liposome. In some embodiments, the first nucleic acid, the second nucleic acid, or both are RNA or DNA. In some embodiments, the first nucleic acid, the second nucleic acid, or both are dispersed within the hydrophobic core. In some embodiments, the first nucleic acid, the second nucleic acid, or both are bound to the hydrophilic surface of the nanoparticle. In some embodiments, the first nucleic acid, the second nucleic acid, or both are in complex with the membrane. In some embodiments, the nanoparticle comprises a single nucleic acid comprising at least one of the first nucleic acid and at least one of the second nucleic acid. In some embodiments, the nanoparticle comprises a plurality of nucleic acid, wherein each of the plurality of nucleic acid comprises at least one of the first nucleic acid, at least one of the second nucleic acid, or combinations thereof. In some embodiments, the expression enhancer is a kinase inhibitor. In some embodiments, the kinase inhibitor is a casein kinase inhibitor, a cyclin-dependent kinase (CDK) inhibitor, an extracellular signal-regulated kinase (ERK) inhibitor, a growth factor inhibitor, a glycogen synthase kinase inhibitor, an immune checkpoint inhibitor, a Janus kinase (JAK) inhibitor, a IκB kinase (IKK) inhibitor, a glycogen synthase kinase-3β (GSK-3β) inhibitor, a lipid kinase inhibitor, a mitogen-activated protein kinase (MAPK) family inhibitor, a phosphatidylinositol 4-kinase (P14K) inhibitor, a polo-like kinase (PLK) inhibitor, a protein kinase D (PKD) inhibitor, a tyrosine kinase inhibitor, a T-lymphokine-activated killer cell-originated protein kinase (TOPK) inhibitor, a salt inducible kinase (SIK) inhibitor, or a Wnt signaling inhibitor. In some embodiments, the kinase inhibitor is the CDK inhibitor. In some embodiments, the CDK inhibitor comprises an amino acid sequence that has at least 80% sequence identity with any one of the sequences of SEQ ID NO: 41 to 47. In some embodiments, the first nucleic acid further codes for an RNA polymerase. In some embodiments, the RNA polymerase is a Venezuelan equine encephalitis virus (VEEV) RNA polymerase. In some embodiments, the VEEV RNA polymerase comprises the amino acid sequence of SEQ ID NO: 39 OR SEQ ID NO: 40. In some embodiments, the first nucleic acid coding the RNA polymerase comprises the nucleic acid sequence of SEQ ID NO: 38. In some embodiments, the protein is an antigen or an antigen-binding protein. In some embodiments, the antigen is in a viral antigen. In some embodiments, the antigen is in a tumor antigen. In some embodiments, the first nucleic acid coding for an antibody or a functional fragment thereof is in complex with the nanoparticle. In some embodiments, the protein is an antibody or a functional fragment thereof. In some embodiments, the antibody is a monoclonal antibody. In some embodiments, the antibody is a murine antibody, a humanized antibody, or a fully human antibody. In some embodiments, the antibody is an immunoglobulin (Ig) molecule. In some embodiments, the immunoglobulin molecule is an IgG, IgE, IgM, IgD, IgA, or an IgY isotype immunoglobulin molecule. In some embodiments, the immunoglobulin molecule is an IgG1, an IgG2, an IgG3, an IgG4, an IgGA1, or an IgGA2 subclass immunoglobulin molecule. In some embodiments, the antibody is a recombinant antibody, a chimeric antibody, or a multivalent antibody. In some embodiments, the multivalent antibody is a bispecific antibody, a trispecific antibody, or a multispecific antibody. In some embodiments, the antibody or functional fragment is an antigen-binding fragment (Fab), and Fab2 a F(ab′), a F(ab′)2, an dAb, an Fc, a Fv, a disulfide linked Fv, a scFv, a tandem scFv, a free LC, a half antibody, a single domain antibody (dAb), a diabody, or a nanobody. In some embodiments, the antibody or functional fragment thereof specifically binds to a tumor antigen or a microbial antigen. In some embodiments, the antibody or functional fragment thereof is a SARS-CoV-2 virus antibody. In some embodiments, the SARS-CoV-2 virus antibody is bamlanivimab, casirivimab, imdevimab, or sotrovimab. In some embodiments, the antibody or functional fragment thereof specifically binds to a viral antigen. In some embodiments, the viral antigen is a Zika virus antigen. In some embodiments, the Zika virus antigen is the envelope (E) protein. In some embodiments, the antibody or functional fragment thereof is a Zika virus antibody. In some embodiments, the Zika virus antibody is ZIKV-117, Z3L1, Z20, Z23, ZV67, Z006, or 2A10G6. In some embodiments, the Zika virus antibody or functional fragment thereof is a ZIKV-117 antibody. In some embodiments, the ZIKV-117 antibody or functional fragment comprises a heavy chain CDR1 amino acid sequence of GFTFKNYG (SEQ ID NO: 48), a heavy chain CDR2 amino acid sequence of VRYDGNNK (SEQ ID NO: 49), and a heavy chain CDR3 amino acid sequence of ARDPETFGGFDY (SEQ ID NO: 50), and a light chain CDR1 amino acid sequence of ESVSSN (SEQ ID NO: 51), light chain CDR2 amino acid sequence of GAS, and light chain CDR3 amino acid sequence of QQYYYSPRT (SEQ ID NO: 52). In some embodiments, the antibody or functional fragment thereof is a cancer therapeutic antibody. In some embodiments, the cancer therapeutic antibody is atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab. In some embodiments, the nanoparticle is a cationic lipid carrier, an ionizable lipid carrier, a gold carrier, a magnetic carrier, a polyethylene glycol (PEG)-functionalized carrier, a cholesterol-functionalized carrier, a polylactic acid (PLA)-functionalized carrier, a polylactic-co-glycolic acid (PLGA)-functionalized lipid carrier, or a liposome. In some embodiments, the enhancer is a modulator of a level or activity of NFκB relative to levels or activity interferon-α in the cell. In some embodiments, the composition further comprises a compound. In some embodiments, the composition further comprises a plurality of compound. In some embodiments, at least two of the plurality of compounds are the same. In some embodiments, the at least two of the plurality of compounds are different. In some embodiments, the compound is conjugated to the nanoparticle. In some embodiments, the compound is dispersed in a hydrophobic core of the nanoparticle. In some embodiments, the compound is a kinase inhibitor. In some embodiments, the kinase inhibitor is a casein kinase inhibitor, a cyclin-dependent kinase (CDK) inhibitor, an extracellular signal-regulated kinase (ERK) inhibitor, a growth factor inhibitor, a glycogen synthase kinase inhibitor, an immune checkpoint inhibitor, a Janus kinase (JAK) inhibitor, a IκB kinase (IKK) inhibitor, a glycogen synthase kinase-3β (GSK-3β) inhibitor, a lipid kinase inhibitor, a mitogen-activated protein kinase (MAPK) family inhibitor, a phosphatidylinositol 4-kinase (P14K) inhibitor, a polo-like kinase (PLK) inhibitor, a protein kinase D (PKD) inhibitor, a tyrosine kinase inhibitor, a T-lymphokine-activated killer cell-originated protein kinase (TOPK) inhibitor, a salt inducible kinase (SIK) inhibitor, or a Wnt signaling inhibitor. In some embodiments, the kinase inhibitor is the CDK inhibitor. In some embodiments, the CDK inhibitor is (−)-5-fluoro-4-(4-fluoro-2-methoxyphenyl)-N-[4-[(methylsulfonimidoyl)methyl]pyridin-2-yl]pyridin-2-amine, (+)-5-fluoro-4-(4-fluoro-2-methoxy phenyl)-N-[4-[(methylsulfonimidoyl))methyl]pyridin-2-yl]pyridin-2-anine, (+)-5-fluoro-4-(4-fluoro-2-methoxyphenyl)-N-[4-[(methylsulfonimidoyl)methyl]pyridin-2-yl]pyridin-2-amine, 2-[2-chloro-4-(trifluoromethyl)phenyl]-5,7-dihydroxy-8-[(2S,3R)-2-(hydroxymethyl)-1-methylpyrrolidin-3-yl]chromen-4-one; hydrochloride, 4-[(2,6-dichlorobenzoyl)amino]-N-piperidin-4-yl-TH-pyrazole-5-carboxamide; hydrochloride, 1-[4-(2-aminopyrimidin-4-yl)oxyphenyl]-3-[4-[(4-methylpiperazin-1-yl)methyl]-3-(trifluoromethyl)phenyl]urea, 4-(1-isopropyl-2-methyl-1H-imidazol-5-yl)-N-(4-(methylsulfonyl)phenyl)pyrimidin-2-amine, (1S,3R)-3-acetamido-N-[5-chloro-4-(5,5-dimethyl-4,6-dihydropyrrolo[1,2-b]pyrazol-3-yl)pyridin-2-yl]cyclohexane-1-carboxamide, (3R)-N-[5-chloro-4-(5-fluoro-2-methoxyphenyl)pyridin-2-yl]piperidine-3-carboxamide, 2-[(2S)-1-[6-[(4,5-difluoro-1H-benzimidazol-2-yl)methylamino]-9-propan-2-ylpurin-2-yl]piperidin-2-yl]ethanol, 1-N-[4-[[7-cyclopentyl-6-(dimethylcarbamoyl)pyrrolo[2,3-d]pyrimidin-2-yl]amino]phenyl]-1-N-(4-fluorophenyl)cyclopropane-1,1-dicarboxamide, 3-[[5-fluoro-4-[4-methyl-2-(methylamino)-1,3-thiazol-5-yl]pyrimidin-2-yl]amino]benzenesulfonamide, 2-[(2S)-1-[3-ethyl-7-[(1-oxidopyridin-1-ium-3-yl)methylamino]pyrazolo[1,5-a]pyrimidin-5-yl]piperidin-2-yl]ethanol, 2-(2-chlorophenyl)-5,7-dihydroxy-8-[(3S,4R)-3-hydroxy-1-methylpiperidin-4-yl]chromen-4-one, 5-amino-N-(2,6-difluorophenyl)-3-(4-sulfamoylanilino)-1,2,4-triazole-1-carbothioamide, (1S,3S)-3-N-(5-pentan-3-ylpyrazolo[1,5-a]pyrimidin-7-yl)cyclopentane-1,3-diamine; dihydrochloride, 2-piperidin-3-yloxy-8-propan-2-yl-N-[(2-pyrazol-1-ylphenyl)methyl]pyrazolo[1,5-a][1,3,5]triazin-4-amine, LSN3106729, 4-A-[4-(2-methyl-3-propan-2-ylindazol-5-yl)pyrimidin-2-yl]-1-N-(oxan-4-yl)cyclohexane-1,4-diamine, [4-amino-2-[[(1S,2S,4R)-2-bicyclo[2.2.1]heptanyl]amino]-1,3-thiazol-5-yl]-(2-nitrophenyl)methanone, 4-[(2,6-dichlorobenzoyl)amino]-N-(1-methylsulfonylpiperidin-4-yl)-1H-pyrazole-5-carboxamide, 6-(difluoromethyl)-8-[(1R,2R)-2-hydroxy-2-methylcyclopentyl]-2-[(1-methylsulfonylpiperidin-4-yl)amino]pyrido[2,3-d]pyrimidin-7-one, 2-pyridin-4-yl-1,5,6,7-tetrahydropyrrolo[3,2-c]pyridin-4-one, N-[6,6-dimethyl-5-(1-methylpiperidine-4-carbonyl)-1,4-dihydropyrrolo[3,4-c]pyrazol-3-yl]-3-methylbutanamide, N-(5-cyclobutyl-1H-pyrazol-3-yl)-2-[4-[5-[2-(2,6-dioxopiperidin-3-yl)-1,3-dioxoisoindol-4-yl]oxypentoxy]phenyl]acetamide, 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrochloride, (2R)-2-[[6-(benzylamino)-9-propan-2-ylpurin-2-yl]amino]butan-1-ol, 2-[(2S)-1-azabicyclo[2.2.2]octan-2-yl]-6-(5-methyl-TH-pyrazol-4-yl)-3H-thieno[3,2-d]pyrimidin-4-one, N-[5-[(5-tert-butyl-1,3-oxazol-2-yl)methylsulfanyl]-1,3-thiazol-2-yl]piperidine-4-carboxamide, (3Z)-3-(1H-imidazol-5-ylmethylidene)-5-methoxy-1H-indol-2-one, N-[3-[[5-chloro-4-(1H-indol-3-yl)pyrindin-2-yl]amino]phenyl]-3-[[(E)-4-(dimethylamino)but-2-enoyl]amino]benzamide, 2-[2-chloro-4-(trifluoromethyl)phenyl]-5,7-dihydroxy-8-[(2R,3S)-2-(hydroxymethyl)-1-methylpyrrolidin-3-yl]chromen-4-one, free base thereof, salt thereof, or combinations thereof. In some embodiments, the kinase inhibitor comprises (+)-5-fluoro-4-(4-fluoro-2-methoxyphenyl)-N-[4-[(methylsulfonimidoyl)methyl]pyridin-2-yl]pyridin-2-amine, (1S,3R)-3-acetamido-N-[5-chloro-4-(5,5-dimethyl-4,6-dihydropyrrolo[1,2-b]pyrazol-3-yl)pyridin-2-yl]cyclohexane-1-carboxamide, (3R)-N-[5-chloro-4-(5-fluoro-2-methoxyphenyl)pyridin-2-yl]piperidine-3-carboxamide, 2-[(2S)-1-[6-[(4,5-difluoro-1H-benzimidazol-2-yl)methylamino]-9-propan-2-ylpurin-2-yl]piperidin-2-yl]ethanol, 3-[[5-fluoro-4-[4-methyl-2-(methylamino)-1,3-thiazol-5-yl]pyrindin-2-yl]amino]benzenesulfonamide, 2-[(2S)-1-[3-ethyl-7-[(1-oxidopyridin-1-ium-3-yl)methylamino]pyrazolo[1,5-a]pyrimidin-5-yl]piperidin-2-yl]ethanol, 2-(2-chlorophenyl)-5,7-dihydroxy-8-[(3S,4R)-3-hydroxy-1-methylpiperidin-4-yl]chromen-4-one, 4-N-[4-(2-methyl-3-propan-2-ylindazol-5-yl)pyrimidin-2-yl]-1-N-(oxan-4-yl)cyclohexane-1,4-diamine, [4-amino-2-[[(1S,2S,4R)-2-bicyclo[2.2.1]heptanyl]amino]-1,3-thiazol-5-yl]-(2-nitrophenyl)methanone, 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrochloride, free base thereof, salt thereof, or combinations thereof. In some embodiments, the kinase inhibitor comprises 2-[2-chloro-4-(trifluoromethyl)phenyl]-5,7-dihydroxy-8-[(2S,3R)-2-(hydroxymethyl)-1-methylpyrrolidin-3-yl]chromen-4-one; hydrochloride, LSN3106729, 6-(difluoromethyl)-8-[(1R,2R)-2-hydroxy-2-methylcyclopentyl]-2-[(1-methylsulfonylpiperidin-4-yl)amino]pyrido[2,3-d]pyrimidin-7-one, 4-(1-isopropyl-2-methyl-1H-imidazol-5-yl)-N-(4-(methylsulfonyl)phenyl)pyrimidin-2-amine, (2R)-2-[[6-(benzylamino)-9-propan-2-ylpurin-2-yl]amino]butan-1-ol, 2-[(2S)-1-azabicyclo[2.2.2]octan-2-yl]-6-(5-methyl-H-pyrazol-4-yl)-3H-thieno[3,2-d]pyrimidin-4-one, 4-[(2,6-dichlorobenzoyl)amino]-N-piperidin-4-yl-1H-pyrazole-5-carboxamide; hydrochloride, (3R)-N-[5-chloro-4-(5-fluoro-2-methoxyphenyl)pyridin-2-yl]piperidine-3-carboxamide, free base thereof, salt thereof, or combinations thereof. In some embodiments, the kinase inhibitor comprises 2-[(2S)-1-[3-ethyl-7-[(1-oxidopyridin-1-ium-3-yl)methylamino]pyrazolo[1,5-a]pyrimidin-5-yl]piperidin-2-yl]ethanol, free base thereof, salt thereof, or combinations thereof. In some embodiments, the kinase inhibitor comprises 3-[[5-fluoro-4-[4-methyl-2-(methylamino)-1,3-thiazol-5-yl]pyrimidin-2-yl]amino]benzenesulfonamide, free base thereof, salt thereof, or combinations thereof. In some embodiments, the kinase inhibitor comprises (1S,3R)-3-acetamido-N-[5-chloro-4-(5,5-dimethyl-4,6-dihydropyrazolo[1,2-b]pyrazol-3-yl)pyridin-2-yl]cyclohexane-1-carboxamide, free base thereof, salt thereof, or combinations thereof. In some embodiments, the kinase inhibitor is a MAP kinase inhibitor. In some embodiments, the MAP kinase inhibitor is 5-[4-(2-methoxyethoxy)phenyl]-7-phenyl-3H-pyrrolo[2,3-d]pyrimidin-4-one, 5-(4-cyclopropylimidazol-1-yl)-2-fluoro-4-methyl-N-[6-(4-propan-2-yl-1,2,4-triazol-3-yl)pyridin-2-yl]benzamide, 4-[2-(3H-benzimidazol-5-ylamino)quinazolin-8-yl]oxycyclohexan-1-ol, 1-(5-tert-butyl-2-methylpyrazol-3-yl)-3-(4-pyridin-4-yloxyphenyl)urea, free base thereof, salt thereof, or combinations thereof. In some embodiments, the kinase inhibitor is growth factor inhibitor. In some embodiments, the growth factor inhibitor is 2-[4-[(E)-2-[5-[(1R)-1-(3,5-dichloropyridin-4-yl)ethoxy]-1H-indazol-3-yl]ethenyl]pyrazol-1-yl]ethanol, I-N-[4-[2-(cyclopropanecarbonylamino)pyridin-4-yl]oxy-2,5-difluorophenyl]-1-N-(4-fluorophenol)cyclopropane-1,1-dicarboxamide, 6-chloro-N-(5-methyl-1H-pyrazol-3-3-yl)-2-(4-nitrophenoxy)pyrimidin-4-amine, 1-[4-[(4-ethylpiperazin-1-yl)methyl]-3-(trifluoromethyl)phenyl]-3-[4-[6-(methylamino)pyrimidin-4-yl]oxyphenyl]urea, N-[4-(2-amino-3-chloropyridin-4-yl)oxy-3-fluorophenyl]-5-(4-fluorophenyl)-4-oxo-1-pyridine-3-carboxamide, 5-amino-N-(2,6-difluorophenyl)-3-(4-sulfamoylanilino)-12,4-triazole-1-carbothioamide, [3-[[4-(2-amino-3-chloropyridin-4-yl)oxy-3-fluorophenyl]carbamoyl]-5-(4-fluorophenyl)-4-oxopyridin-1-yl]methyl dihydrogen phosphate; 2-amino-2-(hydroxymethyl)propane-1,3-diol, (3Z)-5-[(1-ethylpiperidin-4-yl)amino]-3-[(3-fluorophenyl)-(5-methyl-1H-imidazol-2-yl)methylidene]-1H-indol-2-one, 2-N-[4-(3-aminopropylamino)phenyl]-4-N-(5-cyclopropyl-1H-pyrazol-3-yl)pyrimidine-2,4-diamine, 4-N-(5-cyclopropyl-1H-pyrazol-3-yl)-6-(4-methylpiperazin-1-yl)-2-N-[(3-propan-2-yl-1,2-oxazol-5-yl)methyl]pyrimidine-2,4-diamine, 1-[4-[methyl-[2-(3-sulfamoylanilino)pyrimidin-4-yl]amino]phenyl]-3-[4-(trifluoromethoxy)phenyl]urea, free base thereof, salt thereof, or combinations thereof. In some embodiments, the kinase inhibitor is a Janus kinase (JAK) inhibitor. In some embodiments, the JAK inhibitor is 5-fluoro-2-[[(1S)-1-(4-fluorophenyl)ethyl]amino]-6-[(5-methyl-1H-pyrazol-3-yl)amino]pyridine-3-carbonitrile, 6-N-[(1S)-1-(4-fluorophenyl)ethyl]-4-(1-methylpyrazol-4-yl)-2-N-pyrazin-2-ylpyridine-2,6-diamine, 6-N-[(1S)-1-(4-fluorophenyl)ethyl]-4-(1-methylpyrazol-4-yl)-2-N-pyrazin-2-ylpyridine-2,6-diamine; hydrochloride, N-(cyanomethyl)-4-[2-(4-morpholin-4-ylanilino)pyrimidin-4-yl]benzamide, N-(cyanomethyl)-4-[2-(4-morpholin-4-ylanilino)pyrimidin-4-yl]benzamide; sulfuric acid, 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrocbloride, free base thereof, salt thereof, or combinations thereof. In some embodiments, the kinase inhibitor is an extracellular signal-regulated kinase (ERK) inhibitor. In some embodiments, the ERK inhibitor is 1-[(1S)-1-(4-chloro-3-fluorophenyl)-2-hydroxyethyl]-4-[2-[(2-methylpyrazol-3-yl)amino]pyrindin-4-yl]pyridin-2-one, 4-[2-(2-chloro-4-fluoroanilino)-5-methylpyrimidin-4-yl]-N-[(1S)-1-(3-chlorophenyl)-2-hydroxyethyl]-1H-pyrrole-2-carboxamide, free base thereof, salt thereof, or combinations thereof. In some embodiments, the kinase inhibitor is a polo-like kinase (PLK) inhibitor. In some embodiments, the PLK inhibitor is N-[[4-[(6-chloropyridin-3-yl)methoxy]-3-methoxyphenyl]methyl]-2-(3,4-dimethoxyphenyl)ethanamine, V-(4-methoxyphenyl)sulfonyl-N-[2-[(E)-2-(1-oxidopyridin-1-ium-4-yl)ethenyl]phenyl]acetamide, free base thereof, salt thereof, or combinations thereof. In some embodiments, the kinase inhibitor is a phosphatidylinositol 4-kinase (PI4K) inhibitor. In some embodiments, the PI4K inhibitor is 2-fluoro-4-[2-methyl-8-[(3-methylsulfonylphenyl)methylamino]imidazo[1,2-a]pyrazin-3-yl]phenol, free base thereof, salt thereof, or combinations thereof. In some embodiments, the kinase inhibitor is a tyrosine kinase inhibitor. In some embodiments, the tyrosine kinase inhibitor is 3-[[5-fluoro-2-(3-hydroxyanilino)pyrimidin-4-Yl]_amino]phenol, free base thereof, salt thereof, or combinations thereof. In some embodiments, the kinase inhibitor is a T-lymphokine-activated killer cell-originated protein kinase (TOPK) inhibitor. In some embodiments, the TOPK inhibitor is 9-[4-[(2R)-1-aminopropan-2-yl]phenyl]-8-hydroxy-6-methyl-5H-thieno[2,3-c]quinolin-4-one, free base thereof, salt thereof, or combinations thereof. In some embodiments, the kinase inhibitor is a Wnt signaling pathway inhibitor. In some embodiments, the Wnt signaling inhibitor is 6-[2-[[4-(2,4-dichlorophenyl)-5-(5-methyl-1H-imidazol-2-yl)pyrimidin-2-yl]amino]ethylamino]pyridine-3-carbonitrile, 4-[2-(3H-benzimidazol-5-ylamino)quinazolin-8-yl]oxycyclohexan-1-ol, free base thereof, salt thereof, or combinations thereof. In some embodiments, the kinase inhibitor is a IκB kinase (IKK) inhibitor. In some embodiments, the IKK inhibitor is 2-amino-6-[2-(cyclopropylnethoxy)-6-hydroxyphenyl]-4-piperidin-4-ylpyridine-3-carbonitrile, 1-[4-[(1R)-1-[2-[[6-[6-(dimethylamino)pyrimidin-4-yl]-1H-benzimidazol-2-yl]amino]pyridin-4-yl]ethyl]piperazin-1-yl]-3,3,3-trifluoropropan-1-one, N-(1,8-dimethylimidazo[1,2-a]quinoxalin-4-yl)ethane-1,2-diamine, free base thereof, salt thereof, or combinations thereof. In some embodiments, the kinase inhibitor is a protein kinase D (PKD) inhibitor. In some embodiments, the PKD inhibitor is 2-[4-[[(2R)-2-aminobutyl]amino]pyrimidin-2-yl]-4-(1-methylpyrazol-4-yl)phenol; dihydrochloride, 9-hydroxy-3,4-dihydro-2H-[1]benzothiolo[2,3-f][1,4]thiazepin-5-one, free base thereof, salt thereof, or combinations thereof. In some embodiments, the kinase inhibitor is a salt inducible kinase (SIK) inhibitor. In some embodiments, the SIK inhibitor is 3-(2,4-dimethoxyphenyl)-4-thiophen-3-yl-1H-pyrrolo[2,3-b]pyridine, free base thereof, salt thereof, or combinations thereof. In some embodiments, the kinase inhibitor is a casein kinase inhibitor. In some embodiments, the casein kinase inhibitor is 3-[3-[2-(3,4,5-trimethoxyanilino)pyrrolo[2,3-d]pyrimidin-7-yl]phenyl]propanenitrile, (3E)-3-[(2,4,6-trimethoxyphenyl)methylidene]-11H-indol-2-one, N-[(4,5-difluoro-1H-benzimidazol-2-yl)methyl]-9-(3-fluorophenyl)-2-morpholin-4-ylpurin-6-amine, free base thereof, salt thereof, or combinations thereof. In some embodiments, the kinase inhibitor is a glycogen synthase kinase-3β (GSK-3β) inhibitor. In some embodiments, the glycogen synthase kinase-3β (GSK-3β) inhibitor is 1-[(4-methoxyphenyl)methyl]-3-(5-nitro-1,3-thiazol-2-yl)urea, 6-[2-[[4-(2,4-dichlorophenyl)-5-(5-methyl-1H-imidazol-2-yl)pyrimidin-2-yl]amino]ethylamino]pyridine-3-carbonitrile, CP21R7, GSK-3 inhibitor 1, Indirubin-3′-monoxime, 5-amino-N-(2,6-difluorophenyl)-3-(4-sulfamoylanilino)-1,2,4-triazole-1-carbothioamide, 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrochloride, free base thereof, salt thereof, or combinations thereof. In some embodiments, the composition is lyophilized. Provided herein are suspensions comprising compositions described herein. Provided herein are pharmaceutical compositions comprising compositions described herein and a pharmaceutical excipient.

Provided herein are methods comprising administering to a subject the composition, the suspension, or the pharmaceutical composition described herein in an amount sufficient to modify NFκB expression or activity relative to interferon-α activity in the subject. Provided herein are methods for treatment of infection, the method comprising administering to a subject having an infection the composition, the suspension, or the pharmaceutical composition described herein. Provided herein are methods for treatment of cancer, the method comprising administering to a subject having an infection the composition, the suspension, or the pharmaceutical composition described herein. In some embodiments, the administering is local administration or systemic administration. In some embodiments, the administering is via intramuscular injection, intranasal administration, oral administration, subcutaneous administration, intratumoral administration, or intravenous injection. In some embodiments, the subject has a solid tumor or a blood cancer. In some embodiments, the solid tumor is a carcinoma, a melanoma, or a sarcoma. In some embodiments, the blood cancer is lymphoma or leukemia. In some embodiments, the subject has lung cancer. In some embodiments, the lung cancer is adenocarcinoma, squamous cell carcinoma, small cell cancer or non-small cell cancer.

Provided herein is a method comprising contacting a cell with the composition described herein, wherein the contacting modifies the level or activity of NFκB relative to interferon-α levels or activity in the cell. In some embodiments, the contacting is ex vivo, in vivo, or in vitro. In some embodiments, the cell is a cancer cell or a blood cell. In some embodiments, the cancer cell is a lung cancer cell. In some embodiments, the blood cell is a dendritic cell or a natural killer cell.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1W show schematic representations of nanoparticle (NP) carriers. FIG. 1A shows an oil-in-water emulsion and a nucleic acid. FIG. 1B shows a nanostructured lipid carrier (NLC) and a nucleic acid. FIG. 1C shows an inorganic nanoparticle and a nucleic acid. FIG. 1D shows an oil-in-water emulsion with small molecule enhancers for enhancing the protein expression from a nucleic acid. FIG. 1E shows a nanoparticle containing an inorganic nanoparticles small molecule enhancers of protein expression within a membrane of the nanoparticle, and a nucleic acid. FIG. 1F shows a nanoparticle comprising inorganic solid particles, small molecule enhancers of protein expression bound or conjugated to inside of the membrane of the nanoparticle, and a nucleic acid. FIG. 1G shows a nanoparticle comprising inorganic solid particles, small molecule enhancers of protein expression bound or conjugated to the outside of the membrane, and a nucleic acid. FIG. 1H shows a nanoparticle comprising small molecule enhancers of protein expression bound or conjugated to inside of the membrane of the nanoparticle, and a nucleic acid. FIG. 1I shows a nanoparticle comprising small molecule enhancers of protein expression bound or conjugated to the outside of the membrane and a nucleic acid. FIGS. 1J and 1Q show a nanoparticle having a cationic lipid membrane, a liquid oil core, inorganic nanoparticles, and a nucleic acid. FIGS. 1K and 1R show an oil-in-water emulsion with a nanoparticle having a cationic lipid membrane, a liquid oil core, small molecule enhancers of protein expression within the membrane of the nanoparticle, and a nucleic acid. FIGS. 1L and 1S show a nanoparticle comprising a cationic lipid membrane, a liquid oil core, inorganic nanoparticles and small molecule enhancers of protein expression within the membrane of the nanoparticle, and a nucleic acid. FIGS. 1M and 1T show a nanoparticle comprising a cationic lipid membrane, a liquid oil core, inorganic nanoparticles within the membrane of the nanoparticle, small molecule enhancers of protein expression bound or conjugated to the inside of the membrane, and a nucleic acid. FIGS. 1N and 1U show a nanoparticle comprising a cationic lipid membrane, a liquid oil core, inorganic nanoparticles within the membrane of the nanoparticle, small molecule enhancers of protein expression bound or conjugated to the outside of the membrane, and a nucleic acid. FIGS. 1O and 1V show a nanoparticle comprising a cationic lipid membrane, a liquid oil core, small molecule enhancers of protein expression bound or conjugated to the inside of the membrane, and a nucleic acid. FIGS. 1P and 1W show a nanoparticle comprising a cationic lipid membrane, a liquid oil core, small molecule enhancers of protein expression bound or conjugated to the outside of the membrane, and a nucleic acid. Schematics are not to scale.

FIG. 2 shows the time measurements of nanoparticle size as measured by dynamic light scattering (DLS). X axis is weeks and Y axis is nm diameter. Three-time courses correspond to storage at 4, 25, and 42 degrees Celsius.

FIG. 3 shows a graph showing that mRNA dose-dependent nLuc expression is achieved in A549-Dual cells transfected with NP-1+mRNA-nLuc at N:P ratio of 15. The X axis is the nanograms of RNA/well and the Y axis is nLuc expression (log₁₀ RLUs).

FIG. 4A shows a graph showing that nLuc expression in the supernatant of A549-Dual cells transfected with 20 ng of a nanoparticle (NP)-formulated mRNA-nLuc per well measured using the Nano-GLO® Luciferase kit (Promega cat #N1110). Cells received dilution series of a neutralizing antibody cocktail targeting both human type I IFNs and IFN receptors (PBL Assay Science), referred to as “antibody” in the legend. To confirm antibody activity, nLuc and IFIT responses were measured both with (antibody+IFN) and without (antibody only) human IFN-alpha (IFN). Horizontal lines label reference levels in untreated cells (Media), cells stimulated with human IFN-alpha alone (IFN only), cells transfected with NP-1+mRNA-nLuc with IFN but without the neutralizing antibody (RNA+IFN), and finally cells transfected with NP-1+RNA-nLuc alone (RNA only).

FIG. 4B shows that in the same supernatants as described above in the description for FIG. 4A, IFIT activity was measured by assaying for Lucia luciferase using the QUANTI-LucTM kit (Invivogen). Horizontal lines label reference levels in untreated cells (Media), cells stimulated with human IFN-alpha alone (IFN only), cells transfected with NP+mRNA-nLuc with IFN but without the neutralizing antibody (RNA+IFN), and finally cells transfected with NP-1+mRNA-nLuc alone (RNA only).

FIG. 5 shows the compound down selection strategy for the identification of compounds that enhance mRNA-encoded protein expression.

FIG. 6 shows a graph summarizing the number of compounds that upregulated nLuc expression above the mean expression level in cells transfected with NP-1+repRNA-nLuc alone based on their target pathways (N=92).

FIGS. 7A-7C are graphs of relative light units (RLU) (Y axis) measured for various injection conditions of DNA or RNA mixed with various nanoparticle conditions, at days 4, 6 and 8 post inoculation.

FIGS. 8A-8C are graphs of relative light units (RLU) (Y axis) measured for various injection conditions of DNA or RNA mixed with various nanoparticle conditions, at days 4, 6 and 8 post inoculation.

FIGS. 9A-9B shows scatterplot matrix showing pairwise correlations. FIG. 9A shows scatterplot matrix of pairwise correlation between responses for the full library screen with 99 compounds identified as hits (<100-fold enhancement in nLuc expression over RNA+IFN) marked in dark gray. FIG. 9B shows scatterplot matrix of just the hits with the 32 CDK targeting inhibitors marked as open circles.

FIG. 10 shows potency (EC50; μM) and magnitude of nLuc expression enhancement over RNA+IFN treated cells for candidate compounds.

FIGS. 11A-11B shows in vitro and in vivo activity of candidate inhibitor compounds after formulation in nanoparticle emulsion. FIG. 11A shows protein (nLuc) expression in IFN-treated A549-Dual cells after delivery with inhibitors formulated in nanoparticles compared with empty nanoparticles (no inhibitor) in the presence and absence of IFN. FIG. 11B shows SEAP expression in C57BL/6 mice after IM injection with repRNA-SEAP formulated with inhibitors formulated in nanoparticles or empty nanoparticles. Positive control for enhancement included a group with systemic anti-IFNAR-1 blockade one day prior to IM injection with repRNA. Statistical significance determined by ordinary one-way ANOVA and comparing all groups with the “Empty nanoparticles-anti-IFN.

FIGS. 12A-12E shows IM administration of NP-1/repRNA-SEAP encapsulating CDK inhibitors. FIG. 12A shows total SEAP expression for each of the dose levels compared to no compound group. FIG. 12B-12E shows SEAP expression levels in serum shown as a function of days after IM injection. Statistical analysis in FIG. 12A was performed on log10 transformed data using 2way ANOVA and Dunnett's multiple comparisons test.

FIGS. 13A-13B shows systemic administration of NP-1/repRNA-ZIKV-117 encapsulating CDK inhibitors. FIG. 13A shows serum concentration of ZIKV-117 as a function of days after NP-1/repRNA injection by IM route, and FIG. 13B shows total expression in compound of anti-IFNAR-1 treated groups compared to no compound group. Statistical analysis in FIG. 13B was performed on untransformed data using ordinary one-way ANOVA and Dunnett's multiple comparisons test. P-values: *<0.05, **<0.005.

FIGS. 14A-14H shows expression of nLuc in A549-Dual cells doped with CDK inhibitor were transfected with NP-35 nanoparticles and repRNA. FIGS. 14A-14H show expression of nLuc in transfected cells doped with MC180295, CDKI-73, CDK-IN-2, LY2857785, Dinaciclib, CDK12-IN-3, AZD4573, and (±)-BAY-1251152 respectively at different concentrations.

FIGS. 15A-15C show Measure of (A) cell viability, (B) IFIT2 induction, and (C) NF-kB induction in A549-Dual cells respectively.

FIG. 16 shows Expression of nLuc in A549-Dual cells transfected with NP-35 nanoparticles formulated with repRNA as a function of RNA transfection amount [ng] per well. The cells were transfected with NP-35 co-encapsulating repRNA and Dinaciclib (triangles), or NP-35 co-encapsulating only repRNA (squares).

### DETAILED DESCRIPTION OF THE INVENTION

Provided herein are compositions, kits, methods, and uses thereof for treatment of various conditions. Briefly, further described herein are (1) nanoparticle carriers systems; (2) nucleic acids coding for proteins, antibodies, and RNA polymerases; (3) protein expression enhancer compounds; (4) combination compositions; (5) pharmaceutical compositions; (6) dosing; (7) administration; (8) therapeutic applications; and (9) kits. Further described herein are (1) nanoparticle carriers systems; (2) first nucleic acids coding for proteins, antibodies, and RNA polymerases; (3) second nucleic acids coding for expression enhancers; (4) combination compositions; (5) pharmaceutical compositions; (6) dosing; (7) administration; (8) therapeutic applications; and (9) kits.

Compositions provided herein provide several advantages over preceding therapeutic formulations such as a protective nanoparticle configuration for safe and efficient nucleic acid delivery, a self-replicating RNA polymerase for the translation of the nucleic acid, and compounds that enhance expression of a nucleic acid-encoded protein or antibody to therapeutic levels in a mammalian cell.

### Definitions

Throughout this disclosure, various embodiments can be presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 15%, 14%, 13%, 12%, 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.

The term “effective amount” or “therapeutically effective amount” refers to an amount that is sufficient to achieve or at least partially achieve the desired effect.

### Nanoparticle Carrier Systems

Provided herein are various compositions comprising a nanoparticle or a plurality of nanoparticles. Nanoparticles are also referred to herein as carriers or abbreviated as NPs. Nanoparticle provided herein may be an organic, inorganic, or a combination of inorganic and organic materials that are less than about 1 micrometer (μm) in diameter. In some embodiments, nanoparticles provided herein are used as a delivery system for a bioactive agent (e.g., a nucleic acid encoding a protein, antigen, antibody, expression enhancer, RNA polymerase, or functional fragment thereof as provided herein and/or a compound provided herein).

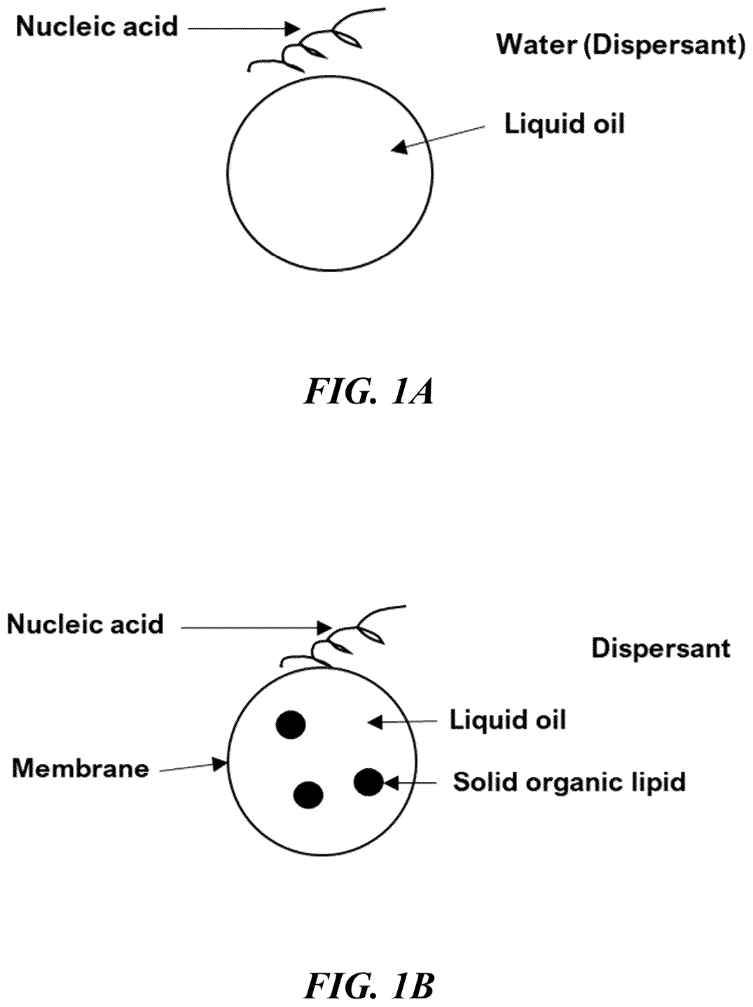

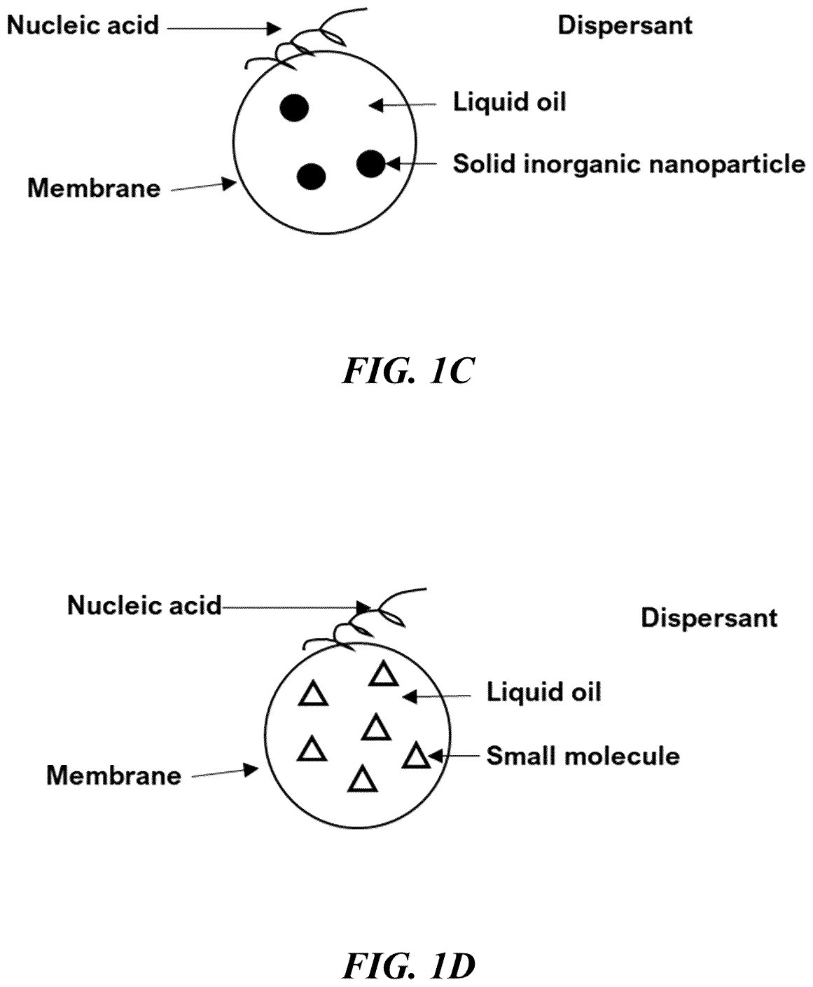

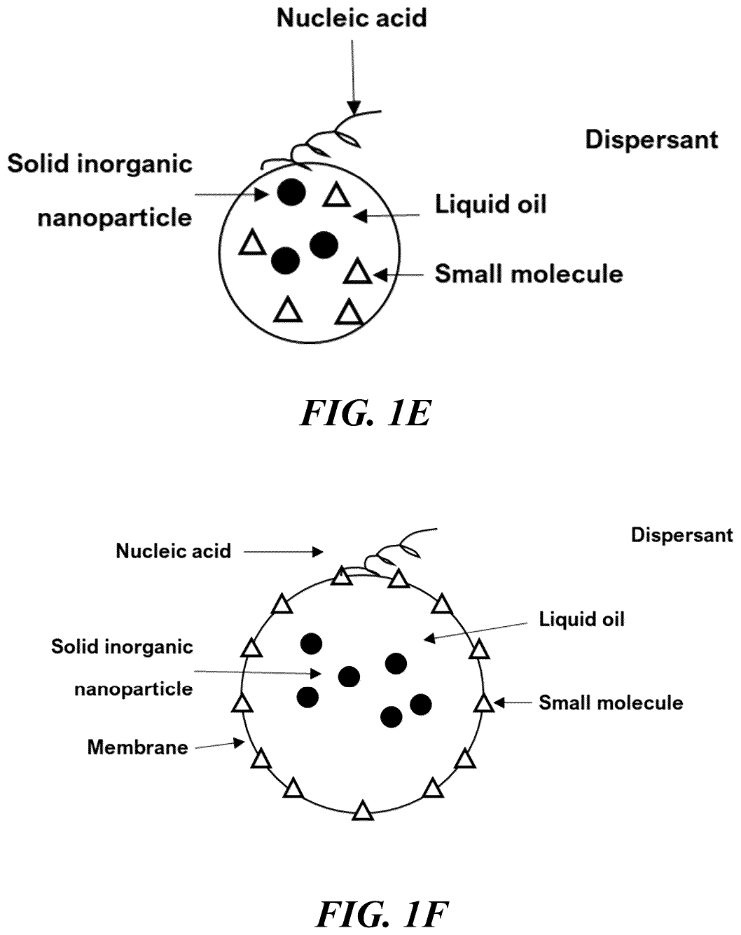

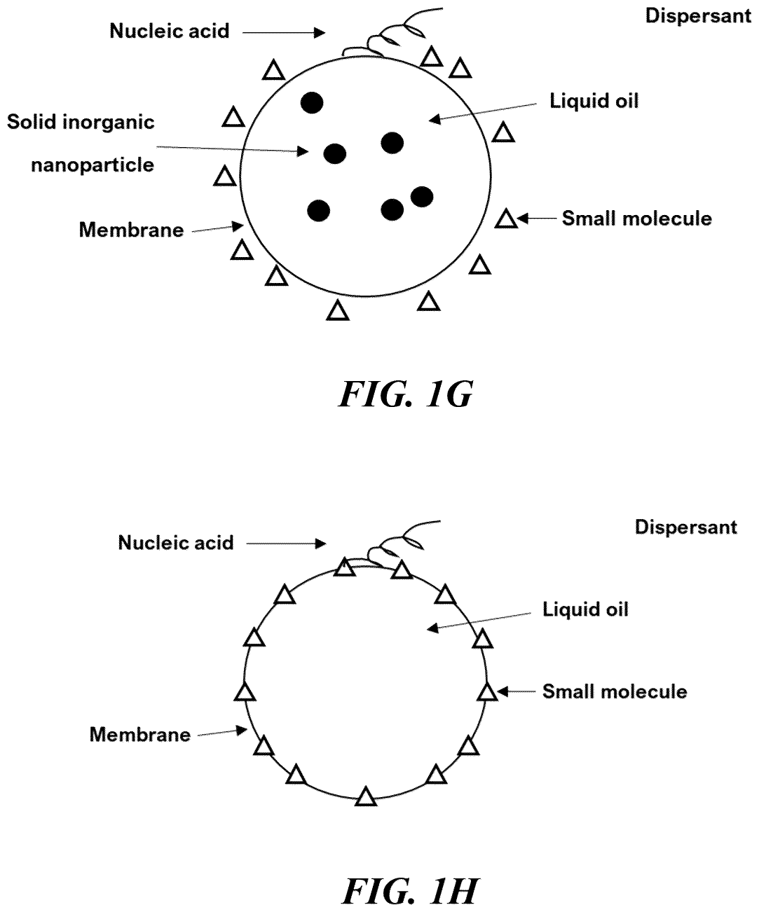

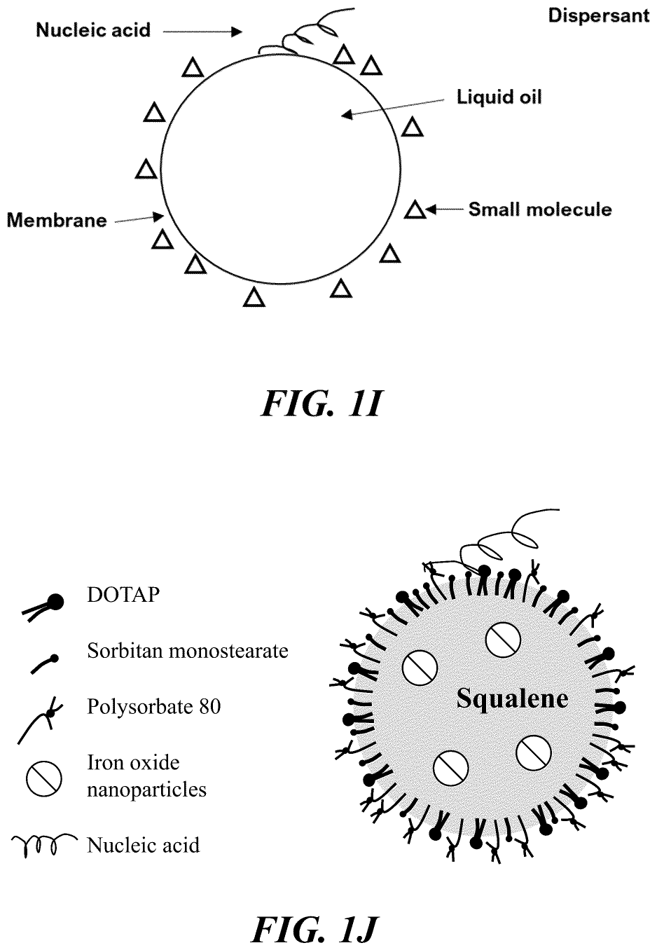

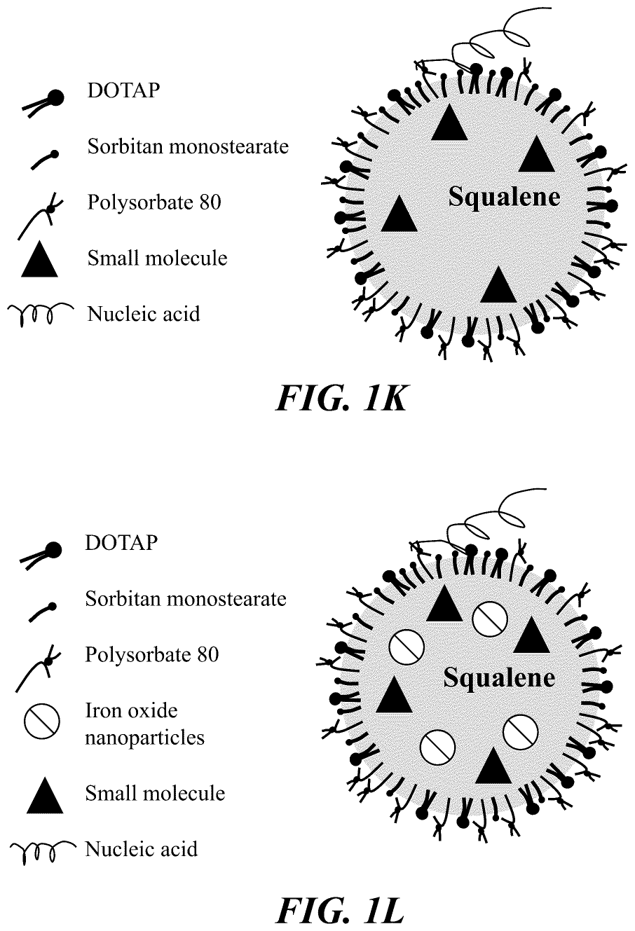

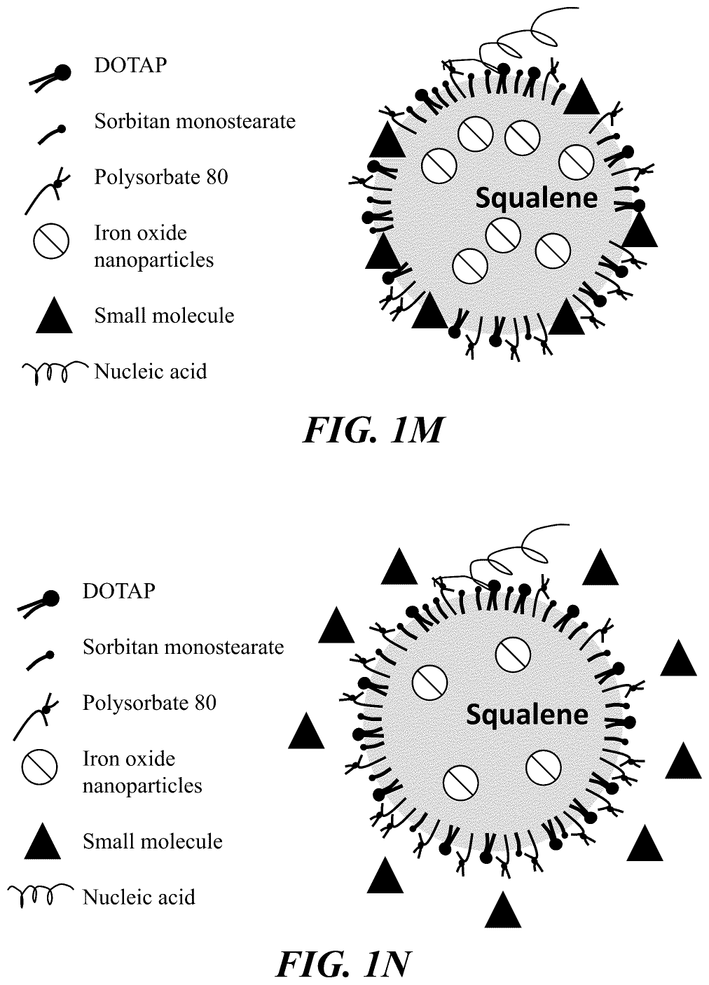

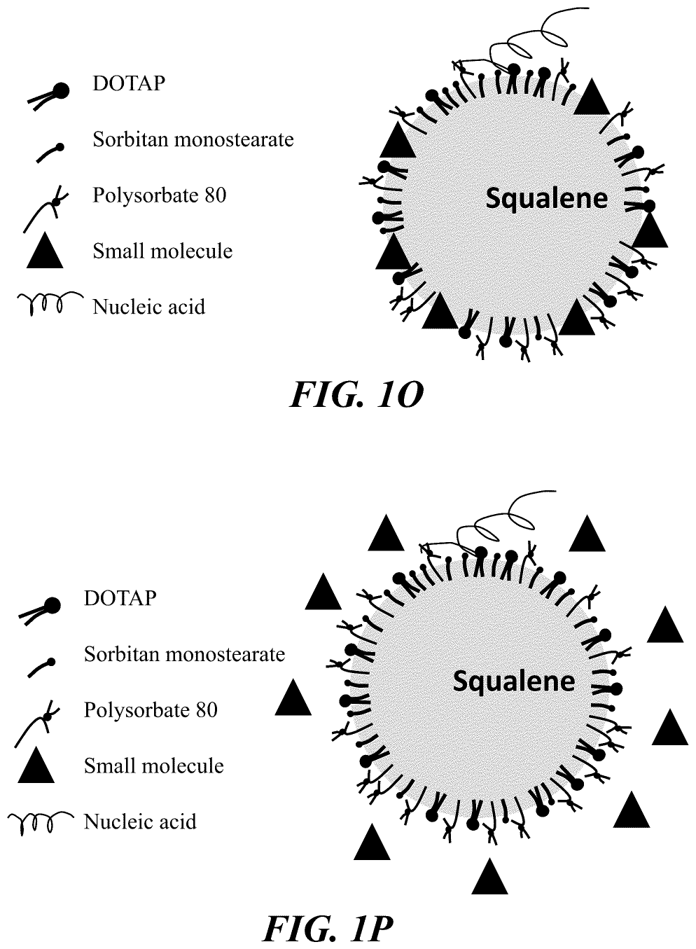

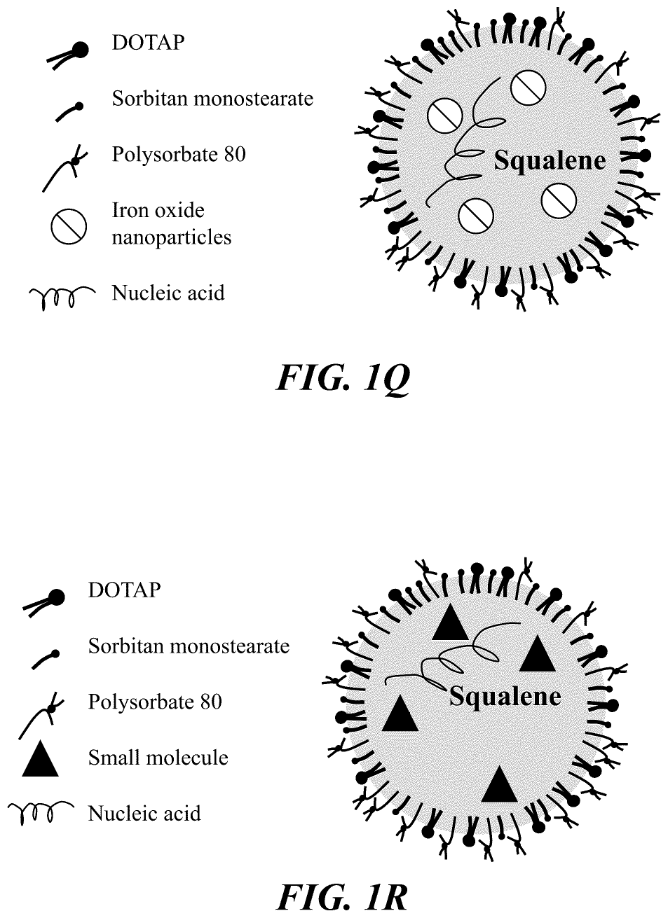

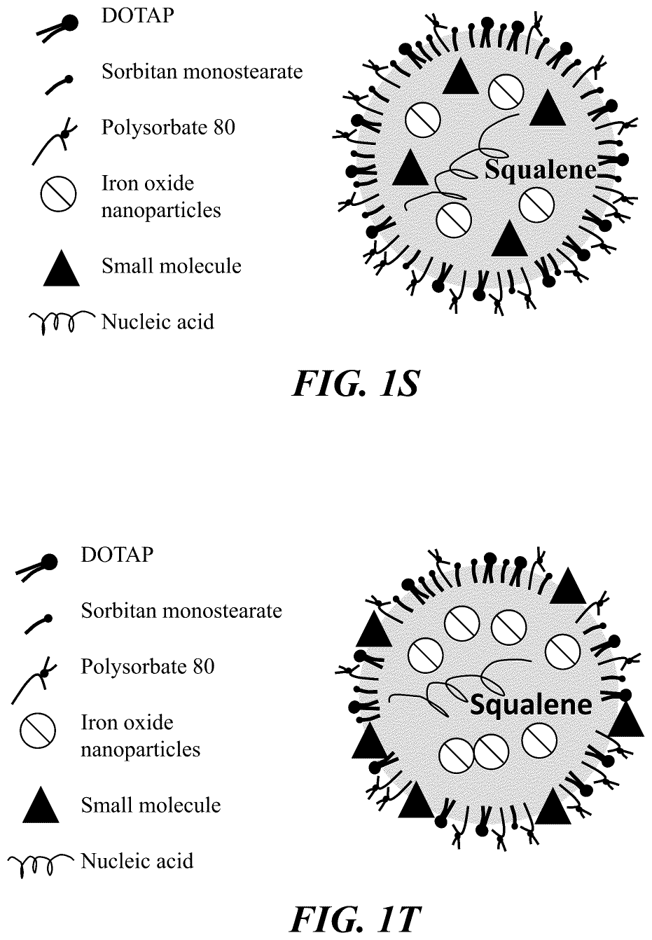

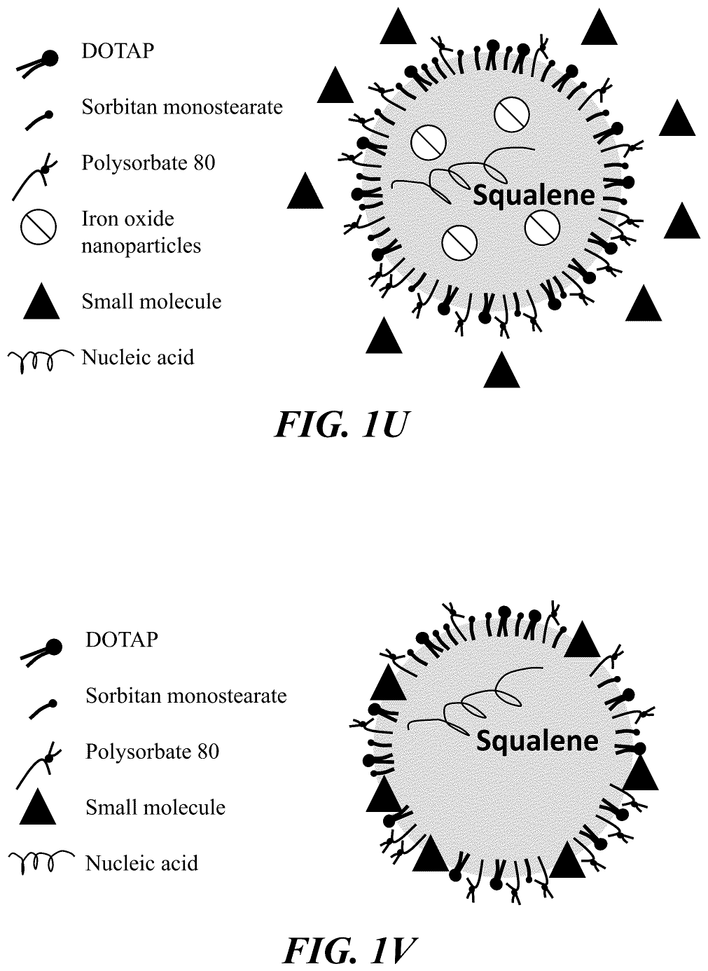

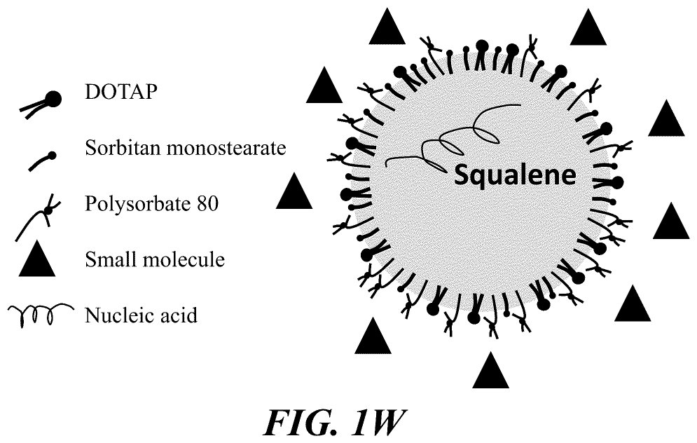

Various nanoparticles and formulations of nanoparticles (i.e., nanoemulsions) are employed. Exemplary nanoparticles are illustrated in FIGS. 1A-1W herein. Nanoparticles or carriers provided herein can include but are not limited to: oil in water emulsions, nanostructured lipid carriers (NLCs), cationic nanoemulsions (CNEs), vesicular phospholipid gels (VPG), polymeric nanoparticles, cationic lipid nanoparticles, liposomes, gold nanoparticles, solid lipid nanoparticles (LNPs or SLNs), mixed phase core NLCs, ionizable lipid carriers, magnetic carriers, polyethylene glycol (PEG)-functionalized carriers, cholesterol-functionalized carriers, polylactic acid (PLA)-functionalized carriers, and polylactic-co-glycolic acid (PLGA)-functionalized lipid carriers.

Oil in water emulsions, as illustrated in FIG. 1A (not to scale), are stable, immiscible fluids containing an oil droplet dispersed in water or aqueous phase. FIG. 1B (not to scale) illustrates a nanostructured lipid carrier (NLCs) which can comprise a blend of solid organic lipids (e.g., trimyristin) and liquid oil (e.g., squalene). In NLCs, the solid lipid is dispersed in the liquid oil. The entire nanodroplet is dispersed in the aqueous (water) phase. In some embodiments, the nanoparticle comprises inorganic nanoparticles, as illustrated in FIG. 1C (not to scale), as solid inorganic nanoparticles (e.g., iron oxide nanoparticles) dispersed in liquid oil. The entire nanodroplet is then dispersed as a colloid in the aqueous (water) phase. In some embodiments, the nanoparticles provided herein are dispersed in an aqueous solution. Non-limiting examples of aqueous solutions include water (e.g., sterilized, distilled, deionized, ultra-pure, RNAse-free, etc.), saline solutions (e.g., Kreb's, Ascaris, Dent's, Tet's saline), or 1% (w/v) dimethyl sulfoxide (DMSO) in water.

In some embodiments, the nanoparticles provided herein comprise a compound. Provided herein are compounds that are dispersed/dissolved within a liquid core of the nanoparticle, as illustrated in FIG. 1D (not to scale). Provided herein are nanoparticles comprising solid inorganic nanoparticles and compounds that are dispersed/dissolved within the liquid oil core, as illustrated in FIG. 1E (not to scale). In alternative embodiments, the compounds are within the membrane as illustrated in FIG. 1F and FIG. 1H (not to scale), the compounds are bound to the surface as illustrated in FIG. 1G and FIG. 1I (not to scale), or dispersed/dissolved within the liquid core (FIG. 1D). FIG. 1J-1W (not to scale), illustrates an exemplary embodiment, wherein the lipid carrier comprises a membrane and a liquid oil core. In some embodiments, the membrane comprises a blend of lipid and surfactant. In some embodiments, the lipid comprises DOTAP. In some embodiments, the surfactant comprises a blend of sorbitan monostearate, and Polysorbate 80. In some embodiments, the liquid oil core comprises squalene. In some embodiments, as illustrated in FIGS. 1J and 1Q (not to scale), the nanoparticle comprises iron oxide nanoparticles dispersed in a liquid oil (e.g., squalene). The entire nanodroplet can be then dispersed as a colloid in the aqueous (water) phase. In some embodiments, the nanoparticles provided herein are dispersed in an aqueous solution. Non-limiting examples of aqueous solutions include water (e.g., sterilized, distilled, deionized, ultra-pure, RNAse-free, etc.), saline solutions (e.g., Kreb's, Ascaris, Dent's, Tet's saline), or 1% (w/v) dimethyl sulfoxide (DMSO) in water.

In some embodiments, the nanoparticles provided herein comprise a small molecule. Provided herein are small molecules that are dispersed/dissolved in a liquid oil (e.g., squalene), as illustrated in FIGS. 1K and 1R (not to scale). Provided herein are nanoparticles comprising solid iron oxide nanoparticles and small molecules that are dispersed/dissolved in a liquid oil (e.g., squalene), as illustrated in FIGS. 1L and 1S (not to scale). In alternative embodiments, the small molecules are within the membrane as illustrated in FIGS. 1M, 1O, 1T, and 1V (not to scale), the small molecules are bound to the surface as illustrated in FIGS. 1N, 1P, 1U, and 1W (not to scale), or dispersed/dissolved a liquid oil (e.g., squalene) (FIG. 1D).

In some embodiments, the nanoparticles provided herein comprise a hydrophilic surface. In some embodiments, the hydrophilic surface comprises a cationic lipid. In some embodiments, the hydrophilic surface comprises an ionizable lipid. In some embodiments, the nanoparticle comprises a membrane. In some embodiments, the membrane comprises a cationic lipid. In some embodiments, the nanoparticles provided herein comprise a cationic lipid. Exemplary cationic lipids for inclusion in the hydrophilic surface include, without limitation: 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP), dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Other examples for suitable classes of lipids include, but are not limited to, the phosphatidylcholines (PCs), phosphatidylethanolamines (PEs), phosphatidylglycerol (PGs); and PEGylated lipids including PEGylated version of any of the above lipids (e.g., DSPE-PEGs). In some embodiments, the nanoparticle provided herein comprises DOTAP.

In some embodiments, the nanoparticle provided herein comprises an oil. In some embodiments, the oil is in liquid phase. Non-limiting examples of oils that can be used include α-tocopherol, coconut oil, dihydroisosqualene (DHIS), farnasene, grapeseed oil, lauroyl polyoxylglyceride, mineral oil, monoacylglycerol, palmkernal oil, olive oil, paraffin oil, peanut oil, propolis, squalene, squalane, solanesol, soy lecithin, soybean oil, sunflower oil, a triglyceride, or vitamin E. In some embodiments, the nanoparticle provided herein comprises a triglyceride. Exemplary triglycerides include but are not limited to: capric triglycerides, caprylic triglycerides, a caprylic and capric triglycerides, triglyceride esters, and myristic acid triglycerins.

In some embodiments, the nanoparticles provided herein comprise a liquid organic material and a solid inorganic material. In some embodiments, the nanoparticle provided herein comprises an inorganic particle. In some embodiments, the inorganic particle is a solid inorganic particle. In some embodiments, the nanoparticle provided herein comprises the inorganic particle within the hydrophobic core. In some embodiments, the nanoparticle provided herein comprises a metal. In some embodiments, the nanoparticle provided herein comprises a metal within the hydrophobic core. The metal can be without limitation, a metal salt, a metal oxide, a metal hydroxide, or a metal phosphate. In some embodiments, the nanoparticle provided herein comprises aluminum oxide (Al₂O₃), aluminum oxyhydroxide, iron oxide (Fe₃O₄, Fe₂O₃, FeO, or combinations thereof), titanium dioxide, silicon dioxide (SiO₂), aluminum hydroxyphosphate (Al(OH)ₓ(PO₄)_{y}), calcium phosphate (Ca₃(PO₄)₂), calcium hydroxyapatite (Ca₁₀(PO₄)₆(OH)₂), iron gluconate, or iron sulfate. The inorganic particles may be formed from one or more same or different metals (any metals including transition metal).

In some embodiments, the inorganic particle is a transition metal oxide. In some embodiments, the transition metal is magnetite (Fe₃O₄), maghemite (y-Fe₂O₃), wüstite (FeO), or hematite (alpha (α)-Fe₂O₃).

In some embodiments, the metal is aluminum hydroxide or aluminum oxyhydroxide, and a phosphate-terminated lipid or a surfactant, such as oleic acid, oleylamine, SDS, TOPO or DSPA is used to coat the inorganic solid nanoparticle, before it is mixed with the liquid oil to form the hydrophobic core.

In some embodiments, the metal can comprise a paramagnetic, a superparamagnetic, a ferrimagnetic or a ferromagnetic compound. In some embodiments, the metal is a superparamagnetic iron oxide (Fe₃O₄).

In some embodiments, the nanoparticle provided herein comprises a cationic lipid, and an oil. In some embodiments, the nanoparticle provided herein comprises DOTAP; and squalene and/or glyceryl trimyristate-dynasan.

In some embodiments, the nanoparticle provided herein comprises a cationic lipid, an oil, and an inorganic particle. In some embodiments, the nanoparticle provided herein comprises DOTAP; squalene and/or glyceryl trimyristate-dynasan; and iron oxide.

In some embodiments, the nanoparticle provided herein further comprises a surfactant. Thus, in some embodiments, the nanoparticles provided herein comprise a cationic lipid, an oil, an inorganic particle, and a surfactant. In some embodiments, the nanoparticles provided herein comprise a cationic lipid, an oil, and a surfactant

Surfactants are compounds that lower the surface tension between two liquids or between a liquid and a solid component of the nanoparticles provided herein. Surfactants can be hydrophobic, hydrophilic, or amphiphilic. In some embodiments, the nanoparticle provided herein comprises a hydrophobic surfactant. Exemplary hydrophobic surfactants that can be employed include but are not limited to: sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), and sorbitan trioleate (SPAN® 85). Suitable hydrophobic surfactants include those having a hydrophilic-lipophilic balance (HLB) value of 10 or less, for instance, 5 or less, from 1 to 5, or from 4 to 5. For instance, the hydrophobic surfactant can be a sorbitan ester having a HLB value from 1 to 5, or from 4 to 5.

In some embodiments, the nanoparticle provided herein comprises a hydrophilic surfactant, also called an emulsifier. In some embodiments, the nanoparticle provided herein comprises polysorbate. Polysorbates are oily liquids derived from ethoxylated sorbitan (a derivative of sorbitol) esterified with fatty acids. Exemplary hydrophilic surfactants that can be employed include but are not limited to: polysorbates such as Tween, Kolliphor, Scattics, Alkest, or Canarcel; polyoxyethylene sorbitan ester (polysorbate); polysorbate 80 (polyoxyethylene sorbitan monooleate, or Tween 80); polysorbate 60 (polyoxyethylene sorbitan monostearate, or Tween 60); polysorbate 40 (polyoxyethylene sorbitan monopalmitate, or Tween 40); and polysorbate 20 (polyoxyethylene sorbitan monolaurate, or Tween 20). In one embodiment, the hydrophilic surfactant is polysorbate 80.

Nanoparticles provided herein comprises a hydrophobic core surrounded by a lipid membrane (e.g., a cationic lipid such as DOTAP). In some embodiments, the hydrophobic core comprises: a phosphate-terminated lipid, a surfactant, or a combination thereof. In some embodiments, the hydrophobic core comprises: one or more inorganic particles; a phosphate-terminated lipid; and a surfactant.

Inorganic solid nanoparticles described herein may be surface modified before mixing with the liquid oil. For instance, if the surface of the inorganic solid nanoparticle is hydrophilic, the inorganic solid nanoparticle may be coated with hydrophobic molecules (or surfactants) to facilitate the miscibility of the inorganic solid nanoparticle with the liquid oil in the “oil” phase of the nanoemulsion particle.

In some embodiments, the inorganic particle is coated with a capping ligand, the phosphate-terminated lipid, and/or the surfactant.

In some embodiments the hydrophobic core comprises a phosphate-terminated lipid. Exemplary phosphate-terminated lipids that can be employed include but are not limited to: trioctylphosphine oxide (TOPO) or distearyl phosphatidic acid (DSPA).

In some embodiments, the hydrophobic core comprises a surfactant, wherein the surfactant is a phosphorous-terminated surfactant, a carboxylate-terminated surfactant, a sulfate-terminated surfactant, or an amine-terminated surfactant. Typical carboxylate-terminated surfactants include oleic acid. Typical amine terminated surfactants include oleylamine. In some embodiments, the surfactant is distearyl phosphatidic acid (DSPA), oleic acid, oleylamine or sodium dodecyl sulfate (SDS).

In some embodiments, the inorganic solid nanoparticle is a metal oxide such as an iron oxide, and a surfactant, such as oleic acid, oleylamine, SDS, DSPA, or TOPO, is used to coat the inorganic solid nanoparticle, before it is mixed with the liquid oil to form the hydrophobic core.

In some embodiments, the hydrophobic core comprises: a cationic lipid comprising DOTAP; a hydrophobic surfactant comprising a sorbitan ester (e.g., sorbitan monostearate, sorbitan monooleate, sorbitan trioleate, or a combination thereof); and a hydrophilic surfactant comprising a polysorbate (e.g., polysorbate 80). In some embodiments, the hydrophobic core further comprises one or more of a phosphate-terminated lipid (e.g., TOPO), a surfactant (e.g., a phosphorous-terminated surfactant, a carboxylate-terminated surfactant, a sulfate-terminated surfactant, an amine-terminated surfactant, or a combination thereof), and a liquid oil containing naturally occurring or synthetic squalene.

In some embodiments, the hydrophobic core comprises: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core comprises: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the hydrophobic core consists of: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core consists of: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the nanoparticle provided herein can comprise from about 0.2% to about 40% w/v squalene, from about 0.2% to about 10% w/v DOTAP, from about 0.25% to about 5% w/v sorbitan monostearate, and from about 0.5% to about 10% w/v polysorbate 80. In some embodiments, the nanoparticle provided herein can comprise from about 0.2% to about 40% w/v squalene, from about 0.001% to about 10% w/v iron oxide nanoparticles, from about 0.2% to about 10% w/v DOTAP, from about 0.25% to about 5% w/v sorbitan monostearate, and from about 0.5% to about 10% w/v polysorbate 80.

In some embodiments the nanoparticle provided herein can comprise from about 2% to about 6% w/v squalene, from about 0.2% to about 1% w/v DOTAP, from about 0.25% to about 1% w/v sorbitan monostearate, and from about 0.5%) to about 5% w/v polysorbate 80. In some embodiments the nanoparticle provided herein can comprise from about 2% to about 6% w/v squalene, from about 0.01% to about 1% w/v iron oxide nanoparticles, from about 0.2% to about 1% w/v DOTAP, from about 0.25% to about 1% w/v sorbitan monostearate, and from about 0.5%) to about 5% w/v polysorbate 80.

In some embodiments, the nanoparticle provided herein can comprise from about 0.2% to about 40% w/v squalene, from about 0.2% to about 10% w/v DOTAP, from about 0.25% to about 5% w/v sorbitan monostearate, and from about 0.5% to about 10% w/v polysorbate 80. In some embodiments, the nanoparticle provided herein can comprise from about 0.2% to about 40% w/v squalene, from about 0.001% to about 10% w/v aluminum hydroxide or aluminum oxyhydroxide nanoparticles, from about 0.2% to about 10% w/v DOTAP, from about 0.25% to about 5% w/v sorbitan monostearate, and from about 0.5% to about 10% w/v polysorbate 80.

In some embodiments, the nanoparticle provided herein can comprise from about 2% to about 6% w/v squalene, from about 0.2% to about 1% w/v DOTAP, from about 0.25% to about 1% w/v sorbitan monostearate, and from about 0.5%) to about 5% w/v polysorbate. In some embodiments, the nanoparticle provided herein can comprise from about 2% to about 6% w/v squalene, from about 0.01% to about 1% w/v aluminum hydroxide or aluminum oxyhydroxide nanoparticles, from about 0.2% to about 1% w/v DOTAP, from about 0.25% to about 1% w/v sorbitan monostearate, and from about 0.5%) to about 5% w/v polysorbate 80.

Exemplary nanoparticle formulations include any of the formulations provided in Table 1. In some embodiments, a composition described herein comprises any one of NP-1 to NP-30. In some embodiments, a composition described herein comprises any one of NP-1 to NP-35. In some embodiments, the nanoparticles provided herein comprises LNP (e.g., NP-33, NP-34, and NP-35). In some embodiments, the nanoparticles provided herein are admixed with a nucleic acid provided herein. In some embodiments, nanoparticles provided herein are made by homogenization and ultrasonication techniques.

TABLE 1
Nanoparticle Formulations

| | | | | Additional |
|---|---|---|---|---|
| | | | | Ingredients |
| | Cationic Lipid(s) | Oil(s) | Surfactant(s) | % (w/v), mg/ml, |
| Name | % (w/v) or mg/ml | % (w/v) or mg/ml | %(w/v) or mg/ml | or mM |
| NP-1 | 30 mg/ml 1,2- | 37.5 mg/ml | 37 mg/ml sorbitan | 0.2 mg Fe/ml 12 |
| | dioleoyl-3- | squalene | monostearate, (2R)-2- | nm oleic acid- |
| | trimethylammonium- | | [(2R,3R,4S)-3,4- | coated iron oxide |
| | propane (DOTAP) | | Dihydroxyoxolan-2-yl]-2- | nanoparticles |
| | chloride | | hydroxyethyl octadecenoate, | 10 mM sodium |
| | | | C₂₄H₄₆O₆) (SPAN ® 60) | citrate dihydrate. |
| | | | 37 mg/ml polyoxyethylene | |
| | | | (20) sorbitan monooleate, | |
| | | | C₆₄H₁₂₄O₂₆, Polysorbate 80 | |
| | | | (TWEEN 80 ®) | |
| NP-2 | 30 mg/ml 1,2- | 37.5 mg/ml | 37 mg/ml sorbitan | 1 mg Fe/ml 15 |
| | dioleoyl-3- | squalene | monostearate, (2R)-2- | nm oleic acid- |
| | trimethylammonium- | | [(2R,3R,4S)-3,4- | coated iron oxide |
| | propane (DOTAP) | | Dihydroxyoxolan-2-yl]-2- | nanoparticles |
| | chloride | | hydroxyethyl octadecenoate, | 10 mM sodium |
| | | | C₂₄H₄₆O₆) (SPAN ® 60) | citrate dihydrate. |
| | | | 37 mg/ml polyoxyethylene | |
| | | | (20) sorbitan monooleate, | |
| | | | C₆₄H₁₂₄O₂₆, Polysorbate 80 | |
| | | | (TWEEN 80 ®) | |
| NP-3 | 30 mg/ml 1,2- | 37.5 mg/ml | 37 mg/ml sorbitan | 0.2 mg Fe/ml 15 |
| | dioleoyl-3- | Miglyol 812N | monostearate, (2R)-2- | nm oleic acid- |
| | trimethylammonium- | (triglyceride ester | [(2R,3R,4S)-3,4- | coated iron oxide |
| | propane (DOTAP) | of saturated | Dihydroxyoxolan-2-yl]-2- | nanoparticles |
| | chloride | coconut/palmkern | hydroxyethyl octadecenoate, | 10 mM sodium |
| | | el oil derived | C₂₄H₄₆O₆) (SPAN ® 60) | citrate dihydrate. |
| | | caprylic and | 37 mg/ml polyoxyethylene | |
| | | capric fatty acids | (20) sorbitan monooleate, | |
| | | and plant derived | C₆₄H₁₂₄O₂₆, Polysorbate 80 | |
| | | glycerol) | (TWEEN 80 ®) | |
| NP-4 | 30 mg/ml 1,2- | 37.5 mg/ml | 37 mg/ml sorbitan | 1 mg Fe/ml 15 |
| | dioleoyl-3- | Miglyol 812N | monostearate, (2R)-2- | nm oleic acid- |
| | trimethylammonium- | (triglyceride ester | [(2R,3R,4S)-3,4- | coated iron oxide |
| | propane (DOTAP) | of saturated | Dihydroxyoxolan-2-yl]-2- | nanoparticles |
| | chloride | coconut/palmkern | hydroxyethyl octadecenoate, | 10 mM sodium |
| | | el oil derived | C₂₄H₄₆O₆) (SPAN ® 60) | citrate dihydrate. |
| | | caprylic and | 37 mg/ml polyoxyethylene | |
| | | capric fatty acids | (20) sorbitan monooleate, | |
| | | and plant derived | C₆₄H₁₂₄O₂₆, Polysorbate 80 | |
| | | glycerol) | (TWEEN 80 ®) | |
| NP-5 | 30 mg/ml DOTAP | 37.5 mg/ml | 37 mg/ml sorbitan | 1 mg/ml |
| | chloride | squalene | monostearate (SPAN ® 60) | trioctylphosphine |
| | | | 37 mg/ml polysorbate 80 | oxide (TOPO)- |
| | | | (TWEEN ® 80) | coated aluminum |
| | | | | hydroxide |
| | | | | (Alhydrogel ® |
| | | | | 2%) particles |
| | | | | 10 mM sodium |
| | | | | citrate dihydrate. |
| NP-6 | 30 mg/ml DOTAP | 37.5 mg/ml | 37 mg/ml sorbitan | 0.2 mg Fe/ml |
| | chloride | Solanesol | monostearate (SPAN ® 60) | oleic acid-coated |
| | | (Cayman | 37 mg/ml polysorbate 80 | iron oxide |
| | | chemicals), | (TWEEN ® 80) | nanoparticles |
| | | | | 10 mM sodium |
| | | | | citrate |
| NP-7 | 30 mg/ml DOTAP | 37.5 mg/ml | 37 mg/ml sorbitan | 10 mM sodium |
| | chloride | squalene | monostearate (SPAN ® 60) | citrate |
| | | 2.4 mg/ml | 37 mg/ml polysorbate 80 | |
| | | Dynasan 114 | (TWEEN ® 80 | |
| NP-8 | 4 mg/ml DOTAP | 43 mg/ml | 5 mg/ml Span ® 85 | 10 mM sodium |
| | chloride | squalene | 5 mg/ml Tween ® 80 | citrate |
| NP-9 | 7.5 mg/ml 1,2- | 9.4 mg/ml | 9.3 mg/ml sorbitan | 0.05 mg/ml 15 |
| | dioleoyl-3- | squalene | monostearate, (2R)-2- | nanometer |
| | trimethylammonium- | ((6E,10E,14E,18E)- | [(2R,3R,4S)-3,4- | superparamagnetic |
| | propane (DOTAP) | 2,6,10,15,19,23- | Dihydroxyoxolan-2-yl]-2- | iron oxide |
| | chloride | Hexamethyltetracosa- | hydroxyethyl octadecenoate, | (Fe₃O₄) |
| | | 2,6,10,14,18,22- | C₂₄H₄₆O₆) (SPAN ® 60) | 10 mM sodium |
| | | hexaene, C₃₀H₅₀) | 9.3 mg/ml polyoxyethylene | citrate dihydrate |
| | | 0.63 mg/ml | (20) sorbitan monooleate, | |
| | | glyceryl | C₆₄H₁₂₄O₂₆, Polysorbate 80 | |
| | | trimyristate- | (TWEEN 80 ®) | |
| | | dynasan | | |
| | | (DYNASAN | | |
| | | 114 ®) | | |
| NP-10 | 0.4% DOTAP | 0.25% glyceryl | 0.5% sorbitan monostearate | |
| | | trimyristate- | (SPAN ® 60) | |
| | | dynasan | 0.5% polysorbate 80 | |
| | | (DYNASAN | (TWEEN 80 ®) | |
| | | 114 ®) | | |
| | | 4.75% Squalene | | |
| NP-11 | 3.0% DOTAP | 0.25% glyceryl | 3.7% sorbitan monostearate | |
| | | trimyristate- | (SPAN ® 60) | |
| | | dynasan | | |
| | | (DYNASAN | 3.7% polysorbate 80 | |
| | | 114 ®) | (TWEEN 80 ®) | |
| | | 3.75% Squalene | | |
| NP-12 | 0.4% DOTAP | 4.3% Squalene | 0.5% sorbitan trioleate | |
| | | | (SPAN ® 85) | |
| | | | 0.5% polysorbate 80 | |
| | | | (TWEEN ® 80) | |
| NP-13 | 0.4% DOTAP | 0.25% glyceryl | 2.0% polysorbate 80 | |
| | | trimyristate- | (TWEEN ® 80) | |
| | | dynasan | | |
| | | (DYNASAN | | |
| | | 114 ®) | | |
| | | 4.08% squalene | | |
| NP-14 | 0.4% DOTAP | 0.25% glyceryl | 0.5% sorbitan trioleate | |
| | | trimyristate- | (SPAN ® 85) | |
| | | dynasan | 2.0% polysorbate 80 | |
| | | (DYNASAN | (TWEEN ® 80) | |
| | | 114 ®) | | |
| | | 4.08% squalene | | |
| NP-15 | 0.4% DOTAP | 0.25% glyceryl | 0.25% sorbitan trioleate | |
| | | trimyristate- | (SPAN ® 85) | |
| | | dynasan | 2.0% polysorbate 80 | |
| | | (DYNASAN | (TWEEN ® 80) | |
| | | 114 ®) | | |
| | | 4.08% squalene | | |
| NP-16 | 0.4% DOTAP | 5% squalene | 0.5% sorbitan trioleate | |
| | | | (SPAN ® 85) | |
| | | | 2.0% polysorbate 80 | |
| | | | (TWEEN ® 80) | |
| NP-17 | 0.4% DOTAP | 5% squalene | 0.5% sorbitan monostearate | |
| | | | (SPAN ® 60) | |
| | | | 2% poly sorbate 80 | |
| | | | (TWEEN ® 80) | |
| NP-18 | 0.4% DOTAP | 0.25% glyceryl | 2% sorbitan trioleate | |
| | | trimyristate- | (SPAN ® 85) | |
| | | dynasan | 2% polysorbate 80 | |
| | | (DYNASAN | (TWEEN ® 80) | |
| | | 114 ®) | | |
| | | 4.08% squalene | | |
| NP-19 | 0.4% DOTAP | 0.25% glyceryl | 0.5% sorbitan monostearate | 1% aluminum |
| | | trimyristate- | (SPAN ® 60) | hydroxide |
| | | dynasan | 0.5% polysorbate 80 | |
| | | (DYNASAN | (TWEEN 80 ®) | |
| | | 114 ®) | | |
| | | 4.75% Squalene | | |
| NP-20 | 3.0% DOTAP | 0.25% glyceryl | 3.7% sorbitan monostearate | 1% aluminum |
| | | trimyristate- | (SPAN ® 60) | hydroxide |
| | | dynasan | 3.7% polysorbate 80 | |
| | | (DYNASAN | (TWEEN ® 80) | |
| | | 114 ®) | | |
| | | 3.75% Squalene | | |
| NP-21 | 0.4% DOTAP | 4.3% Squalene | 0.5% sorbitan trioleate | 1% aluminum |
| | | | (SPAN ® 85) | hydroxide |
| | | | 0.5% polysorbate 80 | |
| | | | (TWEEN ® 80 | |
| NP-22 | 0.4% DOTAP | 0.25% glyceryl | 2.0% polysorbate 80 | 1% aluminum |
| | | trimyristate- | (TWEEN ® 80) | hydroxide |
| | | dynasan | | |
| | | (DYNASAN | | |
| | | 114 ®) | | |
| | | 4.08% squalene | | |
| NP-23 | 0.4% DOTAP | 0.25% glyceryl | 0.5% sorbitan trioleate | 1% aluminum |
| | | trimyristate- | (SPAN ® 85) | hydroxide |
| | | dynasan | 2.0% polysorbate 80 | |
| | | (DYNASAN | (TWEEN ® 80 | |
| | | 114 ®) | | |
| | | 4.08% squalene | | |
| NP-24 | 0.4% DOTAP | 0.25% glyceryl | 0.25% sorbitan trioleate | 1% aluminum |
| | | trimyristate- | (SPAN ® 85) | hydroxide |
| | | dynasan | 2.0% polysorbate 80 | |
| | | (DYNASAN | (TWEEN ® 80 | |
| | | 114 ®) | | |
| | | 4.08% squalene | | |
| NP-25 | 0.4% DOTAP | 5% squalene | 0.5% sorbitan trioleate | 1% aluminum |
| | | | (SPAN ® 85) | hydroxide |
| | | | 2.0% polysorbate 80 | |
| | | | (TWEEN ® 80 | |
| NP-26 | 0.4% DOTAP | 5% squalene | 0.5% sorbitan monostearate | 1% aluminum |
| | | | (SPAN ® 60) | hydroxide |
| | | | 2% polysorbate 80 | |
| | | | (TWEEN ® 80) | |
| NP-27 | 0.4% DOTAP | 0.25% glyceryl | 2% sorbitan trioleate | 1% aluminum |
| | | trimyristate- | (SPAN ® 85) | hydroxide |
| | | dynasan | 2% polysorbate 80 | |
| | | (DYNASAN | (TWEEN ® 80) | |
| | | 114 ®) | | |
| | | 4.08% squalene | | |
| NP-28 | 0.5-5.0 mg/ml | 0.2-10% (v/v) | 0.01-2.5% (v/v) polysorbate | |
| | DOTAP | squalene | 80 (TWEEN ® 80) | |
| NP-29 | 0.4% (w/w) DOTAP | 4.3% (w/w) | 0.5% (w/w) sorbitan | |
| | | squalene | trioleate (SPAN ® 85) | |
| | | | 0.5% (w/w) polysorbate 80 | |
| | | | (TWEEN ® 80) | |
| NP-30 | 30 mg/ml DOTAP | 37.5 mg/ml | 37 mg/ml sorbitan | 10 mM sodium |
| | chloride | squalene | monostearate (SPAN ® 60) | citrate |
| | | | 37 mg/ml polysorbate 80 | |
| | | | (TWEEN ® 80) | |
| NP-31 | 30 mg/ml DOTAP | 37.5 mg/ml | 37 mg/ml sorbitan | 0.4 mg Fe/ml 5 |
| | chloride | squalene | monostearate (SPAN ® 60) | nm oleic acid- |
| | | | 37 mg/ml polysorbate 80 | coated iron oxide |
| | | | (TWEEN ® 80) | nanoparticles |
| | | | | 10 mM sodium |
| | | | | citrate dihydrate |
| NP-32 | 0.8-1.6 mg/ml | 4.5% squalene | 0.5% (w/w) sorbitan trioleate | 10 mM sodium |
| | DOTAP chloride | | (SPAN 85 ®) | citrate |
| | | | 0.5% (w/w) polysorbate 80 | |
| | | | (TWEEN ® 80) | |
| NP-33 | 45-55 mol % | 35-42 mol % | 1.25-1.75 mol % PEG2000- | |
| | ionizable cationic | cholesterol | DMG | |
| | lipid | | | |
| | 8-12 mol % | | | |
| | distearoylphosphatidyl- | | | |
| | choline (DSPC) | | | |
| NP-34 | 50 mol % D-Lin- | 38.5% cholesterol | 1.5% PEG-lipid | |
| | MC3-DMA (MC3) | | | |
| | 10 mol % | | | |
| | distearoylphosphatidyl- | | | |
| | choline (DSPC) | | | |
| NP-35 | 50 mol % Lipid H | 38.5% cholesterol | 1.5 mol % PEG2000-DMG | |
| | (SM-102) | | | |
| | 10 mol % | | | |
| | distearoylphosphatidyl- | | | |
| | choline (DSPC) | | | |

Nanoparticles provided herein can be of various average diameters in size. In some embodiments, nanoparticles provided herein have an average diameter (z-average hydrodynamic diameter, measured by dynamic light scattering) ranging from about 20 nm to about 200 nm. In some embodiments, the z-average diameter of the nanoparticle ranges from about 20 nm to about 150 nm, from about 20 nm to about 100 nm, from about 20 nm to about 80 nm, from about 20 nm to about 60 nm. In some embodiments, the z-average diameter of the nanoparticle ranges from about 40 nm to about 200 nm, from about 40 nm to about 150 nm, from about 40 nm to about 100 nm, from about 40 nm to about 90 nm, from about 40 nm to about 80 nm, or from about 40 nm to about 60 nm. In one embodiment, the z-average diameter of the nanoparticle is from about 40 nm to about 80 nm. In some embodiments, the z-average diameter of the nanoparticle is from about 40 nm to about 60 nm. In some embodiments, the nanoparticle is up to 200 nm in diameter. In some embodiments, the nanoparticle is 50 to 70 nm in diameter. In some embodiments, the nanoparticle is 40 to 80 nm in diameter. In some embodiments, the nanoparticle is 20 to 80 nm in diameter.

In some embodiments, the inorganic particle within the hydrophobic core of the nanoparticle can be an average diameter (number weighted average diameter) ranging from about 3 nm to about 50 nm. In some embodiments, the inorganic particle comprises an average diameter of about 5 nm, about 10 nm, about 15 nm, about 20 nm, about 25 nm, about 30 nm, about 35 nm, about 40 nm, about 45 nm, or about 50 nm.

Nanoparticles provided herein may be characterized by the polydispersity index (PDI), which is an indication of their quality with respect to size distribution. In some embodiments, the average polydispersity index (PDI) of the nanoparticles provided herein ranges from about 0.1 to about 0.5. In some embodiments, the average PDI of the nanoparticles can range from about 0.2 to about 0.5, from about 0.1 to about 0.4, from about 0.2 to about 0.4, from about 0.2 to about 0.3, or from about 0.1 to about 0.3.

In some embodiments, the nanoparticles provided herein comprise an oil-to-surfactant molar ratio ranging from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1.

In some embodiments, the nanoparticles provided herein comprise a hydrophilic surfactant-to-lipid ratio ranging from about 0.1:1 to about 2:1, from about 0.2:1 to about 1.5:1, from about 0.3:1 to about 1:1, from about 0.5:1 to about 1:1, or from about 0.6:1 to about 1:1. In some embodiments, the nanoparticles provided herein comprise a hydrophobic surfactant-to-lipid ratio ranging from about 0.1:1 to about 5:1, from about 0.2:1 to about 3:1, from about 0.3:1 to about 2:1, from about 0.5:1 to about 2:1, or from about 1:1 to about 2:1.

In some embodiments, the nanoparticles provided herein comprise from about 0.2% to about 40% w/v liquid oil, from about 0.2% to about 10% w/v lipid, from about 0.25% to about 5% w/v hydrophobic surfactant, and from about 0.5% to about 10% w/v hydrophilic surfactant. In some embodiments, the lipid comprises a cationic lipid, and the oil comprises squalene, and/or the hydrophobic surfactant comprises sorbitan ester. In some embodiments, the nanoparticles provided herein comprise from about 0.2% to about 40% w/v liquid oil, from about 0.001% to about 10% w/v inorganic solid nanoparticle, from about 0.2% to about 10% w/v lipid, from about 0.25% to about 5% w/v hydrophobic surfactant, and from about 0.5% to about 10% w/v hydrophilic surfactant. In some embodiments, the lipid comprises a cationic lipid, and the oil comprises squalene, and/or the hydrophobic surfactant comprises sorbitan ester.

### Nucleic Acids

Provided herein is a composition comprising a nucleic acid. Provided herein is a composition comprising a nucleic acid coding for a protein, an antibody, or a functional fragment thereof. In some embodiments, the nucleic acid is in complex with the nanoparticle. In some embodiments, the nucleic acid is in complex with the membrane of the nanoparticle. In some embodiments, the nucleic acid is in complex with the hydrophilic surface of the nanoparticle. For example, FIGS. 1A-1P (not to scale) illustrates the nucleic acid is bound to the hydrophilic surface of the nanoparticle. In some embodiments, the nucleic acid is within the nanoparticle. In some embodiments, the nucleic acid is within the hydrophobic core. For example, FIGS. 1Q-1W (not to scale) illustrates an exemplary nanoparticle, wherein the nucleic acid is dispersed in the hydrophobic core (e.g., squalene oil). In some embodiments, the nucleic acid is in complex with the hydrophobic surface of the membrane. In some embodiments, the nucleic acid is in complex with the hydrophilic surface of the membrane. In some embodiments, the nucleic acid is in complex with the hydrophilic surface of the membrane.

In some embodiments, the nanoparticles provided herein comprise a plurality of the nucleic acid. In some embodiments, at least two of the plurality of nucleic acid comprise different nucleotide sequences relative to each other. In some embodiments, at least two of the plurality of nucleic acids are the same nucleotide sequence. In some embodiments, the nanoparticle comprises a single nucleic acid comprising at least one first nucleic acid encoding a protein or functional fragment thereof, and at least one second nucleic acid encoding an expression enhancer or functional fragment thereof. In some embodiments, the nanoparticle comprises a plurality of nucleic acid, wherein each of the plurality of nucleic acid comprises at least one first nucleic acid, at least one second nucleic acid, or combinations thereof.

In some embodiments, the nucleic acid is deoxyribonucleic acid (DNA) or ribonucleic acid (RNA). The nucleic acid may be linear or include a secondary structure (e.g., a hair pin). In some embodiments, the nucleic acid is a polynucleotide comprising modified nucleotides or bases, and/or their analogs. A polynucleotide may comprise modified nucleotides, such as methylated nucleotides and their analogs. If present, modification to the nucleotide structure may be imparted before or after assembly of compositions provided herein. In some embodiments, compositions provided herein comprise one or more nucleic acids. In some embodiments, compositions provided herein comprise two or more nucleic acids. In some embodiments, compositions provided herein comprise at least one DNA. In some embodiments, compositions provided herein comprise at least one RNA. In some embodiments, compositions provided herein comprise at least one DNA and at least one RNA. In some embodiments, nucleic acids provided herein are present in an amount of above 5 ng to about 1 mg. In some embodiments, nucleic acids provided herein are present in an amount of up to about 25, 50, 75, 100, 150, 175 ng. In some embodiments, nucleic acids provided herein are present in an amount of up to about 1 mg. In some embodiments, nucleic acids provided herein are present in an amount of about 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, nucleic acids provided herein are present in an amount of 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, the nucleic acid is at least about 200, 250, 500, 750, 1000, 2000, 3000, 4000, 5000, 6000, 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is up to about 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is about 7500, 10,000, 15,000, or 20,000 nucleotides in length.

Provided here is a composition comprising a nucleic acid coding for a protein or a functional fragment thereof. In some embodiments, the protein is an antigen, an antigen-binding protein, or a functional fragment thereof. In some embodiments, the antigen is an antigen from an microbial organism. In some embodiments, the antigen is a microbial antigen. In some embodiments, the antigen is a viral antigen. In some embodiments, the viral antigen is a surface protein or a transmembrane protein. In some embodiments, the viral antigen is a spike protein, a glycoprotein, or an envelope protein. In some embodiments, the viral antigen is derived from: an alphavirus, a retrovirus, a coronavirus, a flavivirus, a picornavirus, a rhabdovirus, a rotavirus, a norovirus, a paramyxovirus, a orthomyxovirus, a bunyavirus, an arenavirus, a reovirus, a retrovirus, a rabies virus, a papillomavirus, a parvovirus, a herpesvirus, a poxyirus, a hepadnavirus, a spongiform virus, an iridovirus, an influenza virus, a morbillivirus, a togavirus, a variola virus, a varicella virus, a zika virus, a SARs-CoV-2 virus, a respiratory syncytial virus (RSV), a Middle East Respiratory Syndrome (MERS) coronavirus, human immunodeficiency virus (HIV), a human T-Cell leukemia virus, an Epstein-Barr virus, a cytomegalovirus, a papovavirus, an adenovirus, Non-limiting examples of viral antigens include: Zika virus envelope protein (ZIKV E), Zika virus precursor membrane and envelope proteins (prM-ENV), SARS-CoV2 spike (S) protein and envelope (E) proteins, HIV p24 antigen and Nef protein, influenza virus hemagglutinin (HA) antigen (H2, H3, H5, H6, H7, H8 and H9), influenza virus neuraminidase, rubella E1 and E2 antigens, rotavirus VP7sc antigen, RSV M2 protein, cytomegalovirus envelope glycoprotein B, the S, M, and L proteins of hepatitis B virus, rabies glycoprotein, and rabies nucleoprotein.

In some embodiments, a nucleic acid provided herein encodes for a protein or antibody sequence or a functional fragment thereof which specifically binds an antigen listed in Table 2. In some embodiments, compositions provided herein comprises two or more nucleic acids coding different sequences which specifically binds an antigen listed in Table 2. In some embodiments, the nucleic acid comprises at least 80%, 85%, 90%, 95%, 96%, 97%, 98%, or 99% sequence identity to a sequence which specifically binds an antigen listed in Table 2. In some embodiments, compositions provided herein comprises two or more nucleic acids coding different sequences which specifically binds an antigen listed in Table 2. In some embodiments, the nucleic acid provided herein comprises at least 80%, 85%, 90%, 95%, 96%, 97%, 98%, or 99% sequence similarity to a sequence which specifically binds an antigen listed in Table 2. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated. Exemplary nucleic acid sequences encoding for exemplary antigens are listed in Table 2.

TABLE 2
Exemplary SARS CoV-2 nucleic acid sequences.

| SEQ ID NO. | Name | Variant |
|---|---|---|
| SEQ ID NO: 1 | Delta V5 | A.1 |
| SEQ ID NO: 2 | K995P-V996P | A.1-preF |
| SEQ ID NO: 3 | D614G | B.1 |
| SEQ ID NO: 4 | B.1.351-PP-D614G | Beta-preF |
| SEQ ID NO: 5 | B.1.1.7-PP-D614G | Alpha-preF |
| SEQ ID NO: 6 | Delta.AY1-S2P-wtFur | Delta-preF |
| SEQ ID NO: 7 | Delta.AY1-S2P-wtFur-newKozak | Delta-preF-kozak |

In some embodiments, the nucleic acid provided herein codes for a tumor antigen. In some embodiments, the tumor antigen is a surface protein or a transmembrane protein. Non-limiting examples of tumor antigens include: epidermal growth factor receptor (EGFR); vascular endothelial growth factor (VEGF); VEGFA; acute myelogenous leukemia Wilms tumor 1 (WT1), preferentially expressed antigen of melanoma (PRAME), PR1, proteinase 3, elastase, cathepsin G, Chronic myelogenous WT1, Myelodysplastic syndrome WT1, Acute lymphoblastic leukemia PRAME, Chronic lymphocytic leukemia survivin, Non-Hodgkin's lymphoma survivin, Multiple myeloma New York esophagus 1 (NY-Eso1), Malignant melanoma MAGE, MART-1/Melan-A, Tyrosinase, GP100, Breast cancer WT1, herceptin, Lung cancer WT1, Prostate-specific antigen (PSA), prostatic acid phosphatase, (PAP) Carcinoembryonic antigen (CEA), mucins (e.g., MUC-1), Renal cell carcinoma (RCC) Fibroblast growth factor (FGF), and programmed cell death protein (PD-1).

Provided here is a composition comprising a nucleic acid coding for an antibody. In some embodiments, the antibody is a monoclonal antibody. Monoclonal antibodies or mAbs include intact molecules, as well as, antibody fragments (such as, Fab and F(ab′)2 fragments) that are capable of specifically binding to an epitope of a protein or antigen. In some embodiments, the antibody is a murine antibody, a humanized antibody, or a fully human antibody.

In some embodiments, the antibody is an immunoglobulin (Ig) molecule. Immunoglobulin (Ig) molecules and immunologically active portions of immunoglobulin molecules (i.e., molecules that contain an antigen binding site that specifically bind an antigen) are comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments of which are discussed below, and include but are not limited to a variety of forms, including full length antibodies and antigen-binding portions thereof; including, for example, an immunoglobulin molecule, a monoclonal antibody, a chimeric antibody, a CDR-grafted antibody, a human antibody, a humanized antibody, a single chain antibody, a Fab, a F(ab′), a F(ab′)2, a Fv antibody, fragments produced by a Fab expression library, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies. In some embodiments, the immunoglobulin molecule is an IgG, IgE, IgM, IgD, IgA, or an IgY isotype immunoglobulin molecule. In some embodiments, the antibody or immunoglobulin molecules provided herein are a specific subclass of immunoglobulin molecule. In some embodiments, the immunoglobulin molecule is an IgG1, an IgG2, an IgG3, an IgG4, an IgGA1, or an IgGA2 subclass immunoglobulin molecule. In a full-length antibody, each heavy chain is comprised of a heavy chain variable domain (abbreviated herein as HCVR or VH) and a heavy chain constant region. The heavy chain constant region is comprised of three domains: CH1, CH2, and CH3. Each light chain is comprised of a light chain variable domain (abbreviated herein LCVR as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. This structure is well-known to those skilled in the art. The chains are usually linked to one another via disulfide bonds. Furthermore, in humans, the light chain may comprise a kappa chain or a lambda chain. Complementarity Determining Regions (“CDRs”), i.e., CDR1, CDR2, and CDR3) are the amino acid residues of a heavy or light chain variable domain specific for antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (LI), 50-56 (L2) and 89-97 (L3) in the light chain variable domain and 31-35 (HI), 50-65 (H2) and 95-102 (H3) in the heavy chain variable domain; Kabat et al, Sequences of Proteins of Immunological Interest, 5th Ed. Public Health Service, National Institutes of Health, Bethesda, Md. (1991)) and/or those residues from a “hypervariable loop” (i.e., about residues 26-32 (LI), 50-52 (L2) and 91-96 (L3) in the light chain variable domain and 26-32 (HI), 53-55 (H2) and 96-101 (H3) in the heavy chain variable domain; Chothia and Lesk J Mol. Biol. 196:901-917 (1987)). In some instances, a complementarity determining region can include amino acids from both a CDR region defined according to Kabat and a hypervariable loop. The exact boundaries of these CDRs have been defined differently according to different systems. The system described by Kabat (Kabat et al, Sequences of Proteins of Immunological Interest (National Institutes of Health, Bethesda, Md. (1987) and (1991)) not only provides an unambiguous residue numbering system applicable to any variable region of an antibody, but also provides the residue boundaries defining the three CDRs. These CDRs may be referred to as Kabat CDRs. Chothia and coworkers (Chothia & Lesk, J. Mol. Biol, 196:901-917 (1987) and Chothia et al., Nature 342:877-883 (1989)) found that certain sub-portions within Kabat CDRs adopt nearly identical peptide backbone conformations, in spite of great diversity at the level of amino acid sequence. These sub-portions were designated as LI, L2 and L3 or HI, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB). 9: 133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or assay result that particular residues or groups of residues or even entire CDRs do not significantly impact antigen binding. The alignment of the CDR sequences can be conducted using publicly available software such as BLAST, Align, and the international ImMunoGeneTics information system (IMGT). Those skilled in the art can determine the appropriate parameters for alignment, but the default parameters for BLAST are specifically contemplated. In some embodiments, an antibody described herein is originally generated by a non-human animal (e.g., sheep, dog, rabbit, mouse, rat, primate, goat, llama, alpaca, and horse) against an antigen described herein and, optionally, humanized as described herein.

In some embodiments, the nucleic acid provided herein codes for a recombinant antibody, a chimeric antibody, or a multivalent antibody. In some embodiments, the multivalent antibody is a bispecific antibody, a trispecific antibody, or a multispecific antibody. In some embodiments, the antibody or functional fragment is an antigen-binding fragment (Fab), and Fab2 a F(ab′), a F(ab′)2, an dAb, an Fc, a Fv, a disulfide linked Fv, a scFv, a tandem scFv, a free LC, a half antibody, a single domain antibody (dAb), a diabody, or a nanobody. In some embodiments, the nanobody comprises a heavy chain variable (VH) region. In further embodiments, the heavy chain variable (VH) region comprises three CDR regions.

In some embodiments, the nucleic acid provided herein codes for a gene transcription regulator. In some embodiments, the gene transcription regulator comprises an expression enhancer or a functional fragment thereof. In some embodiments, the expression enhancer increases expression of a protein or a function fragment thereof, when a cell is co-transfected with a first nucleic acid coding for the protein or the functional fragment thereof, and a second nucleic acid coding for the expression enhancer or the functional fragment thereof.

### Viral Antigen Binding Molecules

In some embodiments, the antibody or functional fragment thereof specifically binds to a microbial antigen. In some embodiments, the microbial antigen is a viral envelope protein. In some embodiments, the antibody or functional fragment thereof is a SARS-CoV-2 virus antibody. In some embodiments, the SARS-CoV-2 virus antibody is bamlanivimab, casirivimab, imdevimab, or sotrovimab. Exemplary amino acid sequences for SARs-CoV-2 antibodies are provided below in Table 3.

In some embodiments, a nucleic acid provided herein codes for a protein or antibody amino acid sequence or a functional fragment thereof listed in Table 3. In some embodiments, compositions provided herein comprise two or more nucleic acids coding different sequences listed in Table 3. In some embodiments, the nucleic acid provided herein codes for a protein or antibody amino acid sequence or a functional fragment thereof comprising at least 80%, 85%, 90%, 95%, 96%, 97%, 98%, or 99% sequence identity to a sequence listed in Table 3. In some embodiments, compositions provided herein comprise two or more nucleic acids coding different sequences listed in Table 3. In some embodiments, the nucleic acid provided herein codes for a protein, antibody, or fragment thereof comprising at least 80%, 85%, 90%, 95%, 96%, 97%, 98%, or 99% sequence similarity to a sequence listed Table 3.

TABLE 3
SARs-COV-2 Antibody Amino Acid Sequences.

| Heavy | | | | | Light |
|---|---|---|---|---|---|
| chain | Antibody | | | | chain |
| SEQ | Name | | | | SEQ |
| ID | (Commercial | Viral | | Light Chain | ID |
| NO: | Name) | Antigen | Heavy Chain Sequences | Sequences | NO: |
| 8 | bamlani | Spike (S) | QVQLVQSGAEVKKPGSSVKVSCKA | DIQMTQSPSSLS | 53 |
| | vimab | glycopro- | SGGTFSNYAISWVRQAPGQGLEWM | ASVGDRVTITCR | |
| | (LY- | tein | GRIIPILGIANYAQKFQGRVTITA | ASQSISSYLSWY | |
| | CoV555) | receptor | DKSTSTAYMELSSLRSEDTAVYYC | QQKPGKAPKLLI | |
| | | binding | ARGYYEARHYYYYYAMDVWGQGTA | YAASSLQSGVPS | |
| | | domain | VTVSSASTKGPSVFPLAPSSKSTS | RFSGSGSGTDFT | |
| | | (RBD) | GGTAALGCLVKDYFPEPVTVSWNS | LTITSLQPEDFA | |
| | | | GALTSGVHTFPAVLQSSGLYSLSS | TYYCQQSYSTPR | |
| | | | VVTVPSSSLGTQTYICNVNHKPSN | TFGQGTKVEIKR | |
| | | | TKVDKRVEPKSCDKTHTCPPCPAP | TVAAPSVFIFPP | |
| | | | ELLGGPSVFLFPPKPKDTLMISRT | SDEQLKSGTASV | |
| | | | PEVTCVVVDVSHEDPEVKFNWYVD | VCLLNNFYPREA | |
| | | | GVEVHNAKTKPREEQYNSTYRVVS | KVQWKVDNALQS | |
| | | | VLTVLHQDWLNGKEYKCKVSNKAL | GNSQESVTEQDS | |
| | | | PAPIEKTISKAKGQPREPQVYTLP | KDSTYSLSSTLT | |
| | | | PSREEMTKNQVSLTCLVKGFYPSD | LSKADYEKHKVY | |
| | | | IAVEWESNGQPENNYKTTPPVLDS | ACEVTHQGLSSP | |
| | | | DGSFFLYSKLTVDKSRWQQGNVFS | VTKSFNRGEC | |
| | | | CSVMHEALHNHYTQKSLSLSPGK | | |
| 9 | casirivimab | Spike (S) | QVQLVESGGGLVKPGGSLRLSCAA | DIQMTQSPSSLS | 54 |
| | (REGEN- | glycopro- | SGFTFSDYYMSWIRQAPGKGLEWV | ASVGDRVTITCQ | |
| | COV™) | tein | SYITYSGSTIYYADSVKGRFTISR | ASQDITNYLNWY | |
| | | receptor | DNAKSSLYLQMNSLRAEDTAVYYC | QQKPGKAPKLLI | |
| | | binding | ARDRGTTMVPFDYWGQGTLVTVSS | YAASNLETGVPS | |
| | | domain | ASTKGPSVFPLAPSSKSTSGGTAA | RFSGSGSGTDFT | |
| | | (RBD) | LGCLVKDYFPEPVTVSWNSGALTS | FTISGLQPEDIA | |
| | | | GVHTFPAVLQSSGLYSLSSVVTVP | TYYCQQYDNLPL | |
| | | | SSSLGTQTYICNVNHKPSNTKVDK | TFGGGTKVEIKR | |
| | | | KVEPKSCDKTHTCPPCPAPELLGG | TVAAPSVFIFPP | |
| | | | PSVFLFPPKPKDTLMISRTPEVTC | SDEQLKSGTASV | |
| | | | VVVDVSHEDPEVKFNWYVDGVEVH | VCLLNNFYPREA | |
| | | | NAKTKPREEQYNSTYRVVSVLTVL | KVQWKVDNALQS | |
| | | | HQDWLNGKEYKCKVSNKALPAPIE | GNSQESVTEQDS | |
| | | | KTISKAKGQPREPQVYTLPPSRDE | KDSTYSLSSTLT | |
| | | | LTKNQVSLTCLVKGFYPSDIAVEW | LSKADYEKHKVY | |
| | | | ESNGQPENNYKTTPPVLDSDGSFF | ACEVTHQGLSSP | |
| | | | LYSKLTVDKSRWQQGNVFSCSVMH | VTKSFNRGEC | |
| | | | EALHNHYTQKSLSLSPG | | |
| 10 | imdevimab | Spike (S) | QVQLVESGGGVVQPGRSLRLSCAA | QSALTQPASVSG | 55 |
| | (REGEN- | glycopro- | SGFTFSNYAMYWVRQAPGKGLEWV | SPGQSITISCTG | |
| | COV™) | tein | AVISYDGSNKYYADSVKGRFTISR | TSSDVGGYNYVS | |
| | | receptor | DNSKNTLYLQMNSLRTEDTAVYYC | WYQQHPGKAPKL | |
| | | binding | ASGSDYGDYLLVYWGQGTLVTVSS | MIYDVSKRPSGV | |
| | | domain | ASTKGPSVFPLAPSSKSTSGGTAA | SNRFSGSKSGNT | |
| | | (RBD) | LGCLVKDYFPEPVTVSWNSGALTS | ASLTISGLQSED | |
| | | | GVHTFPAVLQSSGLYSLSSVVTVP | EADYYCNSLTSI | |
| | | | SSSLGTQTYICNVNHKPSNTKVDK | STWVFGGGTKLT | |
| | | | KVEPKSCDKTHTCPPCPAPELLGG | VLGQPKAAPSVT | |
| | | | PSVFLFPPKPKDTLMISRTPEVTC | LFPPSSEELQAN | |
| | | | VVVDVSHEDPEVKFNWYVDGVEVH | KATLVCLISDFY | |
| | | | NAKTKPREEQYNSTYRVVSVLTVL | PGAVTVAWKADS | |
| | | | HQDWLNGKEYKCKVSNKALPAPIE | SPVKAGVETTTP | |
| | | | KTISKAKGQPREPQVYTLPPSRDE | SKQSNNKYAASS | |
| | | | LTKNQVSLTCLVKGFYPSDIAVEW | YLSLTPEQWKSH | |
| | | | ESNGQPENNYKTTPPVLDSDGSFF | RSYSCQVTHEGS | |
| | | | LYSKLTVDKSRWQQGNVFSCSVMH | TVEKTVAPTECS | |
| | | | EALHNHYTQKSLSLSPGK | | |
| 11 | sotrovimab | Spike (S) | QVQLVQSGAEVKKPGASVKVSCKA | EIVLTQSPGTLS | 56 |
| | (Xevudy) | glycopro- | SGYPFTSYGISWVRQAPGQGLEWM | LSPGERATLSCR | |
| | | tein | GWISTYQGNTNYAQKFQGRVTMTT | ASQTVSSTSLAW | |
| | | receptor | DTSTTTGYMELRRLRSDDTAVYYC | YQQKPGQAPRLL | |
| | | binding | ARDYTRGAWFGESLIGGFDNWGQG | IYGASSRATGIP | |
| | | domain | TLVTVSSASTKGPSVFPLAPSSKS | DRFSGSGSGTDF | |
| | | (RBD) | TSGGTAALGCLVKDYFPEPVTVSW | TLTISRLEPEDF | |
| | | | NSGALTSGVHTFPAVLQSSGLYSL | AVYYCQQHDTSL | |
| | | | SSVVTVPSSSLGTQTYICNVNHKP | TFGGGTKVEIKR | |
| | | | SNTKVDKKVEPKSCDKTHTCPPCP | TVAAPSVFIFPP | |
| | | | APELLGGPSVFLFPPKPKDTLMIS | SDEQLKSGTASV | |
| | | | RTPEVTCVVVDVSHEDPEVKFNWY | VCLLNNFYPREA | |
| | | | VDGVEVHNAKTKPREEQYNSTYRV | KVQWKVDNALQS | |
| | | | VSVLTVLHQDWLNGKEYKCKVSNK | GNSQESVTEQDS | |
| | | | ALPAPIEKTISKAKGQPREPQVYT | KDSTYSLSSTLT | |
| | | | LPPSRDELTKNQVSLTCLVKGFYP | LSKADYEKHKVY | |
| | | | SDIAVEWESNGQPENNYKTTPPVL | ACEVTHQGLSSP | |
| | | | DSDGSFFLYSKLTVDKSRWQQGNV | VTKSFNRGEC | |
| | | | FSCSVLHEALHSHYTQKSLSLSPG | | |
| | | | K | | |

*Sequences in Table 3 were determined by IMGT-monoclonal antibody database

In some embodiments, the antibody or functional fragment thereof is a Zika virus antibody. In some embodiments, the Zika virus antibody is ZIKV-117, Z3L1, Z20, Z23, ZV67, Z006, or 2A10G6. In some embodiments, the ZIKV-117 antibody or functional fragment comprises a heavy chain CDR1 amino acid sequence of GFTFKNYG (SEQ ID NO: 48), a heavy chain CDR2 amino acid sequence of VRYDGNNK (SEQ ID NO: 49), and a heavy chain CDR3 amino acid sequence of ARDPETFGGFDY (SEQ ID NO: 50), and alight chain CDR1 amino acid sequence of ESVSSN (SEQ ID NO: 51), light chain CDR2 amino acid sequence of GAS, and light chain CDR3 amino acid sequence of QQYYYSPRT (SEQ ID NO: 52).

In some embodiments, a nucleic acid provided herein codes for a protein or antibody sequence or a functional fragment thereof listed in Table 4. In some embodiments, compositions provided herein comprises two or more nucleic acids coding different sequences listed in Table 4. In some embodiments, the nucleic acid comprises at least 80%, 85%, 90%, 95%, 96%, 97%, 98%, or 99% sequence identity to a sequence listed in Table 4. In some embodiments, compositions provided herein comprise two or more nucleic acids coding different sequences listed in Table 4. In some embodiments, the nucleic acid provided herein comprises at least 80%, 85%, 90%, 95%, 96%, 97%, 98%, or 99% sequence similarity to a sequence listed Table 4. Exemplary nucleic acid sequences are listed in Table 4 below.

TABLE 4
Zika Virus Antibody Nucleic Acid Sequences.

| | Nucleic Acid Sequence Name |
|---|---|
| | *Sequences are provided following the |
| SEQ ID NO: | Example section of this document |
| SEQ ID NO: 12 | ZIKV-117 Full Length Antibody Sequence |
| SEQ ID NO: 13 | ZIKV-117 Heavy Chain Antibody Sequence |
| SEQ ID NO: 14 | ZIKV-117 Light Chain Antibody Sequence |

### Cancer Antigen Binding Molecules

In some embodiments, the antibody or functional fragment thereof specifically binds to a tumor antigen. In some embodiments, the antibody or functional fragment thereof is a cancer therapeutic antibody. In some embodiments, the cancer therapeutic antibody is atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab. Exemplary amino acid sequences for cancer therapeutic antibodies are provided below in Table 5.

In some embodiments, a nucleic acid provided herein codes for a protein or antibody amino acid sequence or a functional fragment thereof listed in Table 5. In some embodiments, compositions provided herein comprises two or more nucleic acids coding for different sequences listed in Table 5. In some embodiments, the nucleic acid provided herein codes for a protein or antibody amino acid sequence or afunctional fragment thereof comprising at least 80%, 85%, 90%, 95% 96%, 97% 98%, or 99% sequence identity to a sequence listed in Table 5. In some embodiments, compositions provided herein comprise two or more nucleic acids coding different sequences listed in Table 5. In some embodiments, the nucleic acid provided herein codes for a protein, antibody, or afunctional fragment thereof comprising at least 80%, 85%, 90%, 95% 96%, 97%, 98%, or 99% sequence similarity to a sequence listed Table 5.

TABLE 5
Cancer therapeutic antibodies.

| SEQ | Antibody Name | | |
|---|---|---|---|
| ID | (Commercial | | |
| NO: | Name) | Tumor Antigen | Heavy Chain Amino Acid Sequences |
| 15 | atezolizumab | CD274 | EVQLVESGGGLVQPGGSLRLSCAASGFTFSDSWIHWVRQAP |
| | (TECENTRIQ®) | (programmed cell | GKGLEWVAWISPYGGSTYYADSVKGRFTISADTSKNTAYLQ |
| | | death 1 ligand 1, | MNSLRAEDTAVYYCARRHWPGGFDYWGQGTLVTVSSASTKG |
| | | B7H1, B7-H1, | PSVFPLAPSSKSTSGGTAALGCLVKDYFPEPVTVSWNSGAL |
| | | PDL1, PD-L1, | TSGVHTFPAVLQSSGLYSLSSVVTVPSSSLGTQTYICNVNH |
| | | PDCD1L1, B7 | KPSNTKVDKKVEPKSCDKTHTCPPCPAPELLGGPSVFLFPP |
| | | homolog 1, B7 | KPKDTLMISRTPEVTCVVVDVSHEDPEVKFNWYVDGVEVHN |
| | | homologue 1) | AKTKPREEQYASTYRVVSVLTVLHQDWLNGKEYKCKVSNKA |
| | | [Homo sapiens] | LPAPIEKTISKAKGQPREPQVYTLPPSREEMTKNQVSLTCL |
| | | | VKGFYPSDIAVEWESNGQPENNYKTTPPVLDSDGSFFLYSK |
| | | | LTVDKSRWQQGNVFSCSVMHEALHNHYTQKSLSLSPGK |
| 16 | avelumab | CD274 | EVQLLESGGGLVQPGGSLRLSCAASGFTFSSYIMMWVRQAP |
| | (BAVENCIO®) | | GKGLEWVSSIYPSGGITFYADTVKGRFTISRDNSKNTLYLQ |
| | | | MNSLRAEDTAVYYCARIKLGTVTTVDYWGQGTLVTVSSAST |
| | | | KGPSVFPLAPSSKSTSGGTAALGCLVKDYFPEPVTVSWNSG |
| | | | ALTSGVHTFPAVLQSSGLYSLSSVVTVPSSSLGTQTYICNV |
| | | | NHKPSNTKVDKKVEPKSCDKTHTCPPCPAPELLGGPSVFLF |
| | | | PPKPKDTLMISRTPEVTCVVVDVSHEDPEVKFNWYVDGVEV |
| | | | HNAKTKPREEQYNSTYRVVSVLTVLHQDWLNGKEYKCKVSN |
| | | | KALPAPIEKTISKAKGQPREPQVYTLPPSRDELTKNQVSLT |
| | | | CLVKGFYPSDIAVEWESNGQPENNYKTTPPVLDSDGSFFLY |
| | | | SKLTVDKSRWQQGNVFSCSVMHEALHNHYTQKSLSLSPGK |
| 17 | bevacizumab | VEGFA (vascular | EVQLVESGGGLVQPGGSLRLSCAASGYTFTNYGMNWVRQAP |
| | (AVASTIN®) | endothelial growth | GKGLEWVGWINTYTGEPTYAADFKRRFTFSLDTSKSTAYLQ |
| | | factor A, VEGF-A, | MNSLRAEDTAVYYCAKYPHYYGSSHWYFDVWGQGTLVTVSS |
| | | VEGF) [Homo | ASTKGPSVFPLAPSSKSTSGGTAALGCLVKDYFPEPVTVSW |
| | | sapiens] | NSGALTSGVHTFPAVLQSSGLYSLSSVVTVPSSSLGTQTYI |
| | | | CNVNHKPSNTKVDKKVEPKSCDKTHTCPPCPAPELLGGPSV |
| | | | FLFPPKPKDTLMISRTPEVTCVVVDVSHEDPEVKFNWYVDG |
| | | | VEVHNAKTKPREEQYNSTYRVVSVLTVLHQDWLNGKEYKCK |
| | | | VSNKALPAPIEKTISKAKGQPREPQVYTLPPSREEMTKNQV |
| | | | SLTCLVKGFYPSDIAVEWESNGQPENNYKTTPPVLDSDGSF |
| | | | FLYSKLTVDKSRWQQGNVFSCSVMHEALHNHYTQKSLSLSP |
| | | | GK |
| 18 | cemiplimab | PDCD1 | EVQLLESGGVLVQPGGSLRLSCAASGFTFSNFGMTWVRQAP |
| | (LIBTAYO®) | (programmed cell | GKGLEWVSGISGGGRDTYFADSVKGRFTISRDNSKNTLYLQ |
| | | death 1, PD1, PD-1, | MNSLKGEDTAVYYCVKWGNIYFDYWGQGTLVTVSSASTKGP |
| | | CD279) [Homo | SVFPLAPCSRSTSESTAALGCLVKDYFPEPVTVSWNSGALT |
| | | sapiens] | SGVHTFPAVLQSSGLYSLSSVVTVPSSSLGTKTYTCNVDHK |
| | | | PSNTKVDKRVESKYGPPCPPCPAPEFLGGPSVFLFPPKPKD |
| | | | TLMISRTPEVTCVVVDVSQEDPEVQFNWYVDGVEVHNAKTK |
| | | | PREEQFNSTYRVVSVLTVLHQDWLNGKEYKCKVSNKGLPSS |
| | | | IEKTISKAKGQPREPQVYTLPPSQEEMTKNQVSLTCLVKGF |
| | | | YPSDIAVEWESNGQPENNYKTTPPVLDSDGSFFLYSRLTVD |
| | | | KSRWQEGNVFSCSVMHEALHNHYTQKSLSLSLGK |
| 19 | cetuximab | EGFR (epidermal | QVQLKQSGPGLVQPSQSLSITCTVSGFSLTNYGVHWVRQSP |
| | (ERBITUX®) | growth factor | GKGLEWLGVIWSGGNTDYNTPFTSRLSINKDNSKSQVFFKM |
| | | receptor, receptor | NSLQSNDTAIYYCARALTYYDYEFAYWGQGTLVTVSAASTK |
| | | tyrosine-protein | GPSVFPLAPSSKSTSGGTAALGCLVKDYFPEPVTVSWNSGA |
| | | kinase erbB-1, | LTSGVHTFPAVLQSSGLYSLSSVVTVPSSSLGTQTYICNVN |
| | | ERBB1, HER1, | HKPSNTKVDKRVEPKSCDKTHTCPPCPAPELLGGPSVFLFP |
| | | HER-1, ERBB) | PKPKDTLMISRTPEVTCVVVDVSHEDPEVKFNWYVDGVEVH |
| | | [Homo sapiens] | NAKTKPREEQYNSTYRVVSVLTVLHQDWLNGKEYKCKVSNK |
| | | | ALPAPIEKTISKAKGQPREPQVYTLPPSREEMTKNQVSLTC |
| | | | LVKGFYPSDIAVEWESNGQPENNYKTTPPVLDSDGSFFLYS |
| | | | KLTVDKSRWQQGNVFSCSVMHEALHNHYTQKSLSLSPGK |
| 20 | daratumumab | CD38 (ADP- | EVQLLESGGGLVQPGGSLRLSCAVSGFTFNSFAMSWVRQAP |
| | (DARZALEX™; | ribosyl cyclase 1, | GKGLEWVSAISGSGGGTYYADSVKGRFTISRDNSKNTLYLQ |
| | DARZALEX; | cyclic ADP-ribose | MNSLRAEDTAVYFCAKDKILWFGEPVFDYWGQGTLVTVSSA |
| | FASPRO™) | hydrolase 1, cADPr | STKGPSVFPLAPSSKSTSGGTAALGCLVKDYFPEPVTVSWN |
| | | hydrolase 1, T10) | SGALTSGVHTFPAVLQSSGLYSLSSVVTVPSSSLGTQTYIC |
| | | [Homo sapiens] | NVNHKPSNTKVDKRVEPKSCDKTHTCPPCPAPELLGGPSVF |
| | | | LFPPKPKDTLMISRTPEVTCVVVDVSHEDPEVKFNWYVDGV |
| | | | EVHNAKTKPREEQYNSTYRVVSVLTVLHQDWLNGKEYKCKV |
| | | | SNKALPAPIEKTISKAKGQPREPQVYTLPPSREEMTKNQVS |
| | | | LTCLVKGFYPSDIAVEWESNGQPENNYKTTPPVLDSDGSFF |
| | | | LYSKLTVDKSRWQQGNVFSCSVMHEALHNHYTQKSLSLSPG |
| | | | K |
| 21 | dinutuximab | ganglioside GD2 | EVQLLQSGPELEKPGASVMISCKASGSSFTGYNMNWVRQNI |
| | (UNITUXIN™) | (disialoganglioside | GKSLEWIGAIDPYYGGTSYNQKFKGRATLTVDKSSSTAYMH |
| | | GD2) [Homo | LKSLTSEDSAVYYCVSGMEYWGQGTSVTVSSASTKGPSVFP |
| | | sapiens] | LAPSSKSTSGGTAALGCLVKDYFPEPVTVSWNSGALTSGVH |
| | | | TFPAVLQSSGLYSLSSVVTVPSSSLGTQTYICNVNHKPSNT |
| | | | KVDKRVEPKSCDKTHTCPPCPAPELLGGPSVFLFPPKPKDT |
| | | | LMISRTPEVTCVVVDVSHEDPEVKFNWYVDGVEVHNAKTKP |
| | | | REEQYNSTYRVVSVLTVLHQDWLNGKEYKCKVSNKALPAPI |
| | | | EKTISKAKGQPREPQVYTLPPSREEMTKNQVSLTCLVKGFY |
| | | | PSDIAVEWESNGQPENNYKTTPPVLDSDGSFFLYSKLTVDK |
| | | | SRWQQGNVFSCSVMHEALHNHYTQKSLSLSPGK |
| 22 | durvalumab | CD274 | EVQLVESGGGLVQPGGSLRLSCAASGFTFSRYWMSWVRQAP |
| | (IMFINZI™) | | GKGLEWVANIKQDGSEKYYVDSVKGRFTISRDNAKNSLYLQ |
| | | | MNSLRAEDTAVYYCAREGGWFGELAFDYWGQGTLVTVSSAS |
| | | | TKGPSVFPLAPSSKSTSGGTAALGCLVKDYFPEPVTVSWNS |
| | | | GALTSGVHTFPAVLQSSGLYSLSSVVTVPSSSLGTQTYICN |
| | | | VNHKPSNTKVDKRVEPKSCDKTHTCPPCPAPEFEGGPSVFL |
| | | | FPPKPKDTLMISRTPEVTCVVVDVSHEDPEVKFNWYVDGVE |
| | | | VHNAKTKPREEQYNSTYRVVSVLTVLHQDWLNGKEYKCKVS |
| | | | NKALPASIEKTISKAKGQPREPQVYTLPPSREEMTKNQVSL |
| | | | TCLVKGFYPSDIAVEWESNGQPENNYKTTPPVLDSDGSFFL |
| | | | YSKLTVDKSRWQQGNVFSCSVMHEALHNHYTQKSLSLSPGK |
| 23 | elotuzumab | SLAMF7 (SLAM | EVQLVESGGGLVQPGGSLRLSCAASGFDFSRYWMSWVRQAP |
| | (EMPLICITI™) | family member 7, | GKGLEWIGEINPDSSTINYAPSLKDKFIISRDNAKNSLYLQ |
| | | CD2 subset 1, CS1, | MNSLRAEDTAVYYCARPDGNYWYFDVWGQGTLVTVSSASTK |
| | | CD2-like receptor- | GPSVFPLAPSSKSTSGGTAALGCLVKDYFPEPVTVSWNSGA |
| | | activating cytotoxic | LTSGVHTFPAVLQSSGLYSLSSVVTVPSSSLGTQTYICNVN |
| | | cells, CRACC, | HKPSNTKVDKKVEPKSCDKTHTCPPCPAPELLGGPSVFLFP |
| | | 19A24, CD319) | PKPKDTLMISRTPEVTCVVVDVSHEDPEVKFNWYVDGVEVH |
| | | [Homo sapiens] | NAKTKPREEQYNSTYRVVSVLTVLHQDWLNGKEYKCKVSNK |
| | | | ALPAPIEKTISKAKGQPREPQVYTLPPSRDELTKNQVSLTC |
| | | | LVKGFYPSDIAVEWESNGQPENNYKTTPPVLDSDGSFFLYS |
| | | | KLTVDKSRWQQGNVFSCSVMHEALHNHYTQKSLSLSPGK |
| 24 | ipilimumab | CTLA4 (cytotoxic | QVQLVESGGGVVQPGRSLRLSCAASGFTFSSYTMHWVRQAP |
| | (YERVOY®) | T-lymphocyte- | GKGLEWVTFISYDGNNKYYADSVKGRFTISRDNSKNTLYLQ |
| | | associated protein | MNSLRAEDTAIYYCARTGWLGPFDYWGQGTLVTVSSASTKG |
| | | 4, CD152) [Homo | PSVFPLAPSSKSTSGGTAALGCLVKDYFPEPVTVSWNSGAL |
| | | sapiens] | TSGVHTFPAVLQSSGLYSLSSVVTVPSSSLGTQTYICNVNH |
| | | | KPSNTKVDKRVEPKSCDKTHTCPPCPAPELLGGPSVFLFPP |
| | | | KPKDTLMISRTPEVTCVVVDVSHEDPEVKFNWYVDGVEVHN |
| | | | AKTKPREEQYNSTYRVVSVLTVLHQDWLNGKEYKCKVSNKA |
| | | | LPAPIEKTISKAKGQPREPQVYTLPPSRDELTKNQVSLTCL |
| | | | VKGFYPSDIAVEWESNGQPENNYKTTPPVLDSDGSFFLYSK |
| | | | LTVDKSRWQQ GNVFSCSVMHEALHNHYTQKSLSLSPGK |
| 25 | isatuximab | CD38 (ADP- | QVQLVQSGAEVAKPGTSVKLSCKASGYTFTDYWMQWVKQRP |
| | (SARCLISA®) | ribosyl cyclase 1, | GQGLEWIGTIYPGDGDTGYAQKFQGKATLTADKSSKTVYMH |
| | | cyclic ADP-ribose | LSSLASEDSAVYYCARGDYYGSNSLDYWGQGTSVTVSSAST |
| | | hydrolase 1, cADPr | KGPSVFPLAPSSKSTSGGTAALGCLVKDYFPEPVTVSWNSG |
| | | hydrolase 1, T10) | ALTSGVHTFPAVLQSSGLYSLSSVVTVPSSSLGTQTYICNV |
| | | [Homo sapiens] | NHKPSNTKVDKKVEPKSCDKTHTCPPCPAPELLGGPSVFLF |
| | | | PPKPKDTLMISRTPEVTCVVVDVSHEDPEVKFNWYVDGVEV |
| | | | HNAKTKPREEQYNSTYRVVSVLTVLHQDWLNGKEYKCKVSN |
| | | | KALPAPIEKTISKAKGQPREPQVYTLPPSRDELTKNQVSLT |
| | | | CLVKGFYPSDIAVEWESNGQPENNYKTTPPVLDSDGSFFLY |
| | | | SKLTVDKSRWQQGNVFSCSVMHEALHNHYTQKSLSLSPGK |
| 26 | mogamulizumab | CCR4 (chemokine | EVQLVESGGDLVQPGRSLRLSCAASGFIFSNYGMSWVRQAP |
| | (POTELIGEO®) | (C-C motif) | GKGLEWVATISSASTYSYYPDSVKGRFTISRDNAKNSLYLQ |
| | | receptor 4, CC | MNSLRVEDTALYYCGRHSDGNFAFGYWGQGTLVTVSSASTK |
| | | chemokine receptor | GPSVFPLAPSSKSTSGGTAALGCLVKDYFPEPVTVSWNSGA |
| | | 4, CCR-4, CKR4, | LTSGVHTFPAVLQSSGLYSLSSVVTVPSSSLGTQTYICNVN |
| | | k5-5, CD194) | HKPSNTKVDKKVEPKSCDKTHTCPPCPAPELLGGPSVFLFP |
| | | [Homo sapiens] | PKPKDTLMISRTPEVTCVVVDVSHEDPEVKFNWYVDGVEVH |
| | | | NAKTKPREEQYNSTYRVVSVLTVLHQDWLNGKEYKCKVSNK |
| | | | ALPAPIEKTISKAKGQPREPQVYTLPPSRDELTKNQVSLTC |
| | | | LVKGFYPSDIAVEWESNGQPENNYKTTPPVLDSDGSFFLYS |
| | | | KLTVDKSRWQQGNVFSCSVMHEALHNHYTQKSLSLSPGK |
| 27 | necitumumab | EGFR (epidermal | QVQLQESGPGLVKPSQTLSLTCTVSGGSISSGDYYWSWIRQ |
| | (PORTRAZZA™) | growth factor | PPGKGLEWIGYIYYSGSTDYNPSLKSRVTMSVDTSKNQFSL |
| | | receptor, receptor | KVNSVTAADTAVYYCARVSIFGVGTFDYWGQGTLVTVSSAS |
| | | tyrosine-protein | TKGPSVLPLAPSSKSTSGGTAALGCLVKDYFPEPVTVSWNS |
| | | kinase erbB-1, | GALTSGVHTFPAVLQSSGLYSLSSVVTVPSSSLGTQTYICN |
| | | ERBB1, HER1, | VNHKPSNTKVDKRVEPKSCDKTHTCPPCPAPELLGGPSVFL |
| | | HER-1, ERBB) | FPPKPKDTLMISRTPEVTCVVVDVSHEDPEVKFNWYVDGVE |
| | | [Homo sapiens] | VHNAKTKPREEQYNSTYRVVSVLTVLHQDWLNGKEYKCKVS |
| | | | NKALPAPIEKTISKAKGQPREPQVYTLPPSREEMTKNQVSL |
| | | | TCLVKGFYPSDIAVEWESNGQPENNYKTTPPVLDSDGSFFL |
| | | | YSKLTVDKSRWQQGNVFSCSVMHEALHNHYTQKSLSLSPGK |
| 28 | nivolumab | PDCD1 | QVQLVESGGGVVQPGRSLRLDCKASGITFSNSGMHWVRQAP |
| | (OPDIVO®) | (programmed cell | GKGLEWVAVIWYDGSKRYYADSVKGRFTISRDNSKNTLFLQ |
| | | death 1, PD1, PD- | MNSLRAEDTAVYYCATNDDYWGQGTLVTVSSASTKGPSVFP |
| | | 1, CD279) [Homo | LAPCSRSTSESTAALGCLVKDYFPEPVTVSWNSGALTSGVH |
| | | sapiens] | TFPAVLQSSGLYSLSSVVTVPSSSLGTKTYTCNVDHKPSNT |
| | | | KVDKRVESKYGPPCPPCPAPEFLGGPSVFLFPPKPKDTLMI |
| | | | SRTPEVTCVVVDVSQEDPEVQFNWYVDGVEVHNAKTKPREE |
| | | | QFNSTYRVVSVLTVLHQDWLNGKEYKCKVSNKGLPSSIEKT |
| | | | ISKAKGQPREPQVYTLPPSQEEMTKNQVSLTCLVKGFYPSD |
| | | | IAVEWESNGQPENNYKTTPPVLDSDGSFFLYSRLTVDKSRW |
| | | | QEGNVFSCSVMHEALHNHYTQKSLSLSLGK |
| 29 | obinutuzumab | MS4A1 | QVQLVQSGAEVKKPGSSVKVSCKASGYAFSYSWINWVRQAP |
| | (GAZYVA®) | (membrane- | GQGLEWMGRIFPGDGDTDYNGKFKGRVTITADKSTSTAYME |
| | | spanning 4- | LSSLRSEDTAVYYCARNVFDGYWLVYWGQGTLVTVSSASTK |
| | | domains subfamily | GPSVFPLAPSSKSTSGGTAALGCLVKDYFPEPVTVSWNSGA |
| | | A member 1, | LTSGVHTFPAVLQSSGLYSLSSVVTVPSSSLGTQTYICNVN |
| | | CD20) [Homo | HKPSNTKVDKKVEPKSCDKTHTCPPCPAPELLGGPSVFLFP |
| | | sapiens] | PKPKDTLMISRTPEVTCVVVDVSHEDPEVKFNWYVDGVEVH |
| | | | NAKTKPREEQYNSTYRVVSVLTVLHQDWLNGKEYKCKVSNK |
| | | | ALPAPIEKTISKAKGQPREPQVYTLPPSRDELTKNQVSLTC |
| | | | LVKGFYPSDIAVEWESNGQPENNYKTTPPVLDSDGSFFLYS |
| | | | KLTVDKSRWQQGNVFSCSVMHEALHNHYTQKSLSLSPGK |
| 30 | ofatumumab | MS4A1 | EVQLVESGGGLVQPGRSLRLSCAASGFTFNDYAMHWVRQAP |
| | (ARZERRA® | (membrane- | GKGLEWVSTISWNSGSIGYADSVKGRFTISRDNAKKSLYLQ |
| | KESIMPTA®) | spanning 4- | MNSLRAEDTALYYCAKDIQYGNYYYGMDVWGQGTTVTVSSA |
| | | domains subfamily | STKGPSVFPLAPGSSKSTSGTAALGCLVKDYFPEPVTVSWN |
| | | A member 1, | SGALTSGVHTFPAVLQSSGLYSLSSVVTVPSSSLGTQTYIC |
| | | CD20) [Homo | NVNHKPSNTKVDKKVEP |
| | | sapiens] | |
| 31 | olaratumab | PDGFRA (platelet- | QLQLQESGPGLVKPSETLSLTCTVSGGSINSSSYYWGWLRQ |
| | (LARTRUVO™) | derived growth | SPGKGLEWIGSFFYTGSTYYNPSLRSRLTISVDTSKNQFSL |
| | | factor receptor | MLSSVTAADTAVYYCARQSTYYYGSGNYYGWFDRWDQGTLV |
| | | alpha subunit, | TVSSASTKGPSVFPLAPSSKSTSGGTAALGCLVKDYFPEPV |
| | | PDGFR2, CD140a) | TVSWNSGALTSGVHTFPAVLQSSGLYSLSSVVTVPSSSLGT |
| | | [Homo sapiens] | QTYICNVNHKPSNTKVDKRVEPKSCDKTHTCPPCPAPELLG |
| | | | GPSVFLFPPKPKDTLMISRTPEVTCVVVDVSHEDPEVKFNW |
| | | | YVDGVEVHNAKTKPREEQYNSTYRVVSVLTVLHQDWLNGKE |
| | | | YKCKVSNKALPAPIEKTISKAKGQPREPQVYTLPPSREEMT |
| | | | KNQVSLTCLVKGFYPSDIAVEWESNGQPENNYKTTPPVLDS |
| | | | DGSFFLYSKLTVDKSRWQQGNVFSCSVMHEALHNHYTQKSL |
| | | | SLSPG |
| 32 | panitumumab | EGFR (epidermal | QVQLQESGPGLVKPSETLSLTCTVSGGSVSSGDYYWTWIRQ |
| | (VECTIBIX®) | growth factor | SPGKGLEWIGHIYYSGNTNYNPSLKSRLTISIDTSKTQFSL |
| | | receptor, receptor | KLSSVTAADTAIYYCVRDRVTGAFDIWGQGTMVTVSSASTK |
| | | tyrosine-protein | GPSVFPLAPCSRSTSESTAALGCLVKDYFPEPVTVSWNSGA |
| | | kinase erbB-1, | LTSGVHTFPAVLQSSGLYSLSSVVTVPSSNFGTQTYTCNVD |
| | | ERBB1, HER1, | HKPSNTKVDKTVERK |
| | | HER-1, ERBB) | |
| | | [Homo sapiens] | |
| 33 | pembrolizumab | PDCD1 | VQLVQSGVEVKKPGASVKVSCKASGYTFTNYYMYWVRQAPG |
| | (KEYTRUDA®) | (programmed cell | QGLEWMGGINPSNGGTNFNEKFKNRVTLTTDSSTTTAYMEL |
| | | death 1, PD1, PD- | KSLQFDDTAVYYCARRDYRFDMGFDYWGQGTTVTVSSASTK |
| | | 1, CD279) [Homo | GPSVFPLAPCSRSTSESTAALGCLVKDYFPEPVTVSWNSGA |
| | | sapiens] | LTSGVHTFPAVLQSSGLYSLSSVVTVPSSSLGTKTYTCNVD |
| | | | HKPSNTKVDKRVESKYGPPCPPCPAPEFLGGPSVFLFPPKP |
| | | | KDTLMISRTPEVTCVVVDVSQEDPEVQFNWYVDGVEVHNAK |
| | | | TKPREEQFNSTYRVVSVLTVLHQDWLNGKEYKCKVSNKGLP |
| | | | SSIEKTISKAKGQPREPQVYTLPPSQEEMTKNQVSLTCLVK |
| | | | GFYPSDIAVEWESNGQPENNYKTTPPVLDSDGSFFLYSRLT |
| | | | VDKSRWQEGNVFSCSVMHEALHNHYTQKSLSLSLGK |
| 34 | pertuzumab | ERBB2 (epidermal | EVQLVESGGGLVQPGGSLRLSCAASGFTFTDYTMDWVRQAP |
| | (PERJETA®) | growth factor | GKGLEWVADVNPNSGGSIYNQRFKGRFTLSVDRSKNTLYLQ |
| | | receptor 2, receptor | MNSLRAEDTAVYYCARNLGPSFYFDYWGQGTLVTVSSASTK |
| | | tyrosine-protein | GPSVFPLAPSSKSTSGGTAALGCLVKDYFPEPVTVSWNSGA |
| | | kinase erbB-2, | LTSGVHTFPAVLQSSGLYSLSSVVTVPSSSLGTQTYICNVN |
| | | EGFR2, HER2, | HKPSNTKVDKKVEPKSCDKTH |
| | | HER-2, p185c- | |
| | | erbB2, NEU, | |
| | | CD340) [Homo | |
| | | sapiens] | |
| 35 | ramucirumab | KDR (kinase insert | EVQLVQSGGGLVKPGGSLRLSCAASGFTFSSYSMNWVRQAP |
| | (CYRAMZA™) | domain receptor, | GKGLEWVSSISSSSSYIYYADSVKGRFTISRDNAKNSLYLQ |
| | | vascular | MNSLRAEDTAVYYCARVTDAFDIWGQGTMVTVSSASTKGPS |
| | | endothelial growth | VLPLAPSSKSTSGGTAALGCLVKDYFPEPVTVSWNSGALTS |
| | | factor receptor 2, | GVHTFPAVLQSSGLYSLSSVVTVPSSSLGTQTYICNVNHKP |
| | | VEGFR2, VEGF- | SNTKVDKRVEPKSCDKTHTCPPCPAPELLGGPSVFLFPPKP |
| | | R2, FLK1, CD309) | KDTLMISRTPEVTCVVVDVSHEDPEVKFNWYVDGVEVHNAK |
| | | [Homo sapiens] | TKPREEQYNSTYRVVSVLTVLHQDWLNGKEYKCKVSNKALP |
| | | | APIEKTISKAKGQPREPQVYTLPPSREEMTKNQVSLTCLVK |
| | | | GFYPSDIAVEWESNGQPENNYKTTPPVLDSDGSFFLYSKLT |
| | | | VDKSRWQQGNVFSCSVMHEALHNHYTQKSLSLSPGK |
| 36 | rituximab | MS4A1 | QVQLQQPGAELVKPGASVKMSCKASGYTFTSYNMHWVKQTP |
| | (RITUXAN®) | (membrane- | GRGLEWIGAIYPGNGDTSYNQKFKGKATLTADKSSSTAYMQ |
| | | spanning 4- | LSSLTSEDSAVYYCARSTYYGGDWYFNVWGAGTTVTVSAAS |
| | | domains subfamily | TKGPSVFPLAPSSKSTSGGTAALGCLVKDYFPEPVTVSWNS |
| | | A member 1, | GALTSGVHTFPAVLQSSGLYSLSSVVTVPSSSLGTQTYICN |
| | | CD20) [Homo | VNHKPSNTKVDKKVEPKSCDKTHTCPPCPAPELLGGPSVFL |
| | | sapiens] | FPPKPKDTLMISRTPEVTCVVVDVSHEDPEVKFNWYVDGVE |
| | | | VHNAKTKPREEQYNSTYRVVSVLTVLHQDWLNGKEYKCKVS |
| | | | NKALPAPIEKTISKAKGQPREPQVYTLPPSRDELTKNQVSL |
| | | | TCLVKGFYPSDIAVEWESNGQPENNYKTTPPVLDSDGSFFL |
| | | | YSKLTVDKSRWQQGNVFSCSVMHEALHNHYTQKSLSLSPGK |
| 37 | trastuzumab | ERBB2 (epidermal | EVQLVESGGGLVQPGGSLRLSCAASGFNIKDTYIHWVRQAP |
| | (HERCEPTIN®) | growth factor | GKGLEWVARIYPTNGYTRYADSVKGRFTISADTSKNTAYLQ |
| | | receptor 2, receptor | MNSLRAEDTAVYYCSRWGGDGFYAMDYWGQGTLVTVSSAST |
| | | tyrosine-protein | KGPSVFPLAPSSKSTSGGTAALGCLVKDYFPEPVTVSWNSG |
| | | kinase erbB-2, | ALTSGVHTFPAVLQSSGLYSLSSVVTVPSSSLGTQTYICNV |
| | | EGFR2, HER2, | NHKPSNTKVDKKVEPKSCDKTHTCPPCPAPELLGGPSVFLF |
| | | HER-2, p185c- | PPKPKDTLMISRTPEVTCVVVDVSHEDPEVKFNWYVDGVEV |
| | | erbB2, NEU, | HNAKTKPREEQYNSTYRVVSVLTVLHQDWLNGKEYKCKVSN |
| | | CD340) [Homo | KALPAPIEKTISKAKGQPREPQVYTLPPSREEMTKNQVSLT |
| | | sapiens] | CLVKGFYPSDIAVEWESNGQPENNYKTTPPVLDSDGSFFLY |
| | | | SKLTVDKSRWQQGNVFSCSVMHEALHNHYTQKSLSLSPG |

*Sequences in Table 5 were determined by IMGT-monoclonal antibody database

As an alternative to, or in addition to the delivery of RNAs as antigens, combinations can be used, e.g., RNA antigens combined with RNAs that stimulate innate immune responses, or RNAs that launch oncolytic viruses, or live-attenuated viruses.

In certain embodiments, the bioactive agent in of a composition provided herein comprises a combination of RNA-encoded antigens with another RNA that can stimulate innate immune responses or can launch oncolytic viruses or live-attenuated viruses. Alternatively, compositions provided herein that contain RNA-encoded antigens can be combined with a formulation that contains another RNA that can stimulate innate immune responses or can launch oncolytic viruses or live-attenuated viruses.

### RNA Coding for a RNA Polymerase

Provided herein are compositions comprising a self-replicating nucleic acid. In some embodiments, compositions provided herein comprise one or more nucleic acids. In some embodiments, compositions provided herein comprise two or more nucleic acids. In some embodiments, nucleic acids provided herein code for an RNA polymerase. In some embodiments, nucleic acids provided herein code for a viral RNA polymerase. In some embodiments, nucleic acids provided herein code for: (1) a viral RNA polymerase; and (2) a protein, antibody, or functional fragment thereof. In some embodiments, compositions provided herein comprise a first nucleic acid coding for a viral RNA polymerase; and a second nucleic acid coding for a protein, antibody, or functional fragment thereof.

Provided herein are compositions comprising a self-replicating RNA. A self-replicating RNA (also called a replicon) includes any genetic element, for example, a plasmid, cosmid, bacmid, phage or virus that is capable of replication largely under its own control. Self-replication provides a system for self-amplification of the nucleic acids provided herein in mammalian cells. In some embodiments, the self-replicating RNA is single stranded. In some embodiments, the self-replicating RNA is double stranded.

An RNA polymerase provided herein can include but is not limited to: an alphavirus RNA polymerase, an Eastern equine encephalitis virus (EEEV) RNA polymerase, a Western equine encephalitis virus (WEEV), Venezuelan equine encephalitis virus (VEEV), Chikungunya virus (CHIKV), Semliki Forest virus (SFV), or Sindbis virus (SINV). In some embodiments, the RNA polymerase is a VEEV RNA polymerase. In some embodiments, the nucleic acid coding for the RNA polymerase comprises at least 85% identity to the nucleic acid sequence of SEQ ID NO: 38. In some embodiments, the nucleic acid coding for the RNA polymerase comprises at least 90% identity to the nucleic acid sequence of SEQ ID NO: 38. In some embodiments, the nucleic acid coding for the RNA polymerase comprises at least 95% identity to the nucleic acid sequence of SEQ ID NO: 38. In some embodiments, the nucleic acid coding for the RNA polymerase comprises at least 99% identity to the nucleic acid sequence of SEQ ID NO: 38. In some embodiments, the nucleic acid coding for the RNA polymerase is SEQ ID NO: 38.

In some embodiments, the amino acid sequence for VEEV RNA polymerase comprises at least 85% identity to RELPVLDSAAFNVECFKKYACNNEYWETFKENPIRLTEEN VVNYITKLKGP (SEQ ID NO: 39) or TQMRELPVLDSAAFNVECFKKYACNNEYWE TFKENPIRLTE (SEQ ID NO: 40). In some embodiments, the amino acid sequence for VEEV RNA polymerase comprises at least 90% identity to SEQ ID NO: 39 or SEQ ID NO: 40. In some embodiments, the amino acid sequence for VEEV RNA polymerase comprises at least 95% identity to SEQ ID NO: 39 or SEQ ID NO: 40. In some embodiments, the amino acid sequence for VEEV RNA polymerase comprises at least 99% identity to SEQ ID NO: 39 or SEQ ID NO: 40. In some embodiments, the amino acid sequence for VEEV RNA polymerase is SEQ ID NO: 39 or SEQ ID NO: 40.

### Protein Expression Enhancer RNA

Provided herein are compositions comprising a nanoparticle; a first nucleic acid coding for at least one protein or fragment thereof; and a second nucleic acid coding for at least one expression enhancer or fragment thereof, wherein expression enhancer increases expression of the protein or the fragment thereof. In some embodiments, the plurality of protein comprises a plurality of protein expression enhancer. In some embodiments, the plurality of protein expression enhancer comprises two, three, four, five, six, seven, eight, nine, or ten protein expression enhancers. In some embodiments, at least two of the plurality of protein expression enhancer are the same. In some embodiments, at least two of the plurality of protein expression enhancer are different. In some embodiments, Also, provided herein are compositions comprising a nanoparticle; and a plurality of nucleic acid, wherein at least one of the plurality of nucleic acid encodes a protein expression enhancer.

In some embodiments, the protein expression enhancer comprises a kinase inhibitor. In some embodiments, the kinase inhibitor comprises a casein kinase inhibitor, a cyclin-dependent kinase (CDK) inhibitor, an extracellular signal-regulated kinase (ERK) inhibitor, a growth factor inhibitor, a glycogen synthase kinase inhibitor, an immune checkpoint inhibitor, a Janus kinase (JAK) inhibitor, a IκB kinase (IKK) inhibitor, a glycogen synthase kinase-3β (GSK-3β) inhibitor, a lipid kinase inhibitor, a mitogen-activated protein kinase (MAPK) family inhibitor, a phosphatidylinositol 4-kinase (P14K) inhibitor, a polo-like kinase (PLK) inhibitor, a protein kinase D (PKD) inhibitor, a tyrosine kinase inhibitor, a T-lymphokine-activated killer cell-originated protein kinase (TOPK) inhibitor, a salt inducible kinase (SIK) inhibitor, or a Wnt signaling inhibitor. In some embodiments, the kinase inhibitor is a cyclin-dependent kinase (CDK) inhibitor. In some embodiments, the CDK inhibitor comprises an amino acid sequence that is encoded by a nucleic acid having at least 70%, 75%, 80%, 85%, 90%, 95%, 98%, or 100% identical to any one of the sequences of SEQ ID NO: 41-47.

### Protein Expression Enhancer Compounds

Provided herein are compositions and kits comprising a compound. Provided herein are compositions comprising a nanoparticle; a nucleic acid encoding a protein, antibody, or fragment thereof; and a compound. In some embodiments, the compound enhances expression of the protein, antibody, or the functional fragment thereof in mammalian cells. In some embodiments, the compound is dispersed in the hydrophobic core of the nanoparticle. In some embodiments, the compound is conjugated to the nanoparticle. Compounds provided herein can have an anti-cancer or anti-viral effect on a mammalian cell or a subject. In some embodiments, compounds provided herein inhibit or stabilize tumor growth. In some embodiments, compounds provided herein decrease cancer cell proliferation or survival. In some embodiments, compounds provided herein inhibit viral fusion with a mammalian cell. In some embodiments, compounds provided herein inhibit viral replication within a mammalian cell. In some embodiments, compounds provided herein are immunostimulatory. In some embodiments, compounds provided herein are immunosuppressive. In some embodiments, compounds provided herein suppress interferon-α expression or activity. In some embodiments, compounds provided herein modify NFκB expression or activity. In some embodiments, compounds provided herein modify NFκB expression or activity over interferon-α expression or activity. In some embodiments, the modification is an increase. In some embodiments, the modification is a decrease.

In some embodiments, the compound is a kinase inhibitor. Kinase inhibitors are compounds that inhibit the enzymatic activity of at least one kinase. In some embodiments, the kinase inhibitor is a flavone or flavonoid derivative.

In some embodiments, the kinase inhibitor is a CDK inhibitor. Cyclin-dependent kinase (CDK) complexes, are protein kinases that are involved in the regulation of cell growth. These complexes comprise at least a catalytic (the CDK itself) and a regulatory (cyclin) subunit. Exemplary complexes for cell cycle regulation include cyclin A (CDK1-also known as cdc2, and CDK2), cyclin B1-B3 (CDK1) and cyclin D1-D3 (CDK2, CDK4, CDK5, CDK6), cyclin E (CDK2). Each of these complexes are involved in a particular phase of the cell cycle. CDKs are involved in cell cycle regulation, gene transcription, insulin secretion, glycogen synthesis and neuronal functions. CDKs that directly promote cell cycle progression include CDK4, CDK6, CDK2 and CDK1. Exemplary CDK inhibitors are provided in Table 5.

In some embodiments, compositions provided herein comprise one or more of the compounds listed in Table 6, Table 7, or Table 13.

TABLE 6
Exemplary Kinase Inhibitors

| | Molecular | | |
|---|---|---|---|
| Name | Formula | CAS No. | IUPAC Name |
| SB1317 | C₂₃H₂₄N₄O | 1204918-72- | (16E)-14-methyl-20-oxa-5,7,14,27- |
| | | 8 | tetrazatetracyclo[19.3.1.12,6.18,12]heptacosa- |
| | | | 1(25),2(27),3,5,8,10,12(26),16,21,23-decaene |
| RGB286638 | C₂₉H₃₇Cl₂N₇O₄ | 784210-87-3 | 1-[3-[4-[[4-(2-methoxyethyl)piperazin-1-yl]methyl]phenyl]-4- |
| | | | oxo-1H-indeno[1,2-c]pyrazol-5-yl]-3-morpholin-4- |
| | | | ylurea;dihydrochloride |
| SNS032 | C₁₇H₂₄N₄O₂S₂ | 345627-80-7 | N-[5-[[5-(1,1-Dimethylethyl)-2-oxazolyl]methyl]thio]-2- |
| | | | thiazolyl]-4-piperidinecarboxamide |
| Palbociclib | C₂₄H₂₉N₇O₂ | 571190-30-2 | 6-acetyl-8-cyclopentyl-5-methyl-2-[(5-piperazin-1-ylpyridin- |
| | | | 2-yl)amino]pyrido[2,3-d]pyrimidin-7-one |
| Voruciclib | C₂₂H₁₉ClF₃NO₅ | 1000023-04- | 2-[2-chloro-4-(trifluoromethyl)phenyl]-5,7-dihydroxy-8- |
| | | 0 | [(2R,3S)-2-(hydroxymethyl)-1-methylpyrrolidin-3- |
| | | | yl]chromen-4-one |
| abemaciclib | C₂₇H₃₂F₂N₈ | 1231929-97- | N-[5-[(4-ethylpiperazin-1-yl)methyl]pyridin-2-yl]-5-fluoro-4- |
| | | 7 | (7-fluoro-2-methyl-3-propan-2-ylbenzimidazol-5- |
| | | | yl)pyrimidin-2-amine |
| PF- | C₂₀H₂₇F₂N₅O₄S | 2185857-97- | 6-(difluoromethyl)-8-[(1R,2R)-2-hydroxy-2- |
| 06873600 | | 8 | methylcyclopentyl]-2-[(1-methylsulfonylpiperidin-4- |
| | | | yl)amino]pyrido[2,3-d]pyrimidin-7-one |
| ribociclib | C₂₃H₃₀N₈O | 1211441-98- | 7-cyclopentyl-N,N-dimethyl-2-[(5-piperazin-1-ylpyridin-2- |
| | | 3 | yl)amino]pyrrolo[2,3-d]pyrimidine-6-carboxamide |
| trilaciclib | C₂₄H₃₀N₈O | 1374743-00- | 4-[[5-(4-methylpiperazin-1-yl)pyridin-2- |
| | | 6 | yl]amino]spiro[1,3,5,11-tetrazatricyclo[7.4.0.02,7]trideca- |
| | | | 2,4,6,8-tetraene-13,1′-cyclohexane]-10-one |
| dinaciclib | C₂₁H₂₈N₆O₂ | 779353-01-4 | 2-[(2S)-1-[3-ethyl-7-[(1-oxidopyridin-1-ium-3- |
| | | | yl)methylamino]pyrazolo[1,5-a]pyrimidin-5-yl]piperidin-2- |
| | | | yl]ethanol |
| seliciclib | C₁₉H₂₆N₆O | 186692-46-6 | (2R)-2-[[6-(benzylamino)-9-propan-2-ylpurin-2- |
| | | | yl]amino]butan-1-ol |
| milciclib | C₂₅H₃₂N₈O | 802539-81-7 | N,1,4,4-tetramethyl-8-[4-(4-methylpiperazin-1-yl)anilino]-5H- |
| | | | pyrazolo[4,3-h]quinazoline-3-carboxamide |
| AZD-5438 | C₁₈H₂₁N₅O₂S | 602306-29-6 | 4-(1-isopropyl-2-methyl-1H-imidazol-5-yl)-N-(4- |
| | | | (methylsulfonyl)phenyl)pyrimidin-2-amine |
| simurosertib | C₁₇H₁₉N₅OS | 1330782-76- | 2-[(2S)-1-azabicyclo[2.2.2]octan-2-yl]-6-(5-methyl-1H- |
| | | 7 | pyrazol-4-yl)-3H-thieno[3,2-d]pyrimidin-4-one |
| AT7519 | C₁₆H₁₈C₁₃N₅O₂ | 902135-91-5 | 4-[(2,6-dichlorobenzoyl)amino]-N-piperidin-4-yl-1H- |
| | | | pyrazole-5-carboxamide;hydrochloride |
| CDK-IN-2 | C₁₈H₁₉ClFN₃O₂ | 1269815-17- | (3R)-N-[5-chloro-4-(5-fluoro-2-methoxyphenyl)pyridin-2- |
| | | 9 | yl]piperidine-3-carboxamide |
| 7- | C₂₈H₂₆N₄O₄ | 112953-11-4 | (2S,3R,4R,6R,18R)-18-hydroxy-3-methoxy-2-methyl-4- |
| hydroxystaurosporine | | | (methylamino)-29-oxa-1,7,17- |
| | | | triazaoctacyclo[12.12.2.1^{2,6}.0^{7,28}.0^{8,13}.0^{15,19}.0^{20,27}.0^{21,26}]nonacosa- |
| | | | 8,10,12,14,19,21,23,25,27-nonaen-16-one |
| alvocidib or | C₂₁H₂₀ClNO₅ | 146426-40-6 | 2-(2-chlorophenyl)-5,7-dihydroxy-8-[(3S,4R)-3-hydroxy-1- |
| flavopiridol | | | methylpiperidin-4-yl]chromen-4-one |
| JNJ- | C₁₅H₁₂F₂N₆O₃S | 443797-96-4 | 4-({5-Amino-1-[(2,6-Difluorophenyl)carbonyl]-1h-1,2,4- |
| 7706621 | | | Triazol-3-Y1}amino)benzenesulfonamide |
| BMS- | C₁₈H₂₆N₄O₂S₂ | | 4-amino-N-[5-[(5-tert-butyl-1,3-oxazol-2-yl)methylsulfanyl]- |
| 387032 | | | 1,3-thiazol-2-yl]cyclohexane-1-carboxamide |
| riviciclib | C₂₁H₂₀ClNO₅ | 920113-02-6 | 2-(2-chlorophenyl)-5,7-dihydroxy-8-[2-(hydroxymethyl)-1- |
| | | | methylpyrrolidin-3-yl]chromen-4-one |
| ZK-304709 | C₁₃H₁₆BrN₅O₃S | 477589-41-6 | 4-[[5-bromo-4-[[(2R)-1-hydroxypropan-2- |
| | | | yl]amino]pyrimidin-2-yl]amino]benzenesulfonamide |
| ON-123300 | C₂₄H₂₇N₇O | 1357470-29- | 8-cyclopentyl-2-[4-(4-methylpiperazin-1-yl)anilino]-7- |
| | | 1 | oxopyrido[2,3-d]pyrimidine-6-carbonitrile |
| CYC-065 | C₂₁H₃₁N₇O | 1070790-89- | (2R,3S)-3-[[6-[(4,6-dimethylpyridin-3-yl)methylamino]-9- |
| | | 4 | propan-2-ylpurin-2-yl]amino]pentan-2-ol |
| LS-007 | C₁₅H₁₅FN₆O₂S₂ | 1421693-22- | 3-[[5-fluoro-4-[4-methyl-2-(methylamino)-1,3-thiazol-5- |
| (CDKI73) | | 2 | yl]pyrimidin-2-yl]amino]benzenesulfonamide |
| PHA793887 | C₁₉H₃₂ClN₅O₂ | 718630-60-5 | N-[6,6-dimethyl-5-(1-methylpiperidine-4-carbonyl)-1,4- |
| | | | dihydropyrrolo[3,4-c]pyrazol-3-yl]-3-methylbutanamide, |
| | | | hydrochloride |
| TG-02 | C₂₉H₃₂N₄O₈ | 1204918-73- | 2-hydroxypropane-1,2,3-tricarboxylic acid;(16E)-14-methyl- |
| | | 9 | 20-oxa-5,7,14,27-tetrazatetracyclo[19.3.1.1^{2,6}.1^{8,12}]heptacosa- |
| | | | 1(25),2(27),3,5,8,10,12(26),16,21,23-decaene |
| R547 | C₁₈H₂₁F₂N₅O₄S | 741713-40-6 | (4-Amino-2-((1-(methylsulfonyl)piperidin-4- |
| | | | yl)amino)pyrimidin-5-yl)(2,3-difluoro-6- |
| | | | methoxyphenyl)methanone |
| RGB286147 | C₂₃H₂₂ClN₄O₃ | 784211-09-2 | 1-(2,6-dichlorophenyl)-6-[[4-(2- |
| | | | hydroxyethoxy)phenyl]methyl]-3-propan-2-yl-5H- |
| | | | pyrazolo[3,4-d]pyrimidin-4-one |
| olomoucine | C₁₅H₁₈N₆O | 101622-51-9 | 2-[[6-(benzylamino)-9-methylpurin-2-yl]amino]ethanol |
| olomoucine | C₁₉H₂₆N₆O₂ | 500735-47-7 | 2-[[2-[[(2R)-1-hydroxybutan-2-yl]amino]-9-propan-2- |
| II | | | ylpurin-6-yl]amino]methyl]phenol |
| purvalanol A | C₁₉H₂₅ClN₆O | 212844-53-6 | (2R)-2-[6-(3-chloroanilino)-9-propan-2-ylpurin-2-yl]amino]- |
| | | | 3-methylbutan-1-ol |
| purvalano1 B | C₂₀H₂₅ClN₆O₃ | 212844-54-7 | 2-chloro-4-[2-[[(2R)-1-hydroxy-3-methylbutan-2-yl]amino]- |
| | | | 9-propan-2-ylpurin-6-yl]amino]benzoic acid |
| AT9311 or | C₁₇H₁₉ClN₅O₄S | 902156-99-4 | 4-[(2,6-dichlorobenzoyl)amino]-N-(1- |
| NVP- | | | methylsulfonylpiperidin-4-yl)-1H-pyrazole-5-carboxamide |
| LCQ195 | | | |
| meriolin 3 | C₁₂H₁₁N₅O | 954143-48-7 | 4-(4-Methoxy-1h-Pyrrolo[2,3-B]pyridin-3-Y1)pyrimidin-2- |
| | | | Amine |
| kenpaullone | C₁₆H₁₁BrN₂O | 142273-20-9 | 9-bromo-7,12-dihydro-5H-indolo[3,2-d][1]benzazepin-6-one |
| indirubin-3′- | C₁₆H₁₁N₃O₂ | 160807-49-8 | 3-(3-nitroso-1H-indol-2-yl)-1H-indol-2-o1 |
| monoxime | | | |
| indirubin | C₁₆H₁₀N₂O₂ | 479-41-4 | 2-(2-hydroxy-1H-indol-3-yl)indol-3-one |
| bohemine | C₁₈H₂₄N₆O | 189232-42-6 | 3-[[6-(benzylamino)-9-propan-2-ylpurin-2-yl]amino]propan- |
| | | | 1-ol |
| butylactone I | C₂₄H₂₄O₇ | 87414-49-1 | methyl (2R)-4-hydroxy-2-[[4-hydroxy-3-(3-methylbut-2- |
| | | | enyl)phenyl]methyl]-3-(4-hydroxyphenyl)-5-oxofuran-2- |
| | | | carboxylate |
| 10Z- | C₁₁H₁₀BrN₅O₂ | 82005-12-7 | (4Z)-4-(2-amino-5-oxo-1H-imidazol-4-ylidene)-2-bromo- |
| hymenialdisine | | | 1,5,6,7-tetrahydropyrrolo[2,3-c]azepin-8-one |
| 5-iodo- | C₁₆H₁₀IN₃O₂ | 331467-03-9 | 5-iodo-3-(3-nitroso-1H-indo1-2-yl)-1H-indol-2-ol |
| indirubin-3′- | | | |
| monoxime | | | |
| AG024322 | C₂₃H₂₀F₂N₆ | 837364-57-5 | N-[[5-[3-(4,6-difluoro-1H-benzimidazol-2-yl)-1H-indazol-5- |
| | | | yl]-4-methylpyridin-3-yl]methyl]ethanamine |
| aloisine A | C₁₆H₁₇N₃O | 496864-16-5 | 4-(7-butyl-5H-pyrrolo[2,3-b]pyrazin-6-yl)phenol |
| aloisine B | C₁₅H₁₄ClN₃ | 496864-14-3 | 6-(4-chlorophenyl)-7-propan-2-yl-5H-pyrrolo[2,3-b]pyrazine |
| alsterpaullone | C₁₆H₁₁N₃O₃ | 237430-03-4 | 9-nitro-7,12-dihydro-5H-indolo[3,2-d][1]benzazepin-6-one |
| aminopurvalanol | C₁₉H₂₆ClNO | 220792-57-4 | (2R)-2-[6-(3-amino-5-chloroanilino)-9-propan-2-ylpurin-2- |
| | | | yl]amino]-3-methylbutan-1-ol |
| R-CR8 | C₂₄H₃₂Cl₃N₇O | 1786438-30- | (2R)-2-[9-propan-2-yl-6-[(4-pyridin-2- |
| | | 9 | ylphenyl)methylamino]purin-2-yl]amino]butan-1- |
| | | | ol; trihydrochloride |
| BAY- | C₁₉H₁₈F₂N₄O₂S | 1610358-59- | 5-fluoro-4-(4-fluoro-2-methoxyphenyl)-N-[4- |
| 1251152 | | 2 | [(methylsulfonimidoy])methyl]pyridin-2-yl]pyridin-2-amine |
| AUZ 454 | C₂₄H₂₆F₃N₇O₂ | 853299-07-7 | 1-[4-(2-aminopyrimidin-4-yl)oxyphenyl]-3-[4-[(4- |
| | | | methylpiperazin-1-yl)methyl]-3-(trifluoromethyl)phenyl]urea |
| AZD4573 | C₂₂H₂₈ClN₅O₂ | 2057509-72- | (IS,3R)-3-acetamido-N-[5-chloro-4-(5,5-dimethyl-4,6- |
| | | 3 | dihydropyrrolo[1,2-b]pyrazol-3-yl)pyridin-2-yl]cyclohexane- |
| | | | 1-carboxamide |
| CDK12-IN-3 | C₂₃H₂₈F₂N₈O | 2220184-50- | 2-[(2S)-1-[6-[(4,5-difluoro-1H-benzimidazol-2- |
| | | 7 | yl)methylamino]-9-propan-2-ylpurin-2-yl]piperidin-2- |
| | | | yl]ethanol |
| CDK9-IN-8 | C₃₁H₃₂FN₇O₃ | 2105956-51- | 1-N-[4-[[7-cyclopentyl-6-(dimethylcarbamoy])pyrrolo[2,3- |
| | | 0 | d]pyrimidin-2-yljamino]phenyl-1-N′-(4. |
| | | | fluorophenyl)cyclopropane-1,1-dicarboxamide |
| K00546 | C₁₅H₁₃F₂N₇O₂S₂ | 443798-47-8 | 5-amino-N-(2,6-difluoropheny])-3-(4-sulfamoylanilino)-1,2,4- |
| | | | triazole-1-carbothioamide |
| KB-0742 | C₁₆H₂₇ClN₅ | 2416874-75- | (1S,3S)-3-N-(5-pentan-3-ylpyrazolo[1,5-a]pyrimidin-7- |
| | | 2 | yl)cyclopentane-1,3-diamine; dihydrochloride |
| LDC4297 | C₂₃H₂₈N₈O | 1453834-21- | 2-piperidin-3-yloxy-8-propan-2-yl-N-[(2-pyrazol-1- |
| | | 3 | ylphenyl)methyl]pyrazolo[1,5-a][1,3,5]triazin-4-amine |
| LSN3106729 | C₂₅H₂₉ClF₂N₈O | | |
| LY2857785 | C₂₆H₃₆N₆O | 1619903-54-6 | 4-N-14-(2-methyl-3-propan-2-ylindazol-5-yl)pyrimidin-2-yl]- |
| | | | 1-N-(oxan-4-yl)cyclohexane-1,4-diamine |
| MC180295 | C₁₇H₁₈N₄O₃S | 2237942-08- | [4-amino-2-[[(1S,2S,4R)-2-bicyclo[2.2.1]heptanyl]amino]-1,3- |
| | | 2 | thiazol-5-yl]-(2-nitrophenyl)methanone |
| PHA- | C₁₂H₁₁N₃O | 845714-00-3 | 2-pyridin-4-yl-1,5,6,7-tetrahydropyrrolo[3,2-c]pyridin-4-one |
| 767491 | | | |
| PHA- | C₁₉H₃₁N₅O₂ | 718630-59-2 | N-[6,6-dimethyl-5-(1-methylpiperidine-4-carbonyl)-1,4- |
| 793887 | | | dihydropyrrolo[3,4-c]pyrazol-3-yl]-3-methylbutanamide |
| PROTAC | C₃₃H₃₅N₅O₇ | 2118356-96- | N-(5-cyclobutyl-1H-pyrazol-3-yl)-2-[4-[5-[2-(2,6- |
| CDK9 | | 8 | dioxopiperidin-3-yl)-1,3-dioxoisoindol-4- |
| Degrader-1 | | | yl]oxypentoxy]phenyl]acetamide |
| SU9516 | C₁₃H₁₁N₃O₂ | 377090-84-1 | (3Z)-3-(1H-imidazol-5-ylmethylidene)-5-methoxy-1H-indol- |
| | | | 2-one |
| THZ2 | C₃₁H₂₈ClN₇O₂ | 1604810-84- | N-[3-[[5-chloro-4-(1H-indol-3-yl)pyrimidin-2- |
| | | 5 | yl]amino]phenyl]-3-[[(E)-4-(dimethylamino)but-2- |
| | | | enoyl]amino]benzamide |
| (2S,3R)- | C₂₂H₂₀Cl₂F₃NO₅ | 1253731-24- | 2-[2-chloro-4-(trifluoromethyl)phenyl]-5,7-dihydroxy-8- |
| voruciclib | | 6 free base | [(2S,3R)-2-(hydroxymethyl)-1-methylpyrrolidin-3- |
| | | | yl]chromen-4-one; hydrochloride |
| DMX-5804 | C₂₁H₁₉N₃O₃ | 2306178-56- | 5-[4-(2-methoxyethoxy)phenyl]-7-phenyl-3H-pyrrolo[2,3- |
| | | 1 | d]pyrimidin-4-one |
| selonsertib | C₂₄H₂₄FN₇O | 1448428-04- | 5-(4-cyclopropylimidazol-1-yl)-2-fluoro-4-methyl-N-[6-(4- |
| | | 3 | propan-2-yl-1,2,4-triazol-3-yl)pyridin-2-yl]benzamide |
| NCB-0846 | C₂₁H₂₁N₅O₂ | 1792999-26- | 4-[2-(3H-benzimidazol-5-ylamino)quinazolin-8- |
| | | 8 | yl]oxycyclohexan-1-ol |
| MAPK13- | C₂₀H₂₃N₅O₂ | 229002-10-2 | 1-(5-tert-butyl-2-methylpyrazol-3-yl)-3-(4-pyridin-4- |
| IN-1 | | | yloxyphenyl)urea |
| LY2874455 | C₂₁H₁₉Cl₂N₅O₂ | 1254473-64- | 2-[4-[(E)-2-[5-[(1R)-1-(3,5-dichloropyridin-4-y])ethoxy]-1H- |
| | | 7 | indazol-3-yl]ethenyl]pyrazol-1-yl]ethanol |
| altiratinib | C₂₆H₂₁F₃N₄O₄ | 1345847-93- | 1-N′-[4-[2-(cyclopropanecarbonylamino)pyridin-4-yl]oxy-2,5- |
| | | 9 | difluorophenyl]-1-N-(4-fluorophenyl)cyclopropane-1,1- |
| | | | dicarboxamide |
| autophinib | C₁₄H₁₁ClN₆O₃ | 1644443-47- | 6-chloro-N-(5-methyl-1H-pyrazol-3-yl)-2-(4- |
| | | 9 | nitrophenoxy)pyrimidin-4-amine |
| AST 487 | C₂₆H₃₀F₃N₇O₂ | 630124-46-8 | 1-[4-[(4-ethylpiperazin-1-y])methyl]-3- |
| | | | (trifluoromethyl)phenyl]-3-[4-[6-(methylamino)pyrimidin-4- |
| | | | yl]oxyphenyl]urea |
| BMS- | C₂₃H₁₅ClF₂N₄O₃ | 1174046-72- | N-[4-(2-amino-3-chloropyridin-4-yl)oxy-3-fluorophenyl]-5- |
| 794833 | | 0 | (4-fluorophenyl)-4-oxo-1H-pyridine-3-carboxamide |
| SCR- | C₂₈H₂₉ClF₂N₅O₁₀ | 1174161-86- | [3-[[4-(2-amino-3-chloropyridin-4-yl)oxy-3- |
| 1481B1 | P | 4 | fluorophenyl]carbamoyl]-5-(4-fluorophenyl)-4-oxopyridin-1- |
| | | | yl]methyl dihydrogen phosphate; 2-amino-2- |
| | | | (hydroxymethyl)propane-1,3-diol |
| tyrosine | C₂₆H₂₈FN₅O | 705946-27-6 | (3Z)-5-[(1-ethylpiperidin-4-yl)amino]-3-[(3-fluorophenyl)-(5- |
| kinase-IN-1 | | | methyl-1H-imidazol-2-yl)methylidene]-1H-indol-2-one |
| VEGFR-2- | C₁₉H₂₄N₈ | 1430089-64- | 2-N-[4-(3-aminopropylamino)phenyl]-4-N-(5-cyclopropyl- |
| IN-5 | | 7 | 1H-pyrazol-3-yl)pyrimidine-2,4-diamine |
| XL228 | C₂₂H₃₁N₉O | 898280-07-4 | 4-N-(5-cyclopropyl-1H-pyrazol-3-yl)-6-(4-methylpiperazin-1- |
| | | | yl)-2-N-[(3-propan-2-yl-1,2-oxazol-5-yl)methyl]pyrimidine- |
| | | | 2,4-diamine |
| GW806742 | C₂₅H₂₂F₃N₇O₄S, | 579515-63-2 | 1-[4-[methyl-[2-(3-sulfamoylanilino)pyrimidin-4- |
| X | | | ylamino]phenyl]-3-[4-(trifluoromethoxy )phenyl]urea |
| AZ960 | C₁₈H₁₆F₂N₆ | 905586-69-8 | 5-fluoro-2-[[(1S)-1-(4-fluorophenyl)ethyl]amino]-6-[(5- |
| | | | methyl-1H-pyrazol-3-yl)amino]pyridine-3-carbonitrile |
| ilginatinib | C₂₁H₂₀FN₇ | 1239358-86- | 6-N-[(1S)-1-(4-fluorophenyl)ethyl]-4-(1-methylpyrazol-4-yl)- |
| | | 1 | 2-N-pyrazin-2-ylpyridine-2,6-diamine |
| Ilginatinib | C₂₁H₂₁ClFN₇ | 1239358-85- | 6-N-[(1S)-1-(4-fluorophenyl)ethyl]-4-(1-methylpyrazol-4-yl)- |
| hydrochloride | | 0 | 2-N-pyrazin-2-ylpyridine-2,6-diamine;hydrochloride |
| momelotinib | C₂₃H₂₂N₆O₂ | 1056634-68- | N-(cyanomethyl)-4-[2-(4-morpholin-4-ylanilino)pyrimidin-4- |
| | | 4 | yl]benzamide |
| momelotinib | C₂₃H₂₆N₆O₁₀S₂ | 1056636-06- | N-(cyanomethyl)-4-[2-(4-morpholin-4-ylanilino)pyrimidin-4- |
| sulfate | | 6 | yl]benzamide; sulfuric acid |
| ravoxertinib | C₂₁H₁₈ClFN₆O₂ | 1453848-26- | 1-[(1S)-1-(4-chloro-3-fluorophenyl)-2-hydroxyethyl]-4-[2-[(2- |
| | | 4 | methylpyrazol-3-yl)amino]pyrimidin-4-yl]pyridin-2-one |
| VX-11e | C₂₄H₂₀ClFN₅O₂ | 896720-20-0 | 4-[2-(2-chloro-4-fluoroanilino)-5-methylpyrimidin-4-yl]-N- |
| | | | [(1S)-1-(3-chlorophenyl)-2-hydroxyethyl]-1H-pyrrole-2- |
| | | | carboxamide |
| SBE13 | C₂₄H₂₇ClN₂O₄ | 775294-82-1 | N-[[4-[(6-chloropyridin-3-yl)methoxy]-3- |
| | | | methoxyphenyl]methyl]-2-(3,4-dimethoxyphenyl)ethanamine |
| HMN-214 | C₂₂H₂₀N₂O₅S | 173529-46-9 | N-(4-methoxyphenyl)sulfonyl-N-[2-[(E)-2-(1-oxidopyridin-1- |
| | | | ium-4-yl)ethenyl]phenyl]acetamide |
| BF738735 | C₂₁H₁₉FN₄O₃S | 1436383-95- | 2-fluoro-4-[2-methyl-8-[(3- |
| | | 7 | methylsulfonylphenyl)methylamino]imidazo{1,2-a]pyrazin-3- |
| | | | yl]phenol |
| R112 | C₁₆H₁₃FN₄O₂ | 575474-82-7 | 3-[[5-fluoro-2-(3-hydroxyanilino)pyrimidin-4- |
| | | | yl]amino]phenol |
| OTS514 | C₂₁H₂₀N₂O₂S | 1338540-63- | 9-[4-[(2R)-1-aminopropan-2-yl]phenyl]-8-hydroxy-6-methyl- |
| | | 8 | 5H-thieno[2,3-c]quinolin-4-one |
| CHIR- | C₂₂H₁₈Cl₂N₈ | 252917-06-9 | 6-[2-[[4-(2,4-dichlorophenyl)-5-(5-methyl-1H-imidazol-2- |
| 99021 | | | yl)pyrimidin-2-yl]amino]ethylamino]pyridine-3-carbonitrile |
| ACHP | C₂₁H₂₄N₄O₂ | 406208-42-2 | 2-amino-6-[2-(cyclopropylmethoxy)-6-bydroxyphenyl]-4- |
| | | | piperidin-4-ylpyridine-3-carbonitrile |
| BAY-985 | C₂₇H₃₀F₃N₉O | 2409479-29- | 1-[4-[(1R)-1-[2-[[6-[6-(dimethylamino)pyrimidin-4-yl]-1H- |
| | | 2 | benzimidazol-2-ylamino]pyridin-4-ylethyl]piperazin-1-yl]- |
| | | | 3,3,3-trifluoropropan-1-one |
| BMS- | C₁₄H₁₇N₅ | 445430-58-0 | N′-(1,8-dimethylimidazo[1,2-a]quinoxalin-4-yl)ethane-1,2- |
| 345541 | | | diamine |
| CRT0066101 | C₁₈H₂₄ClN₆O | 1883545-60- | 2-[4-[[(2R)-2-aminobutyl]amino]pyrimidin-2-yl]-4-(1- |
| | | 5 | methylpyrazol-4-yl)phenol, dihydrochloride |
| kb NB 142- | C₁₁H₉NO₂S₂ | 1233533-04- | 9-hydroxy-3,4-dihydro-2H-[1]benzothiolo[2,3- |
| 70 | | 4 | f][1,4]thiazepin-5-one |
| ARN-3236 | C₁₉H₁₆N₂O₂S | 1613710-01- | 3-(2,4-dimethoxyphenyl)-4-thiophen-3-yl-1H-pyrrolo[2,3- |
| | | 2 | b]pyridine |
| casein | C₂₄H₂₃N₅O₃ | 863598-09-8 | 3-[3-[2-(3,4,5-trimethoxyanilino)pyrrolo[2,3-d]pyrimidin-7- |
| kinase II | | | yl]phenyl]propanenitrile |
| inhibitor IV | | | |
| IC 261 | C₁₈H₁₇NO₄ | 186611-52-9 | (3E)-3-[(2,4,6-trimethoxyphenyl)methylidene]-1H-indol-2- |
| | | | one |
| SR-3029 | C₂₃H₁₉F₃N₈O | 1454585-06- | N-[(4,5-difluoro-1H-benzimidazol-2-yl)methyl]-9-(3- |
| | | 8 | fluorophenyl)-2-morpholin-4-ylpurin-6-amine |
| AR- | C₁₂H₁₂N₄O₄S | 487021-52-3 | 1-[(4-methoxyphenyl)methyl]-3-(5-nitro-1,3-thiazol-2-ylurea |
| A014418 | | | |

In some embodiments, compounds or kinase inhibitors provided herein is a cyclin-dependent kinase (CDK) inhibitor, a mitogen activated protein kinase (MAPK) inhibitor, a growth factor inhibitor, a Janus kinase (JAK) inhibitor, an extracellular signal-regulated kinase (ERK) inhibitor, a polo-like kinase (PLK) inhibitor, a phosphatidylinositol 4-kinase (P14K) inhibitor, a tyrosine kinase inhibitor, a T-lymphokine-activated killer cell-originated protein kinase (TOPK) inhibitor, a Wnt signaling pathway inhibitor, an IκB kinase (IKK) inhibitor, a protein kinase D (PKD) inhibitor, a salt inducible kinase (SIK) inhibitor, a glycogen synthase kinase-3β (GSK-3β) inhibitor, or a casein kinase II inhibitor IV, pharmaceutically acceptable salts, and solvates thereof.

In some embodiments, the kinase inhibitor is a MAP kinase (MAPK) inhibitor. Exemplary MAPK inhibitors include, without limitation, SP600125, PLX4032, GW5074, AZD6244, PD98059, simvastatin, alisertib, teriflunomide, NSC95397, PD325901, PD98059, lovastatin, DMX-5804, selonsertib (C₂₄H₂₄FN₇O, 5-(4-cycloproplirnidazol-1-yl)-2-fluoro-4-methyl-N-[6-(4-propan-2-yl-1,2,4-triazol-3-yl)pyridin-2-yl]benzamide, CAS NO. 1448428-04-3), MAPK13-IN-1 (C₂₀H₂₃N₅O₂, 1-(5-tert-buty-2-methylpyrazol-3-yl)-3-(4-pyridin-4-yloxyphenyl)urea, CAS NO. 229002-10-2).

In some embodiments, the kinase inhibitor is a growth factor inhibitor. Exemplary growth factor inhibitors include, without limitation, LY2874455 (C₂₁H₁₉Cl₂N₅O₂, 2-[4-[(E)-2-[5-[(1R)-1-(3,5-dichloropyridin-4-yl)ethoxy]-1H-indazol-3-yl]ethenyl]pyrazol-1-yl]ethanol, CAS NO. 1254473-64-7), altiratinib (C₂₆H₂₁F₃N₄O₄, 1-N-[4-[2-(cyclopropanecarbonylamino)pyridin-4-yl]oxy-2,5-difluorophenyl]-1-N-(4-fluorophenyl)cyclopropane-1,1-dicarboxamide, CAS NO. 1345847-93-9), autophinib (C₁₄H₁₁ClN₆O₃, 6-chloro-NV-(5-methyl-1H-pyrazol-3-yl)-2-(4-nitrophenoxy)pyrindin-4-amine, CAS NO. 1644443-47-9), AST 487 (C₂₆H₃₀F₃N₇O₂, 1-[4-[(4-ethylpiperazin-1-yl)methyl]-3-(trifluoromethyl)phenyl]-3-[4-[6-(methylamino)pyrimidin-4-yl]oxyphenyl]urea, CAS NO. 63012446-8) or GW806742X (C₂₅H₂₂F₃N₇O₄S, 1-[4-[methyl-[2-(3-sulfamoylanilino)pyrimidin-4-yl]amino]phenyl]-3-[4-(trifluoromethoxy)phenyl]urea, CAS NO. 579515-63-2), pharmaceutically acceptable salts and solvates thereof.

In some embodiments, the kinase inhibitor is a Janus kinase (JAK) inhibitor. JAK inhibitors include, without limitation, ilginatinib (C₂₁H₂₀FN₇, 6-N-[(1S)-1-(4-fluorophenyl)ethyl]-4-(1-methylpyrazol-4-yl)-2-N-pyrazin-2-ylpyridine-2,6-diamine, CAS No. 1239358-86-1), and pharmaceutically acceptable salts and solvates thereof.

In some embodiments, the kinase inhibitor is an extracellular signal-regulated kinase (ERK) inhibitor. ERK inhibitors include, without limitation, ravoxertinib (C₂₁H₁₈ClFN₆O₂, 1-[(1S)-1-(4-chloro-3-fluorophenyl)-2-hydroxy ethyl]-4-[2-[(2-methylpyrazol-3-yl)amino]pyrimidin-4-yl]pyridin-2-one, CAS NO. 1453848-26-4), and pharmaceutically acceptable salts and solvates thereof.

In some embodiments, the kinase inhibitor is a Polo-like kinase (PLK) inhibitor. PLK inhibitors include, without limitation, SBE13 (C₂₄H₂₇ClN₂O₄, N′-[[4-[(6-chloropyridin-3-yl)methoxy]-3-methoxyphenyl]methyl]-2-(3,4-dimethoxyphenyl)ethandiamine, CAS NO. 775294-82-1), and pharmaceutically acceptable salts and solvates thereof.

In some embodiments, the kinase inhibitor is a phosphatidylinositol 4-kinase (PI4K) inhibitor. PI4K inhibitors include, without limitation, BF738735 (C₂₁H₁₉FN₄O₃S, 2-fluoro-4-[2-methyl-8-[(3-methylsulfonylphenyl)methylamino]imidazo[1,2-a]pyrazin-3-yl]phenol, CAS NO. 1436383-95-7), and pharmaceutically acceptable salts and solvates thereof.

In some embodiments, the kinase inhibitor is a tyrosine kinase inhibitor. Tyrosine kinase inhibitors include, without limitation, R112 (C₁₆H₁₃FN₄O₂, 3-[[5-fluoro-2-(3-hydroxyanilino)pyrimidin-4-yl]amino]phenol, CAS NO. 575474-82-7), and pharmaceutically acceptable salts and solvates thereof.

In some embodiments, the kinase inhibitor is a T-lymphokine-activated killer cell-originated protein kinase (TOPK) inhibitor. TOPK inhibitors include, without limitation, OTS514 (C₂₁H₂₀N₂O₂S, 9-[4-[(2R)-1-aminopropan-2-yl]phenyl]-8-hydroxy-6-methyl-5H-thieno[2,3-c]quinolin-4-one, CAS NO. 1338540-63-8), and pharmaceutically acceptable salts and solvates thereof.

In some embodiments, compounds provided herein are a Wnt signaling pathway inhibitor. Wnt signaling pathway inhibitors include, without limitation, CHIR-99021 (C₂₂H₁₈Cl₂N₈, 6-[2-[[4-(2,4-dichlorophenyl)-5-(5-methyl-1H-imidazol-2-yl)pyrimidin-2-yl]amino]ethylamino]pyridine-3-carbonitrile, CAS NO. 252917-06-9), and pharmaceutically acceptable salts and solvates thereof.

In some embodiments, the kinase inhibitor is a IκB kinase (IKK) inhibitor. IKK inhibitors include, without limitation, BMS-345541 (C₁₄H₁₈ClN₅, (1,8-dimethylimidazo[1,2-a]quinoxalin-4-yl)ethane-1,2-diamine, hydrochloride, CAS NO. 445430-59-1) or BMS-345541 hydrochloride, and pharmaceutically acceptable salts and solvates thereof.

In some embodiments, the kinase inhibitor is a glycogen synthase kinase 3 beta (GSK-3β) inhibitor. GSK-3β inhibitors include, without limitation, indirubin-3′-monoxime (C₁₆H₁₀IN₃O₂, 5-iodo-3-(3-nitroso-1H-indol-2-yl)-1H-indol-2-ol, CAS NO. 331467-03-9), GSK3p Inhibitor 1 (C₁₄H₁₀N₂O, CAS NO.: 187325-53-7), GSK3β Inhibitor II (C₁₄H₁₀IN₃OS, 4-[5-[[(3-iodophenyl)methyl]thio]-1,3,4-oxadiazol-2-yl]-pyridine, CAS NO. 478482-75-6), GSK30 Inhibitor VIII (C₁₂H₁₂N₄O₄S, N-[(4-methoxyphenyl)methyl]-N′-(5-nitro-2-thiazolyl)-urea, CAS NO. 487021-52-3).

In some embodiments, the kinase inhibitor is a protein kinase D (PKD) inhibitor. PKD inhibitors include, without limitation, kb NB 142-70 (C₁₁H₉NO₂S₂, 9-hydroxy-3,4-dihydro-2H-[1]benzothiolo[2,3-f][1,4]thiazepin-5-one, CAS NO. 1233533-04-4), and pharmaceutically acceptable salts and solvates thereof.

In some embodiments, the kinase inhibitor is a salt inducible kinase (SIK) inhibitor. SIK inhibitors include, without limitation, ARN-3236 (C₁₉H₁₆N₂O₂S, 3-(2,4-dimethoxy phenyl)-4-thiophen-3-yl-1-pyrrolo[2,3-b]pyridine, CAS NO. 1613710-01-2), and pharmaceutically acceptable salts and solvates thereof.

In some embodiments, the kinase inhibitor is a casein kinase inhibitor. Casein kinase inhibitors include, without limitation, casein kinase II inhibitor IV (C₂₄H₂₃N₅O₃, 3-[3-[2-(3,4,5-trimethoxyanilino)pyrrolo[2,3-d]pyrimidin-7-yl]phenyl]propanenitrile, CAS NO. 863598-09-8), and pharmaceutically acceptable salts and solvates thereof.

### Combination Compositions

Provided herein are compositions comprising a nanoparticle described herein a nucleic acid described herein encoding for a protein, and a compound described herein that enhances protein expression of the protein. Provided herein are compositions comprising a nanoparticle described herein a first nucleic acid described herein encoding for a protein, and a second nucleic acid described herein encoding for an expression enhancer. The expression enhancer increases expression of the protein or the functional fragment thereof. The second nucleic acid comprises a nucleic acid sequence that has at least 80% sequence identity to any one of the SEQ ID NO: 41-47. The nanoparticle described herein comprises a single nucleic acid comprising the nucleic and the expression enhancer nucleic acid. Alternatively, the nanoparticle described herein comprises a plurality of nucleic acid, wherein each of the plurality of nucleic comprises at least one nucleic acid, at least one expression enhancer nucleic acid, or combinations thereof. Also, provided herein the compositions comprising a nanoparticle described herein a first nucleic acid described herein encoding for a protein, a second nucleic acid described herein encoding for an expression enhancer, and a compound described herein that enhances expression of the protein.

Nanoparticles for inclusion include, without limitation, any one of NP-1 to NP-30. Also, nanoparticles for inclusion include, without limitation, any one of NP-31 to NP-35. Nucleic acids for inclusion include, without limitation, comprise a region encoding for any one of SEQ ID NOS: 8-14, or 8-37. The nucleic acids may further compromise a region encoding for a RNA polymerase, e.g., a region comprising a sequence of SEQ ID NO: 38. Compounds for inclusion are those described herein, including without limitation, those in Table 6.

Compositions provided herein can be characterized by an nitrogen:phosphate (N:P) molar ratio. The N:P ratio is determined by the amount of cationic lipid in the nanoparticle which contain nitrogen and the amount of nucleic acid used in the composition which contain negatively charged phosphates. In some embodiments, the compositions provided herein comprise a N:P ratio of up to about 100:1, 150:1, or 200:1. In some embodiments, the compositions provided herein comprise a N:P ratio of 0.2:1 to 25:1. In some embodiments, the compositions provided herein comprise a N:P ratio of about 200:1, 150:1, 100:1, 80:1, 50:1, 40:1, 25:1, 15:1, 10:1, 8:1, 5:1, 1:1 or 0.2:1. In some embodiments, the compositions provided herein comprise a N:P ratio of up to about 200:1, 150:1, 100:1, 80:1, 50:1, 40:1, 25:1, 15:1, 10:1, 8:1, 5:1. In some embodiments, the compositions provided herein comprise a N:P ratio of at least about 150:1, 100:1, 80:1, 50:1, 40:1, 25:1, 15:1, 10:1, 8:1, 5:1, 1:1. In some embodiments, the nanoparticle comprises a nucleic acid provided herein covalently attached to the membrane. In some embodiments, the compounds provided herein are dispersed within the hydrophobic core of the nanoparticle provided herein.

### Pharmaceutical Compositions

Provided herein is a lyophilized composition comprising a composition provided herein. Further provided herein is a suspension comprising a composition provided herein. In some embodiments, suspensions provided herein comprise a plurality of nanoparticles or compositions provided herein. In some embodiments, compositions provided herein are in a suspension, optionally a homogeneous suspension. In some embodiments, compositions provided herein are in an emulsion form.

Also provided herein is a pharmaceutical composition comprising a composition provided herein. In some embodiments, compositions provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

### Dosing

Compositions provided herein may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

### Administration

Provided herein are compositions and pharmaceutical compositions for administering to a subject in need thereof. In some embodiments, pharmaceutical compositions provided here are in a form which allows for compositions provided herein to be administered to a subject.

In some embodiments, the administering is local administration or systemic administration. In some embodiments, a composition described herein is formulated for administration/for use in administration via an intratumoral, subcutaneous, intradermal, intramuscular, intranasal, inhalation, intravenous, intraperitoneal, intracranial, or intrathecal route. In some embodiments, the administering is every 1, 2, 4, 6, 8, 12, 24, 36, or 48 hours. In some embodiments, the administering is daily, weekly, or monthly. In some embodiments, the administering is repeated at least about every 28 days or 56 days. In some embodiments, a composition or pharmaceutical composition provided herein is administered to the subject by two doses. In some embodiments, a second dose of a composition or pharmaceutical composition provided herein is administered about 28 days or 56 days after the first dose. In some embodiments, a third dose of a composition or pharmaceutical composition provided herein is administered to a subject.

### Therapeutic Applications

Provided herein are methods of treating or preventing a disease in a subject. In some embodiments, compositions provided herein are used to modify NFκB expression or activity relative to interferon-α activity in a subject. In some embodiments, compositions provided herein are used to modify NFκB expression or activity relative to interferon-α activity in a mammalian cell.

In some embodiments, compositions described herein are used for the treatment of an infection. In some embodiments, the infection is a viral infection. In some embodiments, the viral infection is from a Coronavirus. In some embodiments, the Coronavirus is SARS-CoV-2. In some embodiments, the Coronavirus is MERS or SARS. In some embodiments, the viral infection is from an influenza virus. In some embodiments, the influenza virus is influenza A or influenza B. In some embodiments, the viral infection is from a Zika virus. In some embodiments, the viral infection is from a Respiratory syncytial virus (RSV). In some embodiments, the virus is EVD68.

In some embodiments, compositions described herein are used for the reduction of severity of an infection in a subject. In some embodiments, compositions described herein provide for reduction of severity or duration of symptoms associated with an infection in a subject. In some embodiments, the infection is a viral infection. In some embodiments, the viral infection is from a Coronavirus. In some embodiments, the Coronavirus is SARS-CoV-2. In some embodiments, administration of a composition describes herein provides for reduction in the severity or duration of COVID-19 symptoms in a subject. In some embodiments, the Coronavirus is MERS or SARS. In some embodiments, the viral infection is from an influenza virus. In some embodiments, the influenza virus is influenza A or influenza B. In some embodiments, the viral infection is from a Zika virus. In some embodiments, the viral infection is from a Respiratory syncytial virus (RSV). In some embodiments, the virus is EVD68.

In some embodiments, compositions described herein are used for the treatment of a cancer. In some embodiments, the cancer is lung cancer. In some embodiments the cancer is a solid cancer or a hematopoietic cancer. In some embodiments, the solid cancer is a melanoma, lung, liver, head and neck, or pancreatic cancer. In some embodiments, the solid cancer is a melanoma cancer. In some embodiments, a composition described herein is used for reduction of a tumor size. In some embodiments, a composition described herein is used for reduction of a tumor volume. In some embodiments, a composition described herein is used for reduction of a cancer recurrence. In some embodiments, a composition described herein is used for reduction of tumor metastasis.

### Kits

In some embodiments, a formulation of a composition described herein is prepared in a single container for administration. In some embodiments, a formulation of a composition described herein is prepared two containers for administration, separating the nucleic acid and/or the compound provided herein from the nanoparticle carrier.

As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some implementations, the containers are RNase free.

Provided herein is kit, wherein the kit comprises: a first container comprising: a lipid carrier, wherein the lipid carrier comprises a hydrophobic core; and a kinase inhibitor; and a second container comprising: a nucleic acid coding for a protein or a functional fragment thereof.

In some embodiments, the kinase inhibitor is within the hydrophobic core of the lipid carrier. In some embodiments, the lipid carrier comprises a cationic lipid, and an oil. In some embodiments, the lipid carrier comprises a cationic lipid, an oil, and an inorganic particle. In some embodiments, the inorganic particle comprises a metal. In some embodiments, the metal comprises metal salts, metal oxides, metal hydroxides, or metal phosphates. In some embodiments, the metal oxide comprises aluminum oxide, aluminum oxyhydroxide, iron oxide, titanium dioxide, or silicon dioxide. In some embodiments, the nucleic acid further codes for a RNA polymerase. In some embodiments, the RNA polymerase is a Venezuelan equine encephalitis virus (VEEV) RNA polymerase. In some embodiments, the nucleic acid sequence coding for the RNA polymerase comprises the sequence of SEQ ID NO: 38. In some embodiments, the kinase inhibitor is listed in Table 6 or Table 7. In some embodiments, the first container is lyophilized.

### Exemplary Embodiments

Provided herein are compositions, wherein the compositions comprise: a nanoparticle, wherein the nanoparticle comprises a hydrophobic core; a nucleic acid coding for a protein or a functional fragment thereof; and a compound, wherein the compound enhances expression of the protein or the functional fragment thereof in mammalian cells. Further provided herein are compositions wherein the hydrophobic core comprises a liquid organic material. Further provided herein are compositions wherein the hydrophobic core comprises a liquid organic material and a solid inorganic material. Further provided herein are compositions wherein the nanoparticle comprises a hydrophilic surface. Further provided herein are compositions wherein the nanoparticle is up to 200 nm in diameter. Further provided herein are compositions wherein the nanoparticle is 50 to 70 nm in diameter. Further provided herein are compositions wherein the nanoparticle is 40 to 80 nm in diameter. Further provided herein are compositions wherein the nanoparticle is dispersed in an aqueous solution. Further provided herein are compositions wherein the nanoparticle comprises a membrane. Further provided herein are compositions wherein the compound is dispersed in the hydrophobic core. Further provided herein are compositions wherein the compound is conjugated to the nanoparticle. Further provided herein are compositions wherein the nanoparticle comprises a cationic lipid. Further provided herein are compositions wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP), dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′, 9″ ″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions wherein the hydrophobic core comprises an oil. Further provided herein are compositions wherein the oil is in liquid phase. Further provided herein are compositions wherein the oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, solanesol, soy lecithin, soybean oil, sunflower oil, a triglyceride, or vitamin E. Further provided herein are compositions wherein the triglyceride is capric triglyceride, caprylic triglyceride, a caprylic and capric triglyceride, a triglyceride ester, or myristic acid triglycerine. Further provided herein are compositions wherein the nanoparticle comprises an inorganic particle. Further provided herein are compositions wherein the inorganic particle is within the hydrophobic core. Further provided herein are compositions wherein the inorganic particle comprises a metal. Further provided herein are compositions wherein the metal comprises a metal salt, a metal oxide, a metal hydroxide, or a metal phosphate. Further provided herein are compositions wherein the metal oxide comprises aluminum oxide, aluminum oxyhydroxide, iron oxide, titanium dioxide, or silicon dioxide. Further provided herein are compositions wherein the nanoparticle comprises a cationic lipid, and an oil. Further provided herein are compositions wherein the nanoparticle comprises a cationic lipid, an oil, and an inorganic particle. Further provided herein are compositions wherein the nanoparticle further comprises a surfactant. Further provided herein are compositions wherein the surfactant is a hydrophobic surfactant. Further provided herein are compositions wherein the hydrophobic surfactant is sorbitan monolaurate, sorbitan monopalmitate, sorbitan monostearate, sorbitan monooleate, or sorbitan trioleate. Further provided herein are compositions wherein the surfactant is a hydrophilic surfactant. Further provided herein are compositions wherein the hydrophilic surfactant is a polysorbate. Further provided herein are compositions wherein the nanoparticle comprises a cationic lipid, an oil, and a surfactant. Further provided herein are compositions wherein the nanoparticle comprises a cationic lipid, an oil, an inorganic particle, and a surfactant. Further provided herein are compositions wherein the hydrophobic core comprises: a phosphate-terminated lipid, a surfactant, or a combination thereof. Further provided herein are compositions wherein the hydrophobic core comprises: one or more inorganic particles; a phosphate-terminated lipid; and a surfactant. Further provided herein are compositions wherein each inorganic particle is coated with a capping ligand or the surfactant. Further provided herein are compositions wherein the phosphate-terminated lipid is trioctylphosphine oxide (TOPO). Further provided herein are compositions wherein the surfactant is a phosphorous-terminated surfactant, a carboxylate-terminated surfactant, a sulfate-terminated surfactant, or an amine-terminated surfactant. Further provided herein are compositions wherein the surfactant is distearyl phosphatidic acid (DSPA), oleic acid, oleylamine or sodium dodecyl sulfate (SDS). Further provided herein are compositions wherein the protein is an antigen or an antigen-binding protein. Further provided herein are compositions wherein the antigen is in a viral antigen. Further provided herein are compositions wherein the antigen is in a tumor antigen. Further provided herein are compositions wherein the nucleic acid is an RNA or a DNA. Further provided herein are compositions wherein the nucleic acid further codes for an RNA polymerase. Further provided herein are compositions wherein the RNA polymerase is a Venezuelan equine encephalitis virus (VEEV) RNA polymerase. Further provided herein are compositions wherein the nucleic acid coding the RNA polymerase comprises the nucleic acid sequence of SEQ ID NO: 38. Further provided herein are compositions wherein the nucleic acids provided herein are present in an amount of up to about 25, 50, 75, 100, 150, 175 ng. Further provided herein are compositions wherein the nucleic acids provided herein are present in an amount of up to about 1 mg. Further provided herein are compositions wherein the nucleic acids provided herein are present in an amount of about 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. Further provided herein are compositions wherein the nucleic acids provided herein are present in an amount of 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. Further provided herein are compositions wherein the nucleic acid is at least about 1000, 2000, 3000, 4000, 5000, 6000, 7000, 8000, 9000, 10,000, 11,000, 12,000, 13,000, 14,000, 15,000, 16,000, 17,000, 18,000, 19,000, or 20,000 nucleotides in length. Further provided herein are compositions wherein the nucleic acid is up to about 7000, 8000, 9000, 10,000, 11,000, 12,000, 13,000, 14,000, 15,000, 16,000, 17,000, 18,000, 19,000, or 20,000 nucleotides in length. Further provided herein are compositions wherein the nucleic acid is about 7500, 10,000, 15,000, or 20,000 nucleotides in length. Further provided herein are compositions wherein the compound is a kinase inhibitor. Further provided herein are compositions wherein the kinase inhibitor is a casein kinase inhibitor, a cyclin-dependent kinase (CDK) inhibitor, an extracellular signal-regulated kinase (ERK) inhibitor, a growth factor inhibitor, a glycogen synthase kinase inhibitor, an immune checkpoint inhibitor, a Janus kinase (JAK) inhibitor, a IκB kinase (IKK) inhibitor, a glycogen synthase kinase-3β (GSK-3β) inhibitor, a lipid kinase inhibitor, a mitogen-activated protein kinase (MAPK) family inhibitor, a phosphatidylinositol 4-kinase (P14K) inhibitor, a polo-like kinase (PLK) inhibitor, a protein kinase D (PKD) inhibitor, a tyrosine kinase inhibitor, a T-lymphokine-activated killer cell-originated protein kinase (TOPK) inhibitor, a salt inducible kinase (SIK) inhibitor, or a Wnt signaling inhibitor. Further provided herein are compositions wherein the composition is lyophilized. Further provided herein are compositions wherein the composition is in a liquid, semi-liquid, solution, propellant, or powder dosage form. Further provided herein are compositions wherein the composition is formulated as a suspension. Further provided herein are compositions wherein in the suspension is a homogeneous suspension. Further provided herein are compositions wherein the nanoparticle is in an aqueous solution. Further provided herein are pharmaceutical compositions comprising the composition provided herein and a pharmaceutical excipient.

Provided herein are compositions, wherein the composition comprises: a nanoparticle, wherein the nanoparticle comprises a hydrophobic core and a hydrophilic surface; a nucleic acid coding for an antibody or a functional fragment thereof, wherein the nucleic acid is in complex with the hydrophilic surface; and a compound, wherein the compound enhances expression of the antibody or the functional fragment thereof in mammalian cells. Further provided herein are compositions, wherein the antibody is a monoclonal antibody. Further provided herein are compositions, wherein the antibody is a murine antibody, a humanized antibody, or a fully human antibody. Further provided herein are compositions, wherein the antibody is an immunoglobulin (Ig) molecule. Further provided herein are compositions, wherein the immunoglobulin molecule is an IgG, IgE, IgM, IgD, IgA, or an IgY isotype immunoglobulin molecule. Further provided herein are compositions, wherein the immunoglobulin molecule is an IgG1, an IgG2, an IgG3, an IgG4, an IgGA1, or an IgGA2 subclass immunoglobulin molecule. Further provided herein are compositions, wherein the antibody is a recombinant antibody, a chimeric antibody, or a multivalent antibody. Further provided herein are compositions, wherein the multivalent antibody is a bispecific antibody, a trispecific antibody, or a multispecific antibody. Further provided herein are compositions, wherein the antibody or functional fragment is an antigen-binding fragment (Fab), and Fab2 a F(ab′), a F(ab′)2, an dAb, an Fc, a Fv, a disulfide linked Fv, a scFv, a tandem scFv, a free LC, a half antibody, a single domain antibody (dAb), a diabody, or a nanobody. Further provided herein are compositions, wherein the antibody or functional fragment thereof specifically binds to a tumor antigen or a microbial antigen. Further provided herein are compositions, wherein the microbial antigen is a viral envelope protein. Further provided herein are compositions, wherein the tumor antigen is a surface protein or a transmembrane protein. Further provided herein are compositions, wherein the antibody or functional fragment thereof is a SARS-CoV-2 virus antibody. Further provided herein are compositions, wherein the SARS-CoV-2 virus antibody is bamlanivimab, casirivimab, imdevimab, or sotrovimab. Further provided herein are compositions, wherein the antibody or functional fragment thereof is a Zika virus antibody. Further provided herein are compositions wherein the Zika virus antibody is ZIKV-117, Z3L1, Z20, Z23, ZV67, Z006, or 2A10G6. Further provided herein are compositions, wherein the antibody or functional fragment thereof is a cancer therapeutic antibody. Further provided herein are compositions, wherein the cancer therapeutic antibody is atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab. Further provided herein are compositions, wherein the nanoparticle is a cationic lipid carrier, a ionizable lipid carrier, a gold carrier, a magnetic carrier, a polyethylene glycol (PEG)-functionalized carrier, a cholesterol-functionalized carrier, a polylactic acid (PLA)-functionalized carrier, a polylactic-co-glycolic acid (PLGA)-functionalized lipid carrier, or a liposome. Further provided herein are compositions, wherein the nucleic acid further codes for a RNA polymerase. Further provided herein are compositions, wherein the RNA polymerase is a Venezuelan equine encephalitis virus (VEEV) RNA polymerase. Further provided herein are compositions, wherein the nucleic acid coding the RNA polymerase is SEQ ID NO: 38. Further provided herein are compositions, wherein the compound is within the hydrophobic core. Further provided herein are compositions, wherein the compound is conjugated to the nanoparticle. Further provided herein are compositions, wherein the nucleic acid coding for an antibody or a functional fragment thereof is in complex with the nanoparticle. Further provided herein are compositions, wherein the nucleic acid coding for an antibody or a functional fragment thereof is within the nanoparticle. Further provided herein are compositions wherein the nucleic acid coding for an antibody or a functional fragment thereof is outside the nanoparticle. Further provided herein are compositions wherein the composition is lyophilized. Further provided herein are compositions wherein the composition is in a liquid, semi-liquid, solution, propellant, or powder dosage form. Further provided herein are compositions wherein the composition is formulated as a suspension. Further provided herein are compositions wherein in the suspension is a homogeneous suspension. Further provided herein are compositions wherein the nanoparticle is in an aqueous solution. Further provided herein are pharmaceutical compositions comprising a composition provided herein and pharmaceutical excipient.

Provided herein are compositions, wherein the composition comprises: a nanoparticle comprising a membrane; a nucleic acid coding for a protein or a functional fragment thereof; and a kinase inhibitor. Further provided herein are compositions wherein the kinase inhibitor is within the membrane. Further provided herein are compositions wherein the kinase inhibitor is conjugated to the membrane. Further provided herein are compositions wherein the kinase inhibitor is a cyclin-dependent kinase (CDK) inhibitor. Further provided herein are compositions wherein the CDK inhibitor is (−)-BAY-1251152, (+)-BAY-1251152, (±)-BAY-1251152, (2S, 3R)-voruciclib, AT7519, AUZ-454, AZD-5438, AZD4573, CDK-IN-2, CDK12-IN-3, CDK9-IN-8, CDKI-73, dinaciclib, flavopiridol, K00546, KB-0742, LDC4297, LSN3106729, LY2857785, MC180295, NVP-LCQ195, PF-06873600, PHA-767491, PHA-793887, PROTAC CDK9 Degrader-1, RGB-286638, seliciclib, simurosertib, SNS-032, SU9516, THZ2, voruciclib, a free base thereof, a salt thereof, or combinations thereof. In some embodiments, the CDK inhibitor comprises a hydrochloride salt of the CDK inhibitor. In some embodiments, the CDK inhibitor is (+)-BAY-1251152, AZD4573, CDK-IN-2, CDK12-IN-3, CDKI-73, dinaciclib, flavopiridol hydrochloride, LY2857785, MC180295, RGB-286638 free base, or combinations thereof. In some embodiments, the CDK inhibitor is LSN3106729 hydrochloride, PF-06873600, (2S, 3R)-voruciclib hydrochloride, AZD-5438, seliciclib, simurosertib, AT7519, or CDK-IN-2. Further provided herein are compositions wherein the kinase inhibitor is a MAP kinase inhibitor. Further provided herein are compositions wherein the MAP kinase inhibitor is DMX-5804, selonsertib, NCB-0846, MAPK13-IN-1, a free base thereof, a salt thereof, or combinations thereof. Further provided herein are compositions wherein the kinase inhibitor is growth factor inhibitor Further provided herein are compositions wherein the growth factor inhibitor is LY2874455, altiratinib, autophinib, AST 487, GW806742X, BMS-794833, K00546, SCR-1481B1, tyrosine kinase-IN-1, VEGFR-2-IN-5 hydrochloride, XL228, a free base thereof, a salt thereof, or combinations thereof. In some embodiments, the growth factor inhibitor comprises a hydrochloride salt of the growth inhibitor (e.g., VEGFR-2-IN-5 hydrochloride, GW806742X hydrochloride). Further provided herein are compositions wherein the kinase inhibitor is a Janus kinase (JAK) inhibitor. Further provided herein are compositions wherein the JAK inhibitor is AZ960, ilginatinib, momelotinib, RGB-286638, a free base thereof, a salt thereof, or combinations thereof. In some embodiments, the JAK inhibitor comprises a hydrochloride or a sulfate of the JAK inhibitor (e.g., ilginatinib hydrochloride, momelotinib sulfate). Further provided herein are compositions wherein the kinase inhibitor is an extracellular signal-regulated kinase (ERK) inhibitor. Further provided herein are compositions wherein the ERK inhibitor is ravoxertinib, VX-Ile, a free base thereof, a salt thereof, or combinations thereof. In some embodiments, the ERK inhibitor comprises a hydrochloride salt of the ERK inhibitor (e.g., ravoxertinib hydrochloride). Further provided herein are compositions wherein the kinase inhibitor is a polo-like kinase (PLK) inhibitor. Further provided herein are compositions wherein the PLK inhibitor is SBE13, HMN-214, a free base thereof, a salt thereof, or combinations thereof. In some embodiments, the PLK inhibitor comprises a hydrochloride salt of the PLK inhibitor (e.g., SBE13 hydrochloride). Further provided herein are compositions wherein the kinase inhibitor is a phosphatidylinositol 4-kinase (PI4K) inhibitor. Further provided herein are compositions wherein the PI4K inhibitor is BF738735, a free base thereof, a salt thereof, or combinations thereof. Further provided herein are compositions wherein kinase inhibitor is a tyrosine kinase inhibitor. Further provided herein are compositions wherein the tyrosine kinase inhibitor is R112, a free base thereof, a salt thereof, or combinations thereof. Further provided herein are compositions wherein the kinase inhibitor is a T-lymphokine-activated killer cell-originated protein kinase (TOPK) inhibitor. Further provided herein are compositions wherein the TOPK inhibitor is OTS514, a free base thereof, a salt thereof, or combinations thereof. Further provided herein are compositions wherein the kinase inhibitor is a Wnt signaling pathway inhibitor. Further provided herein are compositions wherein the Wnt signaling inhibitor is CHIR-99021, NCB-0846, a free base thereof, a salt thereof, or combinations thereof. In some embodiments, the Wnt signaling inhibitor comprises a hydrochloride salt of the Wnt signaling inhibitor (e.g., CHIR-99021 monohydrochloride, CHIR-99021 trihydrochloride). Further provided herein are compositions wherein the kinase inhibitor is a IκB kinase (IKK) inhibitor. Further provided herein are compositions wherein the IKK inhibitor is ACHP, BAY-985, BMS-345541, a free base thereof, a salt thereof, or combinations thereof. In some embodiments, the IKK inhibitor comprises a hydrochloride salt of the IKK inhibitor (e.g., ACHP hydrochloride, BMS-345541 hydrochloride). Further provided herein are compositions wherein the kinase inhibitor is a protein kinase D (PKD) inhibitor. Further provided herein are compositions wherein the PKD inhibitor is CRT0066101, kb NB 142-70, a free base thereof, a salt thereof, or combinations thereof. In some embodiments, the PKD inhibitor comprises a hydrochloride salt of the PKD inhibitor (e.g., CRT0066101 dihydrochloride). Further provided herein are compositions wherein the kinase inhibitor is a salt inducible kinase (SIK) inhibitor. Further provided herein are compositions wherein the SIK inhibitor is ARN-3236, a free base thereof, a salt thereof, or combinations thereof. Further provided herein are compositions wherein the kinase inhibitor is a glycogen synthase kinase-3β (GSK-3β) inhibitor. Further provided herein are compositions wherein the glycogen synthase kinase-3β (GSK-3β) inhibitor is AR-A014418, CHIR-99021, CP21R7, GSK-3 inhibitor 1, Indirubin-3′-monoxime, K00546, RGB-286638, a free base thereof, a salt thereof, or combinations thereof. In some embodiments, the glycogen synthase kinase-3β (GSK-3β) inhibitor comprises a hydrochloride salt of the glycogen synthase kinase-3β (GSK-3β) inhibitor (e.g., CHIR-99021 monohydrochloride, CHIR-99021 trihydrochloride). Further provided herein are compositions wherein the kinase inhibitor is a casein kinase inhibitor. Further provided herein are compositions wherein the casein kinase inhibitor is casein kinase II inhibitor IV, IC 261, SR-3029, a free base thereof, a salt thereof, or combinations thereof. Further provided herein are compositions wherein the membrane comprises a cationic lipid, a ionizable lipid, a polyethylene glycol (PEG) functionalized lipid, a cholesterol-functionalized lipid, a polylactic acid (PLA)-functionalized lipid, a polylactic-co-glycolic acid (PLGA)-functionalized lipid, or a liposome. Further provided herein are compositions wherein the nucleic acid is in complex with the membrane. Further provided herein are compositions wherein the nucleic acid further codes for a RNA polymerase. Further provided herein are compositions wherein the RNA polymerase is a Venezuelan equine encephalitis virus (VEEV) RNA polymerase. Further provided herein are compositions wherein the nucleic acid sequence coding for the RNA polymerase comprises the sequence of SEQ ID NO: 38. Further provided herein are compositions wherein the composition is lyophilized. Further provided herein are compositions wherein the composition is lyophilized. Further provided herein are compositions wherein the composition is in a liquid, semi-liquid, solution, propellant, or powder dosage form. Further provided herein are compositions wherein the composition is formulated as a suspension. Further provided herein are compositions wherein in the suspension is a homogeneous suspension. Further provided herein are compositions wherein the nanoparticle is in an aqueous solution. Further provided herein are compositions wherein the composition is lyophilized. Further provided herein are compositions wherein the composition is in a liquid, semi-liquid, solution, propellant, or powder dosage form. Further provided herein are compositions wherein the composition is formulated as a suspension. Further provided herein are compositions wherein in the suspension is a homogeneous suspension. Further provided herein are compositions wherein the nanoparticle is in an aqueous solution. Further provided herein are pharmaceutical compositions comprising a composition provided herein and pharmaceutical excipient.

Provided herein are compositions, wherein the composition comprises: a nanoparticle, wherein the nanoparticle comprises a membrane and a hydrophobic core; a nucleic acid coding for an antibody or a functional fragment thereof, wherein the nucleic acid is in complex with the nanoparticle; and a compound listed in Table 7, wherein the compound is within the hydrophobic core. Further provided herein are compositions wherein the nanoparticle comprises a cationic lipid, and an oil. Further provided herein are compositions wherein the nanoparticle comprises a cationic lipid, an oil, and an inorganic particle. Further provided herein are compositions wherein the inorganic particle comprises a metal. Further provided herein are compositions wherein the metal comprises metal salts, metal oxides, metal hydroxides, or metal phosphates. Further provided herein are compositions wherein the metal oxide comprises aluminum oxide, aluminum oxyhydroxide, iron oxide, titanium dioxide, or silicon dioxide. Further provided herein are compositions wherein the oil is α-tocopherol, coconut oil, grapeseed oil, lauroyl polyoxylglyceride, mineral oil, monoacylglycerol, palmkernal oil, olive oil, paraffin oil, peanut oil, propolis, squalene, squalane, solanesol, soy lecithin, soybean oil, sunflower oil, a triglyceride, or vitamin E. Further provided herein are compositions wherein the triglyceride is capric triglyceride, caprylic triglyceride, a caprylic and capric triglyceride, a triglyceride ester, or myristic acid triglycerine. Further provided herein are compositions wherein the hydrophobic core further comprises a phosphate-terminated lipid. Further provided herein are compositions wherein the hydrophobic core further comprises a surfactant. Further provided herein are compositions wherein the nucleic acid further codes for an RNA polymerase. Further provided herein are compositions wherein the RNA polymerase is a Venezuelan equine encephalitis virus (VEEV) RNA polymerase. Further provided herein are compositions wherein the nucleic acid coding the RNA polymerase comprises the nucleic acid sequence of SEQ ID NO: 38. Further provided herein are compositions wherein the VEEV RNA polymerase comprises the amino acid sequence of SEQ ID NO: 39 or SEQ ID NO: 40. Further provided herein are compositions wherein the antibody or functional fragment thereof is a monoclonal antibody. Further provided herein are compositions wherein the antibody or functional fragment thereof specifically binds to a viral antigen. Further provided herein are compositions wherein the viral antigen is a Zika virus antigen. Further provided herein are compositions wherein the Zika virus antigen is the envelope (E) protein. Further provided herein are compositions wherein the antibody or functional fragment thereof is a Zika virus antibody. Further provided herein are compositions wherein the Zika virus antibody or functional fragment thereof is a ZIKV-117 antibody. Further provided herein are compositions wherein the ZIKV-117 antibody or functional fragment comprises a heavy chain CDR1 amino acid sequence of GFTFKNYG (SEQ ID NO: 48), a heavy chain CDR2 amino acid sequence of VRYDGNNK (SEQ ID NO: 49), and a heavy chain CDR3 amino acid sequence of ARDPETFGGFDY (SEQ ID NO: 50), and a light chain CDR1 amino acid sequence of ESVSSN (SEQ ID NO: 51), light chain CDR2 amino acid sequence of GAS, and light chain CDR3 amino acid sequence of QQYYYSPRT (SEQ ID NO: 52).

Provided herein are kits, wherein kit comprises: a first container comprising: a lipid carrier, wherein the lipid carrier comprises a hydrophobic core; and a kinase inhibitor; and a second container comprising: a nucleic acid coding for a protein or a functional fragment thereof. Further provided herein are kits, wherein the kinase inhibitor is within the hydrophobic core of the lipid carrier. Further provided herein are kits, wherein the lipid carrier comprises a cationic lipid and an oil. Further provided herein are kits, wherein the lipid carrier comprises a cationic lipid, an oil, and an inorganic particle. Further provided herein are kits, wherein the inorganic particle comprises a metal. Further provided herein are kits, wherein the metal comprises metal salts, metal oxides, metal hydroxides, or metal phosphates. Further provided herein are kits, wherein the metal oxide comprises aluminum oxide, aluminum oxyhydroxide, iron oxide, titanium dioxide, or silicon dioxide. Further provided herein are kits, wherein the nucleic acid further codes for a RNA polymerase. Further provided herein are kits, wherein the RNA polymerase is a Venezuelan equine encephalitis virus (VEEV) RNA polymerase. Further provided herein are kits, wherein the nucleic acid sequence coding the RNA polymerase comprises the sequence of SEQ ID NO: 38. Further provided herein are kits, wherein the kinase inhibitor is listed in Table 6 or Table 7. Further provided herein are kits, wherein the first container is lyophilized. Further provided herein are compositions wherein the composition is lyophilized. Further provided herein are compositions wherein the composition is in a liquid, semi-liquid, solution, propellant, or powder dosage form. Further provided herein are kits wherein the composition is formulated as a suspension. Further provided herein are kits wherein in the suspension is a homogeneous suspension. Further provided herein are kits wherein the nanoparticle is in an aqueous solution. Further provided herein are pharmaceutical compositions comprising a first or second container provided herein and pharmaceutical excipient.

Provided herein are methods, wherein the method comprises: administering to a subject, the composition, the suspension, or the pharmaceutical composition provided herein in an amount sufficient to modify NFκB expression or activity relative to interferon-α activity in the subject. Provided herein are methods, wherein the method comprises: administering to a subject having an infection, the composition provided herein, the suspension provided herein, or the pharmaceutical composition provided herein. Also provided herein are methods, wherein the method comprises: administering to a subject having cancer, the composition provided herein, the suspension provided herein, or the pharmaceutical composition provided herein. Further provided herein are methods, wherein the administering is local administration or systemic administration. Further provided herein are methods, wherein the administering is via intramuscular injection, intranasal administration, inhalation, oral administration, subcutaneous administration, intratumoral administration, or intravenous injection. Further provided herein are methods, wherein the subject has a solid tumor or a blood cancer. Further provided herein are methods, wherein the solid tumor is a carcinoma, a melanoma, or a sarcoma. Further provided herein are methods, wherein the blood cancer is lymphoma or leukemia. Further provided herein are methods, wherein the subject has lung cancer. Further provided herein are methods, wherein the lung cancer is adenocarcinoma, squamous cell carcinoma, small cell cancer or non-small cell cancer.

Provided herein are methods, wherein the method comprises: contacting a cell with the composition provided herein, wherein the contacting modifies the level or activity of NFκB relative to interferon-α levels or activity in the cell. Further provided herein are methods, wherein the contacting is ex vivo, in vivo, or in vitro. Further provided herein are methods, wherein the cell is a cancer cell or a blood cell. Further provided herein are methods, wherein the cancer cell is a lung cancer cell. Further provided herein are methods, wherein the blood cell is a dendritic cell or a natural killer cell.

For any of the above embodiments, the compound may further enhance expression of the protein or the functional fragment thereof in mammalian cells compared to a similar composition lacking the compound.

Provided herein are compositions, wherein the compositions comprise: a nanoparticle, optionally wherein the nanoparticle comprises a hydrophobic core; a nucleic acid coding for a protein or a functional fragment thereof; and a compound, wherein the compound enhances expression of the protein or the functional fragment thereof in mammalian cells.

Provided herein are compositions, wherein the compositions comprise: a nanoparticle, optionally wherein the nanoparticle comprises a hydrophobic core and a hydrophilic surface; a nucleic acid coding for an antibody or a functional fragment thereof, wherein the nucleic acid is in complex with the hydrophilic surface; and a compound, wherein the compound enhances expression of the antibody or the functional fragment thereof in mammalian cells.

Provided herein are compositions, wherein the compositions comprise: a nanoparticle comprising a membrane; a nucleic acid coding for a protein or a functional fragment thereof; and a kinase inhibitor.

Provided herein are compositions, wherein the compositions comprise: a nanoparticle, wherein the nanoparticle comprises a membrane and a hydrophobic core; a nucleic acid coding for an antibody or a functional fragment thereof, wherein the nucleic acid is in complex with the nanoparticle; and a compound listed in Table 7, wherein the compound is within the hydrophobic core.

Further provided herein are kits comprising: a first container comprising: a lipid carrier, wherein the lipid carrier comprises a hydrophobic core; and a kinase inhibitor; and a second container comprising: a nucleic acid coding for a protein or a functional fragment thereof.

Also provided herein are methods, wherein the methods comprise: administering to a subject, a composition provided herein, a suspension provided herein, or a pharmaceutical composition provided herein in an amount sufficient to modify NFκB expression or activity relative to interferon-α activity in the subject.

Provided herein are methods of treating infection comprising administering to a subject having an infection, a composition provided herein, the suspension provided herein, or a pharmaceutical composition provided herein.

Provided herein are methods of treating cancer, wherein the method comprises: administering to a subject having cancer, a composition provided herein, the suspension provided herein, or a pharmaceutical composition provided herein.

Provided herein is a method, wherein the method comprises: contacting a cell with the composition provided herein, wherein the contacting modifies the level or activity of NFκB relative to interferon-α levels or activity in the cell.

For any of the above compositions and methods, the compound may further enhance expression of the protein or the functional fragment thereof in mammalian cells compared to a similar composition lacking the compound.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: Manufacture and Stability of Nanoparticle NP-1

Manufacture of NP-1. NP-1 particles comprise 37.5 mg/ml squalene (SEPPIC), 37 mg/ml Span® 60 (Millipore Sigma), 37 mg/ml Tween® 80 (Fisher Chemical), 30 mg/ml DOTAP chloride (LIPOID), 0.2 mg Fe/ml 12 nm oleic acid-coated iron oxide nanoparticles (ImagionBio) and 10 mM sodium citrate dihydrate (Fisher Chemical). 1 ml of 20 mgFe/ml 12 nm diameter oleic acid-coated iron oxide nanoparticles in chloroform (ImagionBio, lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams span 60, and 3 grams DOTAP were added to produce the oil phase. The oil phase was sonicated for 45 minutes in a 65° C. water bath. Separately, the aqueous phase was prepared by dissolving 19.5 grams Tween 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.25 polydispersity index (PDI). The microfluidized nanoparticle was terminally filtered with a 200 nm pore-size polyethersulfone (PES) filter and stored at 2-8° C. Iron concentration was determined by ICP-OES. DOTAP and Squalene concentration were measured by RP-HPLC.

Manufacture of NP-3. NP-3 particles comprise 37.5 mg/ml Miglyol 812 N (IOI Oleo GmbH), 37 mg/ml Span® 60 (Millipore Sigma), 37 mg/ml Tween® 80 (Fisher Chemical), 30 mg/ml DOTAP chloride (LIPOID), 0.2 mgFe/ml 15 nm oleic acid-coated iron oxide nanoparticles (ImagionBio) and 10 mM sodium citrate dihydrate (Fisher Chemical). 1 ml of 20 mgFe/ml 15 nm diameter oleic acid-coated iron oxide nanoparticles in chloroform (ImagionBio, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams span 60, and 3 grams DOTAP were added to produce the oil phase. The oil phase was sonicated for 45 minutes in a 65° C. water bath. Separately, the aqueous phase was prepared by dissolving 19.5 grams Tween 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized nanoparticle was terminally filtered with a 200 nm pore-size polyethersulfone (PES) filter and stored at 2-8° C. Iron concentration was determined by ICP-OES. DOTAP concentration was measured by RP-HPLC.

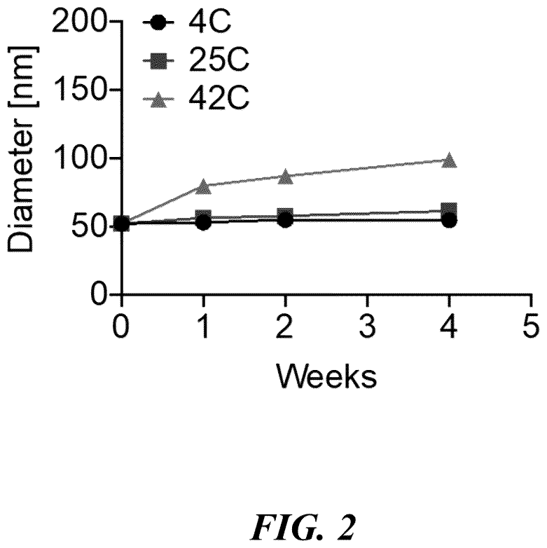

Stability. A nanoparticle according to NP-1 was placed into a stability chamber at the indicated temperatures. The stability was determined by particle size measurement using dynamic light scattering. The results show that the NP-1 formulation formed a stable colloid when stored at 4, 25 and 42 degrees Celsius. Time measurements were taken over 4 weeks. As shown in FIG. 2, the range of nanoparticle size was about 50-100 nm in diameter, and closer to 40-60 nm in diameter for the 4 and 25 degrees Celsius conditions over time.

### Example 2: Self-Replicating mRNA Construct

A plasmid encoding a T7 promoter followed by the 5′ and 3′ UTRs and nonstructural genes of Venezuelan equine encephalitis virus (VEEV) strain TC-83 was generated using standard DNA synthesis and cloning methods. The VEEV replicon mRNA backbone is set forth in SEQ ID NO: 38.

### Example 3: SARS-CoV2 and ZIKV117 Antibodies

The TC-83 repRNA backbone was modified to express ZIKV-117 for intramuscular administration to C57BL/6 wild type or pre-treated intraperitoneal (IP) with anti-mouse IFN alpha/beta receptor (IFNAR) monoclonal antibody to systemically block type I IFN signaling.

The self-replicating mRNA encoding the Zika virus antibody, ZIKV-117, comprises the SEQ ID NO: 12.

Mice were inoculated and bled on days 3, 5 and 7 post-RNA inoculation. Antibody concentration in serum was determined using an enzyme-linked immunosorbent assay (ELISA) with ZIKV Envelope (E) as the target protein to capture ZIKV-117. Serum ZIKV-117 concentration was significantly greater in the anti-IFNAR treated group compared to wild type by an average of three-fold on all days (data not shown). This result demonstrated that protein expression from mRNA can be enhanced by blocking type I IFN signaling.

The self-replicating mRNA encoding the Zika virus antibody, ZIKV-117, was then formulated with NP-1 for delivery with small molecule inhibitors to facilitate local immune suppression.

### Example 4: Co-Delivery of Compounds to Enhance RNA-Encoded Protein Production

A library of small molecule kinase inhibitors was screened for the ability to enhance expression of mRNA-encoded genes. Lead candidates are co-formulated in a nanoparticle vehicle with mRNA-encoding a mAb for localized co-delivery by intramuscular injection. The scope of this work includes: (a) screening a library of 1876 small molecule kinase inhibitors at a single dilution using an automated high throughput method, (b) an expanded dose-ranging assay to confirm hit-to-lead down selection, (c) formulation of candidate compounds in a nanoparticle formulation based on biophysical and in vitro characterization, and (e) the formulation of lead candidates in vivo to evaluate mRNA-encoded mAb production in mice.

Compound screening was performed on the A549-Dual cell line (Invivogen), which are adherent epithelial cells that have been derived from the human A549 lung carcinoma cell line by stable integration of two inducible reporter constructs under the control of the nuclear factor kappa-light-chain-enhancer of activated B cells (NF-κB) and interferon-induced protein with tetratricopeptide repeats 2 (IFIT2) signaling. The cells produce secreted embryonic alkaline phosphatase (SEAP), under the control of the interferon beta (IFN-0) minimal promoter fused to five NF-κB binding sites. The cells also secrete lucia luciferase under the control of IFIT2 promoter.

To measure protein expression from mRNA, which is the primary readout for compound down selection, mRNA encoding a secreted version of nanoluciferase (mRNA-nLuc) was used. Protein expression was measured via a luminometer using standard techniques.

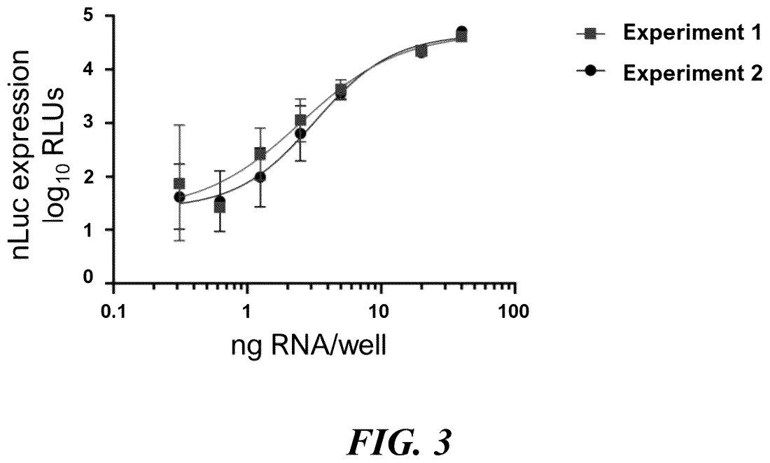

FIG. 3 shows the mRNA dose-response curve. A549-Dual cells were transfected in sextuplet with increasing doses of a nanoparticle (NP-1)+mRNA-nLuc complexed at a nitrogen:phosphate (N:P) ratio of 15. The dose-response curves identified a single mRNA dose for the high throughput screen that is in the linear range and secondly, determine the % coefficient of variation (% CV) for the selected dose level. After testing several dose levels (10,15, 30 and 50 ng per well) in multiple single point transfection, with 4-6 96-well plates per assay, a relatively high degree of plate-to-plate variability was observed in the % CV value, ranging from 25% in one assay to as high as 50% in another. In general, higher doses (>20 ng/well) resulted in lower % CVs (<30%) but because expression levels are near the nonlinear portion of the upper asymptote, there is little dynamic range to identify compounds that upregulate expression. At the lower end of the dose curve (<10 ng/well) the % CVs were too variable and thus increase the probability of false negatives and/or false positives.

Based on the previous findings it was revealed that the variability in the previous approach was too high to perform a reliable high throughput screen. At the same time, a cocktail of monoclonal and polyclonal neutralizing antibodies was evaluated for targeting both human type I IFN and IFN-alpha receptor (IFNAR) from PBL Assay Science (cat #39000-1). Without being bound by a particular theory, it was hypothesized that since systemic IFNAR blocking in mice leads to enhanced mAb production from mRNA in mice, the same approach could be used to enhance protein expression in vitro and thus would serve as a positive control to compare compounds enhancing expression from the replicon.

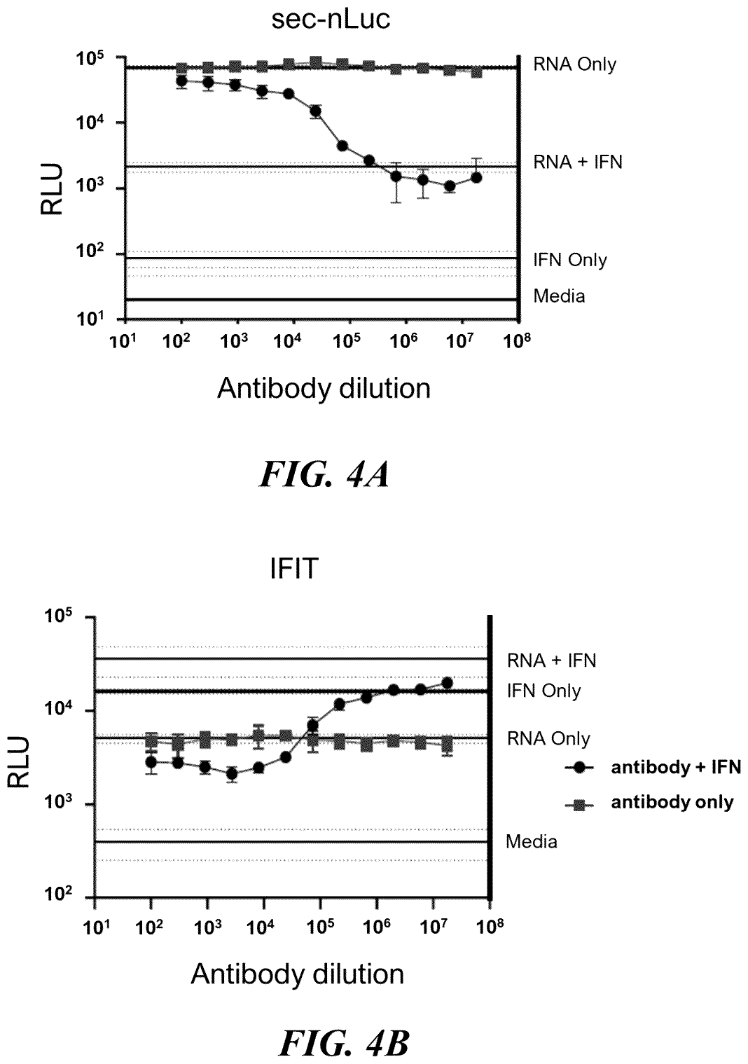

A549-Dual cells were stimulated with a 1:50 dilution of human IFN-alpha (BEI resources cat #NR-3077) and co-delivered 20 ng NP-formulated mRNA-nLuc with a dilution series of the IFN/IFNAR neutralizing antibody. In the absence of exogenous IFN stimulation (RNA only), a steady level of nLuc expression was observed regardless of antibody concentration (FIG. 4A), suggesting that the level of IFN stimulation at the delivered mRNA dose is insignificant, or the lack of a complex innate immune environment in vitro did not recapitulate the response observed in vivo. However, stimulation of A549-Dual cells with IFN significantly reduced nLuc expression (RNA+IFN). With lower dilutions of the antibody (increasing concentration), nLuc expression was completely recovered, suggesting that the antibody neutralized the effects of exogenous IFN stimulation.

The IFIT activity was also measured in the same cells (FIG. 4B) by assaying for lucia luciferase with the QUANTI-Luc™ luciferase detection kit (Invivogen cat #rep-qlc2). Relative to nLuc expression, an inverted dose response was observed. Remarkably, these results confirmed that IFN stimulation potently activated IFIT in A549-Dual cells and significantly downregulated mRNA. Moreover, the anti-IFN antibody cocktail effectively neutralized the effect of IFN in a dose-dependent manner by recovering nLuc expression which inversely correlated with IFIT activation. As such, it was confirmed that using IFN to suppress expression of an antibody or peptide provides a beneficial model system to screen for compounds capable of rescuing the replicon. As a result, this approach was used to screen a large compound library. Since the two secreted luciferase reporter systems can be used in combination, secondary readouts such as IFIT activity and NF-κB activation were used to capture additional compound hits. Finally, cell lysates were assayed with the CellTiter-GLO® assay (Promega) to obtain a luminescent-based measure of cell viability. In summary, the readouts from the high throughput screen included the primary measure of nLuc protein expression and three secondary readouts: IFIT, NF-κB and cell viability.

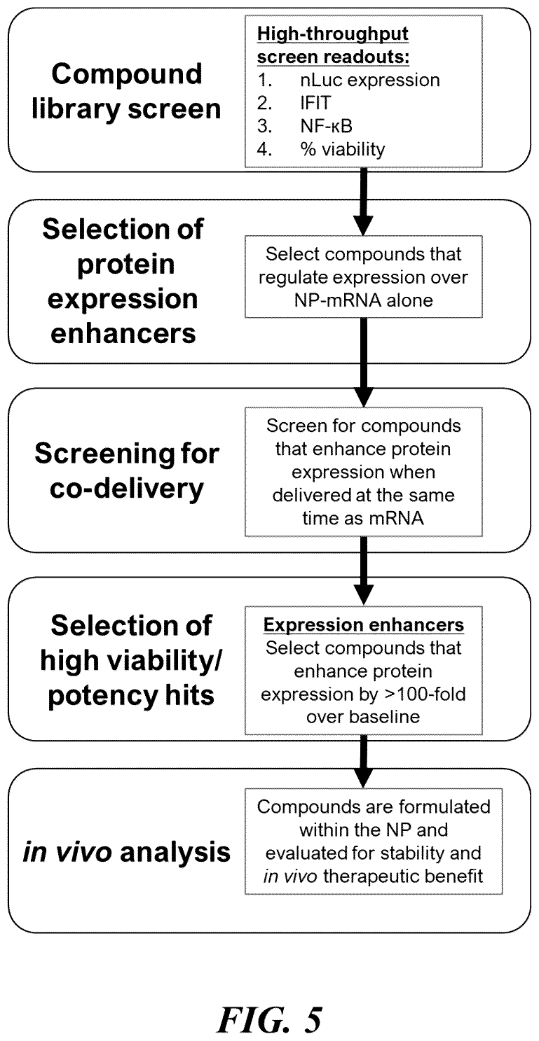

Hits selected for formulation with NP-1 were based on the combination of potency and cytotoxicity. NP-1-formulated compounds can be tested in vitro to verify activity is preserved after formulation in the hydrophobic phase. Hits that preserve activity can be put on stability watch and formulations stable for at least a month will be advanced to mouse studies for evaluation of mRNA-encoded antibody expression in mice. Graphical illustration outlining the screening strategy is shown in FIG. 5.

A primary screen of 1876 compounds from a kinase inhibitor library (MedChemExpress cat #HY-L009) was conducted. All liquid handling steps were performed with the Integra Assist Plus automation system to allow for high throughput screening with minimal operator input. Approximately 600-700 compounds were tested in a single assay (8-9 96-well plates containing a maximum of 80 compounds per plate). For each assay, A549-dual cells were plated at a seeding density of about 5×10⁴ cells per well. Cells were incubated overnight with compounds at 50 microMolar (μM) in 1% v/v dimethyl sulfoxide (DMSO). For control wells, 1% DMSO was added to each well. After about 24 hours, the culture medium was removed, and cells were transfected with NP-1+mRNA at 20 ng/mRNA/well in Opti-MEM® media (Thermofisher). Interferon (IFN) was diluted to 1:50 in all test wells except for the RNA only wells and media only wells. Anti-IFN was diluted 1:1000 in “RNA+anti-IFN+IFN” wells. Cells were incubated for 4 hours, and culture medium was added to the Opti-MEM® medium. After 24 hours, assay supernatants were collected and analyzed for nLuc expression, IFIT2 (qLuc), and NFκB (qBLUE-SEAP™, InvivoGen) activation. Cells were then lysed, and cell viability was determined by CellTiter-Glo® (Promega) assay.

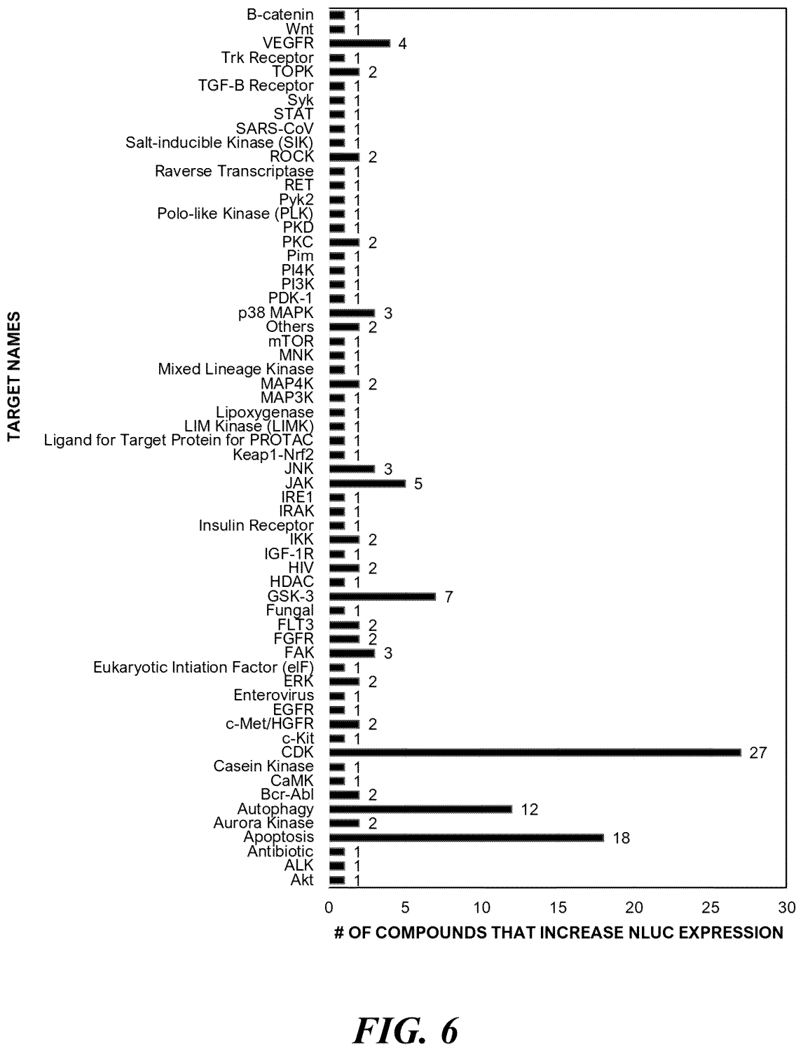

The nLuc expression was plotted as log₁₀ fold change over cells treated with NP+mRNA-nLuc with IFN (RNA+IFN), to identify compounds that upregulated expression from mRNA even in the presence of IFN-mediated suppression. As additional criterion, expression in compound treated cells was compared relative to mRNA-transfected cells without IFN (“RNA only”) (data not shown). 576 compounds were found to upregulate expression of nLuc above RNA+IFN baseline. All of the compounds that upregulated expression over RNA only (i.e., without IFN suppression) were down-selected to 99 compounds. Histograms showing the distribution of responses for the 99 compounds, including their targets, are shown in FIG. 6. The top 32 compounds were then selected. Out of the top 32 compounds, 8 resulted in a >750% viability, 12 resulted in a >500% cell viability, and 12 resulted in <50% viability at the 50 μM treatment dose. A subset of compounds that upregulate nLuc expression at least 100-fold over RNA+IFN are provided in Table 7 below.

TABLE 7
Compound and associated data

| | | | % | fold IFIT | fold nLuc | fold NFkB |
|---|---|---|---|---|---|---|
| # | Compound name | Target(s) | viability | over IFN | over RNA + IFN | over IFN |
| 1 | SBE13 | Apoptosis; Autophagy; | 105% | 0.32 | 179.07 | 0.73 |
| | (Hydrochloride) | Polo-like Kinase (PLK) | | | | |
| 2 | Ravoxertinib | ERK | 96% | 0.24 | 162.39 | 1.17 |
| | hydrochloride | | | | | |
| 3 | BF738735 | PI4K; Reverse | 93% | 0.37 | 238.55 | 0.62 |
| | | Transcriptase | | | | |
| 4 | DMX-5804 | MAP4K | 92% | 0.40 | 267.46 | 1.35 |
| 5 | GSK-3 inhibitor 1 | GSK-3 | 90% | 0.65 | 225.93 | 1.00 |
| 6 | (R)-Simurosertib | Others | 86% | 1.84 | 200.44 | 1.33 |
| 7 | R112 | Syk | 76% | 1.33 | 520.96 | 1.16 |
| 8 | Selonsertib | Apoptosis; MAP3K | 75% | 1.43 | 112.16 | 0.90 |
| 9 | Voruciclib | CDK | 71% | 0.80 | 274.28 | 0.63 |
| | (hydrochloride) | | | | | |
| 10 | Ilginatinib | JAK | 69% | 1.89 | 115.95 | 1.12 |
| 11 | OTS514 | Apoptosis; TOPK | 66% | 0.40 | 348.56 | 0.35 |
| 12 | Indirubin-3′- | GSK-3; | 63% | 0.52 | 331.88 | 0.38 |
| | monoxime | Lipoxygenase | | | | |
| 13 | LSN3106729 | CDK; Ligand for Target | 62% | 1.78 | 327.43 | 1.01 |
| | (hydrochloride) | Protein for PROTAC | | | | |
| 14 | CHIR-99021 | Autophagy; GSK- | 61% | 0.35 | 116.18 | 0.71 |
| | (monohydrochloride) | 3; Wnt; β-catenin | | | | |
| 15 | MAPK13-IN-1 | Autophagy; p38 MAPK | 59% | 1.93 | 207.89 | 0.66 |
| 16 | BMS-345541 | IKK | 59% | 3.09 | 584.94 | 0.34 |
| 17 | LY2874455 | FGFR | 58% | 0.09 | 149.55 | 0.30 |
| 18 | PF-06873600 | CDK | 55% | 1.08 | 119.63 | 0.83 |
| 19 | kb NB 142-70 | PKD | 52% | 2.31 | 186.61 | 0.61 |
| 20 | (2S,3R)-Voruciclib | CDK | 51% | 1.09 | 112.96 | 0.26 |
| | (hydrochloride) | | | | | |
| 21 | Autophinib | Autophagy; PI3K | 49% | 1.09 | 128.89 | 0.78 |
| 22 | Altiratinib | c-Met/HGFR; FLT3; Trk | 46% | 2.56 | 322.80 | 1.32 |
| | | Receptor; VEGFR | | | | |
| 23 | AZD-5438 | CDK | 46% | 2.39 | 352.65 | 0.38 |
| 24 | Seliciclib | CDK | 45% | 1.32 | 118.29 | 0.55 |
| 25 | AT7519 | Apoptosis; CDK | 42% | 0.88 | 123.02 | 0.43 |
| | (Hydrochloride) | | | | | |
| 26 | BMS-345541 | IKK | 42% | 4.46 | 951.46 | 0.32 |
| | (hydrochloride) | | | | | |
| 27 | GW806742X | Mixed Lineage | 41% | 3.93 | 841.85 | 0.91 |
| | (hydrochloride) | Kinase; VEGFR | | | | |
| 28 | ARN-3236 | Salt-inducible | 33% | 1.61 | 322.60 | 0.41 |
| | | Kinase (SIK) | | | | |
| 29 | AST 487 | Bcr-Abl; c-Kit; | 32% | 0.19 | 182.68 | 0.14 |
| | | FLT3; RET; VEGFR | | | | |
| 30 | Ilginatinib | JAK | 24% | 3.17 | 484.10 | 1.39 |
| | hydrochloride | | | | | |
| 31 | CDK-IN-2 | CDK | 23% | 1.06 | 156.94 | 0.28 |
| 32 | Casein Kinase II | Casein Kinase | 18% | 0.79 | 126.12 | 0.46 |
| | Inhibitor IV | | | | | |

*This list of compounds is sorted by decreasing % viability

Remarkably, eight of the compounds assayed that positively impacted nLuc expression were compounds that target cyclin-dependent kinase (CDK) proteins, including six compounds that exclusively target the kinase.

### Example 5: Nanoparticle Delivery of DNA

The assay assessed delivery of various nanoparticles having DNA or RNA admixed therewith. Briefly, DNA encoding secreted embryonic alkaline phosphatase (SEAP) or replicon RNA encoding an RNA polymerase and SEAP were prepared and mixed with a nanoparticle of NP-1 or NP-3. Conditions are provided in Table 8. BALB/c female mice were injected intramuscularly (IM). Nucleic acid preparations for dilutions are provided in Table 9. Nanoparticle preparations are provided in Table 10. Nucleic acid-nanoparticle complexes were formed by adding 150 μl diluted NP-1 or NP-3 to 150 μl diluted DNA or RNA, then incubated for at least 30 minutes.

TABLE 8

| | | | DNA/ | RNA dose | DNA dose | | Inj. Volume | |
|---|---|---|---|---|---|---|---|---|
| Group | N | Formulation | RNA-SEAP | [μg] | [μg] | N:P | [μl] | Route |
| 1 | 5 | Naked | DNA-SEAP | | 20 | n/a | 50 | IM |
| 2 | 5 | NP-1 | DNA-SEAP | | 10 | 15 | 50 | IM |
| 3 | 5 | NP-1 | DNA-SEAP | | 10 | 7.5 | | IM |
| 4 | 5 | NP-1 | DNA-SEAP | | 20 | 15 | | IM |
| 5 | 5 | NP-1 | DNA-SEAP | | 20 | 7.5 | | IM |
| 6 | 5 | NP-1 | RNA-SEAP | 1 | | 15 | 50 | IM |
| 7 | 5 | NP-3 | RNA-SEAP | 1 | | | 50 | IM |

TABLE 9

| | | DNA | | | | Concentrations |
|---|---|---|---|---|---|---|
| | | or | 40% | | | measure prior |
| | DNA- or | RNA | sucrose | water | Total | to complexing |
| Group | RNA-SEAP | [μl] | [μl] | [μl] | [μl] | using NanoDrop |
| 1 | DNA-SEAP | 24.0 | 75.0 | 51.0 | 150.0 | 725 μg/ml |
| 2 | DNA-SEAP | 12.0 | 0.0 | 138.0 | 150.0 | 528 μg/ml |
| 3 | DNA-SEAP | 12.0 | 0.0 | 138.0 | 150.0 | 528 μg/ml |
| 4 | DNA-SEAP | 24.0 | 75.0 | 51.0 | 150.0 | 725 μg/ml |
| 5 | DNA-SEAP | 24.0 | 75.0 | 51.0 | 150.0 | 725 μg/ml |
| 6 | RNA-SEAP | 2.7 | 0.0 | 147.3 | 150.0 | 57 μg/ml |
| 7 | RNA-SEAP | 2.7 | 0.0 | 147.3 | 150.0 | 57 μg/ml |

TABLE 10

| | | | 40% | 100 mM | | |
|---|---|---|---|---|---|---|
| | | NP-1 | sucrose | citrate | Water | Total |
| Group | Formulation | [μl] | [μl] | [μl] | [μl] | [μl] |
| 1 | Naked | 0 | 0 | 15 | 135 | 150 |
| 2 | 100-015 | 72 | 90 | 18 | 0 | 180 |
| 3 | 100-015 | 36 | 90 | 18 | 36 | 180 |
| 4 | 100-015 | 144 | 0 | 18 | 18 | 180 |
| 5 | 100-015 | 72 | 0 | 18 | 90 | 180 |
| 6 | 100-015 | 7.2 | 90 | 18 | 64.8 | 180 |

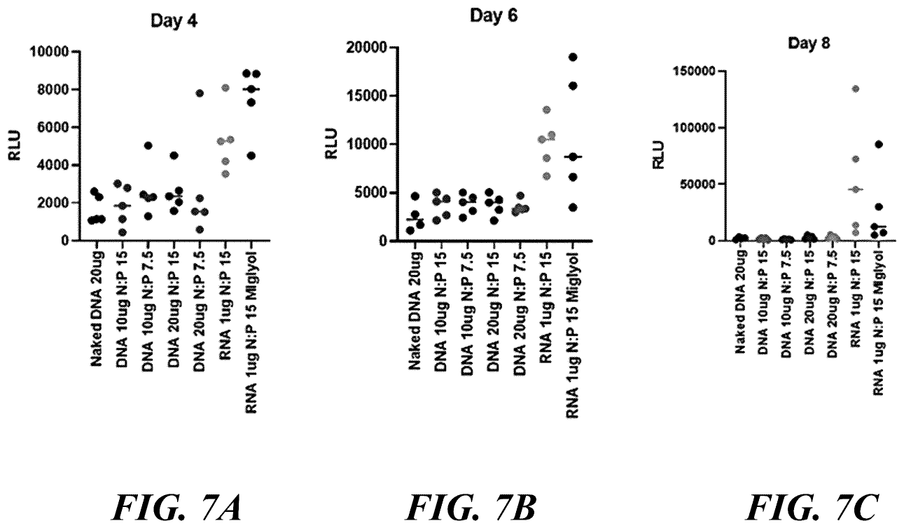

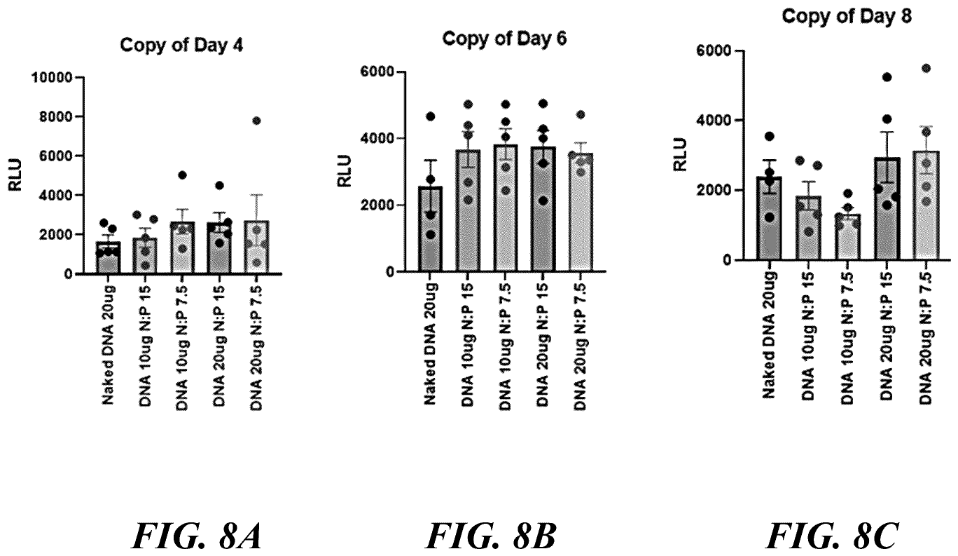

Mice were inoculated on day 0 according to the treatment groups. Blood was collected on days 4, 6 and 8, allowed to clot, and the serum was collected and stored at minus 80 degrees Celsius. Serum samples were thawed, and SEAP detection was assessed. A chemiluminescent substrate of SEAP was provided, and activity was measured based on the light generated, and quantitated as Relative Luminescence Units (RLUs). Results are shown in FIGS. 7A-7C and FIGS. 8A-8C, with a mean, n=5 per group. NP-1 and NP-3 formulations enhanced target protein production over delivery of DNA alone. Inclusion of Miglyol in NP-3 enhanced protein production of RNA over standard NP-1 having squalene.

### Example 6: Screening of Compounds for Co-Delivering with Nanoparticle Delivery of DNA

A library of small molecule kinase inhibitors was screened for the ability to enhance expression of mRNA-encoded genes when co-delivered with mRNA. About 1876 small molecule kinase inhibitors were screened at a single dilution using an automated high throughput method as described in Example 4. TABLE 11 summarizes control treatments from the single-point compound screening.

TABLE 11
Summary of control treatments from the single-point compound screening.

| | | | | Log10[fold IFIT | | Log10[fold nLuc | | Log10[fold NFκβ | |
|---|---|---|---|---|---|---|---|---|---|
| | | % viability | | over IFN] | | over RNA + IFN] | | over IFN] | |
| Treatment | N | Mean | Std. Dev. | Mean | Std. Dev. | Mean | Std. Dev. | Mean | Std. Dev. |
| RNA + IFN | 99 | 97.90 | 0.14 | 0.13 | 0.12 | −0.06 | 0.22 | 0.07 | 0.10 |
| RNA + anti-IFN + IFN | 96 | 98.60 | 0.12 | −0.77 | 0.13 | 1.15 | 0.26 | −0.13 | 0.09 |
| RNA | 92 | 98.90 | 0.11 | −.63 | 0.22 | 1.25 | 0.40 | −0.10 | 0.10 |
| IFN | 49 | 98.90 | 0.09 | 0.00 | 0.02 | −1.58 | 0.58 | 0.00 | 0.04 |
| Media | 48 | 100.00 | 0.06 | −1.36 | 0.32 | −1.76 | 0.62 | −0.16 | 0.07 |

Means and standard deviations reflect aggregate of all responses measured within and between plates. N = the number of individual responses measured to calculate the corresponding mean and standard deviation. Positive treatment controls for each response are in bold letters.

Compounds that enhanced nLuc expression greater than 100-fold (2 log₁₀) over RNA+IFN treated cells were identified as hits. This reduced the number of compounds of interest to 99 (5.300 of library). We then dissected the hits by their intracellular targets (TABLE 12) and found that 32 compounds, approximately one-third of the hits, targeted cyclin-dependent kinases (CDKs).

TABLE 12
Compounds with enhanced nLuc expression greater than 100-fold
(2log10) over RNA + IFN treated cells and their intracellular targets

| Target | No. | Target | No. | Target | No. |
|---|---|---|---|---|---|
| Adenosine | 1 | IGF-1R | 1 | PKC | 2 |
| Kinase | | | | | |
| Apoptosis | 17 | IKK | 4 | PKD | 2 |
| Aurora Kinase | 1 | IRAK | 1 | Polo-like Kinase | 2 |
| | | | | (PLK) | |
| Autophagy | 12 | IRE1 | 1 | PROTAC | 1 |
| Bcr-Abl | 3 | JAK | 6 | Pyk2 | 2 |
| c-Fms | 1 | JNK | 1 | Raf | 1 |
| c-Kit | 1 | Ligand for Target | 1 | RET | 1 |
| | | Protein for | | | |
| | | PROTAC | | | |
| c-Met/HGFR | 3 | Lipoxygenase | 1 | Reverse | 1 |
| | | | | Transcriptase | |
| Casein Kinase | 3 | LRRK2 | 1 | ROCK | 1 |
| CDK | 32 | MAP3K | 1 | Salt-inducible | 1 |
| | | | | Kinase (SIK) | |
| CRISPR/Cas9 | 1 | MAP4K | 2 | Src | 1 |
| DYRK | 1 | MEK | 1 | Syk | 1 |
| ERK | 2 | Microtubule/Tubulin | 1 | TAM Receptor | 1 |
| Eukaryotic | 1 | Mixed Lineage | 2 | TOPK | 1 |
| Initiation | | Kinase | | | |
| Factor (elF) | | | | | |
| FAK | 2 | Others | 3 | TOPOISOMERASE | 1 |
| FGFR | 2 | p38 MAPK | 1 | Trk Receptor | 2 |
| FLT3 | 2 | PAK | 1 | VEGFR | 8 |
| GSK-3 | 9 | Parasite | 1 | Virus Protease | 1 |
| Haspin Kinase | 1 | PDGFR | 1 | WnT | 4 |
| HIV | 1 | PI3K | 2 | B-catenin | 3 |
| HSV | 1 | PI4K | 1 | | |

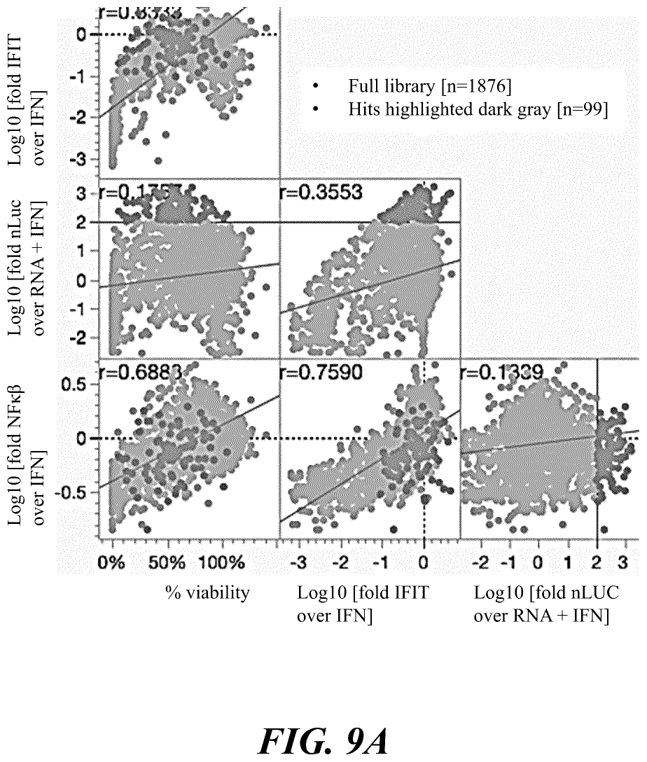

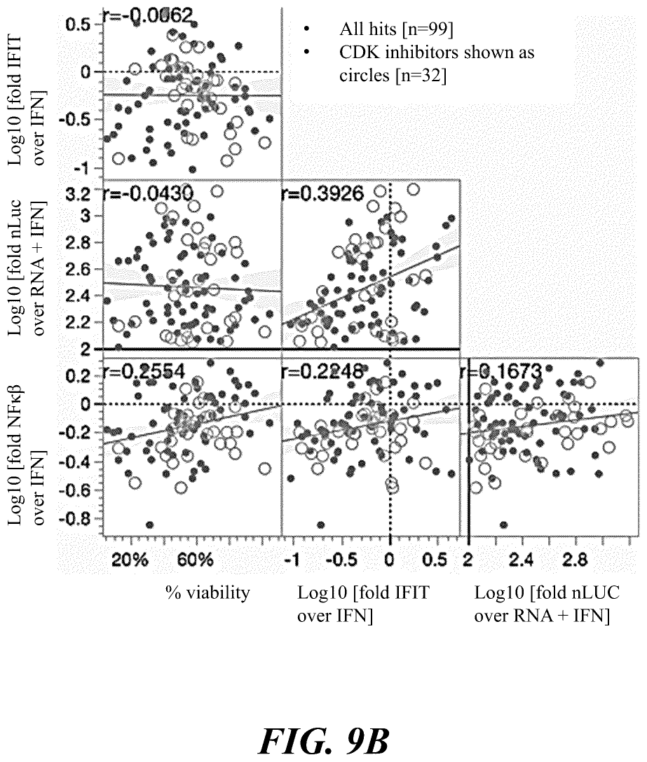

FIG. 9A summarizes pairwise correlations between nLuc expression, IFIT2 induction, NF-κβ induction and % viability. There was significant positive correlation between viability and both inmate immune induction measures (FIG. 9A). In the subset of compounds identified as hits (FIG. 9B), 84/99 compounds (85% of hits) reduced IFIT2 induction compared to cells treated with RNA+IFN. Interestingly, a positive correlation was observed between fold-change in nLuc expression and fold-change in IFIT2 induction (Pearson r=0.39; p-value <0.0001) suggesting expression was higher when the reduction in IFIT2 induction was minimal. In summary, high throughput single-point screening of 1,876 compounds yielded 99 hits that were selected primarily based on their ability to overcome IFN-mediated suppression of repRNA-encoded protein (nLuc) expression in A549-Dual cells by at least 100-fold over baseline expression. The compounds and their targets are summarized is TABLE 13.

TABLE 13
Compounds identified as hits from the single-point screening and their targets

| Compound | Target | Compound | Target |
|---|---|---|---|
| (−)-BAY-1251152 | CDK | Indirubin-3′- | GSK-3; Lipoxygenase |
| | | monoxime | |
| (+)-BAY-1251152 | CDK | IRAK inhibitor 2 | IRAK |
| (±)-BAY-1251152 | CDK | K00546 | CDK; GSK-3; VEGFR |
| (2S,3R)-Voruciclib | CDK | kb NB 142-70 | PKD |
| (hydrochloride) | | | |
| (R)-Simurosertib | Others | KB-0742 | CDK |
| | | (dihydrochloride) | |
| 5-Iodotubercidin | Adenosine Kinase | LDC4297 | CDK; HSV |
| ACHP | IKK | LSN3106729 | CDK; Ligand for Target |
| (Hydrochloride) | | (hydrochloride) | Protein for PROTAC |
| Altiratinib | c-Met/HGFR; FLT3; Trk | LY2857785 | Apoptosis; CDK |
| | Receptor; VEGFR | | |
| APY29 | IRE1 | LY2874455 | FGFR |
| AR-A014418 | GSK-3 | MAPK13-IN-1 | Autophagy; p38 MAPK |
| ARN-3236 | Salt-inducible Kinase (SIK) | MC180295 | CDK |
| AST 487 | Bcr-Abl; c-Kit; FLT3; RET; | Mitoxantrone | PKC; Topoisomerase |
| | VEGFR | (dihydrochloride) | |
| AT7519 | Apoptosis; CDK | Momelotinib | Apoptosis; Autophagy; JAK |
| (Hydrochloride) | | | |
| Autophinib | Autophagy; PI3K | Momelotinib | Apoptosis; Autophagy; JAK |
| | | sulfate | |
| AUZ 454 | CDK | NCB-0846 | MAP4K; Wnt |
| AZ-23 | Trk Receptor | Necrosulfonamide | Mixed Lineage Kinase |
| AZ960 | Apoptosis; JAK; Parasite; | NIH-12848 | Others |
| | Virus Protease | | |
| AZD-5438 | CDK | Nocodazole | Apoptosis; Autophagy; Bcr- |
| | | | Abl; CRISPR/Cas9; |
| | | | Microtubule/Tubulin |
| AZD4573 | CDK | NVP-LCQ195 | CDK |
| BAY-985 | IKK | OTS514 | Apoptosis; TOPK |
| BF738735 | PI4K; Reverse Transcriptase | PF-06873600 | CDK |
| BMS-345541 | IKK | PF-3758309 | Apoptosis; PAK |
| BMS-345541 | IKK | PF-562271 | FAK; Pyk2 |
| (hydrochloride) | | | |
| BMS-794833 | c-Met/HGFR; VEGFR | PF-562271 | FAK; Pyk2 |
| | | (besylate) | |
| Casein Kinase II | Casein Kinase | PHA-767491 | Apoptosis; CDK |
| Inhibitor IV | | (hydrochloride) | |
| CCT196969 | Raf | PHA-793887 | Apoptosis; CDK |
| CDK-IN-2 | CDK | PLX5622 | c-Fms |
| CDK12-IN-3 | CDK | PROTAC CDK9 | CDK; PROTAC |
| | | Degrader-1 | |
| CDK9-IN-8 | CDK | R112 | Syk |
| CDKI-73 | Apoptosis; CDK | Ravoxertinib | ERK |
| | | hydrochloride | |
| CHIR-99021 | Autophagy; GSK-3; Wnt; β- | RGB-286638 (free | CDK; GSK-3; JAK; MEK |
| | catenin | base) | |
| CHIR-99021 | Autophagy; GSK-3; Wnt; β- | RKI-1447 | ROCK |
| (monohydrochloride) | catenin | | |
| CHIR-99021 | Autophagy; GSK-3; Wnt; β- | SBE13 | Apoptosis; Autophagy; Polo- |
| (trihydrochloride) | catenin | (Hydrochloride) | like Kinase (PLK) |
| CHR-6494 | Haspin Kinase | SCR-1481B1 | c-Met/HGFR; VEGFR |
| CP21R7 | GSK-3 | Seliciclib | CDK |
| CRT0066101 | PKD | Selonsertib | Apoptosis; MAP3K |
| dihydrochloride | | | |
| Dinaciclib | Apoptosis; CDK | Simurosertib | CDK |
| DMX-5804 | MAP4K | SNS-032 | Apoptosis; CDK |
| Flavopiridol | Autophagy; CDK; HIV | SR-3029 | Casein Kinase |
| (Hydrochloride) | | | |
| GCN2-IN-6 | Eukaryotic Initiation Factor | SU3327 | JNK |
| | (eIF) | | |
| Go 6983 | PKC | SU9516 | Apoptosis; Autophagy; CDK |
| GSK-3 inhibitor 1 | GSK-3 | THZ2 | CDK |
| GSK-626616 | DYRK | Tyrosine kinase- | FGFR; PDGFR; VEGFR |
| | | IN-1 | |
| GW806742X | Mixed Lineage | Kinase; | UNC2881 |
| (hydrochloride) | VEGFR | | |
| HG-10-102-01 | LRRK2 | VEGFR-2-IN-5 | VEGFR |
| | | (hydrochloride) | |
| HMN-176 | Others | Voruciclib | CDK |
| | | (hydrochloride) | |
| HMN-214 | Polo-like Kinase (PLK) | Vps34-IN-1 | Autophagy; PI3K |
| IC 261 | Apoptosis; Casein Kinase | VX-11e | ERK |
| Ilginatinib | JAK | XL228 | Aurora Kinase; Bcr-Abl; |
| | | | IGF-1R; Src |
| Ilginatinib | JAK | | |
| hydrochloride | | | |

The subset of 99 compounds identified as hits from the single-point screening were evaluated for potency by measuring inhibitor dose-dependent modulation in nLuc expression. Unlike in the single-point screening where cells were pretreated overnight and thus primed with compounds before repRNA delivery, compounds were added with the NP-1/repRNA transfection step to mimic co-delivery conditions as intended in the target application. RepRNA amount was fixed to 20 ng in all wells and based on preliminary dose-response experiments, an optimal dose range for compounds was determined to be of, from high to low, 50-0.02 μM for low potency, 10-0.004 μM for medium potency and 200-0.09 nM (nanomolar) for high potency compounds. Compounds were ranked based on two criteria: (a) their half-maximal effective concentration (EC50) that enhanced nLuc expression over RNA+IFN and (b) fold change in nLuc expression at the top concentration compared to RNA+IFN. 10 candidate compounds, bold in TABLE 13, were picked based on the selection criteria described above. Their EC50 values and fold change in expression over RNA+IFN at the top concentration are plotted in FIG. 10. All 10 compounds targeted the CDK family of kinases, with five ((±)-BAY-1251152, AZD4573, CDK12-IN-3, CDK-IN-2 and MC180295) exclusively targeting a CDK protein. In addition to targeting CDKs, three compounds (Dinaciclib, CDKI-73 and LY2857785) are also known to induce apoptosis in cancer cells, one compound (Flavopiridol hydrochloride) is known to induce autophagy and block HIV-1 replication, and another (RGB-286638) is known to target additional kinases, namely glycogen kinase synthase 3 beta (GSK3B), TGF-beta activated kinase 1 (TAK1), Janus kinase 2 (JAK2) and mitogen-activated kinase 1 (MEK1). The more than 100-fold increase in nLuc expression observed from the pretreatment single-point screening experiment was significantly reduced when compounds were co-delivered with repRNA, but expression levels were still 7 to 20-fold higher than in cells treated with RNA+IFN only.

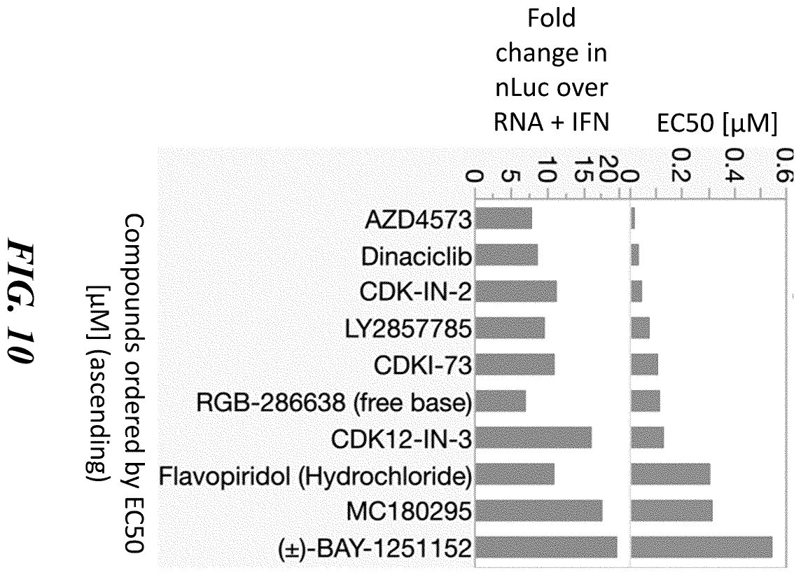

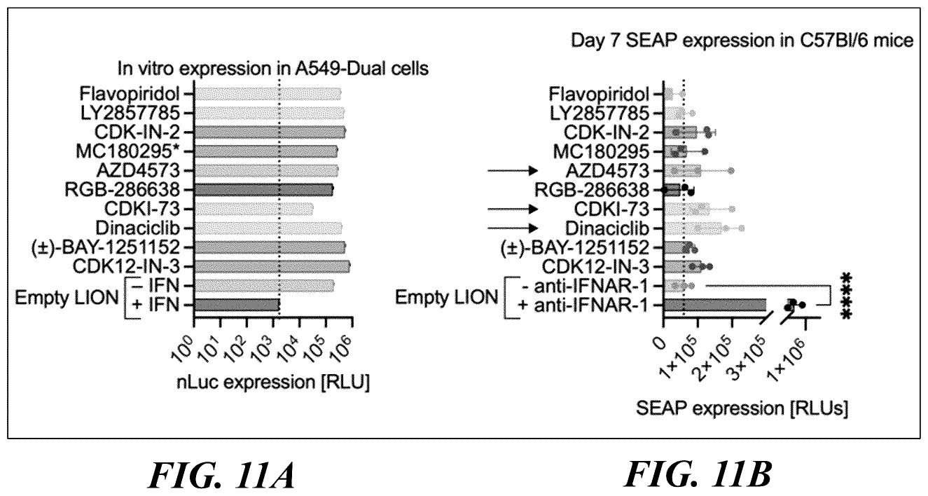

The ten candidate compounds were formulated in NP-1 nanoparticle emulsion to evaluate their effect on expression when co-delivered with repRNA. All compounds were readily soluble in non-polar solvents which enabled dissolution in the lipophilic squalene oil phase of NP-1. A probe sonication process was adapted to formulate candidate compounds in 1 mL batches of nanoparticle emulsions. The z-average nanoparticle diameter was measured by dynamic light scattering (DLS) and ranged from 110-130 nm. Activity of compounds formulated in nanoparticle emulsions was tested both in vitro (FIG. 11A). Cells were transfected with 20 ng repRNA per well in the presence of IFN and delivered with empty nanoparticles (no inhibitor) or nanoparticle encapsulating a compound inhibitor delivered at a calculated 0.5 μM per well. All compounds formulated in nanoparticle emulsions rescued transgene expression from IFN-mediated shutdown, demonstrating that co-delivery of the compounds with repRNA in the same nanoparticle formulation does not abrogate their function. The ten compounds formulated in nanoparticle emulsions were then tested at a single dose and timepoint in mice. Based on previous kinetics studies, a seven day duration was chosen to compare differences in protein expression between groups. C57BL/6 female mice (6-8 weeks old; n=3/group) were injected by IM route with 10 μg repRNA-SEAP complexed with empty nanoparticles (no compound) or nanoparticles encapsulating a small molecule inhibitor at 500 μM. The positive control group received an IFNAR-1 blocking monoclonal antibody (MAR1-5A3) by intraperitoneal (IP) injection one day before IM injection of NP-1/repRNA-SEAP. As shown in FIG. 11B, systemic blockade of IFN-alpha/beta signaling resulted in 10-fold greater SEAP expression on day seven after injection compared to no IFNAR-1 blocking. Moreover, more than half the compound groups (6/10) showed a modest increase in mean SEAP expression compared to NP-1/repRNA alone.

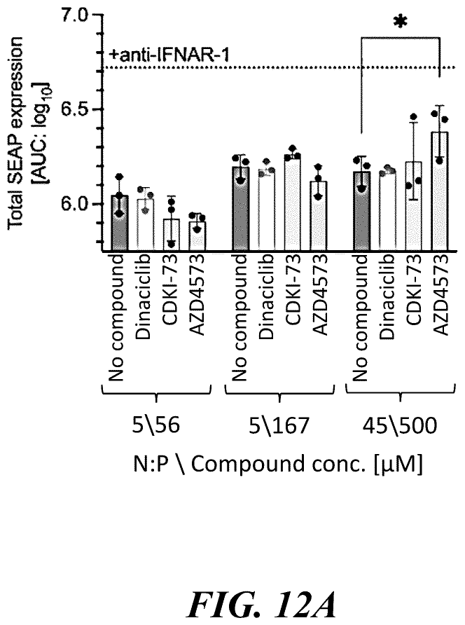

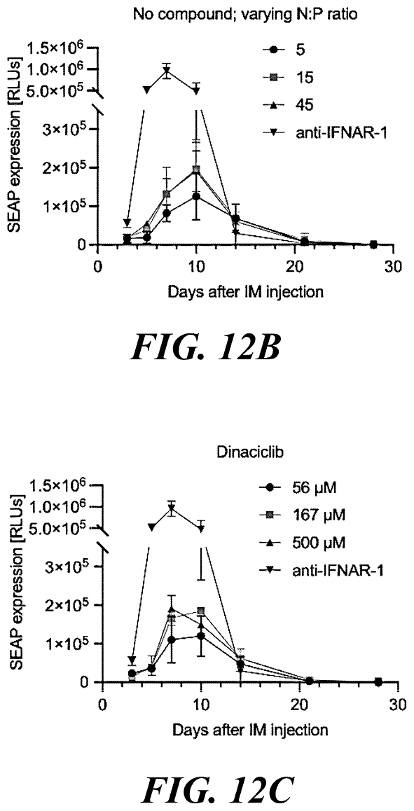

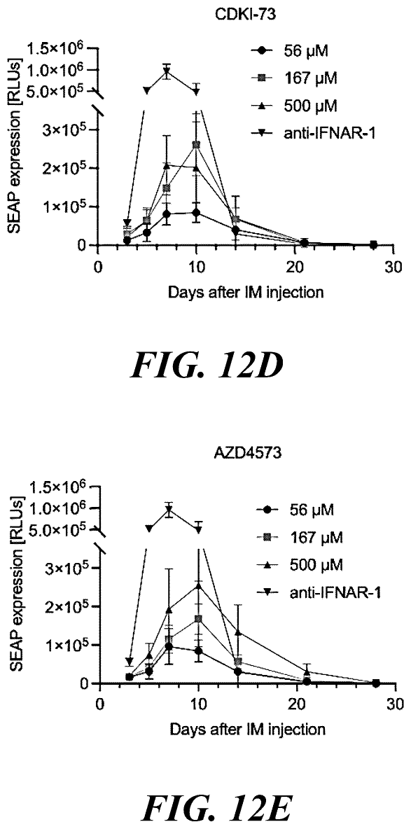

Based on mean levels of SEAP in serum (FIG. 11B), three compounds, Dinaciclib, CDKI-73 and AZD4573, were advanced to evaluate inhibitor dose-dependent response in mice (C57BL/6 females, 6-8 weeks old, n=3 per group). Since compounds are encapsulated in the oil phase of the nanoparticles, dose was adjusted by varying the nanoparticle to repRNA ratio, commonly referred to as the molar ratio of nitrogen to phosphate (N:P). The empty nanoparticles (no compound control) were tested at the corresponding N:P ratios to account for the effect of varying nanoparticles. Mice were bled on days 3, 5, 7, 10, 14, 21 and 28 after IM injection and assayed for SEAP levels in serum. Total protein expression (FIG. 12A) was calculated by measuring area under the expression kinetics curve (AUC) for each group shown in FIGS. 12B-E. The CDK9 inhibitor AZD4573 produced a significant increase in total protein expression at the 500 μM dose level compared to the equivalent “no compound” group. The data for the first time provided proof that co-delivery of a small molecule CDK inhibitor enhances repRNA-encoded transgene expression in vivo.

### Example 7: Systemic Co-Delivery of Compounds with Nanoparticle Delivery of RNA

Three compounds, Dinaciclib, CDKI-73 and AZD4573, dissolved in co-solvents, were administered intravenously by tail-vein injection followed by intramuscular injection of NP-1 nanoparticle emulsion formulated with repRNA-ZIKV-117. The purpose of the experiment was to evaluate the effect of systemically administered compound inhibitors on protein expression.

Briefly, three compounds, Dinaciclib, CDKI-73 and AZD4573, were formulated in nanoparticle emulsion according to the process described in Example 6. A molar ratio of nitrogen to phosphate (N:P) for each of the formulation was adjusted. Female BALB/c mice (6-8 weeks old, n=3 per group) were given Dinaciclib (5 mg/kg), CDKI-73 (5 mg/kg) or AZD4573 (1 mg/kg) by IV injection in the tail vein. Each compound was formulated as an aqueous solution or suspension following instructions from the vendor (MedChemExpress). At the same time as systemic compound treatment, mice were given 10 μg repRNA-encoding ZIKV-117 mAb complexed to nanoparticles by IM injection in the hind leg. Mice were bled on days 3, 5, 7, 10, 14, 21 and 28 after administration of NP-1/repRNA-ZIKV-117, and assayed for ZIKV-117 levels in serum. The positive control group received an IFNAR-1 blocking monoclonal antibody (MAR1-5A3) by intraperitoneal (IP) injection one day before administration of NP-1/repRNA-ZIKV-117.

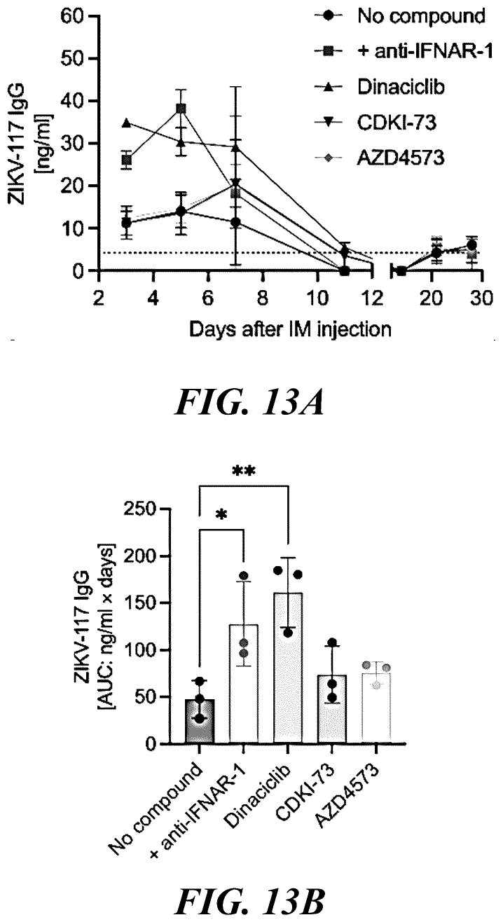

FIGS. 13A-13B summarize systemic administration of CDK inhibitors at a relatively high dose. An analysis of FIGS. 13A-13B indicate that Dinaciclib, significantly enhanced ZIKV-117 expression compared to mice treated with no compounds. Surprisingly, systemic treatment with Dinaciclib resulted in more rapid and higher magnitude expression compared to the positive control mice treated with anti-IFNAR-1. CDKI-73 and AZD4573 also resulted in higher total expression than no compound group but not statistically significant for the sample size tested.

### Example 8: Co-Transfection of NP-35 Formulations with Small Molecule Compounds

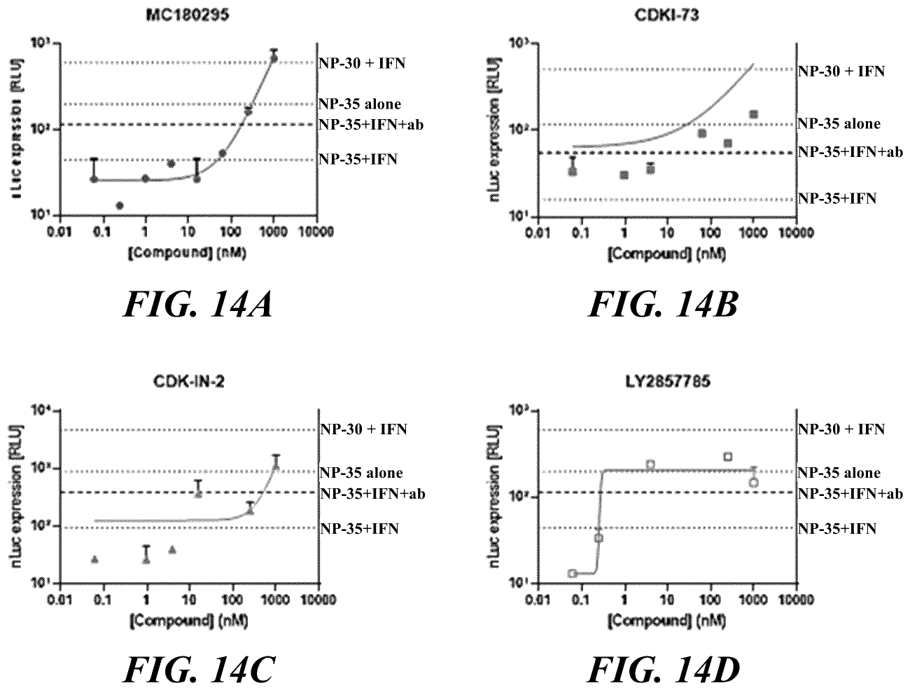

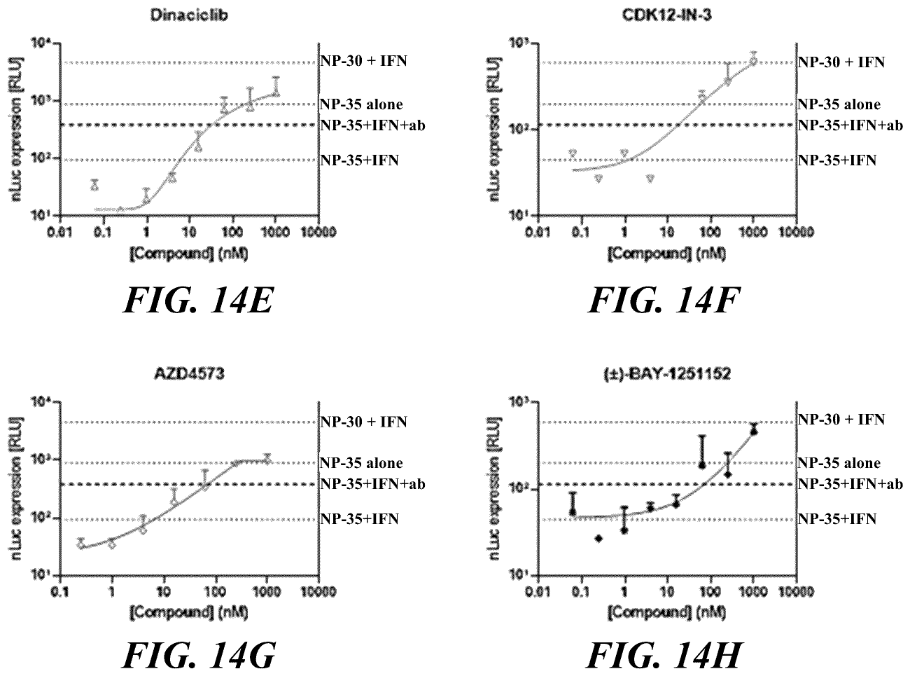

The objective of this experiment was to evaluate co-transfection of LNP (e.g., NP-35) with lead small molecule inhibitor compounds for enhancing protein expression from repRNA in transfected cells. Briefly, eight compounds, MC180295, CDKI-73, CDK-IN-2, LY2857785, Dinaciclib, CDK12-IN-3, AZD4573, and (±)-BAY-1251152 were selected for this experiment. LNP was formulated according to NP-35 formulation. Additionally, nanoparticles were also formulated according to NP-3β formulation. About 5×10⁴ A549-Dual cells were incubated at 37° C. with 5% CO₂ for 18-24 hours with the compound at a concentration ranging from 1 μg to 0.06 ng. The cells were then treated with repRNA-nLuc, IFN, and nanoparticles (NP-35 or NP-30). The treated cells were incubated at 37° C. with 5% CO₂ for 18-24 hours. nLuc expression was measured by Nano-GLO assay and plotted against the concentration of repRNA-nLuc that was used for the transfection (FIG. 14A-14H). EC50 for each of the CDK inhibitors was calculated based on analysis of FIGS. 14A-14H and summarized in TABLE 14.

TABLE 14
EC50 of CDK compound

| Compound | EC50 |
|---|---|
| MC180295 | 996.9 |
| CDKI-73 | 1661 |
| CDK-IN-2 | 1110 |
| LY2857785 | 0.271 |
| Dinaciclib | 0.359 |
| CDK12-IN-3 | 3621 |
| AZD4573 | 106.3 |
| (±)-BAY-1251152 | 1931 |

An analysis of FIGS. 14A-14H and TABLE 14 suggests that each CDK inhibitor co-delivered with NP-35/repRNA-nLuc in the presence of exogenous interferon (IFN) enhanced secreted nanoluciferase (nLuc) expression in a dose-dependent manner relative to cells transfected with NP-35/repRNA-nLuc+IFN (dotted line labeled “NP-35+IFN”) in the absence of CDK inhibitors (FIG. 14A-14H). The analysis further suggests that Co-encapsulation of Dinaciclib in NP-35/repRNA-nLuc significantly enhanced nLuc expression as demonstrated by a decrease in the EC50 (increased potency) and increase in expression at all RNA concentrations relative to NP-35/repRNA-nLuc without Dinaciclib. Overall NP-35 expression was significantly lower than NP-30. Different lots of repRNA were used for NP-35 manufacture and NP-3β complexing so it likely the RNA used in the NP-35 was not as functional. An analysis of TABLE 14 suggests that LY2857785 and Dinaciclib had significantly lower EC50 values compared to other CDK inhibitors, followed by AZD4573.

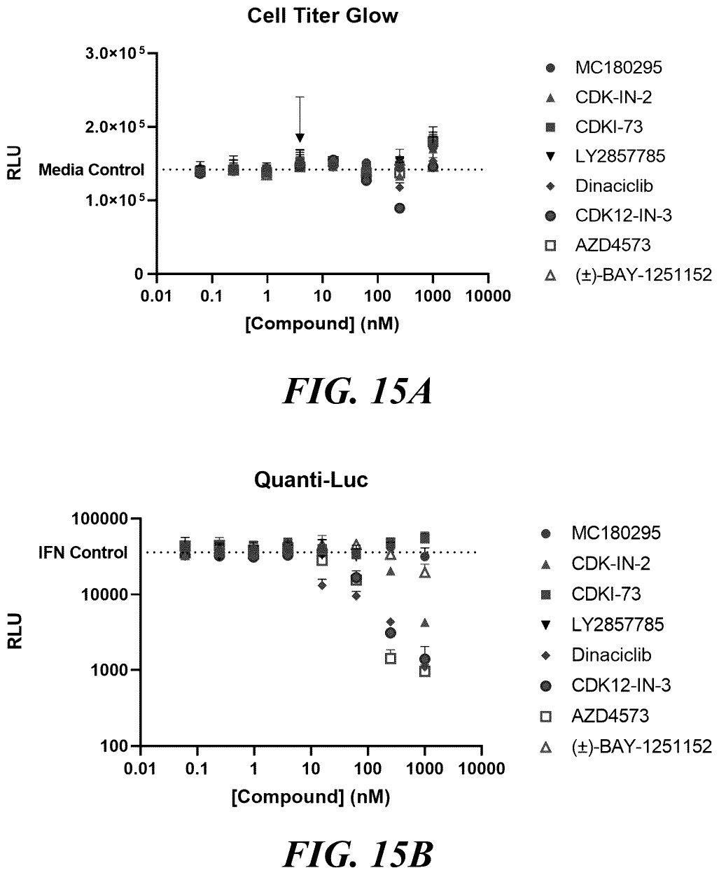

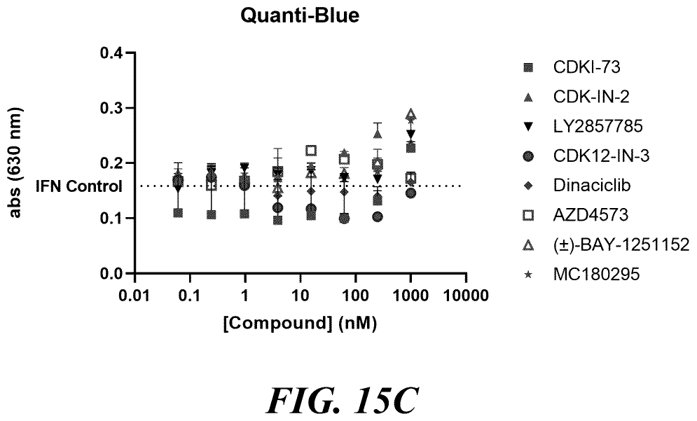

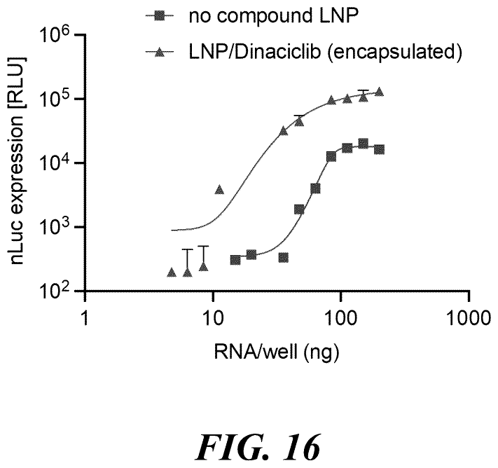

None of the compounds caused significant cell death compared to untreated cells (FIG. 15A). There was significant dose-dependent reduction in IFIT2 induction in cells treated with NP-35-formulated AZD4573, CDK12-IN-3, Dinaciclib or CDK-IN-2 (FIG. 15B). There was a slight increase in IFN-beta mediated NF-κB induction in cells treated with the highest concentrations of NP-35-formulated MC180295, CDKI-73, CDK-IN-2 or LY2857785 relative to cells transfected with NP-35 (FIG. 15C).

### Example 9: Evaluation of NP-35 Formulation for Co-Delivery of Dinaciclib to Enhance Protein Expression from repRNA-nLuc

The objective of this experiment was to evaluate LNP (e.g., NP-35) encapsulated Dinaciclib in transfected cells for enhancing protein expression from repRNA. Briefly, A549-Dual cells were incubated at 37° C. with 5% CO₂ for 18-24 hours. Two formulations were prepared: (1) NP-35 nanoparticles encapsulating repRNA-nLuc and 0.5 mM Dinaciclib; and (2) NP-35 nanoparticles with only repRNA-nLuc (no compound). About 5×10⁴ cells were transfected with the formulation at different concentrations in the presence of IFN. The transfected cells were incubated at 37° C. with 5% CO₂ for 18-24 hours. nLuc expression was measured by Nano-GLO assay and plotted against the concentration of repRNA-nLuc that was used for the transfection (FIG. 15). An analysis of FIG. 15 suggests that NP-35/repRNA-nLuc showed RNA dose-dependent expression of nLuc in A549-Dual cells in both the presence and absence of co-encapsulating Dinaciclib. The analysis further suggests that Co-encapsulation of Dinaciclib in NP-35/repRNA-nLuc significantly enhanced nLuc expression as demonstrated by a decrease in the EC50 (increased potency) and increase in expression at all RNA concentrations relative to NP-35/repRNA-nLuc without Dinaciclib.

### Example 10: Co-Transfection of Nucleic Acids Coding Expression Enhancer and Protein with NP-35 Formulation

Nucleic acids coding expression enhancers, such as kinase inhibitors, are screened for evaluating the ability to increase expression of mRNA-encoded genes when co-delivered with mRNA. Kinase inhibitors coded by mRNA (repRNA-KI) of each of SEQ ID NO: 41-47 are screened. Briefly, seven formulations, each containing NP-35 nanoparticles encapsulating repRNA-nLuc and repRNA-KI are prepared. About 5×10⁴ A549-Dual cells are incubated at 37° C. with 5% CO₂ for 18-24 hours. The cells are transfected with the formulation. During the transfection, IFN is added to the cells. The transfected cells are incubated at 37° C. with 5% CO₂ for 18-24 hours. nLuc expression is measured by Nano-GLO assay.

### Example 11: Evaluation of NP-35 Formulation for Co-Delivery of CDK Inhibitor to Cells Co-Transfected with Nucleic Acids Coding Expression Enhancer and repRNA-nLuc

The objective of this experiment is to evaluate NP-35 co-encapsulating CDK inhibitor, a nucleic acids coding for kinase inhibitor (repRNA-KI) and repRNA-nLuc. The CDK inhibitor includes any one or more of the compounds recited in TABLE 14. The repRNA-KI includes any nucleotide sequence that has at least 80% sequence identity with any one of the sequences of SEQ ID NO: 41-47. Briefly, A549-Dual cells are incubated at 37° C. with 5% CO₂ for 18-24 hours. Four formulations are prepared: (1) NP-35 nanoparticles encapsulating repRNA-nLuc, repRNA-KI, and 0.5 mM CDK inhibitor; (2) NP-35 nanoparticles with only repRNA-nLuc and repRNA-KI; (2) NP-35 nanoparticles with only repRNA-nLuc; and 0.5 mM CDK inhibitor; and (4) NP-35 nanoparticles with only repRNA-nLuc. About 5×10⁴ cells are treated with IFN and transfected with the formulation at different concentrations. The transfected cells are incubated at 37° C. with 5% CO₂ for 18-24 hours. nLuc expression is measured by Nano-GLO assay.

SEQUENCES

SEQ ID NO: 1 SARS CoV-2 A.1 antigen
AUAGGCGGCGCAUGAGAGAAGCCCAGACCAAUUACCUACCCAAAAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCUAACCUGAAUGGACUAC
GACAUAGUCUAGUCCGCCAAGAUGUUUCUGCUCACAACCAAACGCACUAUGUUUGUUUUCCUCGU
GCUGCUCCCUUUGGUAAGUUCUCAGUGUGUAAACCUGACAACACGAACCCAGUUGCCUCCAGCUU
AUACCAACUCAUUUACUCGCGGAGUAUAUUAUCCCGAUAAGGUCUUUAGAAGUAGCGUGUUGCAC
UCUACACAGGAUCUGUUCUUGCCCUUCUUUAGUAACGUUACCUGGUUUCAUGCAAUACAUGUGAG
CGGAACAAAUGGAACAAAAAGAUUUGACAAUCCAGUGCUUCCAUUUAAUGAUGGGGUUUACUUUG
CCAGUACCGAAAAGUCAAACAUAAUCCGGGGGUGGAUCUUUGGAACCACUUUGGACUCUAAGACA
CAGUCUCUCCUCAUAGUAAACAACGCCACCAAUGUUGUCAUAAAAGUAUGCGAAUUUCAGUUUUG
CAACGAUCCCUUUCUCGGGGUGUAUUACCAUAAGAAUAAUAAAUCCUGGAUGGAGUCUGAGUUCC
GGGUUUAUAGUAGUGCUAAUAAUUGCACUUUCGAAUACGUGUCCCAACCAUUCCUCAUGGACCUU
GAGGGCAAACAGGGGAAUUUUAAAAACUUGCGCGAAUUUGUCUUUAAGAAUAUCGACGGAUACUU
UAAGAUCUAUAGUAAACACACUCCUAUCAACCUCGUUCGGGAUCUUCCCCAAGGCUUUUCUGCUC
UCGAACCCCUCGUAGACUUGCCAAUUGGGAUAAAUAUCACUCGCUUUCAAACUUUGCUUGCCCUC
CACAGGAGCUACCUGACACCCGGCGACUCUUCUUCUGGUUGGACCGCCGGCGCCGCUGCCUAUUA
UGUUGGUUACCUUCAGCCACGAACAUUCUUGCUCAAGUAUAACGAGAAUGGCACCAUUACCGACG
CCGUCGAUUGUGCAUUGGAUCCCUUGUCUGAAACAAAAUGUACCUUGAAGUCCUUUACCGUAGAG
AAAGGCAUAUACCAGACUUCCAACUUCCGAGUUCAGCCUACAGAAUCCAUUGUGAGAUUUCCCAA
CAUCACAAACCUCUGCCCUUUCGGUGAAGUAUUUAAUGCUACACGCUUCGCUUCAGUCUAUGCCU
GGAAUAGGAAGCGCAUAUCAAAUUGCGUGGCCGAUUAUUCAGUCCUCUAUAAUAGCGCAUCCUUC
AGUACUUUCAAGUGCUACGGCGUUUCCCCCACCAAACUCAAUGAUCUUUGCUUCACCAACGUCUA
UGCUGACAGUUUUGUCAUACGAGGCGACGAAGUACGCCAGAUUGCCCCCGGGCAGACAGGUAAAA
UUGCUGAUUAUAAUUAUAAACUCCCAGAUGACUUUACUGGAUGCGUCAUAGCCUGGAAUUCCAAC
AAUCUUGAUUCCAAGGUUGGUGGGAAUUAUAAUUACCUUUAUCGACUGUUCAGAAAGAGUAACUU
GAAACCAUUUGAGAGAGACAUAUCCACCGAGAUUUACCAGGCAGGCAGUACUCCUUGUAACGGCG
UUGAGGGAUUUAACUGCUAUUUUCCUUUGCAAUCCUAUGGCUUUCAACCAACAAACGGGGUUGGC
UAUCAACCCUAUCGAGUGGUUGUCCUGAGCUUUGAACUUUUGCACGCUCCCGCCACAGUCUGCGG
ACCAAAAAAGAGUACAAAUCUUGUCAAGAAUAAGUGCGUAAAUUUCAAUUUCAAUGGCCUUACAG
GAACAGGCGUGCUGACUGAGUCAAACAAGAAGUUCCUGCCAUUUCAGCAGUUUGGGCGGGAUAUA
GCAGACACAACUGACGCUGUACGCGAUCCUCAGACUUUGGAGAUCUUGGACAUCACUCCCUGUUC
UUUCGGAGGGGUAUCUGUCAUCACCCCCGGAACUAAUACAUCAAAUCAGGUCGCUGUGUUGUACC
AAGAUGUCAACUGCACAGAAGUCCCCGUUGCUAUACACGCAGACCAGCUCACCCCCACAUGGCGG
GUGUACUCAACUGGCUCAAACGUAUUCCAGACCAGAGCUGGGUGCUUGAUCGGUGCUGAACACGU
AAACAAUAGCUAUGAAUGCGAUAUUCCCAUCGGUGCCGGGAUCUGCGCUAGCUAUCAGACACAGA
CCAAUUCCCCCCGGCGAGCACGAUCUGUAGCAUCCCAGUCUAUUAUUGCCUACACUAUGUCAUUG
GGCGCCGAGAAUAGCGUCGCAUAUUCAAAUAAUUCUAUUGCAAUACCCACCAACUUCACAAUCUC
CGUAACUACAGAAAUACUUCCAGUUUCCAUGACAAAGACAUCAGUGGAUUGUACAAUGUAUAUAU
GCGGAGAUUCCACAGAAUGUUCAAAUUUGCUCUUGCAGUACGGCUCCUUCUGCACCCAGCUCAAC
AGGGCCCUUACAGGUAUUGCUGUCGAACAGGACAAGAACACACAAGAAGUCUUCGCCCAAGUCAA
ACAGAUAUACAAAACUCCUCCCAUAAAGGAUUUUGGCGGCUUCAACUUUAGUCAGAUCCUCCCAG
ACCCUUCAAAACCAUCUAAACGAUCAUUUAUUGAAGAUCUGCUGUUCAACAAGGUCACUCUUGCC
GAUGCUGGAUUCAUUAAGCAAUACGGUGACUGCCUUGGUGAUAUUGCUGCCCGAGAUCUGAUCUG
UGCCCAGAAAUUCAACGGGCUCACUGUACUCCCUCCACUGCUCACAGACGAAAUGAUUGCACAGU
ACACAAGUGCCCUGUUGGCAGGCACAAUCACUAGCGGCUGGACCUUUGGCGCAGGUGCAGCACUC
CAAAUACCUUUUGCCAUGCAGAUGGCCUAUCGGUUUAAUGGGAUAGGCGUGACUCAAAAUGUCCU
CUACGAAAACCAAAAGUUGAUAGCUAACCAAUUCAAUUCAGCAAUCGGGAAGAUACAGGAUUCAC
UGUCUAGUACUGCUAGUGCCCUUGGUAAGCUGCAGGACGUUGUCAACCAGAAUGCUCAAGCUCUG
AAUACAUUGGUUAAGCAGCUCUCUAGUAAUUUUGGGGCCAUCUCUUCAGUACUUAAUGAUAUUUU
GAGCCGAUUGGACAAAGUGGAAGCUGAAGUACAGAUCGACAGGCUGAUAACAGGCCGGCUCCAAU
CCCUCCAAACAUACGUGACACAACAACUCAUACGCGCAGCCGAAAUCCGAGCCAGCGCUAACCUG
GCAGCUACCAAGAUGUCAGAAUGCGUUCUGGGCCAGAGUAAACGCGUAGAUUUCUGCGGGAAAGG
GUACCACCUGAUGUCCUUUCCACAAUCUGCACCUCACGGGGUCGUCUUUUUGCAUGUAACAUAUG
UACCCGCACAAGAGAAGAAUUUUACUACCGCUCCUGCCAUCUGUCAUGACGGGAAAGCUCAUUUU
CCUCGCGAAGGUGUGUUUGUAUCUAAUGGUACACAUUGGUUUGUCACACAGCGGAAUUUCUAUGA
ACCCCAGAUCAUUACAACUGACAACACUUUUGUUUCCGGGAAUUGUGACGUGGUCAUAGGAAUCG
UAAAUAACACUGUAUAUGAUCCCCUCCAACCAGAGCUGGACUCUUUUAAAGAAGAACUGGAUAAA
UAUUUCAAGAACCACACAAGUCCCGACGUGGACCUUGGGGACAUAAGUGGUAUUAACGCAUCUGU
GGUUAACAUUCAAAAGGAAAUCGACAGACUCAACGAGGUGGCCAAAAACCUGAACGAAAGCUUGA
UAGAUCUCCAGGAGUUGGGCAAGUAUGAACAGUACAUUAAAUGGCCAUGGUACAUAUGGCUUGGC
UUUAUCGCUGGCCUUAUCGCCAUCGUAAUGGUUACAAUCAUGCUGUGCUGCAUGACCUCCUGCUG
UUCUUGUUUGAAAGGGUGUUGUUCUUGUGGUAGUUGUUGCAAGUUUGACGAAGAUGAUUCCGAAC
CUGUUCUUAAAGGGGUAAAGCUUCACUAUACAUGAUAACCGCGGUGUCAAAAACCGCGUGGACGU
GGUUAACAUCCCUGCUGGGAGGAUCAGCCGUAAUUAUUAUAAUUGGCUUGGUGCUGGCUACUAUU
GUGGCCAUGUACGUGCUGACCAACCAGAAACAUAAUUGAAUACAGCAGCAAUUGGCAAGCUGCUU
ACAUAGAACUCGCGGCGAUUGGCAUGCCGCCUUAAAAUUUUUAUUUUAUUUUUUCUUUUCUUUUC
CGAAUCGGAUUUUGUUUUUAAUAUUUCAAAAAAAAAAAAAAAAAAAAAAAAAAAAAAAAAAAAAA
AAAAAAA
SEQ ID NO: 2- SARS CoV-2 A.1-preF antigen
AUAGGCGGCGCAUGAGAGAAGCCCAGACCAAUUACCUACCCAAAAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCUAACCUGAAUGGACUAC
GACAUAGUCUAGUCCGCCAAGAUGUUUCUGCUCACAACCAAACGCACUAUGUUUGUUUUCCUCGU
GCUGCUCCCUUUGGUAAGUUCUCAGUGUGUAAACCUGACAACACGAACCCAGUUGCCUCCAGCUU
AUACCAACUCAUUUACUCGCGGAGUAUAUUAUCCCGAUAAGGUCUUUAGAAGUAGCGUGUUGCAC
UCUACACAGGAUCUGUUCUUGCCCUUCUUUAGUAACGUUACCUGGUUUCAUGCAAUACAUGUGAG
CGGAACAAAUGGAACAAAAAGAUUUGACAAUCCAGUGCUUCCAUUUAAUGAUGGGGUUUACUUUG
CCAGUACCGAAAAGUCAAACAUAAUCCGGGGGUGGAUCUUUGGAACCACUUUGGACUCUAAGACA
CAGUCUCUCCUCAUAGUAAACAACGCCACCAAUGUUGUCAUAAAAGUAUGCGAAUUUCAGUUUUG
CAACGAUCCCUUUCUCGGGGUGUAUUACCAUAAGAAUAAUAAAUCCUGGAUGGAGUCUGAGUUCC
GGGUUUAUAGUAGUGCUAAUAAUUGCACUUUCGAAUACGUGUCCCAACCAUUCCUCAUGGACCUU
GAGGGCAAACAGGGGAAUUUUAAAAACUUGCGCGAAUUUGUCUUUAAGAAUAUCGACGGAUACUU
UAAGAUCUAUAGUAAACACACUCCUAUCAACCUCGUUCGGGAUCUUCCCCAAGGCUUUUCUGCUC
UCGAACCCCUCGUAGACUUGCCAAUUGGGAUAAAUAUCACUCGCUUUCAAACUUUGCUUGCCCUC
CACAGGAGCUACCUGACACCCGGCGACUCUUCUUCUGGUUGGACCGCCGGCGCCGCUGCCUAUUA
UGUUGGUUACCUUCAGCCACGAACAUUCUUGCUCAAGUAUAACGAGAAUGGCACCAUUACCGACG
CCGUCGAUUGUGCAUUGGAUCCCUUGUCUGAAACAAAAUGUACCUUGAAGUCCUUUACCGUAGAG
AAAGGCAUAUACCAGACUUCCAACUUCCGAGUUCAGCCUACAGAAUCCAUUGUGAGAUUUCCCAA
CAUCACAAACCUCUGCCCUUUCGGUGAAGUAUUUAAUGCUACACGCUUCGCUUCAGUCUAUGCCU
GGAAUAGGAAGCGCAUAUCAAAUUGCGUGGCCGAUUAUUCAGUCCUCUAUAAUAGCGCAUCCUUC
AGUACUUUCAAGUGCUACGGCGUUUCCCCCACCAAACUCAAUGAUCUUUGCUUCACCAACGUCUA
UGCUGACAGUUUUGUCAUACGAGGCGACGAAGUACGCCAGAUUGCCCCCGGGCAGACAGGUAAAA
UUGCUGAUUAUAAUUAUAAACUCCCAGAUGACUUUACUGGAUGCGUCAUAGCCUGGAAUUCCAAC
AAUCUUGAUUCCAAGGUUGGUGGGAAUUAUAAUUACCUUUAUCGACUGUUCAGAAAGAGUAACUU
GAAACCAUUUGAGAGAGACAUAUCCACCGAGAUUUACCAGGCAGGCAGUACUCCUUGUAACGGCG
UUGAGGGAUUUAACUGCUAUUUUCCUUUGCAAUCCUAUGGCUUUCAACCAACAAACGGGGUUGGC
UAUCAACCCUAUCGAGUGGUUGUCCUGAGCUUUGAACUUUUGCACGCUCCCGCCACAGUCUGCGG
ACCAAAAAAGAGUACAAAUCUUGUCAAGAAUAAGUGCGUAAAUUUCAAUUUCAAUGGCCUUACAG
GAACAGGCGUGCUGACUGAGUCAAACAAGAAGUUCCUGCCAUUUCAGCAGUUUGGGCGGGAUAUA
GCAGACACAACUGACGCUGUACGCGAUCCUCAGACUUUGGAGAUCUUGGACAUCACUCCCUGUUC
UUUCGGAGGGGUAUCUGUCAUCACCCCCGGAACUAAUACAUCAAAUCAGGUCGCUGUGUUGUACC
AAGAUGUCAACUGCACAGAAGUCCCCGUUGCUAUACACGCAGACCAGCUCACCCCCACAUGGCGG
GUGUACUCAACUGGCUCAAACGUAUUCCAGACCAGAGCUGGGUGCUUGAUCGGUGCUGAACACGU
AAACAAUAGCUAUGAAUGCGAUAUUCCCAUCGGUGCCGGGAUCUGCGCUAGCUAUCAGACACAGA
CCAAUUCCCCCCGGCGAGCACGAUCUGUAGCAUCCCAGUCUAUUAUUGCCUACACUAUGUCAUUG
GGCGCCGAGAAUAGCGUCGCAUAUUCAAAUAAUUCUAUUGCAAUACCCACCAACUUCACAAUCUC
CGUAACUACAGAAAUACUUCCAGUUUCCAUGACAAAGACAUCAGUGGAUUGUACAAUGUAUAUAU
GCGGAGAUUCCACAGAAUGUUCAAAUUUGCUCUUGCAGUACGGCUCCUUCUGCACCCAGCUCAAC
AGGGCCCUUACAGGUAUUGCUGUCGAACAGGACAAGAACACACAAGAAGUCUUCGCCCAAGUCAA
ACAGAUAUACAAAACUCCUCCCAUAAAGGAUUUUGGCGGCUUCAACUUUAGUCAGAUCCUCCCAG
ACCCUUCAAAACCAUCUAAACGAUCAUUUAUUGAAGAUCUGCUGUUCAACAAGGUCACUCUUGCC
GAUGCUGGAUUCAUUAAGCAAUACGGUGACUGCCUUGGUGAUAUUGCUGCCCGAGAUCUGAUCUG
UGCCCAGAAAUUCAACGGGCUCACUGUACUCCCUCCACUGCUCACAGACGAAAUGAUUGCACAGU
ACACAAGUGCCCUGUUGGCAGGCACAAUCACUAGCGGCUGGACCUUUGGCGCAGGUGCAGCACUC
CAAAUACCUUUUGCCAUGCAGAUGGCCUAUCGGUUUAAUGGGAUAGGCGUGACUCAAAAUGUCCU
CUACGAAAACCAAAAGUUGAUAGCUAACCAAUUCAAUUCAGCAAUCGGGAAGAUACAGGAUUCAC
UGUCUAGUACUGCUAGUGCCCUUGGUAAGCUGCAGGACGUUGUCAACCAGAAUGCUCAAGCUCUG
AAUACAUUGGUUAAGCAGCUCUCUAGUAAUUUUGGGGCCAUCUCUUCAGUACUUAAUGAUAUUUU
GAGCCGAUUGGACCCACCCGAAGCUGAAGUACAGAUCGACAGGCUGAUAACAGGCCGGCUCCAAU
CCCUCCAAACAUACGUGACACAACAACUCAUACGCGCAGCCGAAAUCCGAGCCAGCGCUAACCUG
GCAGCUACCAAGAUGUCAGAAUGCGUUCUGGGCCAGAGUAAACGCGUAGAUUUCUGCGGGAAAGG
GUACCACCUGAUGUCCUUUCCACAAUCUGCACCUCACGGGGUCGUCUUUUUGCAUGUAACAUAUG
UACCCGCACAAGAGAAGAAUUUUACUACCGCUCCUGCCAUCUGUCAUGACGGGAAAGCUCAUUUU
CCUCGCGAAGGUGUGUUUGUAUCUAAUGGUACACAUUGGUUUGUCACACAGCGGAAUUUCUAUGA
ACCCCAGAUCAUUACAACUGACAACACUUUUGUUUCCGGGAAUUGUGACGUGGUCAUAGGAAUCG
UAAAUAACACUGUAUAUGAUCCCCUCCAACCAGAGCUGGACUCUUUUAAAGAAGAACUGGAUAAA
UAUUUCAAGAACCACACAAGUCCCGACGUGGACCUUGGGGACAUAAGUGGUAUUAACGCAUCUGU
GGUUAACAUUCAAAAGGAAAUCGACAGACUCAACGAGGUGGCCAAAAACCUGAACGAAAGCUUGA
UAGAUCUCCAGGAGUUGGGCAAGUAUGAACAGUACAUUAAAUGGCCAUGGUACAUAUGGCUUGGC
UUUAUCGCUGGCCUUAUCGCCAUCGUAAUGGUUACAAUCAUGCUGUGCUGCAUGACCUCCUGCUG
UUCUUGUUUGAAAGGGUGUUGUUCUUGUGGUAGUUGUUGCAAGUUUGACGAAGAUGAUUCCGAAC
CUGUUCUUAAAGGGGUAAAGCUUCACUAUACAUGAUAACCGCGGUGUCAAAAACCGCGUGGACGU
GGUUAACAUCCCUGCUGGGAGGAUCAGCCGUAAUUAUUAUAAUUGGCUUGGUGCUGGCUACUAUU
GUGGCCAUGUACGUGCUGACCAACCAGAAACAUAAUUGAAUACAGCAGCAAUUGGCAAGCUGCUU
ACAUAGAACUCGCGGCGAUUGGCAUGCCGCCUUAAAAUUUUUAUUUUAUUUUUUCUUUUCUUUUC
CGAAUCGGAUUUUGUUUUUAAUAUUUCAAAAAAAAAAAAAAAAAAAAAAAAAAAAAAAAAAAAAA
AAAAAAA
SEQ ID NO: 3- SARS CoV-2 B.1 antigen
AUAGGCGGCGCAUGAGAGAAGCCCAGACCAAUUACCUACCCAAAAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCUAACCUGAAUGGACUAC
GACAUAGUCUAGUCCGCCAAGAUGUUUCUGCUCACAACCAAACGCACUAUGUUUGUUUUCCUCGU
GCUGCUCCCUUUGGUAAGUUCUCAGUGUGUAAACCUGACAACACGAACCCAGUUGCCUCCAGCUU
AUACCAACUCAUUUACUCGCGGAGUAUAUUAUCCCGAUAAGGUCUUUAGAAGUAGCGUGUUGCAC
UCUACACAGGAUCUGUUCUUGCCCUUCUUUAGUAACGUUACCUGGUUUCAUGCAAUACAUGUGAG
CGGAACAAAUGGAACAAAAAGAUUUGACAAUCCAGUGCUUCCAUUUAAUGAUGGGGUUUACUUUG
CCAGUACCGAAAAGUCAAACAUAAUCCGGGGGUGGAUCUUUGGAACCACUUUGGACUCUAAGACA
CAGUCUCUCCUCAUAGUAAACAACGCCACCAAUGUUGUCAUAAAAGUAUGCGAAUUUCAGUUUUG
CAACGAUCCCUUUCUCGGGGUGUAUUACCAUAAGAAUAAUAAAUCCUGGAUGGAGUCUGAGUUCC
GGGUUUAUAGUAGUGCUAAUAAUUGCACUUUCGAAUACGUGUCCCAACCAUUCCUCAUGGACCUU
GAGGGCAAACAGGGGAAUUUUAAAAACUUGCGCGAAUUUGUCUUUAAGAAUAUCGACGGAUACUU
UAAGAUCUAUAGUAAACACACUCCUAUCAACCUCGUUCGGGAUCUUCCCCAAGGCUUUUCUGCUC
UCGAACCCCUCGUAGACUUGCCAAUUGGGAUAAAUAUCACUCGCUUUCAAACUUUGCUUGCCCUC
CACAGGAGCUACCUGACACCCGGCGACUCUUCUUCUGGUUGGACCGCCGGCGCCGCUGCCUAUUA
UGUUGGUUACCUUCAGCCACGAACAUUCUUGCUCAAGUAUAACGAGAAUGGCACCAUUACCGACG
CCGUCGAUUGUGCAUUGGAUCCCUUGUCUGAAACAAAAUGUACCUUGAAGUCCUUUACCGUAGAG
AAAGGCAUAUACCAGACUUCCAACUUCCGAGUUCAGCCUACAGAAUCCAUUGUGAGAUUUCCCAA
CAUCACAAACCUCUGCCCUUUCGGUGAAGUAUUUAAUGCUACACGCUUCGCUUCAGUCUAUGCCU
GGAAUAGGAAGCGCAUAUCAAAUUGCGUGGCCGAUUAUUCAGUCCUCUAUAAUAGCGCAUCCUUC
AGUACUUUCAAGUGCUACGGCGUUUCCCCCACCAAACUCAAUGAUCUUUGCUUCACCAACGUCUA
UGCUGACAGUUUUGUCAUACGAGGCGACGAAGUACGCCAGAUUGCCCCCGGGCAGACAGGUAAAA
UUGCUGAUUAUAAUUAUAAACUCCCAGAUGACUUUACUGGAUGCGUCAUAGCCUGGAAUUCCAAC
AAUCUUGAUUCCAAGGUUGGUGGGAAUUAUAAUUACCUUUAUCGACUGUUCAGAAAGAGUAACUU
GAAACCAUUUGAGAGAGACAUAUCCACCGAGAUUUACCAGGCAGGCAGUACUCCUUGUAACGGCG
UUGAGGGAUUUAACUGCUAUUUUCCUUUGCAAUCCUAUGGCUUUCAACCAACAAACGGGGUUGGC
UAUCAACCCUAUCGAGUGGUUGUCCUGAGCUUUGAACUUUUGCACGCUCCCGCCACAGUCUGCGG
ACCAAAAAAGAGUACAAAUCUUGUCAAGAAUAAGUGCGUAAAUUUCAAUUUCAAUGGCCUUACAG
GAACAGGCGUGCUGACUGAGUCAAACAAGAAGUUCCUGCCAUUUCAGCAGUUUGGGCGGGAUAUA
GCAGACACAACUGACGCUGUACGCGAUCCUCAGACUUUGGAGAUCUUGGACAUCACUCCCUGUUC
UUUCGGAGGGGUAUCUGUCAUCACCCCCGGAACUAAUACAUCAAAUCAGGUCGCUGUGUUGUACC
AAGAUGUCAACUGCACAGAAGUCCCCGUUGCUAUACACGCAGGCCAGCUCACCCCCACAUGGCGG
GUGUACUCAACUGGCUCAAACGUAUUCCAGACCAGAGCUGGGUGCUUGAUCGGUGCUGAACACGU
AAACAAUAGCUAUGAAUGCGAUAUUCCCAUCGGUGCCGGGAUCUGCGCUAGCUAUCAGACACAGA
CCAAUUCCCCCCGGCGAGCACGAUCUGUAGCAUCCCAGUCUAUUAUUGCCUACACUAUGUCAUUG
GGCGCCGAGAAUAGCGUCGCAUAUUCAAAUAAUUCUAUUGCAAUACCCACCAACUUCACAAUCUC
CGUAACUACAGAAAUACUUCCAGUUUCCAUGACAAAGACAUCAGUGGAUUGUACAAUGUAUAUAU
GCGGAGAUUCCACAGAAUGUUCAAAUUUGCUCUUGCAGUACGGCUCCUUCUGCACCCAGCUCAAC
AGGGCCCUUACAGGUAUUGCUGUCGAACAGGACAAGAACACACAAGAAGUCUUCGCCCAAGUCAA
ACAGAUAUACAAAACUCCUCCCAUAAAGGAUUUUGGCGGCUUCAACUUUAGUCAGAUCCUCCCAG
ACCCUUCAAAACCAUCUAAACGAUCAUUUAUUGAAGAUCUGCUGUUCAACAAGGUCACUCUUGCC
GAUGCUGGAUUCAUUAAGCAAUACGGUGACUGCCUUGGUGAUAUUGCUGCCCGAGAUCUGAUCUG
UGCCCAGAAAUUCAACGGGCUCACUGUACUCCCUCCACUGCUCACAGACGAAAUGAUUGCACAGU
ACACAAGUGCCCUGUUGGCAGGCACAAUCACUAGCGGCUGGACCUUUGGCGCAGGUGCAGCACUC
CAAAUACCUUUUGCCAUGCAGAUGGCCUAUCGGUUUAAUGGGAUAGGCGUGACUCAAAAUGUCCU
CUACGAAAACCAAAAGUUGAUAGCUAACCAAUUCAAUUCAGCAAUCGGGAAGAUACAGGAUUCAC
UGUCUAGUACUGCUAGUGCCCUUGGUAAGCUGCAGGACGUUGUCAACCAGAAUGCUCAAGCUCUG
AAUACAUUGGUUAAGCAGCUCUCUAGUAAUUUUGGGGCCAUCUCUUCAGUACUUAAUGAUAUUUU
GAGCCGAUUGGACAAAGUGGAAGCUGAAGUACAGAUCGACAGGCUGAUAACAGGCCGGCUCCAAU
CCCUCCAAACAUACGUGACACAACAACUCAUACGCGCAGCCGAAAUCCGAGCCAGCGCUAACCUG
GCAGCUACCAAGAUGUCAGAAUGCGUUCUGGGCCAGAGUAAACGCGUAGAUUUCUGCGGGAAAGG
GUACCACCUGAUGUCCUUUCCACAAUCUGCACCUCACGGGGUCGUCUUUUUGCAUGUAACAUAUG
UACCCGCACAAGAGAAGAAUUUUACUACCGCUCCUGCCAUCUGUCAUGACGGGAAAGCUCAUUUU
CCUCGCGAAGGUGUGUUUGUAUCUAAUGGUACACAUUGGUUUGUCACACAGCGGAAUUUCUAUGA
ACCCCAGAUCAUUACAACUGACAACACUUUUGUUUCCGGGAAUUGUGACGUGGUCAUAGGAAUCG
UAAAUAACACUGUAUAUGAUCCCCUCCAACCAGAGCUGGACUCUUUUAAAGAAGAACUGGAUAAA
UAUUUCAAGAACCACACAAGUCCCGACGUGGACCUUGGGGACAUAAGUGGUAUUAACGCAUCUGU
GGUUAACAUUCAAAAGGAAAUCGACAGACUCAACGAGGUGGCCAAAAACCUGAACGAAAGCUUGA
UAGAUCUCCAGGAGUUGGGCAAGUAUGAACAGUACAUUAAAUGGCCAUGGUACAUAUGGCUUGGC
UUUAUCGCUGGCCUUAUCGCCAUCGUAAUGGUUACAAUCAUGCUGUGCUGCAUGACCUCCUGCUG
UUCUUGUUUGAAAGGGUGUUGUUCUUGUGGUAGUUGUUGCAAGUUUGACGAAGAUGAUUCCGAAC
CUGUUCUUAAAGGGGUAAAGCUUCACUAUACAUAGUAACCGCGGUGUCAAAAACCGCGUGGACGU
GGUUAACAUCCCUGCUGGGAGGAUCAGCCGUAAUUAUUAUAAUUGGCUUGGUGCUGGCUACUAUU
GUGGCCAUGUACGUGCUGACCAACCAGAAACAUAAUUGAAUACAGCAGCAAUUGGCAAGCUGCUU
ACAUAGAACUCGCGGCGAUUGGCAUGCCGCCUUAAAAUUUUUAUUUUAUUUUUUCUUUUCUUUUC
CGAAUCGGAUUUUGUUUUUAAUAUUUCAAAAAAAAAAAAAAAAAAAAAAAAAAAAAAAAAAAAAA
AAAAAAA
SEQ ID NO: 4- SARS CoV-2 Beta-preF antigen
AUAGGCGGCGCAUGAGAGAAGCCCAGACCAAUUACCUACCCAAAAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCUAACCUGAAUGGACUAC
GACAUAGUCUAGUCCGCCAAGAUGUUUCUGCUCACAACCAAACGCACUAUGUUUGUUUUCCUCGU
GCUGCUCCCUUUGGUAAGUUCUCAGUGUGUAAACUUCACAACACGAACCCAGUUGCCUCCAGCUU
AUACCAACUCAUUUACUCGCGGAGUAUAUUAUCCCGAUAAGGUCUUUAGAAGUAGCGUGUUGCAC
UCUACACAGGAUCUGUUCUUGCCCUUCUUUAGUAACGUUACCUGGUUUCAUGCAAUACAUGUGAG
CGGAACAAAUGGAACAAAAAGAUUUGCCAAUCCAGUGCUUCCAUUUAAUGAUGGGGUUUACUUUG
CCAGUACCGAAAAGUCAAACAUAAUCCGGGGGUGGAUCUUUGGAACCACUUUGGACUCUAAGACA
CAGUCUCUCCUCAUAGUAAACAACGCCACCAAUGUUGUCAUAAAAGUAUGCGAAUUUCAGUUUUG
CAACGAUCCCUUUCUCGGGGUGUAUUACCAUAAGAAUAAUAAAUCCUGGAUGGAGUCUGAGUUCC
GGGUUUAUAGUAGUGCUAAUAAUUGCACUUUCGAAUACGUGUCCCAACCAUUCCUCAUGGACCUU
GAGGGCAAACAGGGGAAUUUUAAAAACUUGCGCGAAUUUGUCUUUAAGAAUAUCGACGGAUACUU
UAAGAUCUAUAGUAAACACACUCCUAUCAACCUCGUUCGGGGCCUUCCCCAAGGCUUUUCUGCUC
UCGAACCCCUCGUAGACUUGCCAAUUGGGAUAAAUAUCACUCGCUUUCAAACUUUGCACAUCAGC
UACCUGACACCCGGCGACUCUUCUUCUGGUUGGACCGCCGGCGCCGCUGCCUAUUAUGUUGGUUA
CCUUCAGCCACGAACAUUCUUGCUCAAGUAUAACGAGAAUGGCACCAUUACCGACGCCGUCGAUU
GUGCAUUGGAUCCCUUGUCUGAAACAAAAUGUACCUUGAAGUCCUUUACCGUAGAGAAAGGCAUA
UACCAGACUUCCAACUUCCGAGUUCAGCCUACAGAAUCCAUUGUGAGAUUUCCCAACAUCACAAA
CCUCUGCCCUUUCGGUGAAGUAUUUAAUGCUACACGCUUCGCUUCAGUCUAUGCCUGGAAUAGGA
AGCGCAUAUCAAAUUGCGUGGCCGAUUAUUCAGUCCUCUAUAAUAGCGCAUCCUUCAGUACUUUC
AAGUGCUACGGCGUUUCCCCCACCAAACUCAAUGAUCUUUGCUUCACCAACGUCUAUGCUGACAG
UUUUGUCAUACGAGGCGACGAAGUACGCCAGAUUGCCCCCGGGCAGACAGGUAACAUUGCUGAUU
AUAAUUAUAAACUCCCAGAUGACUUUACUGGAUGCGUCAUAGCCUGGAAUUCCAACAAUCUUGAU
UCCAAGGUUGGUGGGAAUUAUAAUUACCUUUAUCGACUGUUCAGAAAGAGUAACUUGAAACCAUU
UGAGAGAGACAUAUCCACCGAGAUUUACCAGGCAGGCAGUACUCCUUGUAACGGCGUUAAGGGAU
UUAACUGCUAUUUUCCUUUGCAAUCCUAUGGCUUUCAACCAACAUACGGGGUUGGCUAUCAACCC
UAUCGAGUGGUUGUCCUGAGCUUUGAACUUUUGCACGCUCCCGCCACAGUCUGCGGACCAAAAAA
GAGUACAAAUCUUGUCAAGAAUAAGUGCGUAAAUUUCAAUUUCAAUGGCCUUACAGGAACAGGCG
UGCUGACUGAGUCAAACAAGAAGUUCCUGCCAUUUCAGCAGUUUGGGCGGGAUAUAGCAGACACA
ACUGACGCUGUACGCGAUCCUCAGACUUUGGAGAUCUUGGACAUCACUCCCUGUUCUUUCGGAGG
GGUAUCUGUCAUCACCCCCGGAACUAAUACAUCAAAUCAGGUCGCUGUGUUGUACCAAGGCGUCA
ACUGCACAGAAGUCCCCGUUGCUAUACACGCAGACCAGCUCACCCCCACAUGGCGGGUGUACUCA
ACUGGCUCAAACGUAUUCCAGACCAGAGCUGGGUGCUUGAUCGGUGCUGAACACGUAAACAAUAG
CUAUGAAUGCGAUAUUCCCAUCGGUGCCGGGAUCUGCGCUAGCUAUCAGACACAGACCAAUUCCC
CCCGGCGAGCACGAUCUGUAGCAUCCCAGUCUAUUAUUGCCUACACUAUGUCAUUGGGCGUGGAG
AAUAGCGUCGCAUAUUCAAAUAAUUCUAUUGCAAUACCCACCAACUUCACAAUCUCCGUAACUAC
AGAAAUACUUCCAGUUUCCAUGACAAAGACAUCAGUGGAUUGUACAAUGUAUAUAUGCGGAGAUU
CCACAGAAUGUUCAAAUUUGCUCUUGCAGUACGGCUCCUUCUGCACCCAGCUCAACAGGGCCCUU
ACAGGUAUUGCUGUCGAACAGGACAAGAACACACAAGAAGUCUUCGCCCAAGUCAAACAGAUAUA
CAAAACUCCUCCCAUAAAGGAUUUUGGCGGCUUCAACUUUAGUCAGAUCCUCCCAGACCCUUCAA
AACCAUCUAAACGAUCAUUUAUUGAAGAUCUGCUGUUCAACAAGGUCACUCUUGCCGAUGCUGGA
UUCAUUAAGCAAUACGGUGACUGCCUUGGUGAUAUUGCUGCCCGAGAUCUGAUCUGUGCCCAGAA
AUUCAACGGGCUCACUGUACUCCCUCCACUGCUCACAGACGAAAUGAUUGCACAGUACACAAGUG
CCCUGUUGGCAGGCACAAUCACUAGCGGCUGGACCUUUGGCGCAGGUGCAGCACUCCAAAUACCU
UUUGCCAUGCAGAUGGCCUAUCGGUUUAAUGGGAUAGGCGUGACUCAAAAUGUCCUCUACGAAAA
CCAAAAGUUGAUAGCUAACCAAUUCAAUUCAGCAAUCGGGAAGAUACAGGAUUCACUGUCUAGUA
CUGCUAGUGCCCUUGGUAAGCUGCAGGACGUUGUCAACCAGAAUGCUCAAGCUCUGAAUACAUUG
GUUAAGCAGCUCUCUAGUAAUUUUGGGGCCAUCUCUUCAGUACUUAAUGAUAUUUUGAGCCGAUU
GGACCCACCCGAAGCUGAAGUACAGAUCGACAGGCUGAUAACAGGCCGGCUCCAAUCCCUCCAAA
CAUACGUGACACAACAACUCAUACGCGCAGCCGAAAUCCGAGCCAGCGCUAACCUGGCAGCUACC
AAGAUGUCAGAAUGCGUUCUGGGCCAGAGUAAACGCGUAGAUUUCUGCGGGAAAGGGUACCACCU
GAUGUCCUUUCCACAAUCUGCACCUCACGGGGUCGUCUUUUUGCAUGUAACAUAUGUACCCGCAC
AAGAGAAGAAUUUUACUACCGCUCCUGCCAUCUGUCAUGACGGGAAAGCUCAUUUUCCUCGCGAA
GGUGUGUUUGUAUCUAAUGGUACACAUUGGUUUGUCACACAGCGGAAUUUCUAUGAACCCCAGAU
CAUUACAACUGACAACACUUUUGUUUCCGGGAAUUGUGACGUGGUCAUAGGAAUCGUAAAUAACA
CUGUAUAUGAUCCCCUCCAACCAGAGCUGGACUCUUUUAAAGAAGAACUGGAUAAAUAUUUCAAG
AACCACACAAGUCCCGACGUGGACCUUGGGGACAUAAGUGGUAUUAACGCAUCUGUGGUUAACAU
UCAAAAGGAAAUCGACAGACUCAACGAGGUGGCCAAAAACCUGAACGAAAGCUUGAUAGAUCUCC
AGGAGUUGGGCAAGUAUGAACAGUACAUUAAAUGGCCAUGGUACAUAUGGCUUGGCUUUAUCGCU
GGCCUUAUCGCCAUCGUAAUGGUUACAAUCAUGCUGUGCUGCAUGACCUCCUGCUGUUCUUGUUU
GAAAGGGUGUUGUUCUUGUGGUAGUUGUUGCAAGUUUGACGAAGAUGAUUCCGAACCUGUUCUUA
AAGGGGUAAAGCUUCACUAUACAUGAUAACCGCGGUGUCAAAAACCGCGUGGACGUGGUUAACAU
CCCUGCUGGGAGGAUCAGCCGUAAUUAUUAUAAUUGGCUUGGUGCUGGCUACUAUUGUGGCCAUG
UACGUGCUGACCAACCAGAAACAUAAUUGAAUACAGCAGCAAUUGGCAAGCUGCUUACAUAGAAC
UCGCGGCGAUUGGCAUGCCGCCUUAAAAUUUUUAUUUUAUUUUUUCUUUUCUUUUCCGAAUCGGA
UUUUGUUUUUAAUAUUUCAAAAAAAAAAAAAAAAAAAAAAAAAAAAAAAAAAAAAAAAAAAAA
SEQ ID NO: 5- SARS CoV-2 Alpha-preF antigen
AUAGGCGGCGCAUGAGAGAAGCCCAGACCAAUUACCUACCCAAAAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCUAACCUGAAUGGACUAC
GACAUAGUCUAGUCCGCCAAGAUGUUUCUGCUCACAACCAAACGCACUAUGUUUGUUUUCCUCGU
GCUGCUCCCUUUGGUAAGUUCUCAGUGUGUAAACCUGACAACACGAACCCAGUUGCCUCCAGCUU
AUACCAACUCAUUUACUCGCGGAGUAUAUUAUCCCGAUAAGGUCUUUAGAAGUAGCGUGUUGCAC
UCUACACAGGAUCUGUUCUUGCCCUUCUUUAGUAACGUUACCUGGUUUCAUGCAAUAAGCGGAAC
AAAUGGAACAAAAAGAUUUGACAAUCCAGUGCUUCCAUUUAAUGAUGGGGUUUACUUUGCCAGUA
CCGAAAAGUCAAACAUAAUCCGGGGGUGGAUCUUUGGAACCACUUUGGACUCUAAGACACAGUCU
CUCCUCAUAGUAAACAACGCCACCAAUGUUGUCAUAAAAGUAUGCGAAUUUCAGUUUUGCAACGA
UCCCUUUCUCGGGGUGUACCAUAAGAAUAAUAAAUCCUGGAUGGAGUCUGAGUUCCGGGUUUAUA
GUAGUGCUAAUAAUUGCACUUUCGAAUACGUGUCCCAACCAUUCCUCAUGGACCUUGAGGGCAAA
CAGGGGAAUUUUAAAAACUUGCGCGAAUUUGUCUUUAAGAAUAUCGACGGAUACUUUAAGAUCUA
UAGUAAACACACUCCUAUCAACCUCGUUCGGGAUCUUCCCCAAGGCUUUUCUGCUCUCGAACCCC
UCGUAGACUUGCCAAUUGGGAUAAAUAUCACUCGCUUUCAAACUUUGCUUGCCCUCCACAGGAGC
UACCUGACACCCGGCGACUCUUCUUCUGGUUGGACCGCCGGCGCCGCUGCCUAUUAUGUUGGUUA
CCUUCAGCCACGAACAUUCUUGCUCAAGUAUAACGAGAAUGGCACCAUUACCGACGCCGUCGAUU
GUGCAUUGGAUCCCUUGUCUGAAACAAAAUGUACCUUGAAGUCCUUUACCGUAGAGAAAGGCAUA
UACCAGACUUCCAACUUCCGAGUUCAGCCUACAGAAUCCAUUGUGAGAUUUCCCAACAUCACAAA
CCUCUGCCCUUUCGGUGAAGUAUUUAAUGCUACACGCUUCGCUUCAGUCUAUGCCUGGAAUAGGA
AGCGCAUAUCAAAUUGCGUGGCCGAUUAUUCAGUCCUCUAUAAUAGCGCAUCCUUCAGUACUUUC
AAGUGCUACGGCGUUUCCCCCACCAAACUCAAUGAUCUUUGCUUCACCAACGUCUAUGCUGACAG
UUUUGUCAUACGAGGCGACGAAGUACGCCAGAUUGCCCCCGGGCAGACAGGUAAAAUUGCUGAUU
AUAAUUAUAAACUCCCAGAUGACUUUACUGGAUGCGUCAUAGCCUGGAAUUCCAACAAUCUUGAU
UCCAAGGUUGGUGGGAAUUAUAAUUACCUUUAUCGACUGUUCAGAAAGAGUAACUUGAAACCAUU
UGAGAGAGACAUAUCCACCGAGAUUUACCAGGCAGGCAGUACUCCUUGUAACGGCGUUGAGGGAU
UUAACUGCUAUUUUCCUUUGCAAUCCUAUGGCUUUCAACCAACAUACGGGGUUGGCUAUCAACCC
UAUCGAGUGGUUGUCCUGAGCUUUGAACUUUUGCACGCUCCCGCCACAGUCUGCGGACCAAAAAA
GAGUACAAAUCUUGUCAAGAAUAAGUGCGUAAAUUUCAAUUUCAAUGGCCUUACAGGAACAGGCG
UGCUGACUGAGUCAAACAAGAAGUUCCUGCCAUUUCAGCAGUUUGGGCGGGAUAUAGACGACACA
ACUGACGCUGUACGCGAUCCUCAGACUUUGGAGAUCUUGGACAUCACUCCCUGUUCUUUCGGAGG
GGUAUCUGUCAUCACCCCCGGAACUAAUACAUCAAAUCAGGUCGCUGUGUUGUACCAAGGCGUCA
ACUGCACAGAAGUCCCCGUUGCUAUACACGCAGAUCAGCUCACCCCCACAUGGCGGGUGUACUCA
ACUGGCUCAAACGUAUUCCAGACCAGAGCUGGGUGCUUGAUCGGUGCUGAACACGUAAACAAUAG
CUAUGAAUGCGAUAUUCCCAUCGGUGCCGGGAUCUGCGCUAGCUAUCAGACACAGACCAAUUCCC
AUCGGCGAGCACGAUCUGUAGCAUCCCAGUCUAUUAUUGCCUACACUAUGUCAUUGGGCGCCGAG
AAUAGCGUCGCAUAUUCAAAUAAUUCUAUUGCAAUACCCAUCAACUUCACAAUCUCCGUAACUAC
AGAAAUACUUCCAGUUUCCAUGACAAAGACAUCAGUGGAUUGUACAAUGUAUAUAUGCGGAGAUU
CCACAGAAUGUUCAAAUUUGCUCUUGCAGUACGGCUCCUUCUGCACCCAGCUCAACAGGGCCCUU
ACAGGUAUUGCUGUCGAACAGGACAAGAACACACAAGAAGUCUUCGCCCAAGUCAAACAGAUAUA
CAAAACUCCUCCCAUAAAGGAUUUUGGCGGCUUCAACUUUAGUCAGAUCCUCCCAGACCCUUCAA
AACCAUCUAAACGAUCAUUUAUUGAAGAUCUGCUGUUCAACAAGGUCACUCUUGCCGAUGCUGGA
UUCAUUAAGCAAUACGGUGACUGCCUUGGUGAUAUUGCUGCCCGAGAUCUGAUCUGUGCCCAGAA
AUUCAACGGGCUCACUGUACUCCCUCCACUGCUCACAGACGAAAUGAUUGCACAGUACACAAGUG
CCCUGUUGGCAGGCACAAUCACUAGCGGCUGGACCUUUGGCGCAGGUGCAGCACUCCAAAUACCU
UUUGCCAUGCAGAUGGCCUAUCGGUUUAAUGGGAUAGGCGUGACUCAAAAUGUCCUCUACGAAAA
CCAAAAGUUGAUAGCUAACCAAUUCAAUUCAGCAAUCGGGAAGAUACAGGAUUCACUGUCUAGUA
CUGCUAGUGCCCUUGGUAAGCUGCAGGACGUUGUCAACCAGAAUGCUCAAGCUCUGAAUACAUUG
GUUAAGCAGCUCUCUAGUAAUUUUGGGGCCAUCUCUUCAGUACUUAAUGAUAUUUUGGCCCGAUU
GGACCCACCCGAAGCUGAAGUACAGAUCGACAGGCUGAUAACAGGCCGGCUCCAAUCCCUCCAAA
CAUACGUGACACAACAACUCAUACGCGCAGCCGAAAUCCGAGCCAGCGCUAACCUGGCAGCUACC
AAGAUGUCAGAAUGCGUUCUGGGCCAGAGUAAACGCGUAGAUUUCUGCGGGAAAGGGUACCACCU
GAUGUCCUUUCCACAAUCUGCACCUCACGGGGUCGUCUUUUUGCAUGUAACAUAUGUACCCGCAC
AAGAGAAGAAUUUUACUACCGCUCCUGCCAUCUGUCAUGACGGGAAAGCUCAUUUUCCUCGCGAA
GGUGUGUUUGUAUCUAAUGGUACACAUUGGUUUGUCACACAGCGGAAUUUCUAUGAACCCCAGAU
CAUUACAACUCACAACACUUUUGUUUCCGGGAAUUGUGACGUGGUCAUAGGAAUCGUAAAUAACA
CUGUAUAUGAUCCCCUCCAACCAGAGCUGGACUCUUUUAAAGAAGAACUGGAUAAAUAUUUCAAG
AACCACACAAGUCCCGACGUGGACCUUGGGGACAUAAGUGGUAUUAACGCAUCUGUGGUUAACAU
UCAAAAGGAAAUCGACAGACUCAACGAGGUGGCCAAAAACCUGAACGAAAGCUUGAUAGAUCUCC
AGGAGUUGGGCAAGUAUGAACAGUACAUUAAAUGGCCAUGGUACAUAUGGCUUGGCUUUAUCGCU
GGCCUUAUCGCCAUCGUAAUGGUUACAAUCAUGCUGUGCUGCAUGACCUCCUGCUGUUCUUGUUU
GAAAGGGUGUUGUUCUUGUGGUAGUUGUUGCAAGUUUGACGAAGAUGAUUCCGAACCUGUUCUUA
AAGGGGUAAAGCUUCACUAUACAUGAUAACCGCGGUGUCAAAAACCGCGUGGACGUGGUUAACAU
CCCUGCUGGGAGGAUCAGCCGUAAUUAUUAUAAUUGGCUUGGUGCUGGCUACUAUUGUGGCCAUG
UACGUGCUGACCAACCAGAAACAUAAUUGAAUACAGCAGCAAUUGGCAAGCUGCUUACAUAGAAC
UCGCGGCGAUUGGCAUGCCGCCUUAAAAUUUUUAUUUUAUUUUUUCUUUUCUUUUCCGAAUCGGA
UUUUGUUUUUAAUAUUUCAAAAAAAAAAAAAAAAAAAAAAAAAAAAAAAAAAAAAAAAAAAAA
SEQ ID NO: 6- SARS CoV-2 Delta-preF antigen
AUAGGCGGCGCAUGAGAGAAGCCCAGACCAAUUACCUACCCAAAAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCUAACCUGAAUGGACUAC
GACAUAGUCUAGUCCGCCAAGAUGUUUCUGCUCACAACCAAACGCACUAUGUUUGUUUUCCUCGU
GCUGCUCCCUUUGGUAAGUUCUCAGUGUGUAAACCUGAGAACACGAACCCAGUUGCCUCCAGCUU
AUACCAACUCAUUUACUCGCGGAGUAUAUUAUCCCGAUAAGGUCUUUAGAAGUAGCGUGUUGCAC
UCUACACAGGAUCUGUUCUUGCCCUUCUUUAGUAACGUUACCUGGUUUCAUGCAAUACAUGUGAG
CGGAACAAAUGGAACAAAAAGAUUUGACAAUCCAGUGCUUCCAUUUAAUGAUGGGGUUUACUUUG
CCAGUAUCGAAAAGUCAAACAUAAUCCGGGGGUGGAUCUUUGGAACCACUUUGGACUCUAAGACA
CAGUCUCUCCUCAUAGUAAACAACGCCACCAAUGUUGUCAUAAAAGUAUGCGAAUUUCAGUUUUG
CAACGAUCCCUUUCUCGACGUGUAUUACCAUAAGAAUAAUAAAUCCUGGAUGGAGUCUGGGGUUU
AUAGUAGUGCUAAUAAUUGCACUUUCGAAUACGUGUCCCAACCAUUCCUCAUGGACCUUGAGGGC
AAACAGGGGAAUUUUAAAAACUUGCGCGAAUUUGUCUUUAAGAAUAUCGACGGAUACUUUAAGAU
CUAUAGUAAACACACUCCUAUCAACCUCGUUCGGGAUCUUCCCCAAGGCUUUUCUGCUCUCGAAC
CCCUCGUAGACUUGCCAAUUGGGAUAAAUAUCACUCGCUUUCAAACUUUGCUUGCCCUCCACAGG
AGCUACCUGACACCCGGCGACUCUUCUUCUGGUUUGACCGCCGGCGCCGCUGCCUAUUAUGUUGG
UUACCUUCAGCCACGAACAUUCUUGCUCAAGUAUAACGAGAAUGGCACCAUUACCGACGCCGUCG
AUUGUGCAUUGGAUCCCUUGUCUGAAACAAAAUGUACCUUGAAGUCCUUUACCGUAGAGAAAGGC
AUAUACCAGACUUCCAACUUCCGAGUUCAGCCUACAGAAUCCAUCGUACGAUUUCCCAACAUCAC
AAACCUCUGCCCUUUCGGUGAAGUAUUUAAUGCUACACGCUUCGCUUCAGUCUAUGCCUGGAAUA
GGAAGCGCAUAUCAAAUUGCGUGGCCGAUUAUUCAGUCCUCUAUAAUAGCGCAUCCUUCAGUACU
UUCAAGUGCUACGGCGUUUCCCCCACCAAACUCAAUGAUCUUUGCUUCACCAACGUCUAUGCUGA
CAGUUUUGUCAUACGAGGCGACGAAGUACGCCAGAUUGCCCCCGGGCAGACAGGUAAUAUUGCUG
AUUAUAAUUAUAAACUCCCAGAUGACUUUACUGGAUGCGUCAUAGCCUGGAAUUCCAACAAUCUA
GAUUCCAAGGUUGGUGGGAAUUAUAAUUACCGUUAUCGACUGUUCAGAAAGAGUAACUUGAAACC
AUUUGAGAGAGACAUAUCCACCGAGAUUUACCAGGCAGGCAGUAAGCCUUGUAACGGCGUUGAGG
GAUUUAACUGCUAUUUUCCUUUGCAAUCCUAUGGCUUUCAACCAACAAACGGGGUUGGCUAUCAA
CCCUAUCGAGUGGUUGUCCUCAGCUUUGAACUUUUGCACGCUCCCGCCACAGUCUGCGGACCAAA
AAAGAGUACAAAUCUUGUCAAGAAUAAGUGCGUAAAUUUCAAUUUCAAUGGCCUUACAGGAACAG
GCGUGCUGACUGAGUCAAACAAGAAUUUCCUGCCAUUUCAGCAGUUUGGGCGGGAUAUAGCAGAC
ACAACUGACGCUGUACGCGAUCCUCAGACUUUGGAGAUCUUGGACAUCACUCCCUGUUCUUUCGG
AGGGGUAUCUGUCAUCACCCCCGGAACUAAUACAUCAAAUCAGGUCGCUGUGUUGUACCAAGGUG
UCAACUGCACAGAAGUCCCCGUUGCUAUACACGCAGACCAGCUCACCCCCACAUGGGGGGUGUAC
UCAACUGGCUCAAACGUAUUCCAGACCAGAGCUGGGUGCUUGAUCGGUGCUGAACACGUGAACAA
UAGCUAUGAAUGCGAUAUUCCCAUCGGUGCCGGGAUCUGCGCUAGCUAUCAGACACAGACCAAUU
CCCGCAGGCGGGCUCGCUCUGUAGCAUCCCAGUCUAUUAUUGCCUACACUAUGUCAUUGGGCGCC
GAGAAUAGCGUCGCAUAUUCAAAUAAUUCUAUUGCAAUACCCACCAACUUCACAAUCUCCGUAAC
UACAGAAAUACUUCCAGUUUCCAUGACAAAGACAUCAGUGGAUUGUACAAUGUAUAUAUGCGGAG
AUUCCACAGAAUGUUCAAAUUUGCUCUUGCAGUACGGCUCCUUCUGCACCCAGCUCAACAGGGCA
CUUACAGGUAUUGCUGUCGAACAGGACAAGAACACACAAGAAGUCUUCGCCCAAGUCAAACAGAU
AUACAAAACUCCUCCCAUAAAGGAUUUUGGCGGCUUCAACUUUAGUCAGAUCCUCCCAGACCCUU
CAAAACCAUCUAAACGAUCAUUUAUUGAAGAUCUGCUGUUCAACAAGGUCACUCUUGCCGAUGCU
GGAUUCAUUAAGCAAUACGGUGACUGCCUUGGUGAUAUUGCUGCCCGAGAUCUGAUCUGUGCCCA
GAAAUUCAACGGGCUCACUGUACUCCCUCCACUGCUCACAGACGAAAUGAUUGCACAGUACACAA
GUGCCCUGUUGGCAGGCACAAUCACUAGCGGCUGGACCUUUGGCGCAGGUGCAGCACUCCAAAUA
CCUUUUGCCAUGCAGAUGGCCUAUCGGUUUAAUGGGAUAGGCGUGACUCAAAAUGUCCUCUACGA
AAACCAAAAGUUGAUAGCUAACCAAUUCAAUUCAGCAAUCGGGAAGAUACAGGAUUCACUGUCUA
GUACUGCUAGUGCCCUUGGUAAGCUGCAGAACGUUGUCAACCAGAAUGCUCAAGCUCUGAAUACA
UUGGUUAAGCAGCUCUCUAGUAAUUUUGGGGCCAUCUCUUCAGUACUUAAUGAUAUUUUGAGCCG
AUUGGACCCACCUGAAGCUGAAGUACAGAUCGACAGGCUGAUAACAGGCCGGCUCCAAUCCCUCC
AAACAUACGUGACACAACAACUCAUACGCGCAGCCGAAAUCCGAGCCAGCGCUAACCUGGCAGCU
ACCAAGAUGUCAGAAUGCGUUCUGGGCCAGAGUAAACGCGUAGAUUUCUGCGGGAAAGGGUACCA
CCUGAUGUCCUUUCCACAAUCUGCACCUCACGGGGUCGUCUUUUUGCAUGUAACAUACGUACCCG
CACAAGAGAAGAAUUUUACUACCGCUCCUGCCAUCUGUCAUGACGGGAAAGCUCAUUUUCCUCGC
GAAGGUGUGUUUGUAUCUAAUGGUACACAUUGGUUUGUCACACAGCGGAAUUUCUAUGAACCCCA
GAUCAUUACAACUGACAACACUUUUGUUUCCGGGAAUUGUGACGUGGUCAUAGGAAUCGUAAAUA
ACACUGUAUAUGAUCCCCUCCAACCAGAGCUGGACUCUUUUAAAGAAGAACUGGAUAAAUAUUUC
AAGAACCACACAAGUCCCGACGUGGACCUUGGGGACAUAAGUGGUAUUAACGCAUCUGUGGUUAA
CAUUCAAAAGGAAAUCGACAGACUCAACGAGGUGGCCAAAAACCUGAACGAAAGCUUGAUAGAUC
UCCAGGAGUUGGGCAAGUAUGAACAGUACAUUAAAUGGCCAUGGUACAUAUGGCUUGGCUUUAUC
GCUGGCCUUAUCGCCAUCGUAAUGGUUACAAUCAUGCUGUGCUGCAUGACCUCCUGCUGUUCUUG
UUUGAAAGGGUGUUGUUCUUGUGGUAGUUGUUGCAAGUUUGACGAAGAUGAUUCCGAACCUGUUC
UUAAGGGGGUAAAGCUUCACUAUACAUGAUAACCGCGGUGUCAAAAACCGCGUGGACGUGGUUAA
CAUCCCUGCUGGGAGGAUCAGCCGUAAUUAUUAUAAUUGGCUUGGUGCUGGCUACUAUUGUGGCC
AUGUACGUGCUGACCAACCAGAAACAUAAUUGAAUACAGCAGCAAUUGGCAAGCUGCUUACAUAG
AACUCGCGGCGAUUGGCAUGCCGCCUUAAAAUUUUUAUUUUAUUUUUUCUUUUCUUUUCCGAAUC
GGAUUUUGUUUUUAAUAUUUCAAAAAAAAAAAAAAAAAAAAAAAAAAAAAAAAAAAAAAAAAAAA
A
SEQ ID NO: 7- SARS CoV-2 Delta-preF-kozak antigen
AUAGGCGGCGCAUGAGAGAAGCCCAGACCAAUUACCUACCCAAAAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCUAACCUGAAUGGACUAC
GACAUAGUCUAGUCCGCCGCCACCAUGUUUCUGCUCACAACCAAACGCACUAUGUUUGUUUUCCU
CGUGCUGCUCCCUUUGGUAAGUUCUCAGUGUGUAAACCUGAGAACACGAACCCAGUUGCCUCCAG
CUUAUACCAACUCAUUUACUCGCGGAGUAUAUUAUCCCGAUAAGGUCUUUAGAAGUAGCGUGUUG
CACUCUACACAGGAUCUGUUCUUGCCCUUCUUUAGUAACGUUACCUGGUUUCAUGCAAUACAUGU
GAGCGGAACAAAUGGAACAAAAAGAUUUGACAAUCCAGUGCUUCCAUUUAAUGAUGGGGUUUACU
UUGCCAGUAUCGAAAAGUCAAACAUAAUCCGGGGGUGGAUCUUUGGAACCACUUUGGACUCUAAG
ACACAGUCUCUCCUCAUAGUAAACAACGCCACCAAUGUUGUCAUAAAAGUAUGCGAAUUUCAGUU
UUGCAACGAUCCCUUUCUCGACGUGUAUUACCAUAAGAAUAAUAAAUCCUGGAUGGAGUCUGGGG
UUUAUAGUAGUGCUAAUAAUUGCACUUUCGAAUACGUGUCCCAACCAUUCCUCAUGGACCUUGAG
GGCAAACAGGGGAAUUUUAAAAACUUGCGCGAAUUUGUCUUUAAGAAUAUCGACGGAUACUUUAA
GAUCUAUAGUAAACACACUCCUAUCAACCUCGUUCGGGAUCUUCCCCAAGGCUUUUCUGCUCUCG
AACCCCUCGUAGACUUGCCAAUUGGGAUAAAUAUCACUCGCUUUCAAACUUUGCUUGCCCUCCAC
AGGAGCUACCUGACACCCGGCGACUCUUCUUCUGGUUUGACCGCCGGCGCCGCUGCCUAUUAUGU
UGGUUACCUUCAGCCACGAACAUUCUUGCUCAAGUAUAACGAGAAUGGCACCAUUACCGACGCCG
UCGAUUGUGCAUUGGAUCCCUUGUCUGAAACAAAAUGUACCUUGAAGUCCUUUACCGUAGAGAAA
GGCAUAUACCAGACUUCCAACUUCCGAGUUCAGCCUACAGAAUCCAUCGUACGAUUUCCCAACAU
CACAAACCUCUGCCCUUUCGGUGAAGUAUUUAAUGCUACACGCUUCGCUUCAGUCUAUGCCUGGA
AUAGGAAGCGCAUAUCAAAUUGCGUGGCCGAUUAUUCAGUCCUCUAUAAUAGCGCAUCCUUCAGU
ACUUUCAAGUGCUACGGCGUUUCCCCCACCAAACUCAAUGAUCUUUGCUUCACCAACGUCUAUGC
UGACAGUUUUGUCAUACGAGGCGACGAAGUACGCCAGAUUGCCCCCGGGCAGACAGGUAAUAUUG
CUGAUUAUAAUUAUAAACUCCCAGAUGACUUUACUGGAUGCGUCAUAGCCUGGAAUUCCAACAAU
CUAGAUUCCAAGGUUGGUGGGAAUUAUAAUUACCGUUAUCGACUGUUCAGAAAGAGUAACUUGAA
ACCAUUUGAGAGAGACAUAUCCACCGAGAUUUACCAGGCAGGCAGUAAGCCUUGUAACGGCGUUG
AGGGAUUUAACUGCUAUUUUCCUUUGCAAUCCUAUGGCUUUCAACCAACAAACGGGGUUGGCUAU
CAACCCUAUCGAGUGGUUGUCCUCAGCUUUGAACUUUUGCACGCUCCCGCCACAGUCUGCGGACC
AAAAAAGAGUACAAAUCUUGUCAAGAAUAAGUGCGUAAAUUUCAAUUUCAAUGGCCUUACAGGAA
CAGGCGUGCUGACUGAGUCAAACAAGAAUUUCCUGCCAUUUCAGCAGUUUGGGCGGGAUAUAGCA
GACACAACUGACGCUGUACGCGAUCCUCAGACUUUGGAGAUCUUGGACAUCACUCCCUGUUCUUU
CGGAGGGGUAUCUGUCAUCACCCCCGGAACUAAUACAUCAAAUCAGGUCGCUGUGUUGUACCAAG
GUGUCAACUGCACAGAAGUCCCCGUUGCUAUACACGCAGACCAGCUCACCCCCACAUGGGGGGUG
UACUCAACUGGCUCAAACGUAUUCCAGACCAGAGCUGGGUGCUUGAUCGGUGCUGAACACGUGAA
CAAUAGCUAUGAAUGCGAUAUUCCCAUCGGUGCCGGGAUCUGCGCUAGCUAUCAGACACAGACCA
AUUCCCGCAGGCGGGCUCGCUCUGUAGCAUCCCAGUCUAUUAUUGCCUACACUAUGUCAUUGGGC
GCCGAGAAUAGCGUCGCAUAUUCAAAUAAUUCUAUUGCAAUACCCACCAACUUCACAAUCUCCGU
AACUACAGAAAUACUUCCAGUUUCCAUGACAAAGACAUCAGUGGAUUGUACAAUGUAUAUAUGCG
GAGAUUCCACAGAAUGUUCAAAUUUGCUCUUGCAGUACGGCUCCUUCUGCACCCAGCUCAACAGG
GCACUUACAGGUAUUGCUGUCGAACAGGACAAGAACACACAAGAAGUCUUCGCCCAAGUCAAACA
GAUAUACAAAACUCCUCCCAUAAAGGAUUUUGGCGGCUUCAACUUUAGUCAGAUCCUCCCAGACC
CUUCAAAACCAUCUAAACGAUCAUUUAUUGAAGAUCUGCUGUUCAACAAGGUCACUCUUGCCGAU
GCUGGAUUCAUUAAGCAAUACGGUGACUGCCUUGGUGAUAUUGCUGCCCGAGAUCUGAUCUGUGC
CCAGAAAUUCAACGGGCUCACUGUACUCCCUCCACUGCUCACAGACGAAAUGAUUGCACAGUACA
CAAGUGCCCUGUUGGCAGGCACAAUCACUAGCGGCUGGACCUUUGGCGCAGGUGCAGCACUCCAA
AUACCUUUUGCCAUGCAGAUGGCCUAUCGGUUUAAUGGGAUAGGCGUGACUCAAAAUGUCCUCUA
CGAAAACCAAAAGUUGAUAGCUAACCAAUUCAAUUCAGCAAUCGGGAAGAUACAGGAUUCACUGU
CUAGUACUGCUAGUGCCCUUGGUAAGCUGCAGAACGUUGUCAACCAGAAUGCUCAAGCUCUGAAU
ACAUUGGUUAAGCAGCUCUCUAGUAAUUUUGGGGCCAUCUCUUCAGUACUUAAUGAUAUUUUGAG
CCGAUUGGACCCACCUGAAGCUGAAGUACAGAUCGACAGGCUGAUAACAGGCCGGCUCCAAUCCC
UCCAAACAUACGUGACACAACAACUCAUACGCGCAGCCGAAAUCCGAGCCAGCGCUAACCUGGCA
GCUACCAAGAUGUCAGAAUGCGUUCUGGGCCAGAGUAAACGCGUAGAUUUCUGCGGGAAAGGGUA
CCACCUGAUGUCCUUUCCACAAUCUGCACCUCACGGGGUCGUCUUUUUGCAUGUAACAUACGUAC
CCGCACAAGAGAAGAAUUUUACUACCGCUCCUGCCAUCUGUCAUGACGGGAAAGCUCAUUUUCCU
CGCGAAGGUGUGUUUGUAUCUAAUGGUACACAUUGGUUUGUCACACAGCGGAAUUUCUAUGAACC
CCAGAUCAUUACAACUGACAACACUUUUGUUUCCGGGAAUUGUGACGUGGUCAUAGGAAUCGUAA
AUAACACUGUAUAUGAUCCCCUCCAACCAGAGCUGGACUCUUUUAAAGAAGAACUGGAUAAAUAU
UUCAAGAACCACACAAGUCCCGACGUGGACCUUGGGGACAUAAGUGGUAUUAACGCAUCUGUGGU
UAACAUUCAAAAGGAAAUCGACAGACUCAACGAGGUGGCCAAAAACCUGAACGAAAGCUUGAUAG
AUCUCCAGGAGUUGGGCAAGUAUGAACAGUACAUUAAAUGGCCAUGGUACAUAUGGCUUGGCUUU
AUCGCUGGCCUUAUCGCCAUCGUAAUGGUUACAAUCAUGCUGUGCUGCAUGACCUCCUGCUGUUC
UUGUUUGAAAGGGUGUUGUUCUUGUGGUAGUUGUUGCAAGUUUGACGAAGAUGAUUCCGAACCUG
UUCUUAAGGGGGUAAAGCUUCACUAUACAUGAUAACCGCGGUGUCAAAAACCGCGUGGACGUGGU
UAACAUCCCUGCUGGGAGGAUCAGCCGUAAUUAUUAUAAUUGGCUUGGUGCUGGCUACUAUUGUG
GCCAUGUACGUGCUGACCAACCAGAAACAUAAUUGAAUACAGCAGCAAUUGGCAAGCUGCUUACA
UAGAACUCGCGGCGAUUGGCAUGCCGCCUUAAAAUUUUUAUUUUAUUUUUUCUUUUCUUUUCCGA
AUCGGAUUUUGUUUUUAAUAUUUCAAAAAAAAAAAAAAAAAAAAAAAAAAAAAAAAAAAAAAAAA
AAAA
SEQ ID NO: 8-SEQ ID NO: 11- See table 3
SEQ ID NO: 12 - VEEV-ZIKV-117 RNA Sequence
legend
ZIKV-117 HEAVY CHAIN
IRES
ZIKV-117 LIGHT CHAIN
Replicon backbone
auaggcggcgcaugagagaagcccagaccaauuaccuacccaaaauggagaaaguucacguugac
aucgaggaagacagcccauuccucagagcuuugcagcggagcuucccgcaguuugagguagaagc
caagcaggucacugauaaugaccaugcuaaugccagagcguuuucgcaucuggcuucaaaacuga
ucgaaacggagguggacccauccgacacgauccuugacauuggaagugcgcccgcccgcagaaug
uauucuaagcacaaguaucauuguaucuguccgaugagaugugcggaagauccggacagauugua
uaaguaugcaacuaagcugaagaaaaacuguaaggaaauaacugauaaggaauuggacaagaaaa
ugaaggagcuggccgccgucaugagcgacccugaccuggaaacugagacuaugugccuccacgac
gacgagucgugucgcuacgaagggcaagucgcuguuuaccaggauguauacgcgguugacggacc
gacaagucucuaucaccaagccaauaagggaguuagagucgccuacuggauaggcuuugacacca
ccccuuuuauguuuaagaacuuggcuggagcauauccaucauacucuaccaacugggccgacgaa
accguguuaacggcucguaacauaggccuaugcagcucugacguuauggagcggucacquagagg
gauguccauucuuagaaagaaguauuugaaaccauccaacaauguucuauucucuguuggcucga
ccaucuaccacgagaagagggacuuacugaggagcuggcaccugccgucuguauuucacuuacgu
ggcaagcaaaauuacacaugucggugugagacuauaguuaguugcgacggguacgucguuaaaag
aauagcuaucaguccaggccuguaugggaagccuucaggcuaugcugcuacgaugcaccgcgagg
gauucuugugcugcaaagugacagacacauugaacggggagagggucucuuuucccgugugcacg
uaugugccagcuacauugugugaccaaaugacuggcauacuggcaacagaugucagugcggacga
cgcgcaaaaacugcugguugggcucaaccagcguauagucgucaacggucgcacccagagaaaca
ccaauaccaugaaaaauuaccuuuugcccguaguggcccaggcauuugcuaggugggcaaaggaa
uauaaggaagaucaagaagaugaaaggccacuaggacuacgagauagacaguuagucauggggug
uuguugggcuuuuagaaggcacaagauaacaucuauuuauaagcgcccggauacccaaaccauca
ucaaagugaacagcgauuuccacucauucgugcugcccaggauaggcaguaacacauuggagauc
gggcugagaacaagaaucaggaaaauguuagaggagcacaaggagccgucaccucucauuaccgc
cgaggacguacaagaagcuaagugcgcagccgaugaggcuaaggaggugcgugaagccgaggagu
ugcgcgcagcucuaccaccuuuggcagcugauguugaggagcccacucuggaggcagacgucgac
uugauguuacaagaggcuggggccggcucaguggagacaccucguggcuugauaaagguuaccag
cuacgauggcgaggacaagaucggcucuuacgcugugcuuucuccgcaggcuguacucaagagug
aaaaauuaucuugcauccacccucucgcugaacaagucauagugauaacacacucuggccgaaaa
gggcguuaugccguggaaccauaccaugguaaaguaguggugccagagggacaugcaauacccgu
ccaggacuuucaagcucugagugaaagugccaccauuguguacaacgaacgugaguucguaaaca
gguaccugcaccauauugccacacauggaggagcgcugaacacugaugaagaauauuacaaaacu
gucaagcccagcgagcacgacggcgaauaccuguacgacaucgacaggaaacagugcgucaagaa
agaacuagucacugggcuagggcucacaggcgagcugguggauccucccuuccaugaauucgccu
acgagagucugagaacacgaccagccgcuccuuaccaaguaccaaccauagggguguauggcgug
ccaggaucaggcaagucuggcaucauuaaaagcgcagucaccaaaaaagaucuaguggugagcgc
caagaaagaaaacugugcagaaauuauaagggacgucaagaaaaugaaagggcuggacgucaaug
ccagaacuguggacucagugcucuugaauggaugcaaacaccccguagagacccuguauauugac
gaagcuuuugcuugucaugcagguacucucagagcgcucauagccauuauaagaccuaaaaaggc
agugcucugcggggaucccaaacagugcgguuuuuuuaacaugaugugccugaaagugcauuuua
accacgagauuugcacacaagucuuccacaaaagcaucucucgccguugcacuaaaucugugacu
ucggucgucucaaccuuguuuuacgacaaaaaaaugagaacgacgaauccgaaagagacuaagau
ugugauugacacuaccggcaguaccaaaccuaagcaggacgaucucauucucacuuguuucagag
ggugggugaagcaguugcaaauagauuacaaaggcaacgaaauaaugacggcagcugccucucaa
gggcugacccguaaagguguguaugccguucgguacaaggugaaugaaaauccucuguacgcacc
caccucagaacaugugaacguccuacugacccgcacggaggaccgcaucguguggaaaacacuag
ccggcgacccauggauaaaaacacugacugccaaguacccugggaauuucacugccacgauagag
gaguggcaagcagagcaugaugccaucaugaggcacaucuuggagagaccggacccuaccgacgu
cuuccagaauaaggcaaacguguguugggccaaggcuuuagugccggugcugaagaccgcuggca
uagacaugaccacugaacaauggaacacuguggauuauuuugaaacggacaaagcucacucagca
gagauaguauugaaccaacuaugcgugagguucuuuggacucgaucuggacuccggucuauuuuc
ugcacccacuguuccguuauccauuaggaauaaucacugggauaacuccccgucgccuaacaugu
acgggcugaauaaagaagugguccgucagcucucucgcagguacccacaacugccucgggcaguu
gccacuggaagagucuaugacaugaacacugguacacugcgcaauuaugauccgcgcauaaaccu
aguaccuguaaacagaagacugccucaugcuuuaguccuccaccauaaugaacacccacagagug
acuuuucuucauucgucagcaaauugaagggcagaacuguccugguggucggggaaaaguugucc
gucccaggcaaaaugguugacugguugucagaccggccugaggcuaccuucagagcucggcugga
uuuaggcaucccaggugaugugcccaaauaugacauaauauuuguuaaugugaggaccccauaua
aauaccaucacuaucagcagugugaagaccaugccauuaagcuuagcauguugaccaagaaagcu
ugucugcaucugaaucccggcggaaccugugucagcauagguuaugguuacgcugacagggccag
cgaaagcaucauuggugcuauagcgcggcaguucaaguuuucccggguaugcaaaccgaaauccu
cacuugaagagacggaaguucuguuuguauucauuggguacgaucgcaaggcccguacgcacaau
ccuuacaagcuuucaucaaccuugaccaacauuuauacagguuccagacuccacgaagccggaug
ugcacccucauaucauguggugcgaggggauauugccacggccaccgaaggagugauuauaaaug
cugcuaacagcaaaggacaaccuggcggaggggugugcggagcgcuguauaagaaauucccggaa
agcuucgauuuacagccgaucgaaguaggaaaagcgcgacuggucaaaggugcagcuaaacauau
cauucaugccguaggaccaaacuucaacaaaguuucggagguugaaggugacaaacaguuggcag
aggcuuaugaguccaucgcuaagauugucaacgauaacaauuacaagucaguagcgauuccacug
uuguccaccggcaucuuuuccgggaacaaagaucgacuaacccaaucauugaaccauuugcugac
agcuuuagacaccacugaugcagauguagccauauacugcagggacaagaaaugggaaaugacuc
ucaaggaagcaguggcuaggagagaagcaguggaggagauaugcauauccgacgacucuucagug
acagaaccugaugcagagcuggugagggugcauccgaagaguucuuuggcuggaaggaagggcua
cagcacaagcgauggcaaaacuuucucauauuuggaagggaccaaguuucaccaggcggccaagg
auauagcagaaauuaaugccauguggcccguugcaacggaggccaaugagcagguaugcauguau
auccucggagaaagcaugagcaguauuaggucgaaaugccccgucgaagagucggaagccuccac
accaccuagcacgcugccuugcuugugcauccaugccaugacuccagaaagaguacagcgccuaa
aagccucacguccagaacaaauuacugugugcucauccuuuccauugccgaaguauagaaucacu
ggugugcagaagauccaaugcucccagccuauauuguucucaccgaaagugccugcguauauuca
uccaaggaaguaucucguggaaacaccaccgguagacgagacuccggagccaucggcagagaacc
aauccacagaggggacaccugaacaaccaccacuuauaaccgaggaugagaccaggacuagaacg
ccugagccgaucaucaucgaagaggaagaagaggauagcauaaguuugcugucagauggcccgac
ccaccaggugcugcaagucgaggcagacauucacgggccgcccucuguaucuagcucauccuggu
ccauuccucaugcauccgacuuugauguggacaguuuauccauacuugacacccuggagggagcu
agcgugaccagcggggcaacgucagccgagacuaacucuuacuucgcaaagaguauggaguuucu
ggcgcgaccggugccugcgccucgaacaguauucaggaacccuccacaucccgcuccgcgcacaa
gaacaccgucacuugcacccagcagggccugcucgagaaccagccuaguuuccaccccgccaggc
gugaauagggugaucacuagagaggagcucgaggcgcuuaccccgucacgcacuccuagcagguc
ggucucgagaaccagccuggucuccaacccgccaggcguaaauagggugauuacaagagaggagu
uugaggcguucguagcacaacaacaaugacgguuugaugegggugcauacaucuuuuccuccgac
accggucaagggcauuuacaacaaaaaucaguaaggcaaacggugcuauccgaagugguguugga
gaggaccgaauuggagauuucguaugccccgcgccucgaccaagaaaaagaagaauuacuacgca
agaaauuacaguuaaaucccacaccugcuaacagaagcagauaccaguccaggaagguggagaac
augaaagccauaacagcuagacguauucugcaaggccuagggcauuauuugaaggcagaaggaaa
aguggagugcuaccgaacccugcauccuguuccuuuguauucaucuagugugaaccgugccuuuu
caagccccaaggucgcaguggaagccuguaacgccauguugaaagagaacuuuccgacuguggcu
ucuuacuguauuauuccagaguacgaugccuauuuggacaugguugacggagcuucaugcugcuu
agacacugccaguuuuugcccugcaaagcugcgcagcuuuccaaagaaacacuccuauuuggaac
ccacaauacgaucggcagugccuucagcgauccagaacacgcuccagaacguccuggcagcugcc
acaaaaagaaauugcaaugucacgcaaaugagagaauugcccguauuggauucggcggccuuuaa
uguggaaugcuucaagaaauaugcguguaauaaugaauauugggaaacguuuaaagaaaacccca
ucaggcuuacugaagaaaacgugguaaauuacauuaccaaauuaaaaggaccaaaagcugcugcu
cuuuuugcgaagacacauaauuugaauauguugcaggacauaccaauggacagguuuguaaugga
cuuaaagagagacgugaaagugacuccaggaacaaaacauacugaagaacggcccaagguacagg
ugauccaggcugccgauccgcuagcaacagcguaucugugcggaauccaccgagagcugguuagg
agauuaaaugcgguccugcuuccgaacauucauacacuguuugauaugucggcugaagacuuuga
cgcuauuauagccgagcacuuccagccuggggauuguguucuggaaacugacaucgcgucguuug
auaaaagugaggacgacgccauggcucugaccgcguuaaugauucuggaagacuuagguguggac
gcagagcuguugacgcugauugaggcggcuuucggcgaaauuucaucaauacauuugcccacuaa
aacuaaauuuaaauucggagccaugaugaaaucuggaauguuccucacacuguuugugaacacag
ucauuaacauuguaaucgcaagcagaguguugagagaacggcuaaccggaucaccaugugcagca
uucauuggagaugacaauaucgugaaaggagucaaaucggacaaauuaauggcagacaggugcgc
caccugguugaauauggaagucaagauuauagaugcuguggugggcgagaaagcgccuuauuucu
guggaggguuuauuuugugugacuccgugaccggcacagcgugccguguggcagacccccuaaaa
aggcuguuuaagcuuggcaaaccucuggcagcagacgaugaacaugaugaugacaggagaagggc
auugcaugaagagucaacacgcuggaaccgaguggguauucuuucagagcugugcaaggcaguag
aaucaagguaugaaaccguaggaacuuccaucauaguuauggccaugacuacucuagcuagcagu
guuaaaucauucagcuaccugagaggggccccuauaacucucuacggcuaaccugaauggacuac
gacauagucuaguccgccaagAUGGAGUUCGGUCUUAGCUGGGUGUUUCUUGUCGCCCUGUUCAG
AGGGGUACAAUGCCAAGUGCAACUUGUAGAGAGUGGCGGCGGCGUAGUUCGACCAGGUGGGAGUC
UGAGGCUGUCAUGUGCAGCAUCCGGGUUCACCUUUAAAAACUACGGGAUUCACUGGGUGAGGCAG
GCUCCUGGUAAGGGACCAGAGUGGGUCGCCUUCGUGCGCUAUGAUGGGAAUAACAAAUAUUACGC
UGACUCCGUCAAAGGUCGCUUCACAAUAUCCAGGGACAAUGCAAAAAAUACACUGAGCUUGCAAA
UGAAUUCUUUGCGGGUGGAAGAUACUGCUGUAUAUUUCUGUGCUCGGGAUCCAGAAACUUUUGGA
GGGUUUGAUUAUUGGGGGCAAGGGACACUCGUUACUGUCAGUAGCGCCUCCACAAAGGGUCCAAG
UGUCUUCCCACUGGCUCCCAGCAGCAAGAGUACUUCAGGUGGGACUGCAGCUCUCGGGUGCCUGG
UCAAGGACUACUUUCCCGAGCCCGUAACAGUAUCUUGGAACUCCGGUGCUCUGACAAGUGGAGUG
CAUACUUUCCCAGCUGUGUUGCAGUCAAGCGGGUUGUACUCCCUCAGUAGUGUAGUUACUGUCCC
UUCAUCUUCACUGGGGACUCAAACCUACAUUUGUAACGUGAAUCACAAACCAAGCAAUACUAAAG
UAGAUAAGAAGGUGGAGCCAAAAAGUUGUGAUAAAACUCAUACUUGUCCCCCCUGUCCUGCACCA
GAGCUGUUGGGCGGUCCCAGUGUAUUUUUGUUUCCCCCUAAACCCAAAGACACACUGAUGAUUUC
UCGAACUCCCGAAGUGACCUGCGUCGUCGUUGAUGUAAGUCACGAAGACCCCGAGGUAAAAUUCA
AUUGGUACGUAGACGGCGUAGAGGUGCAUAACGCUAAAACCAAACCAAGGGAGGAACAAUACAAC
AGCACUUACAGAGUCGUAUCUGUACUGACUGUUCUCCACCAAGACUGGCUUAAUGGGAAGGAGUA
UAAAUGCAAGGUGUCAAACAAGGCUCUGCCUGCCCCCAUAGAGAAAACCAUAAGUAAAGCUAAAG
GACAACCUAGAGAGCCCCAGGUUUAUACUCUUCCCCCCUCCCGAGAAGAGAUGACCAAGAACCAA
GUUUCCUUGACCUGUCUGGUUAAGGGUUUUUAUCCAAGCGAUAUAGCCGUAGAAUGGGAGAGCAA
CGGACAACCUGAGAAUAACUACAAAACAACUCCCCCAGUGCUGGACUCAGAUGGCUCAUUUUUCC
UGUAUUCAAAGCUCACCGUGGACAAAUCUCGGUGGCAGCAAGGGAAUGUAUUCUCCUGUUCCGUC
AUGCACGAGGCGCUGCAUAAUCACUAUACCCAGAAAUCUCUGUCCCUUUCUCCUGGAAAGUGAua
aucuagaccccucucccuccccccccccuaacguuacuggccgaagccgcuuggaauaaggccgg
ugugcguuugucuauauguuauuuuccaccauauugccgucuuuuggcaaugugagggcccggaa
accuggcccugucuucuugacgagcauuccuaggggucuuuccccucucgccaaaggaaugcaag
gucuguugaaugucgugaaggaagcaguuccucuggaagcuucuugaagacaaacaacgucugua
gcgacccuuugcaggcagcggaaccccccaccuggcgacaggugccucugcggccaaaagccacg
uguauaagauacaccugcaaaggcggcacaaccccagugccacguugugaguuggauaguugugg
aaagagucaaauggcucuccucaagcguauucaacaaggggcugaaggaugcccagaagguaccc
cauuguaugggaucugaucuggggccucggugcacaugcuuuacauguguuuagucgagguuaaa
aaacgucuaggccccccgaaccacggggacgugguuuuccuuugaaaaacacgaugauaauaugg
ccacaaccAUGGACAUGCGAGUCCCUGCCCAACUGCUGGGUCUUCUCCUCCUUUGGCUGUCAGGA
GCCCGCUGCGAAAUUGUAAUGACACAAUCUCCAGCAACACUUUCCGUCAGUCCAGGCGAGAGAGG
GACCUUGUCAUGCAGGGCUUCCGAAAGCGUUUCCAGCAACCUUGCUUGGUAUCAACAAAAACCUG
GAAAGGCCCCCAGACUGCUGAUCUAUGGCGCUUCCACACGCGCAACUGGUAUUCCUGACAGAUUC
UCCGGGUCUGGGUCUGGCACUGAGUUCACACUCACAAUUUCCAGUCUGCAGUCCGAGGAUUUUGC
UGUAUAUUAUUGCCAACAGUAUUACUAUAGUCCUAGGACCUUUGGUCAAGGGACUAAGGUCGAGG
UAAAGCGGACCGUGGCUGCACCAUCCGUUUUUAUUUUUCCACCAAGUGAUGAGCAGCUUAAAAGU
GGUACUGCCUCCGUGGUGUGCUUGUUGAACAACUUCUACCCACGCGAGGCCAAGGUGCAAUGGAA
GGUAGAUAAUGCCUUGCAGAGUGGAAAUUCUCAAGAGUCAGUCACCGAACAGGAUAGUAAAGACU
CUACAUAUUCUCUUAGCUCUACCCUCACUUUGUCUAAAGCAGAUUAUGAAAAGCAUAAAGUGUAU
GCAUGCGAAGUGACCCACCAGGGGCUGAGGUCUCCUGUCACCAAAAGUUUUAACAGGGGAGAGUG
UUGAUAAccgcggugucaaaaaccgcguggacgugguuaacaucccugcugggaggaucagccgu
aauuauuauaauuggcuuggugcuggcuacuauuguggccauguacgugcugaccaaccagaaac
auaauugaauacagcagcaauuggcaagcugcuuacauagaacucgcggcgauuggcaugccgcc
uuaaaauuuuuuuuuuuuuuuuucuuuucuuuuccgaaucggauuuuguuuuuaauauuucaaaa
aaaaaaaaaaaaaaaaaaaaaaaaaaaaaaaaaaaaaaaaa
SEQ ID NO: 13 - ZIKV-117 Heavy Chain
AUGGAGUUCGGUCUUAGCUGGGUGUUUCUUGUCGCCCUGUUCAGAGGGGUACAAUGCCAAGUGCA
ACUUGUAGAGAGUGGCGGCGGCGUAGUUCGACCAGGUGGGAGUCUGAGGCUGUCAUGUGCAGCAU
CCGGGUUCACCUUUAAAAACUACGGGAUUCACUGGGUGAGGCAGGCUCCUGGUAAGGGACCAGAG
UGGGUCGCCUUCGUGCGCUAUGAUGGGAAUAACAAAUAUUACGCUGACUCCGUCAAAGGUCGCUU
CACAAUAUCCAGGGACAAUGCAAAAAAUACACUGAGCUUGCAAAUGAAUUCUUUGCGGGUGGAAG
AUACUGCUGUAUAUUUCUGUGCUCGGGAUCCAGAAACUUUUGGAGGGUUUGAUUAUUGGGGGCAA
GGGACACUCGUUACUGUCAGUAGCGCCUCCACAAAGGGUCCAAGUGUCUUCCCACUGGCUCCCAG
CAGCAAGAGUACUUCAGGUGGGACUGCAGCUCUCGGGUGCCUGGUCAAGGACUACUUUCCCGAGC
CCGUAACAGUAUCUUGGAACUCCGGUGCUCUGACAAGUGGAGUGCAUACUUUCCCAGCUGUGUUG
CAGUCAAGCGGGUUGUACUCCCUCAGUAGUGUAGUUACUGUCCCUUCAUCUUCACUGGGGACUCA
AACCUACAUUUGUAACGUGAAUCACAAACCAAGCAAUACUAAAGUAGAUAAGAAGGUGGAGCCAA
AAAGUUGUGAUAAAACUCAUACUUGUCCCCCCUGUCCUGCACCAGAGCUGUUGGGCGGUCCCAGU
GUAUUUUUGUUUCCCCCUAAACCCAAAGACACACUGAUGAUUUCUCGAACUCCCGAAGUGACCUG
CGUCGUCGUUGAUGUAAGUCACGAAGACCCCGAGGUAAAAUUCAAUUGGUACGUAGACGGCGUAG
AGGUGCAUAACGCUAAAACCAAACCAAGGGAGGAACAAUACAACAGCACUUACAGAGUCGUAUCU
GUACUGACUGUUCUCCACCAAGACUGGCUUAAUGGGAAGGAGUAUAAAUGCAAGGUGUCAAACAA
GGCUCUGCCUGCCCCCAUAGAGAAAACCAUAAGUAAAGCUAAAGGACAACCUAGAGAGCCCCAGG
UUUAUACUCUUCCCCCCUCCCGAGAAGAGAUGACCAAGAACCAAGUUUCCUUGACCUGUCUGGUU
AAGGGUUUUUAUCCAAGCGAUAUAGCCGUAGAAUGGGAGAGCAACGGACAACCUGAGAAUAACUA
CAAAACAACUCCCCCAGUGCUGGACUCAGAUGGCUCAUUUUUCCUGUAUUCAAAGCUCACCGUGG
ACAAAUCUCGGUGGCAGCAAGGGAAUGUAUUCUCCUGUUCCGUCAUGCACGAGGCGCUGCAUAAU
CACUAUACCCAGAAAUCUCUGUCCCUUUCUCCUGGAAAGUGA
SEQ ID NO: 14 - ZIKV-117 Light Chain
AUGGACAUGCGAGUCCCUGCCCAACUGCUGGGUCUUCUCCUCCUUUGGCUGUCAGGAGCCCGCUG
CGAAAUUGUAAUGACACAAUCUCCAGCAACACUUUCCGUCAGUCCAGGCGAGAGAGGGACCUUGU
CAUGCAGGGCUUCCGAAAGCGUUUCCAGCAACCUUGCUUGGUAUCAACAAAAACCUGGAAAGGCC
CCCAGACUGCUGAUCUAUGGCGCUUCCACACGCGCAACUGGUAUUCCUGACAGAUUCUCCGGGUC
UGGGUCUGGCACUGAGUUCACACUCACAAUUUCCAGUCUGCAGUCCGAGGAUUUUGCUGUAUAUU
AUUGCCAACAGUAUUACUAUAGUCCUAGGACCUUUGGUCAAGGGACUAAGGUCGAGGUAAAGCGG
ACCGUGGCUGCACCAUCCGUUUUUAUUUUUCCACCAAGUGAUGAGCAGCUUAAAAGUGGUACUGC
CUCCGUGGUGUGCUUGUUGAACAACUUCUACCCACGCGAGGCCAAGGUGCAAUGGAAGGUAGAUA
AUGCCUUGCAGAGUGGAAAUUCUCAAGAGUCAGUCACCGAACAGGAUAGUAAAGACUCUACAUAU
UCUCUUAGCUCUACCCUCACUUUGUCUAAAGCAGAUUAUGAAAAGCAUAAAGUGUAUGCAUGCGA
AGUGACCCACCAGGGGCUGAGGUCUCCUGUCACCAAAAGUUUUAACAGGGGAGAGUGUUGAUAA
SEQ ID NO: 15-37- See Table 4
SEQ ID NO: 38- VEEV RNA Sequence
auaggcggcgcaugagagaagcccagaccaauuaccuacccaaaauggagaaaguucacguugac
aucgaggaagacagcccauuccucagagcuuugcagcggagcuucccgcaguuugagguagaagc
caagcaggucacugauaaugaccaugcuaaugccagagcguuuucgcaucuggcuucaaaacuga
ucgaaacggagguggacccauccgacacgauccuugacauuggaagugcgcccgcccgcagaaug
uauucuaagcacaaguaucauuguaucuguccgaugagaugugcggaagauccggacagauugua
uaaguaugcaacuaagcugaagaaaaacuguaaggaaauaacugauaaggaauuggacaagaaaa
ugaaggagcuggccgccgucaugagcgacccugaccuggaaacugagacuaugugccuccacgac
gacgagucgugucgcuacgaagggcaagucgcuguuuaccaggauguauacgcgguugacggacc
gacaagucucuaucaccaagccaauaagggaguuagagucgccuacuggauaggcuuugacacca
ccccuuuuauguuuaagaacuuggcuggagcauauccaucauacucuaccaacugggccgacgaa
accguguuaacggcucguaacauaggccuaugcagcucugacguuauggagcggucacguagagg
gauguccauucuuagaaagaaguauuugaaaccauccaacaauguucuauucucuguuggcucga
ccaucuaccacgagaagagggacuuacugaggagcuggcaccugccgucuguauuucacuuacgu
ggcaagcaaaauuacacaugucggugugagacuauaguuaguugcgacggguacgucguuaaaag
aauagcuaucaguccaggccuguaugggaagccuucaggcuaugcugcuacgaugcaccgcgagg
gauucuugugcugcaaagugacagacacauugaacggggagagggucucuuuucccgugugcacg
uaugugccagcuacauugugugaccaaaugacuggcauacuggcaacagaugucagugcggacga
cgcgcaaaaacugcugguugggcucaaccagcguauagucgucaacggucgcacccagagaaaca
ccaauaccaugaaaaauuaccuuuugcccguaguggcccaggcauuugcuaggugggcaaaggaa
uauaaggaagaucaagaagaugaaaggccacuaggacuacgagauagacaguuagucauggggug
uuguugggcuuuuagaaggcacaagauaacaucuauuuauaagcgcccggauacccaaaccauca
ucaaagugaacagcgauuuccacucauucgugcugcccaggauaggcaguaacacauuggagauc
gggcugagaacaagaaucaggaaaauguuagaggagcacaaggagccgucaccucucauuaccgc
cgaggacguacaagaagcuaagugcgcagccgaugaggcuaaggaggugcgugaagccgaggagu
ugcgcgcagcucuaccaccuuuggcagcugauguugaggagcccacucuggaggcagacgucgac
uugauguuacaagaggcuggggccggcucaguggagacaccucguggcuugauaaagguuaccag
cuacgauggcgaggacaagaucggcucuuacgcugugcuuucuccgcaggcuguacucaagagug
aaaaauuaucuugcauccacccucucgcugaacaagucauagugauaacacacucuggccgaaaa
gggcguuaugccguggaaccauaccaugguaaaguaguggugccagagggacaugcaauacccgu
ccaggacuuucaagcucugagugaaagugccaccauuguguacaacgaacgugaguucguaaaca
gguaccugcaccauauugccacacauggaggagcgcugaacacugaugaagaauauuacaaaacu
gucaagcccagcgagcacgacggcgaauaccuguacgacaucgacaggaaacagugcgucaagaa
agaacuagucacugggcuagggcucacaggcgagcugguggauccucccuuccaugaauucgccu
acgagagucugagaacacgaccagccgcuccuuaccaaguaccaaccauagggguguauggcgug
ccaggaucaggcaagucuggcaucauuaaaagcgcagucaccaaaaaagaucuaguggugagcgc
caagaaagaaaacugugcagaaauuauaagggacgucaagaaaaugaaagggcuggacgucaaug
ccagaacuguggacucagugcucuugaauggaugcaaacaccccguagagacccuguauauugac
gaagcuuuugcuugucaugcagguacucucagagcgcucauagccauuauaagaccuaaaaaggc
agugcucugcggggaucccaaacagugcgguuuuuuuaacaugaugugccugaaagugcauuuua
accacgagauuugcacacaagucuuccacaaaagcaucucucgccguugcacuaaaucugugacu
ucggucgucucaaccuuguuuuacgacaaaaaaaugagaacgacgaauccgaaagagacuaagau
ugugauugacacuacoggcaguaccaaaccuaagcaggacgaucucauucucacuuguuucagag
ggugggugaagcaguugcaaauagauuacaaaggcaacgaaauaaugacggcagcugccucucaa
gggcugacccguaaagguguguaugccguucgguacaaggugaaugaaaauccucuguacgcacc
caccucagaacaugugaacguccuacugacccgcacggaggaccgcaucguguggaaaacacuag
ccggcgacccauggauaaaaacacugacugccaaguacccugggaauuucacugccacgauagag
gaguggcaagcagagcaugaugccaucaugaggcacaucuuggagagaccggacccuaccgacgu
cuuccagaauaaggcaaacguguguugggccaaggcuuuagugccggugcugaagaccgcuggca
uagacaugaccacugaacaauggaacacuguggauuauuuugaaacggacaaagcucacucagca
gagauaguauugaaccaacuaugcgugagguucuuuggacucgaucuggacuccggucuauuuuc
ugcacccacuguuccguuauccauuaggaauaaucacugggauaacuccccgucgccuaacaugu
acgggcugaauaaagaagugguccgucagcucucucgcagguacccacaacugccucgggcaguu
gccacuggaagagucuaugacaugaacacugguacacugcgcaauuaugauccgcgcauaaaccu
aguaccuguaaacagaagacugccucaugcuuuaguccuccaccauaaugaacacccacagagug
acuuuucuucauucgucagcaaauugaagggcagaacuguccugguggucggggaaaaguugucc
gucccaggcaaaaugguugacugguugucagaccggccugaggcuaccuucagagcucggcugga
uuuaggcaucccaggugaugugcccaaauaugacauaauauuuguuaaugugaggaccccauaua
aauaccaucacuaucagcagugugaagaccaugccauuaagcuuagcauguugaccaagaaagcu
ugucugcaucugaaucccggcggaaccugugucagcauagguuaugguuacgcugacagggccag
cgaaagcaucauuggugcuauagcgcggcaguucaaguuuucccggguaugcaaaccgaaauccu
cacuugaagagacggaaguucuguuuguauucauuggguacgaucgcaaggcccguacgcacaau
ccuuacaagcuuucaucaaccuugaccaacauuuauacagguuccagacuccacgaagccggaug
ugcacccucauaucauguggugcgaggggauauugccacggccaccgaaggagugauuauaaaug
cugcuaacagcaaaggacaaccuggcggaggggugugcggagcgcuguauaagaaauucccggaa
agcuucgauuuacagccgaucgaaguaggaaaagcgcgacuggucaaaggugcagcuaaacauau
cauucaugccguaggaccaaacuucaacaaaguuucggagguugaaggugacaaacaguuggcag
aggcuuaugaguccaucgcuaagauugucaacgauaacaauuacaagucaguagcgauuccacug
uuguccaccggcaucuuuuccgggaacaaagaucgacuaacccaaucauugaaccauuugcugac
agcuuuagacaccacugaugcagauguagccauauacugcagggacaagaaaugggaaaugacuc
ucaaggaagcaguggcuaggagagaagcaguggaggagauaugcauauccgacgacucuucagug
acagaaccugaugcagagcuggugagggugcauccgaagaguucuuuggcuggaaggaagggcua
cagcacaagcgauggcaaaacuuucucauauuuggaagggaccaaguuucaccaggcggccaagg
auauagcagaaauuaaugccauguggcccguugcaacggaggccaaugagcagguaugcauguau
auccucggagaaagcaugagcaguauuaggucgaaaugccccgucgaagagucggaagccuccac
accaccuagcacgcugccuugcuugugcauccaugccaugacuccagaaagaguacagcgccuaa
aagccucacguccagaacaaauuacugugugcucauccuuuccauugccgaaguauagaaucacu
ggugugcagaagauccaaugcucccagccuauauuguucucaccgaaagugccugcguauauuca
uccaaggaaguaucucguggaaacaccaccgguagacgagacuccggagccaucggcagagaacc
aauccacagaggggacaccugaacaaccaccacuuauaaccgaggaugagaccaggacuagaacg
ccugagccgaucaucaucgaagaggaagaagaggauagcauaaguuugcugucagauggcccgac
ccaccaggugcugcaagucgaggcagacauucacgggccgcccucuguaucuagcucauccuggu
ccauuccucaugcauccgacuuugauguggacaguuuauccauacuugacacccuggagggagcu
agcgugaccagcggggcaacgucagccgagacuaacucuuacuucgcaaagaguauggaguuucu
ggcgcgaccggugccugcgccucgaacaguauucaggaacccuccacaucccgcuccgcgcacaa
gaacaccgucacuugcacccagcagggccugcucgagaaccagccuaguuuccaccccgccaggc
gugaauagggugaucacuagagaggagcucgaggcgcuuaccccgucacgcacuccuagcagguc
ggucucgagaaccagccuggucuccaacccgccaggcguaaauagggugauuacaagagaggagu
uugaggcguucguagcacaacaacaaugacgguuugaugcgggugcauacaucuuuuccuccgac
accggucaagggcauuuacaacaaaaaucaguaaggcaaacggugcuauccgaagugguguugga
gaggaccgaauuggagauuucguaugccccgcgccucgaccaagaaaaagaagaauuacuacgca
agaaauuacaguuaaaucccacaccugcuaacagaagcagauaccaguccaggaagguggagaac
augaaagccauaacagcuagacguauucugcaaggccuagggcauuauuugaaggcagaaggaaa
aguggagugcuaccgaacccugcauccuguuccuuuguauucaucuagugugaaccgugccuuuu
caagccccaaggucgcaguggaagccuguaacgccauguugaaagagaacuuuccgacuguggcu
ucuuacuguauuauuccagaguacgaugccuauuuggacaugguugacggagcuucaugcugcuu
agacacugccaguuuuugcccugcaaagcugcgcagcuuuccaaagaaacacuccuauuuggaac
ccacaauacgaucggcagugccuucagcgauccagaacacgcuccagaacguccuggcagcugcc
acaaaaagaaauugcaaugucacgcaaaugagagaauugcccguauuggauucggcggccuuuaa
uguggaaugcuucaagaaauaugcguguaauaaugaauauugggaaacguuuaaagaaaacccca
ucaggcuuacugaagaaaacgugguaaauuacauuaccaaauuaaaaggaccaaaagcugcugcu
cuuuuugcgaagacacauaauuugaauauguugcaggacauaccaauggacagguuuguaaugga
cuuaaagagagacgugaaagugacuccaggaacaaaacauacugaagaacggcccaagguacagg
ugauccaggcugccgauccgcuagcaacagcguaucugugcggaauccaccgagagcugguuagg
agauuaaaugcgguccugcuuccgaacauucauacacuguuugauaugucggcugaagacuuuga
cgcuauuauagccgagcacuuccagccuggggauuguguucuggaaacugacaucgcgucguuug
auaaaagugaggacgacgccauggcucugaccgcguuaaugauucuggaagacuuagguguggac
gcagagcuguugacgcugauugaggcggcuuucggcgaaauuucaucaauacauuugcccacuaa
aacuaaauuuaaauucggagccaugaugaaaucuggaauguuccucacacuguuugugaacacag
ucauuaacauuguaaucgcaagcagaguguugagagaacggcuaaccggaucaccaugugcagca
uucauuggagaugacaauaucgugaaaggagucaaaucggacaaauuaauggcagacaggugcgc
caccugguugaauauggaagucaagauuauagaugcuguggugggcgagaaagcgccuuauuucu
guggaggguuuauuuugugugacuccgugaccggcacagcgugccguguggcagacccccuaaaa
aggcuguuuaagcuuggcaaaccucuggcagcagacgaugaacaugaugaugacaggagaagggc
auugcaugaagagucaacacgcuggaaccgaguggguauucuuucagagcugugcaaggcaguag
aaucaagguaugaaaccguaggaacuuccaucauaguuauggccaugacuacucuagcuagcagu
guuaaaucauucagcuaccugagaggggccccuauaacucucuacggcuaaccugaauggacuac
gacauagucuaguccgccaag
SEQ ID NO: 39- VEEV RNA polymerase Amino Acid Sequence (NCBI Accession:
AXP98866.1)
RELPVLDSAAFNVECFKKYACNNEYWETFKENPIRLTEENVVNYITKLKGP
SEQ ID NO: 40- VEEV RNA polymerase Amino Acid Sequence (NCBI Accession:
AXP98867.1)
TQMRELPVLDSAAFNVECFKKYACNNEYWETFKENPIRLTE
SEQ ID NO: 41- 845-VEErep-CDKN2a-8522
auaggcggcgcaugagagaagcccagaccaauuaccuacccaaaAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCuaaccugaauggacuac
gacauagucuaguccgccgccaccAUGGGGAGAAGAUUCCUGGUCACCGUGCGGAUCCAGAGAGC
UGGCAGACCUCUGCAAGAAAGAGUGUUUCUGGUCAAGUUCGUGCGGAGCAGAAGGCCCAGAACAG
CCUCUUGUGCUCUGGCCUUCGUGAACAUGCUGCUGAGACUGGAAAGGAUCCUGAGAAGAGGCCCU
CACAGAAACCCUGGACCUGGCGACGAUGACGGCCAGAGAAGCAGAUCUUCUAGCAGCGCCCAGCU
GAGAUGCAGAUUCGAGCUGAGGGGCCCUCACUACUUGCUUCCACCUGGCGCUAGAAGAAGCGCCG
GUAGACUUCCUGGACAUGCUGGCGGAGCUGCUAGAGUCAGAGGCUCUGCUGGCUGUGCUAGAUGU
CUGGGCUCUCCUGCUGCAAGACUGGGCCCUAGAGCCGGCACAUCUAGACACCGGGCUAUCUUCGC
CUUCAGAUGGGUGCUGUUCGUGUUUAGAUGGGUCGUGUUUGUGUACCGCUGGGAGAGAAGGCCUG
ACAGAAGGGCUUGAUAACCGCGGUGUCAAAAACCGCGUGGACGUGGUUAACAUCCCUGCUGGGAG
GAUCAGCCGUAAUUAUUAUAAUUGGCUUGGUGCUGGCUACUAUUGUGGCCAUGUACGUGCUGACC
AACCAGAAACAUAAuugaauacagcagcaauuggcaagcugcuuacauagaacucgcggcgauug
gcaugccgccuuaaaauuuuuauuuuauuuuuucuuuucuuuuccgaaucggauuuuguuuuuaa
uauuucaaaaaaaaaaaaaaaaaaaaaaaaaaaaaaaaaaaaaaaaaaaaa
SEQ ID NO: 42- 846-VEErep-CDKN2b-8521
auaggcggcgcaugagagaagcccagaccaauuaccuacccaaaAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCuaaccugaauggacuac
gacauagucuaguccgccgccaccAUGCUCGGCGGAUCUUCUGAUGCUGGCCUGGCUACAGCUGC
UGCUAGAGGACAGGUGGAAACAGUGCGGCAGCUGCUUGAAGCUGGCGCCGAUCCUAACGCUCUGA
ACAGAUUUGGCAGACGGCCCAUCCAAGUGAUGAUGAUGGGCUCUGCUCAGGUGGCCGAACUGCUG
CUUCUUCACGGCGCUGAGCCUAACUGUGCCGAUCCUGCCACACUGACCAGACCUGUGCAUGAUGC
CGCUAGAGAGGGCUUCCUGGACACACUGGUGGUGCUGCAUAGAGCCGGCGCUAGACUGGAUGUGU
GUGAUGCUUGGGGCAGACUGCCUGUGGAUCUGGCUGAGGAACAGGGCCACAGAGAUAUCGCCAGA
UACCUGCACGCCGCCACAGGUGAUUGAUAACCGCGGUGUCAAAAACCGCGUGGACGUGGUUAACA
UCCCUGCUGGGAGGAUCAGCCGUAAUUAUUAUAAUUGGCUUGGUGCUGGCUACUAUUGUGGCCAU
GUACGUGCUGACCAACCAGAAACAUAAuugaauacagcagcaauuggcaagcugcuuacauagaa
cucgcggcgauuggcaugccgccuuaaaauuuuuauuuuauuuuuucuuuucuuuuccgaaucgg
auuuuguuuuuaauauuucaaaaaaaaaaaaaaaaaaaaaaaaaaaaaaaaaaaaaaaaaaaaa
SEQ ID NO: 43- 847-VEErep-CDKN2c-8520
auaggcggcgcaugagagaagcccagaccaauuaccuacccaaaAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCuaaccugaauggacuac
gacauagucuaguccgccgccaccAUGGCUGAGCCUUGGGGAAACGAGCUGGCUUCUGCUGCUGC
UAGAGGCGACCUGGAACAGCUGACAAGCCUGCUGCAGAACAACGUGAACGUCAACGCCCAGAACG
GCUUCGGCAGAACAGCCCUGCAAGUGAUGAAGCUGGGCAACCCUGAGAUCGCCAGAAGGCUGCUU
CUGAGAGGCGCUAACCCCAACCUGAAGGACGGCACAGGCUUCGCCGUGAUCCAUGAUGCUGCCAG
AGCCGGCUUCCUGGAUACAGUGCAGGCUCUGCUGGAAUUUCAGGCCGACGUGAACAUCGAGGACA
ACGAGGGAAACCUGCCUCUGCACCUGGCUGCCAAAGAGGGACAUCUGCCCGUCGUGGAAUUCCUG
AUGAAGCACACCGCCUGCAACGUGGGCCACAGAAACCACAAGGGCGACACAGCCUUCGACCUGGC
CAGAUUCUACGGCAGAAACGAAGUGAUCAGCCUGAUGGAAGCCAACGGCGUCGGCGGAGCUACAU
CUCUUCAGUGAuaaccgcggugucaaaaaccgcguggacgugguuaacaucccugcugggaggau
cagccguaauuauuauaauuggcuuggugcuggcuacuauuguggccauguacgugcugaccaac
cagaaacauaauugaauacagcagcaauuggcaagcugcuuacauagaacucgcggcgauuggca
ugccgccuuaaaauuuuuauuuuauuuuuucuuuucuuuuccgaaucggauuuuguuuuuaauau
uucaaaaaaaaaaaaaaaaaaaaaaaaaaaaaaaaaaaaaaaaaaaaa
SEQ ID NO: 44- 848-VEErep-CDKN2d-8523
auaggcggcgcaugagagaagcccagaccaauuaccuacccaaaAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCuaaccugaauggacuac
gacauagucuaguccgccgccaccAUGCUGCUGGAAGAAGUGUGCGUCGGCGACAGACUUUCUGG
CGCUGCUGCUAGAGGCGACGUGCAAGAAGUUCGGAGACUGCUGCACAGAGAACUGGUGCACCCUG
ACGCUCUGAACAGAUUCGGCAAGACAGCCCUGCAAGUGAUGAUGUUCGGCAGCCCUGCUGUGGCC
CUGGAACUGCUUAAACAGGGCGCCUCUCCUAACGUGCAGGACGCCUCUGGAACAAGCCCUGUGCA
UGAUGCCGCCAGAACAGGCUUCCUGGACACCCUGAAAGUGCUGGUGGAACACGGCGCCGAUGUGA
ACGCUCUGGAUUCUACCGGCAGCCUGCCUAUCCACCUGGCCAUCAGAGAAGGCCACAGCUCCGUG
GUGUCUUUCCUGGCUCCUGAGAGCGAUCUGCACCACAGAGAUGCCUCUGGCCUGACACCACUGGA
ACUGGCUAGACAGAGAGGCGCUCAGAACCUGAUGGACAUCCUGCAGGGACACAUGAUGAUCCCCA
UGUGAuaaccgcggugucaaaaaccgcguggacgugguuaacaucccugcugggaggaucagccg
uaauuauuauaauuggcuuggugcuggcuacuauuguggccauguacgugcugaccaaccagaaa
cauaauugaauacagcagcaauuggcaagcugcuuacauagaacucgcggcgauuggcaugccgc
cuuaaaauuuuuauuuuauuuuuucuuuucuuuuccgaaucggauuuuguuuuuaauauuucaaa
aaaaaaaaaaaaaaaaaaaaaaaaaaaaaaaaaaaaaaaaaa
SEQ ID NO: 45- 849-VEErep-CDN1A-8526
auaggcggcgcaugagagaagcccagaccaauuaccuacccaaaAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCuaaccugaauggacuac
gacauagucuaguccgccgccaccAUGUCUAACCCUGGGGAUGUUCGGCCCGUGCCUCACAGAUC
CAAAGUGUGCAGAUGCCUGUUUGGCCCCGUGGACUCUGAGCAGCUGAGAAGAGAUUGCGACGCUC
UGAUGGCCGGCUGUCUGCAAGAGGCUAGAGAGAGAUGGAACUUCGACUUCGUGACCGAGACACCC
CUGGAAGGCAACUUCGUGUGGGAAAGAGUCAGAAGCCUGGGCCUGCCUAAAGUGUACCUGUCUCC
UGGCAGCAGAAGCAGGGACGAUCUCGGCGGAGAUAAGAGGCCUUCUACAAGCUCUGCUCUGCUGC
AGGGACCUGCUCCUGAGGAUCAUGUGGCCCUGAGCCUGAGCUGUACCCUGGUGUCUGAAAGACCC
GAGGACUCUCCUGGCGGCCCUGGAACAUCUCAGGGCAGAAAGAGAAGGCAGACCAGCCUGACCGA
CUUCUACCACAGCAAGAGGCGGCUGGUGUUCUGCAAGCGAAAGCCUUGAuaaccgcggugucaaa
aaccgcguggacgugguuaacaucccugcugggaggaucagccguaauuauuauaauuggcuugg
ugcuggcuacuauuguggccauguacgugcugaccaaccagaaacauaauugaauacagcagcaa
uuggcaagcugcuuacauagaacucgcggcgauuggcaugccgccuuaaaauuuuuauuuuauuu
uuucuuuucuuuuccgaaucggauuuuguuuuuaauauuucaaaaaaaaaaaaaaaaaaaaaaaa
aaaaaaaaaaaaaaaaaaaaa
SEQ ID NO: 46- 850-VEErep-CDN1B-8525
auaggcggcgcaugagagaagcccagaccaauuaccuacccaaaAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCuaaccugaauggacuac
gacauagucuaguccgccgccaccAUGAGCAAUGUGCGGGUGUCCAACGGCAGCCCCAGCCUGGA
AAGAAUGGAUGCCAGACAGGCUGAGCACCCCAAGCCUAGCGCUUGCAGAAACCUGUUCGGCCCCG
UGAACCACGAGGAACUGACCAGAGAUCUGGAAAAGCACUGCCGCGACAUGGAAGAGGCCAGCCAG
AGAAAGUGGAACUUCGACUUCCAAAACCACAAGCCUCUGGAAGGCAGAUACGAGUGGCAAGAGGU
GGAAAGAGGCAGCCUGCCUGAGUUCUACUACAGACCUCCUAGGCCUCCUAAGAGCGCCUGCAAGG
UGCUGGCUCAAGAGUCUCAGGAUGUGUCCGGCAGCAGACAGGCCGUGCCUCUGAUUGGAUCUCAG
GCCAACAGCGAGGACAGACACCUGGUGGACCAGAUGCCUGACAGCAGCGAUAACCCUGCUGGACU
GGCUGAGCAGUGCCCCGGAAUGAGAAAAAGACCUGCCGCCGAGGACAGCAGCAGCCAGAACAAGA
GAGCCAACAGAACCGAGGAAAACGUGUCCGACGGCUCUCCUAACGCCGGCACAGUUGAGCAGACC
CCUAAGAAACCAGGCCUGAGAAGGCAGACCUGAuaaccgcggugucaaaaaccgcguggacgugg
uuaacaucccugcugggaggaucagccguaauuauuauaauuggcuuggugcuggcuacuauugu
ggccauguacgugcugaccaaccagaaacauaauugaauacagcagcaauuggcaagcugcuuac
auagaacucgcggcgauuggcaugccgccuuaaaauuuuuauuuuauuuuuucuuuucuuuuccg
aaucggauuuuguuuuuaauauuucaaaaaaaaaaaaaaaaaaaaaaaaaaaaaaaaaaaaaaaa
aaaaa
SEQ ID NO: 47- 851-VEErep-CDN1c-8524
auaggcggcgcaugagagaagcccagaccaauuaccuacccaaaAUGGAGAAAGUUCACGUUGAC
AUCGAGGAAGACAGCCCAUUCCUCAGAGCUUUGCAGCGGAGCUUCCCGCAGUUUGAGGUAGAAGC
CAAGCAGGUCACUGAUAAUGACCAUGCUAAUGCCAGAGCGUUUUCGCAUCUGGCUUCAAAACUGA
UCGAAACGGAGGUGGACCCAUCCGACACGAUCCUUGACAUUGGAAGUGCGCCCGCCCGCAGAAUG
UAUUCUAAGCACAAGUAUCAUUGUAUCUGUCCGAUGAGAUGUGCGGAAGAUCCGGACAGAUUGUA
UAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGAC
GACGAGUCGUGUCGCUACGAAGGGCAAGUCGCUGUUUACCAGGAUGUAUACGCGGUUGACGGACC
GACAAGUCUCUAUCACCAAGCCAAUAAGGGAGUUAGAGUCGCCUACUGGAUAGGCUUUGACACCA
CCCCUUUUAUGUUUAAGAACUUGGCUGGAGCAUAUCCAUCAUACUCUACCAACUGGGCCGACGAA
ACCGUGUUAACGGCUCGUAACAUAGGCCUAUGCAGCUCUGACGUUAUGGAGCGGUCACGUAGAGG
GAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGU
GGCAAGCAAAAUUACACAUGUCGGUGUGAGACUAUAGUUAGUUGCGACGGGUACGUCGUUAAAAG
AAUAGCUAUCAGUCCAGGCCUGUAUGGGAAGCCUUCAGGCUAUGCUGCUACGAUGCACCGCGAGG
GAUUCUUGUGCUGCAAAGUGACAGACACAUUGAACGGGGAGAGGGUCUCUUUUCCCGUGUGCACG
UAUGUGCCAGCUACAUUGUGUGACCAAAUGACUGGCAUACUGGCAACAGAUGUCAGUGCGGACGA
CGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAA
UAUAAGGAAGAUCAAGAAGAUGAAAGGCCACUAGGACUACGAGAUAGACAGUUAGUCAUGGGGUG
UUGUUGGGCUUUUAGAAGGCACAAGAUAACAUCUAUUUAUAAGCGCCCGGAUACCCAAACCAUCA
UCAAAGUGAACAGCGAUUUCCACUCAUUCGUGCUGCCCAGGAUAGGCAGUAACACAUUGGAGAUC
GGGCUGAGAACAAGAAUCAGGAAAAUGUUAGAGGAGCACAAGGAGCCGUCACCUCUCAUUACCGC
CGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGAC
UUGAUGUUACAAGAGGCUGGGGCCGGCUCAGUGGAGACACCUCGUGGCUUGAUAAAGGUUACCAG
CUACGAUGGCGAGGACAAGAUCGGCUCUUACGCUGUGCUUUCUCCGCAGGCUGUACUCAAGAGUG
AAAAAUUAUCUUGCAUCCACCCUCUCGCUGAACAAGUCAUAGUGAUAACACACUCUGGCCGAAAA
GGGCGUUAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACU
GUCAAGCCCAGCGAGCACGACGGCGAAUACCUGUACGACAUCGACAGGAAACAGUGCGUCAAGAA
AGAACUAGUCACUGGGCUAGGGCUCACAGGCGAGCUGGUGGAUCCUCCCUUCCAUGAAUUCGCCU
ACGAGAGUCUGAGAACACGACCAGCCGCUCCUUACCAAGUACCAACCAUAGGGGUGUAUGGCGUG
CCAGGAUCAGGCAAGUCUGGCAUCAUUAAAAGCGCAGUCACCAAAAAAGAUCUAGUGGUGAGCGC
CAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGAC
GAAGCUUUUGCUUGUCAUGCAGGUACUCUCAGAGCGCUCAUAGCCAUUAUAAGACCUAAAAAGGC
AGUGCUCUGCGGGGAUCCCAAACAGUGCGGUUUUUUUAACAUGAUGUGCCUGAAAGUGCAUUUUA
ACCACGAGAUUUGCACACAAGUCUUCCACAAAAGCAUCUCUCGCCGUUGCACUAAAUCUGUGACU
UCGGUCGUCUCAACCUUGUUUUACGACAAAAAAAUGAGAACGACGAAUCCGAAAGAGACUAAGAU
UGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAA
GGGCUGACCCGUAAAGGUGUGUAUGCCGUUCGGUACAAGGUGAAUGAAAAUCCUCUGUACGCACC
CACCUCAGAACAUGUGAACGUCCUACUGACCCGCACGGAGGACCGCAUCGUGUGGAAAACACUAG
CCGGCGACCCAUGGAUAAAAACACUGACUGCCAAGUACCCUGGGAAUUUCACUGCCACGAUAGAG
GAGUGGCAAGCAGAGCAUGAUGCCAUCAUGAGGCACAUCUUGGAGAGACCGGACCCUACCGACGU
CUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCA
GAGAUAGUAUUGAACCAACUAUGCGUGAGGUUCUUUGGACUCGAUCUGGACUCCGGUCUAUUUUC
UGCACCCACUGUUCCGUUAUCCAUUAGGAAUAAUCACUGGGAUAACUCCCCGUCGCCUAACAUGU
ACGGGCUGAAUAAAGAAGUGGUCCGUCAGCUCUCUCGCAGGUACCCACAACUGCCUCGGGCAGUU
GCCACUGGAAGAGUCUAUGACAUGAACACUGGUACACUGCGCAAUUAUGAUCCGCGCAUAAACCU
AGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCC
GUCCCAGGCAAAAUGGUUGACUGGUUGUCAGACCGGCCUGAGGCUACCUUCAGAGCUCGGCUGGA
UUUAGGCAUCCCAGGUGAUGUGCCCAAAUAUGACAUAAUAUUUGUUAAUGUGAGGACCCCAUAUA
AAUACCAUCACUAUCAGCAGUGUGAAGACCAUGCCAUUAAGCUUAGCAUGUUGACCAAGAAAGCU
UGUCUGCAUCUGAAUCCCGGCGGAACCUGUGUCAGCAUAGGUUAUGGUUACGCUGACAGGGCCAG
CGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAU
CCUUACAAGCUUUCAUCAACCUUGACCAACAUUUAUACAGGUUCCAGACUCCACGAAGCCGGAUG
UGCACCCUCAUAUCAUGUGGUGCGAGGGGAUAUUGCCACGGCCACCGAAGGAGUGAUUAUAAAUG
CUGCUAACAGCAAAGGACAACCUGGCGGAGGGGUGUGCGGAGCGCUGUAUAAGAAAUUCCCGGAA
AGCUUCGAUUUACAGCCGAUCGAAGUAGGAAAAGCGCGACUGGUCAAAGGUGCAGCUAAACAUAU
CAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUG
UUGUCCACCGGCAUCUUUUCCGGGAACAAAGAUCGACUAACCCAAUCAUUGAACCAUUUGCUGAC
AGCUUUAGACACCACUGAUGCAGAUGUAGCCAUAUACUGCAGGGACAAGAAAUGGGAAAUGACUC
UCAAGGAAGCAGUGGCUAGGAGAGAAGCAGUGGAGGAGAUAUGCAUAUCCGACGACUCUUCAGUG
ACAGAACCUGAUGCAGAGCUGGUGAGGGUGCAUCCGAAGAGUUCUUUGGCUGGAAGGAAGGGCUA
CAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAU
AUCCUCGGAGAAAGCAUGAGCAGUAUUAGGUCGAAAUGCCCCGUCGAAGAGUCGGAAGCCUCCAC
ACCACCUAGCACGCUGCCUUGCUUGUGCAUCCAUGCCAUGACUCCAGAAAGAGUACAGCGCCUAA
AAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACG
CCUGAGCCGAUCAUCAUCGAAGAGGAAGAAGAGGAUAGCAUAAGUUUGCUGUCAGAUGGCCCGAC
CCACCAGGUGCUGCAAGUCGAGGCAGACAUUCACGGGCCGCCCUCUGUAUCUAGCUCAUCCUGGU
CCAUUCCUCAUGCAUCCGACUUUGAUGUGGACAGUUUAUCCAUACUUGACACCCUGGAGGGAGCU
AGCGUGACCAGCGGGGCAACGUCAGCCGAGACUAACUCUUACUUCGCAAAGAGUAUGGAGUUUCU
GGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGC
GUGAAUAGGGUGAUCACUAGAGAGGAGCUCGAGGCGCUUACCCCGUCACGCACUCCUAGCAGGUC
GGUCUCGAGAACCAGCCUGGUCUCCAACCCGCCAGGCGUAAAUAGGGUGAUUACAAGAGAGGAGU
UUGAGGCGUUCGUAGCACAACAACAAUGACGGUUUGAUGCGGGUGCAUACAUCUUUUCCUCCGAC
ACCGGUCAAGGGCAUUUACAACAAAAAUCAGUAAGGCAAACGGUGCUAUCCGAAGUGGUGUUGGA
GAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAAC
AUGAAAGCCAUAACAGCUAGACGUAUUCUGCAAGGCCUAGGGCAUUAUUUGAAGGCAGAAGGAAA
AGUGGAGUGCUACCGAACCCUGCAUCCUGUUCCUUUGUAUUCAUCUAGUGUGAACCGUGCCUUUU
CAAGCCCCAAGGUCGCAGUGGAAGCCUGUAACGCCAUGUUGAAAGAGAACUUUCCGACUGUGGCU
UCUUACUGUAUUAUUCCAGAGUACGAUGCCUAUUUGGACAUGGUUGACGGAGCUUCAUGCUGCUU
AGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCC
ACAAAAAGAAAUUGCAAUGUCACGCAAAUGAGAGAAUUGCCCGUAUUGGAUUCGGCGGCCUUUAA
UGUGGAAUGCUUCAAGAAAUAUGCGUGUAAUAAUGAAUAUUGGGAAACGUUUAAAGAAAACCCCA
UCAGGCUUACUGAAGAAAACGUGGUAAAUUACAUUACCAAAUUAAAAGGACCAAAAGCUGCUGCU
CUUUUUGCGAAGACACAUAAUUUGAAUAUGUUGCAGGACAUACCAAUGGACAGGUUUGUAAUGGA
CUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGG
AGAUUAAAUGCGGUCCUGCUUCCGAACAUUCAUACACUGUUUGAUAUGUCGGCUGAAGACUUUGA
CGCUAUUAUAGCCGAGCACUUCCAGCCUGGGGAUUGUGUUCUGGAAACUGACAUCGCGUCGUUUG
AUAAAAGUGAGGACGACGCCAUGGCUCUGACCGCGUUAAUGAUUCUGGAAGACUUAGGUGUGGAC
GCAGAGCUGUUGACGCUGAUUGAGGCGGCUUUCGGCGAAAUUUCAUCAAUACAUUUGCCCACUAA
AACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCA
UUCAUUGGAGAUGACAAUAUCGUGAAAGGAGUCAAAUCGGACAAAUUAAUGGCAGACAGGUGCGC
CACCUGGUUGAAUAUGGAAGUCAAGAUUAUAGAUGCUGUGGUGGGCGAGAAAGCGCCUUAUUUCU
GUGGAGGGUUUAUUUUGUGUGACUCCGUGACCGGCACAGCGUGCCGUGUGGCAGACCCCCUAAAA
AGGCUGUUUAAGCUUGGCAAACCUCUGGCAGCAGACGAUGAACAUGAUGAUGACAGGAGAAGGGC
AUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGU
GUUAAAUCAUUCAGCUACCUGAGAGGGGCCCCUAUAACUCUCUACGGCuaaccugaauggacuac
gacauagucuaguccgccgccaccAUGGGCAUGAGCGACGUGUACCUGAGAAGCAGAACCGCCAU
GGAAAGACUGGCCAGCAGCGACACAUUCCCCGUGAUCGCUAGAAGCAGCGCCUGCAGAUCUCUGU
UCGGCCCUGUGGAUCACGAGGAACUGGGCAGAGAACUGAGAAUGAGACUGGCCGAGCUGAACGCC
GAGGACCAGAACAGAUGGGACUUCAACUUCCAGCAGGACGUGCCCCUUAGAGGCCCUGGUAGACU
GCAGUGGAUGGAAGUGGACAGCGAGAGCGUGCCAGCCUUCUACAGAGAAACCGUGCAAGUGGGCA
GAUGCAGACUGCAGCUGGGACCUAGACCUCCUCCUGUGGCUGUGGCCGUGAUUCCUAGAUCUGGA
CCUCCUGCUGGCGAGGCUCCUGAUGGACUUGAGGAAGCUCCUGAGCAGCCUCCUUCUGCUCCUGC
UUCUGCUGUGGUGGCUGAGCCUACACCUCCAGCUACACCAGCUCCAGCCAGCGACCUGACAAGCG
ACCCUAUUCCUGAAGUGACCCUGGUGGCCACUAGCGACCCAACACCUGAUCCUAUUCCAGACGCU
AACCCCGACGUGGCCACAAGAGAUGGCGAAGAACAGGUGCCCGAGCAGGUUUCCGAACAGGGCGA
AGAAUCUGGCGCUGAGCCUGGGGAUGAGCUGGGAACAGAACCUGUGUCUGAGCAAGGCGAGGAAC
AAGGCGCCGAGCCUGUGGAAGAGAAGGACGAGGAACCCGAAGAAGAACAAGGGGCUGAGCCCGUU
GAAGAACAGGGCGCUGAACCUGUCGAGGAACAGAAUGGCGAGCCAGUUGAGGAACAAGACGAGAA
UCAAGAGCAGAGAGGCCAAGAGCUGAAGGACCAGCCUCUGUCUGGAAUCCCUGGCAGACCUGCUC
CUGGAACAGCUGCCGCUAACGCCAACGACUUCUUCGCUAAGAGAAAGAGGACAGCCCAAGAGAAC
AAGGCCAGCAACGAUGUGCCUCCUGGCUGCCCUUCUCCUAAUGUUGCUCCUGGCGUGGGCGCCGU
GGAACAGACACCUAGAAAGAGACUGAGAUGAuaaccgcggugucaaaaaccgcguggacgugguu
aacaucccugcugggaggaucagccguaauuauuauaauuggcuuggugcuggcuacuauugugg
ccauguacgugcugaccaaccagaaacauaauugaauacagcagcaauuggcaagcugcuuacau
agaacucgcggcgauuggcaugccgccuuaaaauuuuuauuuuauuuuuucuuuucuuuuccgaa
ucggauuuuguuuuuaauauuucaaaaaaaaaaaaaaaaaaaaaaaaaaaaaaaaaaaaaaaaaa
aaa

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition, wherein the composition comprises:
(a) a nanoparticle;
(b) a first nucleic acid coding for a protein or a functional fragment thereof; and
(c) at least one of:
a compound, wherein the compound enhances expression of the protein or the functional fragment thereof in mammalian cells, and
a second nucleic acid coding for an expression enhancer or a functional fragment thereof, wherein the expression enhancer or the functional fragment thereof increases expression of the protein or the functional fragment thereof in mammalian cells.

2. The composition of claim 1, wherein the compound or the expression enhancer is a kinase inhibitor.

3. The composition of claim 1, wherein the compound or the expression enhancer is a casein kinase inhibitor, a cyclin-dependent kinase (CDK) inhibitor, an extracellular signal-regulated kinase (ERK) inhibitor, a growth factor inhibitor, a glycogen synthase kinase inhibitor, an immune checkpoint inhibitor, a Janus kinase (JAK) inhibitor, a IκB kinase (IKK) inhibitor, a glycogen synthase kinase-3β (GSK-3β) inhibitor, a lipid kinase inhibitor, a mitogen-activated protein kinase (MAPK) family inhibitor, a phosphatidylinositol 4-kinase (PI4K) inhibitor, a polo-like kinase (PLK) inhibitor, a protein kinase D (PKD) inhibitor, a tyrosine kinase inhibitor, a T-lymphokine-activated killer cell-originated protein kinase (TOPK) inhibitor, a salt inducible kinase (SIK) inhibitor, or a Wnt signaling inhibitor.

4. The composition of claim 3, wherein the compound is:
(a) the CDK inhibitor, wherein the CDK inhibitor is (−)-5-fluoro-4-(4-fluoro-2-methoxyphenyl)-N-[4-[(methylsulfonimidoyl)methyl]pyridin-2-yl]pyridin-2-amine, (+)-5-fluoro-4-(4-fluoro-2-methoxyphenyl)-N-[4-[(methylsulfonimidoyl)methyl]pyridin-2-yl]pyridin-2-amine, (+)-5-fluoro-4-(4-fluoro-2-methoxyphenyl)-N-[4-[(methylsulfonimidoyl)methyl]pyridin-2-yl]pyridin-2-amine, 2-[2-chloro-4-(trifluoromethyl)phenyl]-5,7-dihydroxy-8-[(2S,3R)-2-(hydroxymethyl)-1-methylpyrrolidin-3-yl]chromen-4-one; hydrochloride, 4-[(2,6-dichlorobenzoyl)amino]-N-piperidin-4-yl-TH-pyrazole-5-carboxamide; hydrochloride, 1-[4-(2-aminopyrimidin-4-yl)oxyphenyl]-3-[4-[(4-methylpiperazin-1-yl)methyl]-3-(trifluoromethyl)phenyl]urea, 4-(1-isopropyl-2-methyl-1H-imidazol-5-yl)-N-(4-(methylsulfonyl)phenyl)pyrimidin-2-amine, (1S,3R)-3-acetamido-N-[5-chloro-4-(5,5-dimethyl-4,6-dihydropyrrolo[1,2-b]pyrazol-3-yl)pyridin-2-yl]cyclohexane-1-carboxamide, (3R)-N-[5-chloro-4-(5-fluoro-2-methoxyphenyl)pyridin-2-yl]piperidine-3-carboxamide, 2-[(2S)-1-[6-[(4,5-difluoro-1H-benzimidazol-2-yl)methylamino]-9-propan-2-ylpurin-2-yl]piperidin-2-yl]ethanol, 1-N-[4-[[7-cyclopentyl-6-(dimethylcarbamoyl)pyrrolo[2,3-d]pyrimidin-2-yl]amino]phenyl]-1-N′-(4-fluorophenyl)cyclopropane-1,1-dicarboxamide, 3-[[5-fluoro-4-[4-methyl-2-(methylamino)-1,3-thiazol-5-yl]pyrimidin-2-yl]amino]benzenesulfonamide, 2-[(2S)-1-[3-ethyl-7-[(1-oxidopyridin-1-ium-3-yl)methylamino]pyrazolo[1,5-a]pyrimidin-5-yl]piperidin-2-yl]ethanol, 2-(2-chlorophenyl)-5,7-dihydroxy-8-[(3S,4R)-3-hydroxy-1-methylpiperidin-4-yl]chromen-4-one, 5-amino-N-(2,6-difluorophenyl)-3-(4-sulfamoylanilino)-1,2,4-triazole-1-carbothioamide, (1S,3S)-3-N-(5-pentan-3-ylpyrazolo[1,5-a]pyrimidin-7-yl)cyclopentane-1,3-diamine; dihydrochloride, 2-piperidin-3-yloxy-8-propan-2-yl-N-[(2-pyrazol-1-ylphenyl)methyl]pyrazolo[1,5-a][1,3,5]triazin-4-amine, LSN3106729, 4-N-[4-(2-methyl-3-propan-2-ylindazol-5-yl)pyrimidin-2-yl]-1-N-(oxan-4-yl)cyclohexane-1,4-diamine, [4-amino-2-[[(1S,2S,4R)-2-bicyclo[2.2.1]heptanyl]amino]-1,3-thiazol-5-yl]-(2-nitrophenyl)methanone, 4-[(2,6-dichlorobenzoyl)amino]-N-(1-methylsulfonylpiperidin-4-yl)-1H-pyrazole-5-carboxamide, 6-(difluoromethyl)-8-[(1R,2R)-2-hydroxy-2-methylcyclopentyl]-2-[(1-methylsulfonylpiperidin-4-yl)amino]pyrido[2,3-d]pyrimidin-7-one, 2-pyridin-4-yl-1,5,6,7-tetrahydropyrrolo[3,2-c]pyridin-4-one, N-[6,6-dimethyl-5-(1-methylpiperidine-4-carbonyl)-1,4-dihydropyrrolo[3,4-c]pyrazol-3-yl]-3-methylbutanamide, N-(5-cyclobutyl-TH-pyrazol-3-yl)-2-[4-[5-[2-(2,6-dioxopiperidin-3-yl)-1,3-dioxoisoindol-4-yl]oxypentoxy]phenyl]acetamide, 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrochloride, (2R)-2-[[6-(benzylamino)-9-propan-2-ylpurin-2-yl]amino]butan-1-ol, 2-[(2S)-1-azabicyclo[2.2.2]octan-2-yl]-6-(5-methyl-TH-pyrazol-4-yl)-3H-thieno[3,2-d]pyrimidin-4-one, N-[5-[(5-tert-butyl-1,3-oxazol-2-yl)methylsulfanyl]-1,3-thiazol-2-yl]piperidine-4-carboxamide, (3Z)-3-(1H-imidazol-5-ylmethylidene)-5-methoxy-1H-indol-2-one, N-[3-[[5-chloro-4-(1H-indol-3-yl)pyrimidin-2-yl]amino]phenyl]-3-[[(E)-4-(dimethylamino)but-2-enoyl]amino]benzamide, 2-[2-chloro-4-(trifluoromethyl)phenyl]-5,7-dihydroxy-8-[(2R,3S)-2-(hydroxymethyl)-1-methylpyrrolidin-3-yl]chromen-4-one, free base thereof, salt thereof, or combinations thereof;
(b) the MAP kinase inhibitor, wherein the MAP kinase inhibitor is 5-[4-(2-methoxyethoxy)phenyl]-7-phenyl-3H-pyrrolo[2,3-d]pyrimidin-4-one, 5-(4-cyclopropylimidazol-1-yl)-2-fluoro-4-methyl-N-[6-(4-propan-2-yl-1,2,4-triazol-3-yl)pyridin-2-yl]benzamide, 4-[2-(3H-benzimidazol-5-ylamino)quinazolin-8-yl]oxycyclohexan-1-ol, 1-(5-tert-butyl-2-methylpyrazol-3-yl)-3-(4-pyridin-4-yloxyphenyl)urea, free base thereof, salt thereof, or combinations thereof;
(c) the growth factor inhibitor, wherein the growth factor inhibitor is 2-[4-[(E)-2-[5-[(1R)-1-(3,5-dichloropyridin-4-yl)ethoxy]-1H-indazol-3-yl]ethenyl]pyrazol-1-yl]ethanol, 1-N′-[4-[2-(cyclopropanecarbonylamino)pyridin-4-yl]oxy-2,5-difluorophenyl]-1-N-(4-fluorophenyl)cyclopropane-1,1-dicarboxamide, 6-chloro-N-(5-methyl-1H-pyrazol-3-yl)-2-(4-nitrophenoxy)pyrimidin-4-amine, 1-[4-[(4-ethylpiperazin-1-yl)methyl]-3-(trifluoromethyl)phenyl]-3-[4-[6-(methylamino)pyrimidin-4-yl]oxyphenyl]urea, N-[4-(2-amino-3-chloropyridin-4-yl)oxy-3-fluorophenyl]-5-(4-fluorophenyl)-4-oxo-1H-pyridine-3-carboxamide, 5-amino-N-(2,6-difluorophenyl)-3-(4-sulfamoylanilino)-1,2,4-triazole-1-carbothioamide, [3-[[4-(2-amino-3-chloropyridin-4-yl)oxy-3-fluorophenyl]carbamoyl]-5-(4-fluorophenyl)-4-oxopyridin-1-yl]methyl dihydrogen phosphate; 2-amino-2-(hydroxymethyl)propane-1,3-diol, (3Z)-5-[(1-ethylpiperidin-4-yl)amino]-3-[(3-fluorophenyl)-(5-methyl-TH-imidazol-2-yl)methylidene]-1H-indol-2-one, 2-N-[4-(3-aminopropylamino)phenyl]-4-N-(5-cyclopropyl-TH-pyrazol-3-yl)pyrimidine-2,4-diamine, 4-N-(5-cyclopropyl-1H-pyrazol-3-yl)-6-(4-methylpiperazin-1-yl)-2-N-[(3-propan-2-yl-1,2-oxazol-5-yl)methyl]pyrimidine-2,4-diamine, 1-[4-[methyl-[2-(3-sulfamoylanilino)pyrimidin-4-yl]amino]phenyl]-3-[4-(trifluoromethoxy)phenyl]urea, free base thereof, salt thereof, or combinations thereof;
(d) the JAK inhibitor, wherein the JAK inhibitor is 5-fluoro-2-[[(1S)-1-(4-fluorophenyl)ethyl]amino]-6-[(5-methyl-TH-pyrazol-3-yl)amino]pyridine-3-carbonitrile, 6-N-[(1S)-1-(4-fluorophenyl)ethyl]-4-(1-methylpyrazol-4-yl)-2-N-pyrazin-2-ylpyridine-2,6-diamine, 6-N-[(1S)-1-(4-fluorophenyl)ethyl]-4-(1-methylpyrazol-4-yl)-2-N-pyrazin-2-ylpyridine-2,6-diamine; hydrochloride, N-(cyanomethyl)-4-[2-(4-morpholin-4-ylanilino)pyrimidin-4-yl]benzamide, N-(cyanomethyl)-4-[2-(4-morpholin-4-ylanilino)pyrimidin-4-yl]benzamide; sulfuric acid, 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrochloride, free base thereof, salt thereof, or combinations thereof;
(e) the ERK inhibitor, wherein the ERK inhibitor is 1-[(1S)-1-(4-chloro-3-fluorophenyl)-2-hydroxyethyl]-4-[2-[(2-methylpyrazol-3-yl)amino]pyrimidin-4-yl]pyridin-2-one, 4-[2-(2-chloro-4-fluoroanilino)-5-methylpyrimidin-4-yl]-N-[(1S)-1-(3-chlorophenyl)-2-hydroxyethyl]-1H-pyrrole-2-carboxamide, free base thereof, salt thereof, or combinations thereof;
(f) the PLK inhibitor, wherein the PLK inhibitor is N-[[4-[(6-chloropyridin-3-yl)methoxy]-3-methoxyphenyl]methyl]-2-(3,4-dimethoxyphenyl)ethanamine, N-(4-methoxyphenyl)sulfonyl-N-[2-[(E)-2-(1-oxidopyridin-1-ium-4-yl)ethenyl]phenyl]acetamide, free base thereof, salt thereof, or combinations thereof;
(g) the PI4K inhibitor, wherein the PI4K inhibitor is 2-fluoro-4-[2-methyl-8-[(3-methylsulfonylphenyl)methylamino]imidazo[1,2-a]pyrazin-3-yl]phenol, free base thereof, salt thereof, or combinations thereof;
(h) the tyrosine kinase inhibitor, wherein the tyrosine kinase inhibitor is 3-[[5-fluoro-2-(3-hydroxyanilino)pyrimidin-4-yl]amino]phenol, free base thereof, salt thereof, or combinations thereof;
(i) the TOPK inhibitor, wherein the TOPK inhibitor is 9-[4-[(2R)-1-aminopropan-2-yl]phenyl]-8-hydroxy-6-methyl-5H-thieno[2,3-c]quinolin-4-one, free base thereof, salt thereof, or combinations thereof;
(j) the Wnt signaling inhibitor, wherein the Wnt signaling inhibitor is 6-[2-[[4-(2,4-dichlorophenyl)-5-(5-methyl-1H-imidazol-2-yl)pyrimidin-2-yl]amino]ethylamino]pyridine-3-carbonitrile, 4-[2-(3H-benzimidazol-5-ylamino)quinazolin-8-yl]oxycyclohexan-1-ol, free base thereof, salt thereof, or combinations thereof;
(k) the IKK inhibitor, wherein the IKK inhibitor is 2-amino-6-[2-(cyclopropylmethoxy)-6-hydroxyphenyl]-4-piperidin-4-ylpyridine-3-carbonitrile, 1-[4-[(1R)-1-[2-[[6-[6-(dimethylamino)pyrimidin-4-yl]-1H-benzimidazol-2-yl]amino]pyridin-4-yl]ethyl]piperazin-1-yl]-3,3,3-trifluoropropan-1-one, N′-(1,8-dimethylimidazo[1,2-a]quinoxalin-4-yl)ethane-1,2-diamine, free base thereof, salt thereof, or combinations thereof;
(l) the PKD inhibitor, wherein the PKD inhibitor is 2-[4-[[(2R)-2-aminobutyl]amino]pyrimidin-2-yl]-4-(1-methylpyrazol-4-yl)phenol; dihydrochloride, 9-hydroxy-3,4-dihydro-2H-[1]benzothiolo[2,3-f][1,4]thiazepin-5-one, free base thereof, salt thereof, or combinations thereof;
(m) the SIK inhibitor, wherein the SIK inhibitor is 3-(2,4-dimethoxyphenyl)-4-thiophen-3-yl-TH-pyrrolo[2,3-b]pyridine, free base thereof, salt thereof, or combinations thereof;
(n) the casein kinase inhibitor, wherein the casein kinase inhibitor is 3-[3-[2-(3,4,5-trimethoxyanilino)pyrrolo[2,3-d]pyrimidin-7-yl]phenyl]propanenitrile, (3E)-3-[(2,4,6-trimethoxyphenyl)methylidene]-1H-indol-2-one, N-[(4,5-difluoro-TH-benzimidazol-2-yl)methyl]-9-(3-fluorophenyl)-2-morpholin-4-ylpurin-6-amine, free base thereof, salt thereof, or combinations thereof; or
(o) the GSK-3β inhibitor, wherein the GSK-3β inhibitor is 1-[(4-methoxyphenyl)methyl]-3-(5-nitro-1,3-thiazol-2-yl)urea, 6-[2-[[4-(2,4-dichlorophenyl)-5-(5-methyl-1H-imidazol-2-yl)pyrimidin-2-yl]amino]ethylamino]pyridine-3-carbonitrile, CP21R7, GSK-3 inhibitor 1, Indirubin-3′-monoxime, 5-amino-N-(2,6-difluorophenyl)-3-(4-sulfamoylanilino)-1,2,4-triazole-1-carbothioamide, 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrochloride, free base thereof, salt thereof, or combinations thereof.

5. The composition of claim 3, wherein the expression enhancer is the CDK inhibitor, wherein the CDK inhibitor comprises an amino acid sequence that has at least 80% sequence identity with any one of the sequences of SEQ ID NO: 41 to 47.

6. The composition of claim 1, wherein the first nucleic acid further codes for an RNA polymerase.

7. The composition of claim 6, wherein the RNA polymerase is a Venezuelan equine encephalitis virus (VEEV) RNA polymerase.

8. The composition of claim 7, wherein the VEEV RNA polymerase comprises an amino acid sequence of SEQ ID NO: 39 or SEQ ID NO: 40.

9. The composition of claim 6, wherein the first nucleic acid coding the RNA polymerase comprises a nucleic acid sequence of SEQ ID NO: 38.

10. The composition of claim 1, wherein the protein is an antigen or an antigen-binding protein.

11. The composition of claim 10, wherein the antigen is in a viral antigen or a tumor antigen.

12. The composition of claim 1, wherein the protein is an antibody or a functional fragment thereof.

13. The composition of claim 1, wherein the nanoparticle comprises a hydrophobic core.

14. The composition of claim 13, wherein the hydrophobic core comprises a liquid organic material, a solid inorganic material, or a combination thereof.

15. The composition of claim 13, wherein the hydrophobic core comprises an oil.

16. The composition of claim 15, wherein the oil is α-tocopherol, coconut oil, grapeseed oil, lauroyl polyoxylglyceride, mineral oil, monoacylglycerol, palmkernal oil, olive oil, paraffin oil, peanut oil, propolis, squalene, squalane, solanesol, soy lecithin, soybean oil, sunflower oil, a triglyceride, or vitamin E.

17. The composition of claim 16, wherein the triglyceride is capric triglyceride, caprylic triglyceride, a caprylic and capric triglyceride, a triglyceride ester, or myristic acid triglycerine.

18. The composition of claim 13, wherein the hydrophobic core comprises a phosphate-terminated lipid.

19. The composition of claim 18, wherein the phosphate-terminated lipid is trioctylphosphine oxide (TOPO).

20. The composition of claim 1, wherein the nanoparticle comprises a cationic lipid.

21. The composition of claim 20, wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP), dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O₁₆B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; or TT3, N1,N3,N5-tris(3-(didodecylamino)propyl)benzene-1,3,5-tricarboxamide.

22. The composition of claim 1, wherein the nanoparticle comprises an inorganic particle.

23. The composition of claim 22, wherein the inorganic particle comprises a metal salt, a metal oxide, a metal hydroxide, or a metal phosphate.

24. The composition of claim 23, wherein the metal oxide comprises aluminum oxide, aluminum oxyhydroxide, iron oxide, titanium dioxide, or silicon dioxide.

25. The composition of claim 1, wherein the nanoparticle comprises a hydrophobic surfactant, a hydrophilic surfactant, or both.

26. The composition of claim 1, wherein the nanoparticle comprises a phosphorous-terminated surfactant, a carboxylate-terminated surfactant, a sulfate-terminated surfactant, or an amine-terminated surfactant.
